(19) United States
(12) Patent Application Publication
(10) Pub. No.: US 2023/0270750 A1
(43) Pub. Date: Aug. 31, 2023

(54) CARBALDEHYDE OXIMES AS BUTYRYLCHOLINESTERASE REACTIVATORS

(71) Applicants: ÉTAT FRANÇAIS REPRÉSENTÉ PAR LA DIRECTION CENTRALE DU SERVICE DE SANTÉ DES ARMÉES, Paris (FR); CENTRE NATIONAL DE LA RECHERCHE SCIENTIFIQUE, Paris (FR); UNIVERSITE DE ROUEN-NORMANDIE, Mont-Saint-Aignan (FR); INSTITUT NATIONAL DES SCIENCES APPLIQUÉES DE ROUEN, Saint-Etienne-Du-Rouvray (FR)

(72) Inventors: Nicolas PROBST, Rouen (FR); Anissa BRAÏKI, Sceaux (FR); Pierre DUBOIS-GEOFFROY, Rouen (FR); Ludovic JEAN, Mont-Saint-Aignan Cedex (FR); Pierre-Yves RENARD, Paris (FR); José Dias, Bretigny-sur-Orge (FR); Florian NACHON, Bretigny-sur-Orge (FR)

(21) Appl. No.: 18/006,830

(22) PCT Filed: Jul. 27, 2021
(86) PCT No.: PCT/EP2021/070953

§ 371 Date: Jan. 25, 2023

(30) Foreign Application Priority Data
Jul. 27, 2020 (EP) 20305860.7

(51) Int. Cl. A61K 31/55; A61K 31/4709; A61K 31/4545; A61K 31/496; A61K 31/506; A61K 31/5377; A61K 31/695; A61K 31/437; A61K 31/4375

(52) U.S. Cl. CPC A61K 31/55; A61K 31/4709; A61K 31/4545; A61K 31/496; A61K 31/506; A61K 31/5377; A61K 31/695; A61K 31/437; A61K 31/4375

(57) ABSTRACT

Compounds are used in the reactivation of butyrylcholinesterase. Such compounds are useful in the treatment or prevention of intoxication with at least one organophosphorus nerve agent. Pharmaceutical compositions and kits include the compounds, and compounds per se.

The present invention relates to compounds for their use in the reactivation of butyrylcholinesterase. Such compounds are useful in the treatment or prevention of the intoxication with at least one organophosphorus nerve agent. The invention also relates to pharmaceutical compositions and kits comprising said compounds, and compounds per se.

Organophosphorous nerve agents (OPNA) are extremely toxic compounds that comprise chemical warfare agents (CWA) including sarin, soman, cyclosarin, tabun, VX and pesticides such as paraoxon, parathion and tetraethyl pyrophosphate (TEPP). Their acute toxicity results from the irreversible inhibition of acetylcholinesterase (AChE) through phosphylation of its catalytic serine, which results in the inability of the enzyme to hydrolyze acetylcholine (ACh). Accumulation of this neurotransmitter at cholinergic synapses occurs, leading to a permanent saturation of the muscarinic and nicotinic receptors which ultimately will result in a cholinergic toxidrome including seizures and respiratory distress. Depending on the class of OPNA and on the administrated dose, death can occur within a few minutes to a day.

Due to the similarity between the chemical precursors of CWA and pesticides, and to the relatively simple chemistry involved in their synthesis, efforts to control the proliferation of these agents have proved of limited success. Illustrative examples include the terrorist attack in the Tokyo subway in 1995, the bombing of Kurd civilians during the Iraq-Iran war in 1988, and that of civilians in Syria, as reported in August 2013. Additionally, despite the international efforts aimed at regulating and lessening the use of these environmentally toxic compounds, ca. 100 different OPNA are still used intensively as pest control agents, with only anecdotal monitoring. This results in about 3,000,000 acute intoxications per year, 200,000 of which lead to death. Moreover, intoxications may also occur during the destruction of chemical weapons stockpiles. Therefore, the development of effective measures to counteract OPNA poisoning remains a challenging issue to protect and treat both civilian and military populations.

The current treatment for OPNA poisoning consists in the administration of a combination of atropine (antimuscarinic agent) and diazepam (anticonvulsant drug), to limit convulsions, and of a standard pyridinium oxime (pralidoxime, trimedoxime, HI-6, obidoxime, or HLo-7) to reactivate AChE. Oximes exert their action on OPNA-inhibited AChE by attacking the phosphorous atom of the phosphylated serine, leading to the removal of the phosphylate and restoration of the enzyme catalytic activity.

These oximes work in the peripheral nervous system, but they have a limited bioavailability in the central nervous system due to their poor blood-brain barrier permeability.

Butyrylcholinesterase (BChE) is a circulating plasma enzyme that is also targeted by OPNAs. BChE is an enzyme that is not very selective and is capable of reacting irreversibly with OPNAs very rapidly, neutralizing them regardless of their nature. It has therefore been developed as a bioscavenger of OPNAs to protect against the effects of acute intoxication. However, in spite of its recognized effectiveness, BChE is a stoichiometric bioscavenger (an equivalent of enzyme is needed to neutralize an equivalent of OPNAs) and therefore requires the injection of a large quantity of enzyme to effectively protect a subject. Despite progress made to improve its production by transgenic organisms or to improve its purification from human plasma, the cost of using this enzyme, especially on a large scale, remains high.

Surprisingly, the inventors have now discovered that compounds of the invention are able to reactivate BChE, thus regenerate the phosphylated enzyme, while standard pyridinium oximes are poorly efficient to reactivate said BChE.

Notably, the compounds of the invention may be used as antidotes against OPNA intoxications or as detoxifying agents against organophosphorus compounds, thanks to their effective and fast reactivation of BChE, alone or in combination with the use of BChE.

The present invention thus concerns a compound of following formula (I):

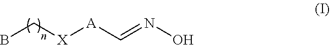

wherein:
n is an integer from 1 to 6;
at least one of the carbon atoms of

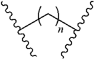

being optionally replaced by an atom chosen from nitrogen, oxygen and sulfur, said nitrogen being optionally substituted by a methyl or ethyl group;
X is a single bond or chosen from —O—, —S—, —NH—, and —NR_{c}—, with R_{c} being methyl or ethyl;
A is chosen from the group comprising arene diyles and 5 to 6 membered heteroarene diyles, said heteroarene being chosen from the group comprising pyridine, thiophene, thiadiazole, oxathiazole, in particular pyridine;
A is optionally substituted by at least one group R chosen from —OH, C₁-C₆ alkyl, —O—C₁-C₆ alkyl, -halogen, notably —Cl, —Br, —F, in particular —OH;
B is chosen:
(i) from heterocyclic groups with 4 to 10 carbon atoms comprising at least one nitrogen atom, and heteroaryl groups chosen from indole, pyrrazole, imidazole, oxazole, thiazole, oxadiazole and thiadiazole,
the heteroaryl cycle B is optionally fused with at least an arene, in particular a benzene, to form a polycycle B′;
the cycle B or B′ is optionally substituted by at least one group Z chosen from C₁-C₆ alkyl, in particular methyl or ethyl; O—C₁-C₆ alkyl, in particular —OMe; aryl, in particular phenyl; heteroaryl, in particular pyridyl, pyrimidinyl; benzyl; benzhydryl; and —NRₐR_{b} groups, wherein Rₐ and R_{b} are independently chosen from H and C₁-C₆ alkyls, Rₐ and R_{b} being in particular H;
said cycle B not being benzhydryl-piperazine, when n is 4, 5 or 6, or X is a single bond, or none of the carbon atoms of

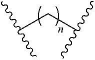

is replaced by an atom chosen from nitrogen, oxygen and sulfur, or said nitrogen does not form a quaternary ammonium, wherein said nitrogen optionally forms a quaternary ammonium by being further substituted by a C₁-C₆ alkyl or by a C₁-C₆ alkane diyl also bound to said cycle B or B′, in particular to said heterocyclic groups with 4 to 10 carbon atoms comprising at least one nitrogen atom;
(ii) from —NR_{Y}R_{Z}, wherein R_{Y} and R_{Z} are independently chosen from H and C₁-C₆ alkyl groups, in particular from C₁-C₆ alkyl groups;
and wherein said nitrogen optionally forms a quaternary ammonium by being further substituted by a C₁-C₆ alkyl;
and
(iii) when n is an integer is 1, 2 or 3, in particular 1 or 2, or X is chosen from —O—, —S—, —NH—, and —NR_{c}—, or at least one of the carbon atoms of

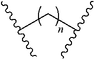

is replaced by an atom chosen from nitrogen, oxygen and sulfur, said nitrogen being optionally substituted by a methyl or ethyl group, or said nitrogen of group B forms a quaternary ammonium, from heterocyclic groups with 4 to 10 carbon atoms comprising at least one nitrogen atom, the cycle B is fused with at least an arene, in particular a benzene, to form a polycycle B′; the cycle B or B′ is optionally substituted by at least one group Z chosen from C₁-C₆ alkyl, in particular methyl or ethyl; O—C₁-C₆ alkyl, in particular —OMe; aryl, in particular phenyl; heteroaryl, in particular pyridyl, pyrimidinyl; benzyl; benzhydryl; and —NRₐR_{b} groups, wherein Rₐ and R_{b} are independently chosen from H and C₁-C₆ alkyls, Rₐ and R_{b} being in particular H;
wherein said nitrogen optionally forms a quaternary ammonium by being further substituted by a C₁-C₆ alkyl or by a C₁-C₆ alkane diyl also bound to said cycle B or B′, in particular to said heterocyclic groups with 4 to 10 carbon atoms comprising at least one nitrogen atom;
(iv) when n is an integer is 1 or 2, in particular 1, or X is chosen from —O—, —S—, —NH—, and —NR_{c}—, or at least one of the carbon atoms of

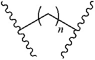

is replaced by an atom chosen from nitrogen, oxygen and sulfur, said nitrogen being optionally substituted by a methyl or ethyl group, or said nitrogen of group B forms a quaternary ammonium, from heterocyclic groups with 4 to 10 carbon atoms comprising at least one nitrogen atom, said cycle B being fused with at least a heteroarene, in particular an indole;
wherein said nitrogen optionally forms a quaternary ammonium by being further substituted by a C₁-C₆ alkyl or by a C₁-C₆ alkane diyl also bound to said cycle B or B′; or a stereoisomeric form, a mixture of stereoisomeric forms or a pharmaceutically acceptable salt or solvate form thereof,
for use as a reactivator of human or animal butyrylcholinesterase, in particular human butyrylcholinesterase for the treatment or prevention of an intoxication with at least one organophosphorus nerve agent, said butyrylcholinesterase being prior to treatment or after prevention inhibited by at least one organophosphorus nerve agent.

The present invention also concerns a compound of following formula (I):

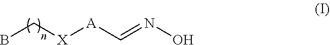

wherein:
n is an integer from 1 to 6;
at least one of the carbon atoms of

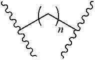

being optionally replaced by an atom chosen from nitrogen and oxygen;
X is a single bond or chosen from —O—, —S— and NH—;
A is chosen from the group comprising arene diyles and 5 to 6 membered heteroarene diyles, said heteroarene being chosen from the group comprising pyridine, thiophene, thiadiazole, oxathiazole, in particular pyridine;
A is optionally substituted by at least one group R chosen from —OH, C₁-C₆ alkyl, —O—C₁-C₆ alkyl, -halogen, notably —Cl, —Br, —F, in particular —OH;
B is chosen from heterocyclic groups with 4 to 10 carbon atoms comprising at least one nitrogen atom, and heteroaryl groups chosen from indole, pyrrazole, imidazole, oxazole, thiazole, oxadiazole and thiadiazole,
the cycle B is optionally fused with at least an arene, in particular a benzene, to form a polycycle B′;
the cycle B or B′ is optionally substituted by at least one group Z chosen from C₁-C₆ alkyl, in particular methyl or ethyl; O—C₁-C₆ alkyl, in particular —OMe; aryl, in particular phenyl; heteroaryl, in particular pyridyl, pyrimidinyl; benzyl; benzhydryl; and —NRₐR_{b} groups, wherein Rₐ and R_{b} are independently chosen from H and C₁-C₆ alkyls, Rₐ and R_{b} being in particular H;
or a stereoisomeric form, a mixture of stereoisomeric forms or a pharmaceutically acceptable salt or solvate form thereof,
for use as a reactivator of human or animal butyrylcholinesterase, in particular human butyrylcholinesterase for the treatment or prevention of an intoxication with at least one organophosphorus nerve agent, said butyrylcholinesterase being prior to treatment or after prevention inhibited by at least one organophosphorus nerve agent.

Said BChE can be from any species. In a particular embodiment, the BChE is mammalian, particularly human.

Human BChE is for instance described in GenBank Gene ID: 590 and GenBank Accession Nos. NM_000055.2 and NP_000046.1 provide examples of amino acid and nucleotide sequences for human BChE.

For the purpose of the invention, the term “pharmaceutically acceptable” is intended to mean what is useful to the preparation of a pharmaceutical composition, and what is generally safe and non-toxic, for a pharmaceutical use.

The term “pharmaceutically acceptable salt or solvate” is intended to mean, in the framework of the present invention, a salt or solvate of a compound which is pharmaceutically acceptable, as defined above, and which possesses the pharmacological activity of the corresponding compound.

The pharmaceutically acceptable salts comprise:
(1) acid addition salts formed with inorganic acids such as hydrochloric, hydrobromic, sulfuric, nitric and phosphoric acid and the like; or formed with organic acids such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphtalenesulfonic, propionic, succinic, dibenzoyl-L-tartaric, tartaric, p-toluenesulfonic, trimethylacetic, and trifluoroacetic acid and the like, and
(2) base addition salts formed when an acid proton present in the compound is either replaced by a metal ion, such as an alkali metal ion, an alkaline-earth metal ion, or an aluminum ion; or coordinated with an organic or inorganic base. Acceptable organic bases comprise diethanolamine, ethanolamine, N-methylglucamine, triethanolamine, tromethamine and the like. Acceptable inorganic bases comprise aluminium hydroxide, calcium hydroxide, potassium hydroxide, sodium carbonate and sodium hydroxide.

Acceptable solvates for the therapeutic use of the compounds of the present invention include conventional solvates such as those formed during the last step of the preparation of the compounds of the invention due to the presence of solvents. As an example, mention may be made of solvates due to the presence of water (these solvates are also called hydrates) or ethanol.

It is recognized that compounds of the present invention may exist in various stereoisomeric forms. As such, the compounds of the present invention include both diastereomers and enantiomers. The compounds are normally prepared as racemates and can conveniently be used as such, but individual enantiomers can be isolated or synthesized by conventional techniques if so desired. Such racemates and individual enantiomers and mixtures thereof form part of the present invention.

It is well known in the art how to prepare and isolate such optically active forms. Specific stereoisomers can be prepared by stereospecific synthesis using enantiomerically pure or enantiomerically enriched starting materials. The specific stereoisomers of either starting materials or products can be resolved and recovered by techniques known in the art, such as resolution of racemic forms, normal, reverse-phase, and chiral chromatography, recrystallization, enzymatic resolution, or fractional recrystallization of addition salts formed by reagents used for that purpose. Useful methods of resolving and recovering specific stereoisomers described in Eliel, E. L.; Wilen, S. H. Stereochemistry of Organic Compounds; Wiley: New York, 1994. and Jacques, J, et al. Enantiomers, Racemates, and Resolutions; Wiley: New York, 1981, each incorporated by reference herein in their entireties.

The term “(C₁-C₆)alkyl”, as used in the present invention, refers to a straight or branched saturated hydrocarbon chain containing from 1 to 6 carbon atoms including, but not limited to, methyl, ethyl, n-propyl, iso-propyl, n-butyl, iso-butyl, sec-butyl, t-butyl, n-pentyl, n-hexyl, and the like.

The term “aryl”, as used in the present invention, refers to an aromatic hydrocarbon group comprising preferably 6 to 10 carbon atoms and comprising one or more, notably 1 or 2, fused rings, such as, for example, a phenyl or naphtyl group. Advantageously, it will be a phenyl group.

The term “heterocycle” as used in the present invention refers in particular to a saturated or unsaturated, more particularly saturated, hydrocarbon monocycle or polycycle (comprising fused, bridged or spiro rings), such as a bicycle, in which one or more, advantageously 1 to 4, and more advantageously 1 or 2, carbon atoms have each been replaced with a heteroatom selected from nitrogen, oxygen, sulphur and silicium atoms, and notably being a nitrogen atom. Advantageously, the heterocycle comprises 5 to 15, notably 5 to 10 atoms in the ring(s). The ring(s) of the heterocycle has/have advantageously 5 or 6 members.

By “heterocyclic groups comprising at least one nitrogen atom”, it is in particular meant an azaheterocyclic group, in which, optionally. one or more, advantageously 1 to 3, and more advantageously 1 or 2, carbon atoms have each been further replaced with a heteroatom selected from oxygen, sulphur and silicium atoms.

According to a particular embodiment, the heterocycle is a saturated or unsaturated, more particularly saturated, hydrocarbon monocycle or bicycle (comprising fused, bridged or spiro rings, notably fused rings), each cycle having 5 or 6 members and 1 to 4, notably 1 or 2, carbon atoms having each been replaced with a nitrogen or oxygen atom, notably a nitrogen atom.

A heterocycle can be notably piperidine, piperazine, triazinane, morpholine, thiomorpholine, pyrrolidine, azepane, azocane, azonane, dihydropyridines, dihydropyrimidines (notably 1,2-dihydropyrimidine), dihydropyridazines, dihydropyrazines, dihydrotriazines, tetrahydropyridines, tetrahydropyrimidines, tetrahydropyridazines, tetrahydropyrazines, tetrahydrotriazines, etc.

The term “heteroaryl” as used in the present invention refers in particular to an aromatic heterocycle as defined above.

According to a particular embodiment, the heteroaryl is an aromatic hydrocarbon monocycle or bicycle (i.e. comprising fused rings), each cycle having 5 or 6 members, notably 6 members, and 1 to 4, notably 1 or 2, carbon atoms having each been replaced with a nitrogen or oxygen atom, notably a nitrogen atom.

A heteroaryl can be notably thiophene, furan, pyrrole, imidazole, pyrazole, oxazole, isoxazole, thiazole, isothiazole, triazoles (1,2,3-triazole and 1,2,4-triazole), benzofuran, indole, benzothiophene, benzimidazole, indazole, benzoxazole, benzisoxazole, benzothiazole, benzisothiazole, pyridine, pyrimidine, pyridazine, pyrazine, triazine, quinoline, isoquinoline, quinoxaline, quinazoline, etc.

The term “halogen”, as used in the present invention, refers in particular to a fluorine, bromine, chlorine or iodine atom.

According to a particular embodiment, n is an integer from 1 to 4, in particular from 1 to 3, more particularly 1 or 2.

According to a particular embodiment, at least one of the carbon atoms of

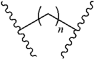

is optionally replaced by an atom chosen from nitrogen, oxygen and sulfur, and/or X is chosen from —O—, —S—, —NH— and —NR_{c}—, with R_{c} being methyl or ethyl.

According to a particular embodiment, X is chosen from —O—, —S—, —NH— and —NR_{c}—, with R_{c} being methyl or ethyl.

According to a particular embodiment, the compound of formula (I) as defined above is such as:
n is an integer from 1 to 4, in particular from 1 to 3, more particularly 1 or 2; or
at least one of the carbon atoms of

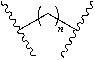

is optionally replaced by an atom chosen from nitrogen, oxygen and sulfur; or
X is chosen from —O—, —S—, —NH— and —NR_{c}—, with R_{c} being methyl or ethyl; or
said nitrogen of group B forms a quaternary ammonium.

According to a particular embodiment, the compound of formula (I) as defined above, wherein B is chosen:
(i) from heterocyclic groups with 4 to 10 carbon atoms comprising at least one nitrogen atom, and heteroaryl groups chosen from indole, pyrrazole, imidazole, oxazole, thiazole, oxadiazole and thiadiazole,
the heteroaryl cycle B is optionally fused with at least an arene, in particular a benzene, to form a polycycle B′;
the cycle B or B′ is optionally substituted by at least one group Z chosen from C₁-C₆ alkyl, in particular methyl or ethyl; O—C₁-C₆ alkyl, in particular —OMe; aryl, in particular phenyl; heteroaryl, in particular pyridyl, pyrimidinyl; benzyl; benzhydryl; and —NRₐRₕ groups, wherein Rₐ and R_{b} are independently chosen from H and C₁-C₆ alkyls, Rₐ and R_{b} being in particular H;
said cycle B being not benzhydryl-piperazine, when n is 4, 5 or 6, or X is a single bond, or one of the carbon atoms of

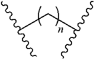

is not replaced by an atom chosen from nitrogen, oxygen and sulfur, or said nitrogen of group B does not form a quaternary ammonium, wherein said nitrogen forms a quaternary ammonium by being further substituted by a C₁-C₆ alkyl or by a C₁-C₆ alkane diyl also bound to said cycle B or B′, in particular to said heterocyclic groups with 4 to 10 carbon atoms comprising at least one nitrogen atom; (ii) from —NR_{Y}R_{Z}, wherein R_{Y} and R_{Z} are independently chosen from H and C₁-C₆ alkyl groups, in particular from C₁-C₆ alkyl groups;
and wherein said nitrogen forms a quaternary ammonium by being further substituted by a C₁-C₆ alkyl;
and
(iii)) when n is an integer is 1, 2 or 3, in particular 1, or X is chosen from —O—, —S—, —NH—, and —NR_{c}—, or at least one of the carbon atoms of

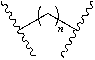

is replaced by an atom chosen from nitrogen, oxygen and sulfur, said nitrogen being optionally substituted by a methyl or ethyl group, or said nitrogen of group B forms a quaternary ammonium, from heterocyclic groups with 4 to 10 carbon atoms comprising at least one nitrogen atom, the cycle B is fused with at least an arene, in particular a benzene, to form a polycycle B′; the cycle B or B′ is optionally substituted by at least one group Z chosen from C₁-C₆ alkyl, in particular methyl or ethyl; O—C₁-C₆ alkyl, in particular —OMe; aryl, in particular phenyl; heteroaryl, in particular pyridyl, pyrimidinyl; benzyl; benzhydryl; and —NRₐR_{b} groups, wherein Rₐ and R_{b} are independently chosen from H and C₁-C₆ alkyls, Rₐ and R_{b} being in particular H;
wherein said nitrogen forms a quaternary ammonium by being further substituted by a C₁-C₆ alkyl or by a C₁-C₆ alkane diyl also bound to said cycle B or B′, in particular to said heterocyclic groups with 4 to 10 carbon atoms comprising at least one nitrogen atom;
(iv) when n is an integer is 1 or 2, in particular 1, or X is chosen from —O—, —S—, —NH—, and —NR_{c}— or at least one of the carbon atoms of

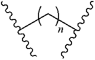

is replaced by an atom chosen from nitrogen, oxygen and sulfur, said nitrogen being optionally substituted by a methyl or ethyl group, or said nitrogen of group B forms a quaternary ammonium, from heterocyclic groups with 4 to 10 carbon atoms comprising at least one nitrogen atom, said cycle B being fused with at least a heteroarene, in particular an indole; wherein said nitrogen forms a quaternary ammonium by being further substituted by a C₁-C₆ alkyl or by a C₁-C₆ alkane diyl also bound to said cycle B or B′.

The present invention also concerns a compound of formula (I) as defined above for use as a reactivator of human or animal butyrylcholinesterase, in particular human butyrylcholinesterase for the treatment or prevention of a nervous and/or respiratory failure due to an intoxication with at least one organophosphorus nerve agent, said butyrylcholinesterase being prior to treatment of after prevention inhibited by at least one organophosphorus nerve agent.

The present invention also concerns a compound of formula (I) as defined above for use in the treatment or prevention of an blood intoxication with at least one organophosphorus nerve agent.

The present invention also concerns a compound of formula (I) as defined above or a pharmaceutical composition as defined below for use as an in vivo reactivator of human or animal butyrylcholinesterase, in particular human butyrylcholinesterase, said butyrylcholinesterase being prior to use inhibited by at least one organophosphorus nerve agent.

The present invention also concerns the use of a compound of formula (I) as defined above as an in vitro or ex vivo reactivator of human or animal butyrylcholinesterase, in particular human butyrylcholinesterase, said butyrylcholinesterase being prior to use inhibited by at least one organophosphorus nerve agent.

The present invention also concerns a method of treatment or prevention of an intoxication with at least one organophosphorus nerve agent comprising the administration of an effective dose of a compound of formula (I) as defined above or a pharmaceutical composition as defined below to a subject in need thereof.

Said administration can be performed prior to the intoxication, in particular prior to exposure to said organophosphorus nerve agent, or after exposure to said organophosphorus nerve agent but prior to intoxication, said method being in this case a method of prevention.

Said administration can be performed simultaneously to or after the intoxication, said method being in this case a method of treatment.

The present invention also concerns a method of treatment or prevention of a nervous and/or respiratory failure due to intoxication with at least one organophosphorus nerve agent comprising the administration of an effective dose of a compound of formula (I) as defined above or a pharmaceutical composition as defined below to a subject in need thereof.

“Effective dose” notably refers to an amount of a BChE, or BChE and AChE reactivation oxime or oxime containing composition for treatment purposes such that BChE, or BChE and AChE enzymes are reactivated in a therapeutically meaningful outcome. Determining an effective amount of such an oxime or combination of oximes for administering to a subject in need thereof can be done based on in vitro and/or animal data using routine computational methods well-known in the medical arts. A skilled person in the medical arts can determine what amount is sufficient for a therapeutically meaningful outcome. In one embodiment, the effective amount contains between about 200 g and 0.1 mg of one or more of the disclosed oximes. In another embodiment, the effective amount contains between about 100 g and 500 mg of one or more of the disclosed oximes. In a further embodiment, the effective amount contains between about 50 g and 1 g of one or more of the disclosed oximes, and preferably about 1-5 g thereof. A person of skill in the art will understand that the effective amount will depend on the mass of the subject and the extent of the exposure to the OP. In a still further embodiment, atropine is co-administered with the one or more of the disclosed oximes.

As used herein, the term “patient” or “subject” refers to a warm blooded animal such as a mammal, preferably a human, or a human child, which is afflicted with, or has the potential to be afflicted with one or more diseases and conditions described herein.

A “subject” can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs (“GP”), and the like).

A “subject in need” refers to a subject that is at risk for exposure to OPs or that in need of a medical assistance to treat, reverse, counteract, and/or prevent poisoning, damage, and/or other harmful effects of exposure to OPs, whether intentional or accidental.

“Treat” and “treatment,” with respect to the exposure of a subject to an organophosphorus compound, refer to a medical intervention which attenuates, prevents, and/or counteracts the effects of such exposure. The foregoing terms can refer to the prophylactic administration of the present compounds and compositions, preferably in the form of a therapeutic composition comprising one or more of the disclosed oximes and one or more pharmaceutical carriers, to a subject at risk of exposure to an organophosphorus compound prior to an anticipated exposure, and/or can refer to the administration of the present compounds and compositions following such exposure.

In a particular embodiment, X represents a single bond.

In a particular embodiment, the residue

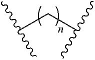

is such as none of its carbon atoms are replaced by an atom chosen from nitrogen and oxygen and sulfur.

In a particular embodiment, at least one of the carbon atoms of

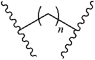

is replaced by an atom chosen from nitrogen, oxygen and sulfur, said nitrogen being optionally substituted by a methyl or ethyl group.

In a particular embodiment, n is 4 or 5.

According to a particular embodiment, n is an integer from 1 to 4, in particular from 1 to 3, more particularly 1 or 2.

According to a particular embodiment, at least one of the carbon atoms of

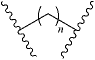

is optionally replaced by an atom chosen from nitrogen, oxygen and sulfur, and/or X is chosen from —O—, —S—, —NH— and —NR_{c}—, with R_{c} being methyl or ethyl.

According to a particular embodiment, X is chosen from —O—, —S—, —NH— and —NR_{c}—, with R_{c} being methyl or ethyl.

According to a particular embodiment, said nitrogen of group B forms a quaternary ammonium.

According to a particular embodiment, the compound of formula (I) as defined above is such as:
n is an integer from 1 to 4, in particular from 1 to 3, more particularly 1 or 2; or
at least one of the carbon atoms of

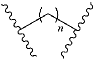

is optionally replaced by an atom chosen from nitrogen, oxygen and sulfur; or
X is chosen from —O—, —S—, —NH— and —NR_{c}—, with R_{c} being methyl or ethyl; or
said nitrogen of group B forms a quaternary ammonium.

According to a particular embodiment, the compound of formula (I) as defined above, wherein B is chosen:
(i) from heterocyclic groups with 4 to 10 carbon atoms comprising at least one nitrogen atom, and heteroaryl groups chosen from indole, pyrrazole, imidazole, oxazole, thiazole, oxadiazole and thiadiazole, the heteroaryl cycle B is optionally fused with at least an arene, in particular a benzene, to form a polycycle B′;
the cycle B or B′ is optionally substituted by at least one group Z chosen from C₁-C₆ alkyl, in particular methyl or ethyl; O—C₁-C₆ alkyl, in particular —OMe; aryl, in particular phenyl; heteroaryl, in particular pyridyl, pyrimidinyl; benzyl; benzhydryl; and —NRₐR_{b} groups, wherein Rₐ and R_{b} are independently chosen from H and C₁-C₆ alkyls, Rₐ and R_{b} being in particular H;
said cycle B being not benzhydryl-piperazine, when n is 4, 5 or 6, or X is a single bond, or one of the carbon atoms of

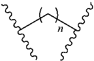

is not replaced by an atom chosen from nitrogen, oxygen and sulfur, or said nitrogen of group B does no form a quaternary ammonium, wherein said nitrogen forms a quaternary ammonium by being further substituted by a C₁-C₆ alkyl or by a C₁-C₆ alkane diyl also bound to said cycle B or B′, in particular to said heterocyclic groups with 4 to 10 carbon atoms comprising at least one nitrogen atom; (ii) from —NR_{Y}R_{Z}, wherein R_{Y} and R_{Z} are independently chosen from H and C₁-C₆ alkyl groups, in particular from C₁-C₆ alkyl groups;
and wherein said nitrogen forms a quaternary ammonium by being further substituted by a C₁-C₆ alkyl;
and
(iii)) when n is an integer is 1, 2 or 3, in particular 1, or X is chosen from —O—, —S—, —NH—, and —NR_{c}—, or at least one of the carbon atoms of

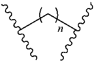

is replaced by an atom chosen from nitrogen, oxygen and sulfur, said nitrogen being optionally substituted by a methyl or ethyl group, or said nitrogen of group B forms a quaternary ammonium, from heterocyclic groups with 4 to 10 carbon atoms comprising at least one nitrogen atom, the cycle B is fused with at least an arene, in particular a benzene, to form a polycycle B′; the cycle B or B′ is optionally substituted by at least one group Z chosen from C₁-C₆ alkyl, in particular methyl or ethyl; O—C₁-C₆ alkyl, in particular —OMe; aryl, in particular phenyl; heteroaryl, in particular pyridyl, pyrimidinyl; benzyl; benzhydryl; and —NRₐR_{b} groups, wherein Rₐ and R_{b} are independently chosen from H and C₁-C₆ alkyls, Rₐ and R_{b} being in particular H;
wherein said nitrogen forms a quaternary ammonium by being further substituted by a C₁-C₆ alkyl or by a C₁-C₆ alkane diyl also bound to said cycle B or B′, in particular to said heterocyclic groups with 4 to 10 carbon atoms comprising at least one nitrogen atom; (iv) when n is an integer is 1 or 2, in particular 1, or X is chosen from —O—, —S—, —NH—, and —NR_{c}—, or at least one of the carbon atoms of

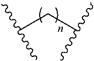

is replaced by an atom chosen from nitrogen, oxygen and sulfur. said nitrogen being optionally substituted by a methyl or ethyl group, or said nitrogen of group B forms a quaternary ammonium, from heterocyclic groups with 4 to 10 carbon atoms comprising at least one nitrogen atom, said cycle B being fused with at least a heteroarene, in particular an indole;
   wherein said nitrogen forms a quaternary ammonium by being further substituted by a C₁-C₆ alkyl or by a C₁-C₆ alkane diyl also bound to said cycle B or B′, in particular to said heterocyclic groups with 4 to 10 carbon atoms comprising at least one nitrogen atom.

In a particular embodiment, cycle B is chosen from pyrrolidine, piperidine, azepane, azocane, azonane, piperazine, thiomorpholine and morpholine, said cycle B being optionally substituted and/or fused as defined above, in particular substituted as defined above.

In a particular embodiment, cycle B is fused with a benzene, to form in particular a tetrahydroisoquinoline, more particularly an unsubstituted tetrahydroisoquinoline.

In a particular embodiment, cycle B is an indole, notably an unsubstituted indole.

In a particular embodiment, cycle B is chosen from pyrrazole, imidazole, oxazole, thiazole, oxadiazole and thiadiazole, said cycle B being optionally substituted and/or fused as defined above, in particular fused as defined above.

In a particular embodiment, B is chosen from indole, piperidine, morpholine, 2,3,4,9-tetrahydro-1H-pyrido[3,4-b]indole, and —NR_{Y}R_{Z}, with in particular n being an integer is 1, 2 or 3, in particular 1, or X being chosen from —O—, —S—, —NH—, and —NR_{c}—, or at least one of the carbon atoms of

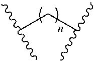

being replaced by an atom chosen from nitrogen, oxygen and sulfur, said nitrogen being optionally substituted by a methyl or ethyl group, or said nitrogen of group B forms a quaternary ammonium.

In a particular embodiment, the compound of formula (I) as defined above is of following formula (Ia):

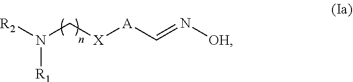

and in particular of following formula (Ib):

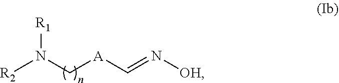

wherein n, X and A are as defined above and R₁ and R₂ form together with the nitrogen to which they are attached a cycle B chosen from the heterocyclic groups with 4 to 10 carbon atoms and indole.

In a particular embodiment, —NR₁R₂ represents a pyrrolidine, piperidine, azepane, azocane, azonane, piperazine, thiomorpholine or morpholine, being optionally substituted and/or fused as defined above, in particular substituted as defined above.

In a particular embodiment, the compound of formula (I) as defined above is of following formula (Ic):

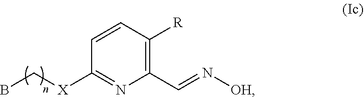

and in particular of following formula (Id):

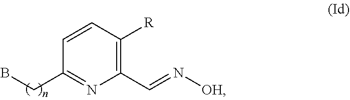

wherein n, B, X and R are as defined above.

In a particular embodiment. the compound of formula (I) as defined above is of following formula (Ie):

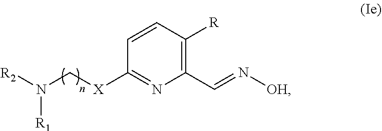

and in particular of following formula (If):

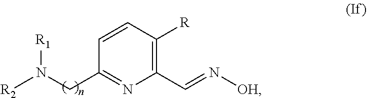

wherein X, R, R₁ and R₂ are as defined above, R being in particular —OH.

In a particular embodiment, more particularly about a compound of formula (Ia), (Ib), (Ie) or (If), —NR₁R₂ represents a residue chosen from:

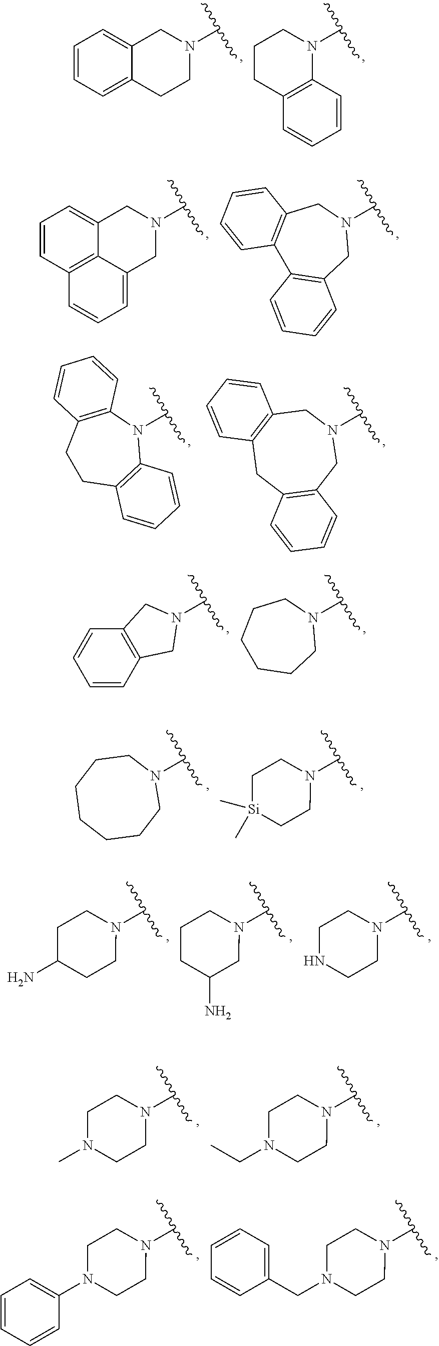

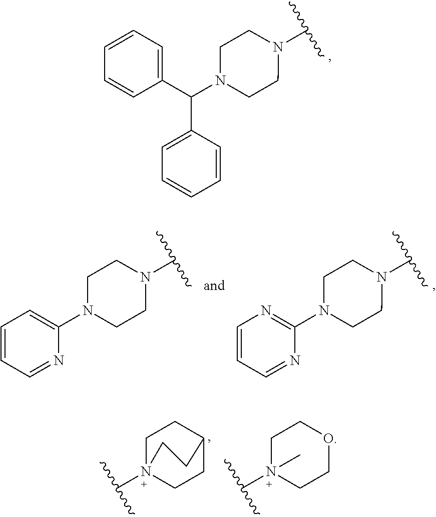

In a particular embodiment, more particularly about a compound of formula (Ta), (Ib), (Ie) or (If), NR₁R₂ represents a residue chosen from:

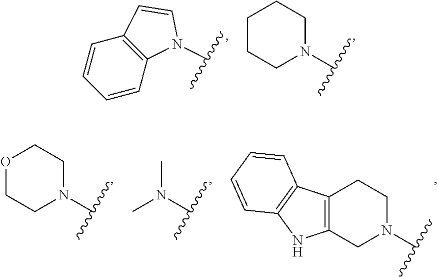

in particular when n is an integer is 1, 2 or 3, in particular 1 or 2, or X is chosen from —O—, —S—, —NH—, and —NR_{c}—, or at least one of the carbon atoms of

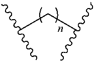

is replaced by an atom chosen from nitrogen, oxygen and sulfur, said nitrogen being optionally substituted by a methyl or ethyl group, or said nitrogen of group B forms a quaternary ammonium.

In a particular embodiment, the compound of formula (I) of the invention is chosen from:

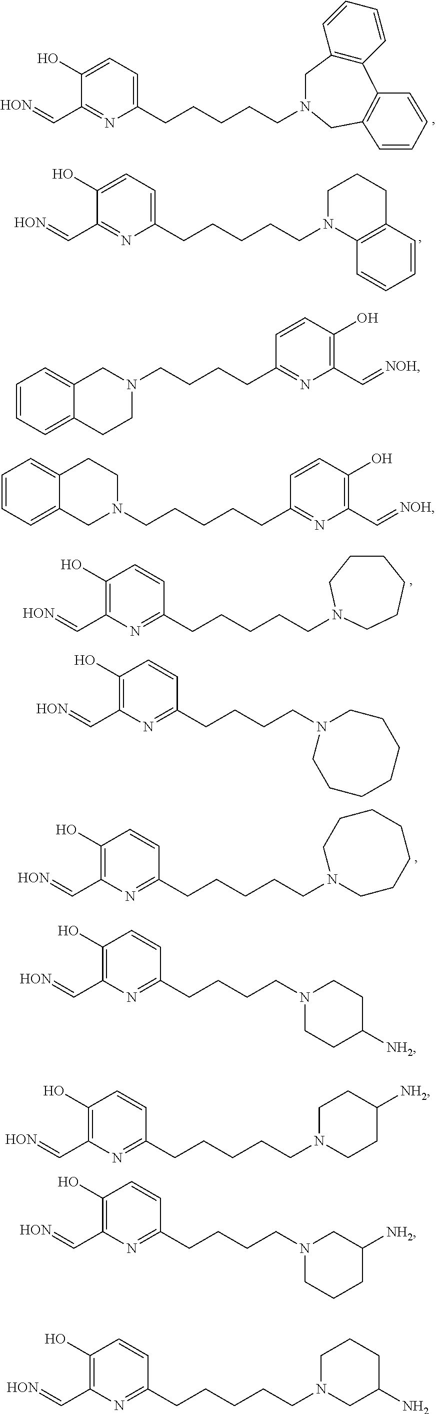

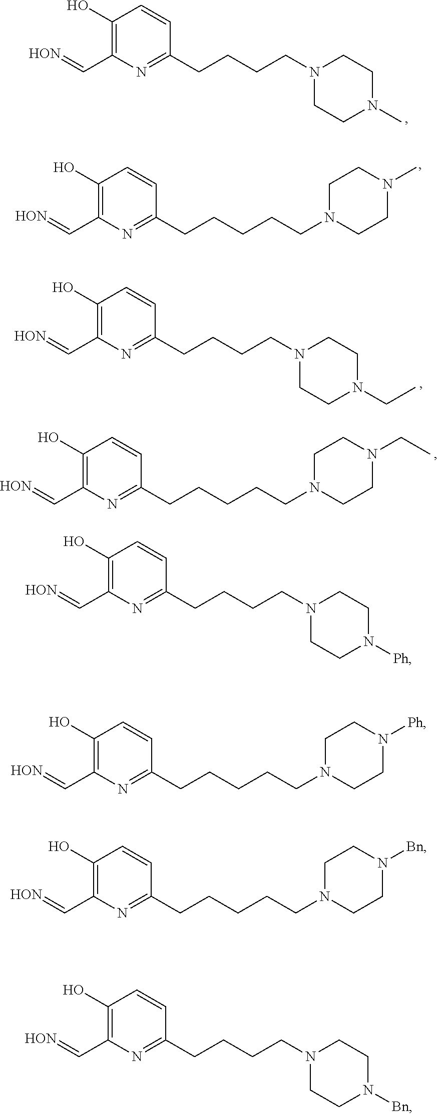

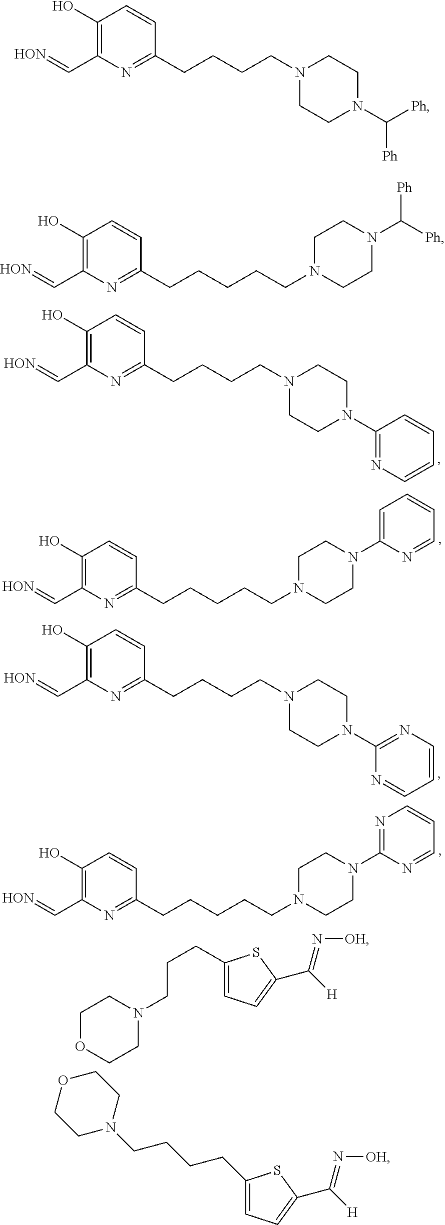

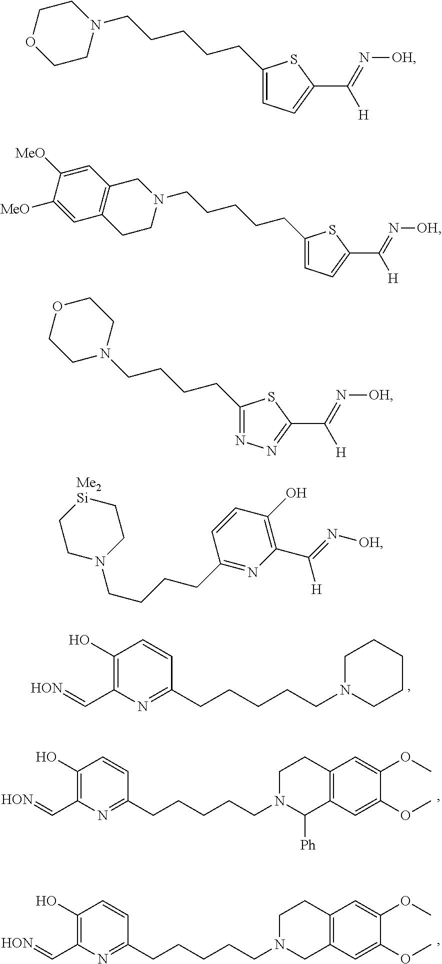

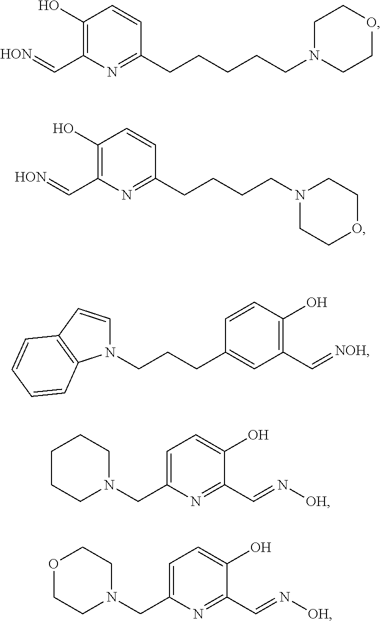

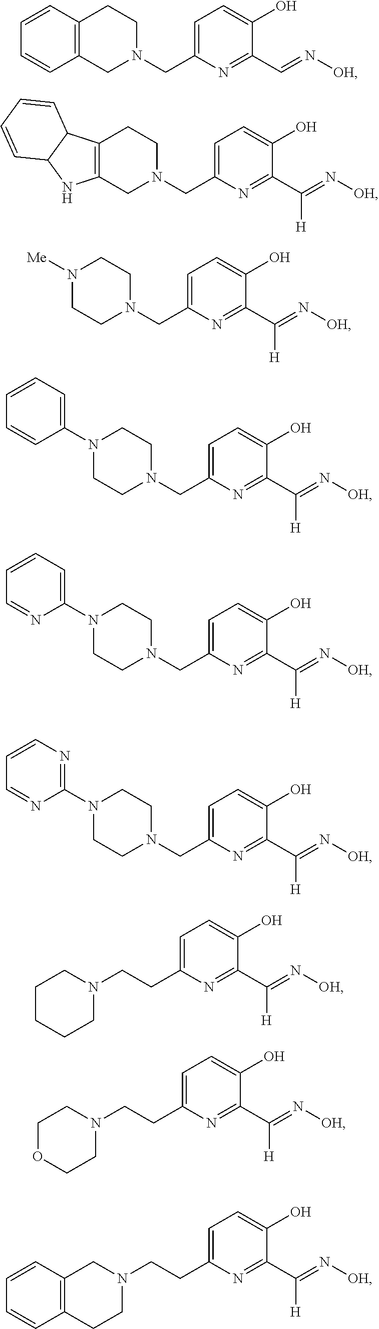

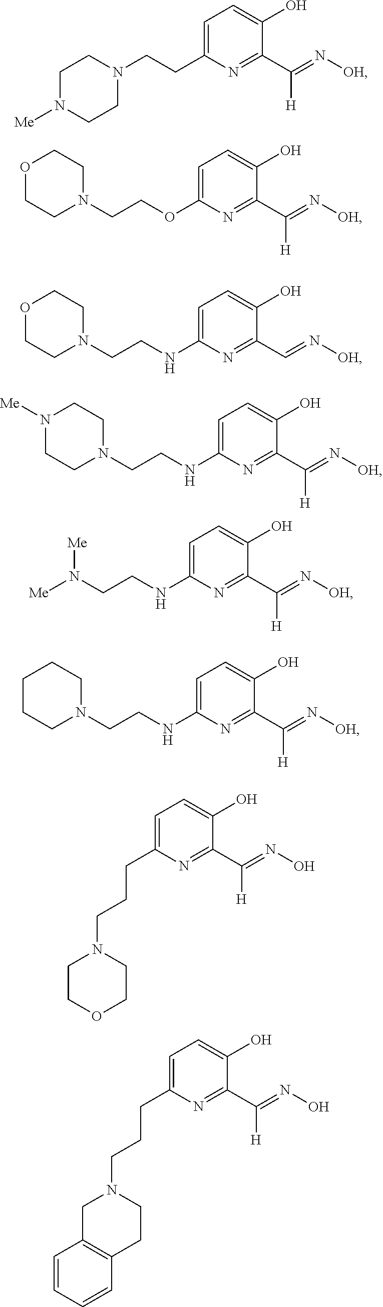

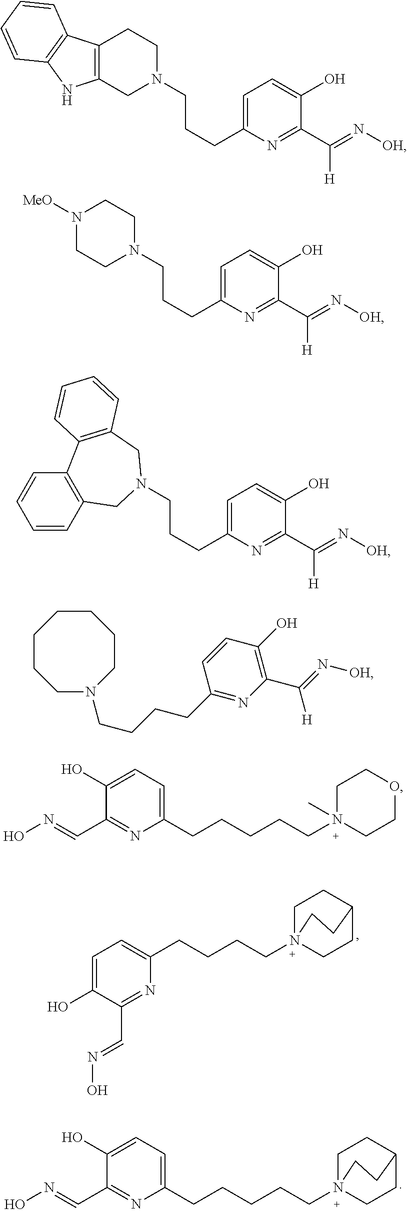

In a particular embodiment, said organophosphorus nerve agent is selected from warfare agents such as Tammelin esters including O-ethyl-S-[2-(diisopropylamino)ethyl]methylphosphonothioate (VX), O-Ethyl-S-2-(diisopropylamino)ethylethylphosphonothiolate (VS), amiton (VG), 2-[ethoxy(ethyl)phosphoryl]sulfanyl-N,N-diethylethanamine (VE), edemo (VM), N,N-diethyl-2-(methyl-(2-methylpropoxy)phosphoryl)sulfanylethanamine (VR) and O-cyclopentyl S-(2-diethylaminoethyl) methylphosphonothiolate (EA-3148); tabun; sarin; cyclosarin; soman; Novichok agents; and pesticides such as paraoxon, parathion, tetraethyl pyrophosphate (TEPP), dichlorvos, phosmet, malathion, fenitrothion, methyl parathion, tetrachlorvinphos, chlorpyrifos, azamethiphos, diazinon, azinphos-methyl, terbufos.

In a more particular embodiment, said organophosphorus nerve agent is selected from warfare agents such as Tammelin esters including O-ethyl-S-[2-(diisopropylamino)ethyl]methylphosphonothioate (VX), 0-Ethyl-S-2-(diisopropylamino)ethylethylphosphonothiolate (VS), amiton (VG), 2-[ethoxy(ethyl)phosphoryl]sulfanyl-N,N-diethylethanamine (VE), edemo (VM), N,N-diethyl-2-(methyl-(2-methylpropoxy)phosphoryl)sulfanylethanamine (VR) and O-cyclopentyl S-(2-diethylaminoethyl) methylphosphonothiolate (EA-3148); tabun; sarin; cyclosarin; soman; Novichok agents.

In a particular embodiment, said organophosphorus nerve agent is selected pesticides such as paraoxon, parathion, tetraethyl pyrophosphate (TEPP), dichlorvos, phosmet, malathion, fenitrothion, methyl parathion, tetrachlorvinphos, chlorpyrifos, azamethiphos, diazinon, azinphos-methyl, terbufos.

In another aspect, the invention concerns a compound of following formula (II):

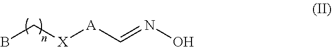

wherein:
n is an integer from 1 to 6;
at least one of the carbon atoms of

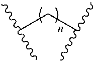

being optionally replaced by an atom chosen from nitrogen, oxygen and sulfur, said nitrogen being optionally substituted by a methyl or ethyl group; X is a single bond or chosen from —O—, —S—, —NH—, and —NR_{c}—, with R_{c} being methyl or ethyl;
A is chosen from the group comprising arene diyles and 5 to 6 membered heteroarene diyles, said heteroarene being chosen from the group comprising pyridine, thiophene, thiadiazole, oxathiazole, in particular pyridine;
A is optionally substituted by a group R chosen from —OH, C₁-C₆ alkyl, —O—C₁-C₆ alkyl, -halogen, notably —Cl, —Br, —F, in particular —OH;
B is chosen from:
   NR₁R₂ groups wherein R₁ and R₂ form together with the nitrogen to which they are attached a cycle B chosen from the heterocyclic groups with 4 to 10 carbon atoms, cycle B not being piperidine or morpholine; and
   heteroaryl groups chosen from pyrrazole, imidazole, oxazole, thiazole, oxadiazole and thiadiazole;
the cycle B is optionally fused with at least an arene, in particular a benzene, to form a polycycle B′;
the cycle B or B′ is optionally substituted by at least one group Z chosen from C₁-C₆ alkyl, in particular methyl or ethyl; O—C₁-C₆ alkyl, in particular —OMe; aryl, in particular phenyl; heteroaryl, in particular pyridyl, pyrimidinyl; benzyl; benzhydryl; and —NRₐR_{b} groups, wherein Rₐ and R_{b} are independently chosen from H and C₁-C₆ alkyls, Rₐ and R_{b} being in particular H;
with the proviso that, in particular when A is a pyridine:
   cycle B is fused and/or substituted when said cycle B is a piperidine;
   cycle B′ is not substituted by one or more O—C₁-C₆ alkyl groups when B′ represents a tetrahydroisoquinoline;
or when n is an integer is 1, 2 or 3, in particular 1, or X is chosen from —O—, —S—, —NH—, and —NR_{c}—, or at least one of the carbon atoms of

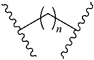

is replaced by an atom chosen from nitrogen, oxygen and sulfur, said nitrogen being optionally substituted by a methyl or ethyl group, or said nitrogen of group B forms a quaternary ammonium, B is chosen:
(i) from heterocyclic groups with 4 to 10 carbon atoms comprising at least one nitrogen atom, and heteroaryl groups chosen from indole, pyrrazole, imidazole, oxazole, thiazole, oxadiazole and thiadiazole,
the cycle B is optionally fused with at least an arene, in particular a benzene, to form a polycycle B′;
the cycle B or B′ is optionally substituted by at least one group Z chosen from C₁-C₆ alkyl, in particular methyl or ethyl; O—C₁-C₆ alkyl, in particular —OMe; aryl, in particular phenyl; heteroaryl, in particular pyridyl, pyrimidinyl; benzyl; benzhydryl; and —NRₐR_{b} groups, wherein Rₐ and R_{b} are independently chosen from H and C₁-C₆ alkyls, Rₐ and R_{b} being in particular H;
wherein said nitrogen optionally forms a quaternary ammonium by being further substituted by a C₁-C₆ alkyl or by a C₁-C₆ alkane diyl also bound to said cycle B or B′, in particular to said heterocyclic groups with 4 to 10 carbon atoms comprising at least one nitrogen atom;
(ii) from —NR_{Y}R_{Z}, wherein R_{Y} and R_{Z} are independently chosen from H and C₁-C₆ alkyl groups, in particular from C₁-C₆ alkyl groups;
and wherein said nitrogen optionally forms a quaternary ammonium by being further substituted by a C₁-C₆ alkyl;
and
(iii) when n is an integer is 1 or 2, in particular 1, or X is chosen from —O—, —S—, —NH—, and —NR_{c}—, or at least one of the carbon atoms of

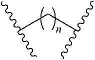

is replaced by an atom chosen from nitrogen, oxygen and sulfur, said nitrogen being optionally substituted by a methyl or ethyl group, or said nitrogen of group B forms a quaternary ammonium, from heterocyclic groups with 4 to 10 carbon atoms comprising at least one nitrogen atom, said cycle B being fused with at least a heteroarene, in particular an indole; wherein said nitrogen optionally forms a quaternary ammonium by being further substituted by a C₁-C₆ alkyl or by a C₁-C₆ alkane diyl also bound to said cycle B or B′, in particular to said heterocyclic groups with 4 to 10 carbon atoms comprising at least one nitrogen atom;
or a stereoisomeric form, a mixture of stereoisomeric forms or a pharmaceutically acceptable salt or solvate form thereof, for use in the treatment or prevention of an intoxication with at least one organophosphorus nerve agent.

In another aspect, the invention concerns a compound of following formula (II):

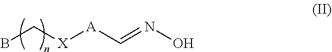

wherein:
n is an integer from 1 to 6;
at least one of the carbon atoms of

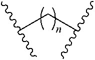

being optionally replaced by an atom chosen from nitrogen and oxygen;
X is a single bond or chosen from —O—, —S— and —NH—;
A is chosen from the group comprising arene diyles and 5 to 6 membered heteroarene diyles, said heteroarene being chosen from the group comprising pyridine, thiophene, thiadiazole, oxathiazole, in particular pyridine;
A is optionally substituted by a group R chosen from —OH, C₁-C₆ alkyl, —O—C₁-C₆ alkyl, -halogen, notably —Cl, —Br, —F, in particular —OH;
B is chosen from:
   NR₁R₂ groups wherein R₁ and R₂ form together with the nitrogen to which they are attached a cycle B chosen from the heterocyclic groups with 4 to 10 carbon atoms; and
   heteroaryl groups chosen from pyrrazole, imidazole, oxazole, thiazole, oxadiazole and thiadiazole;
the cycle B is optionally fused with at least an arene, in particular a benzene, to form a polycycle B′;
the cycle B or B′ is optionally substituted by at least one group Z chosen from C₁-C₆ alkyl, in particular methyl or ethyl; O—C₁-C₆ alkyl, in particular —OMe; aryl, in particular phenyl; heteroaryl, in particular pyridyl, pyrimidinyl; benzyl; benzhydryl; and —NRₐR_{b} groups, wherein Rₐ and R_{b} are independently chosen from H and C₁-C₆ alkyls, Rₐ and R_{b} being in particular H;
with the proviso that, in particular when A is a pyridine:
   cycle B is fused and/or substituted when said cycle B is a piperidine;
   cycle B′ is not substituted by one or more O—C₁-C₆ alkyl groups when B′ represents a tetrahydroisoquinoline;
or a stereoisomeric form, a mixture of stereoisomeric forms or a pharmaceutically acceptable salt or solvate form thereof,
for use in the treatment or prevention of an intoxication with at least one organophosphorus nerve agent.

The present invention also concerns a compound of formula (II) as defined above for use in the treatment or prevention of a nervous and/or respiratory failure due to intoxication with at least one organophosphorus nerve agent.

All embodiments mentioned above, for example related to formula (I), (Ia), (Ib), (Ic), (Id), (Ie) or (If), apply here as well, alone or in combination.

According to a particular embodiment, n is an integer from 1 to 4, in particular from 1 to 3, more particularly 1 or 2.

According to a particular embodiment, at least one of the carbon atoms of

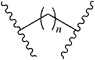

is optionally replaced by an atom chosen from nitrogen, oxygen and sulfur, and/or X is chosen from —O—, —S—, —NH— and —NR_{c}—, with R_{c} being methyl or ethyl.

According to a particular embodiment, X is chosen from —O—, —S—, —NH— and —NR_{c}—, with R_{c} being methyl or ethyl.

According to a particular embodiment, the compound of formula (I) as defined above is such as:
n is an integer from 1 to 4, in particular from 1 to 3, more particularly 1 or 2; or
at least one of the carbon atoms of

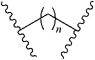

is optionally replaced by an atom chosen from nitrogen, oxygen and sulfur; or
X is chosen from —O—, —S—, —NH— and —NR_{c}—, with R_{c} being methyl or ethyl; or
or said nitrogen of group B forms a quaternary ammonium.

According to a particular embodiment, the compound of formula (I) as defined above, wherein B is chosen:
(i) from heterocyclic groups with 4 to 10 carbon atoms comprising at least one nitrogen atom, and heteroaryl groups chosen from indole, pyrrazole, imidazole, oxazole, thiazole, oxadiazole and thiadiazole,
the cycle B is optionally fused with at least an arene, in particular a benzene, to form a polycycle B′;
the cycle B or B′ is optionally substituted by at least one group Z chosen from C₁-C₆ alkyl, in particular methyl or ethyl; O—C₁-C₆ alkyl, in particular —OMe; aryl, in particular phenyl; heteroaryl, in particular pyridyl, pyrimidinyl; benzyl; benzhydryl; and —NRₐR_{b} groups, wherein Rₐ and R_{b} are independently chosen from H and C₁-C₆ alkyls, Rₐ and R_{b} being in particular H;
wherein said nitrogen forms a quaternary ammonium by being further substituted by a C₁-C₆ alkyl or by a C₁-C₆ alkane diyl also bound to said cycle B or B′, in particular to said heterocyclic groups with 4 to 10 carbon atoms comprising at least one nitrogen atom;
(ii) when n is an integer from 1 to 4, in particular from 1 to 3, more particularly 1 or 2, from —NR_{Y}R_{Z}, wherein R_{Y} and R_{Z} are independently chosen from H and C₁-C₆ alkyl groups, in particular from C₁-C₆ alkyl groups;
and wherein said nitrogen forms a quaternary ammonium by being further substituted by a C₁-C₆ alkyl;
and
(iii) when n is an integer is 1 or 2, in particular 1, from heterocyclic groups with 4 to 10 carbon atoms comprising at least one nitrogen atom, said cycle B being fused with at least a heteroarene, in particular an indole;
wherein said nitrogen orms a quaternary ammonium by being further substituted by a C₁-C₆ alkyl or by a C₁-C₆ alkane diyl also bound to said cycle B or B′, in particular to said heterocyclic groups with 4 to 10 carbon atoms comprising at least one nitrogen atom.

In a particular embodiment, the compound of formula (II) of the invention is chosen from:

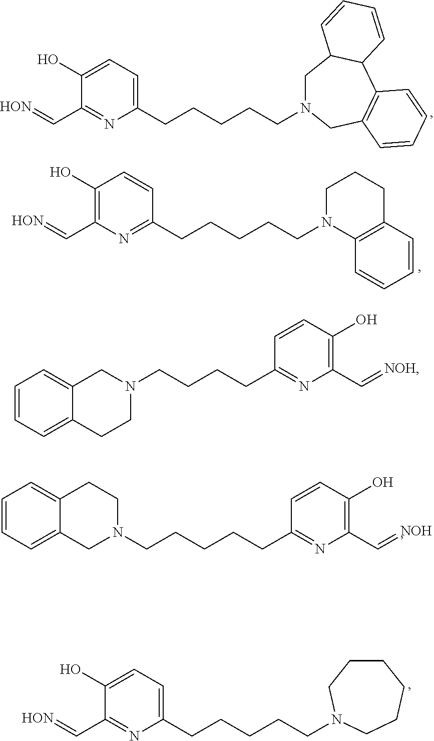

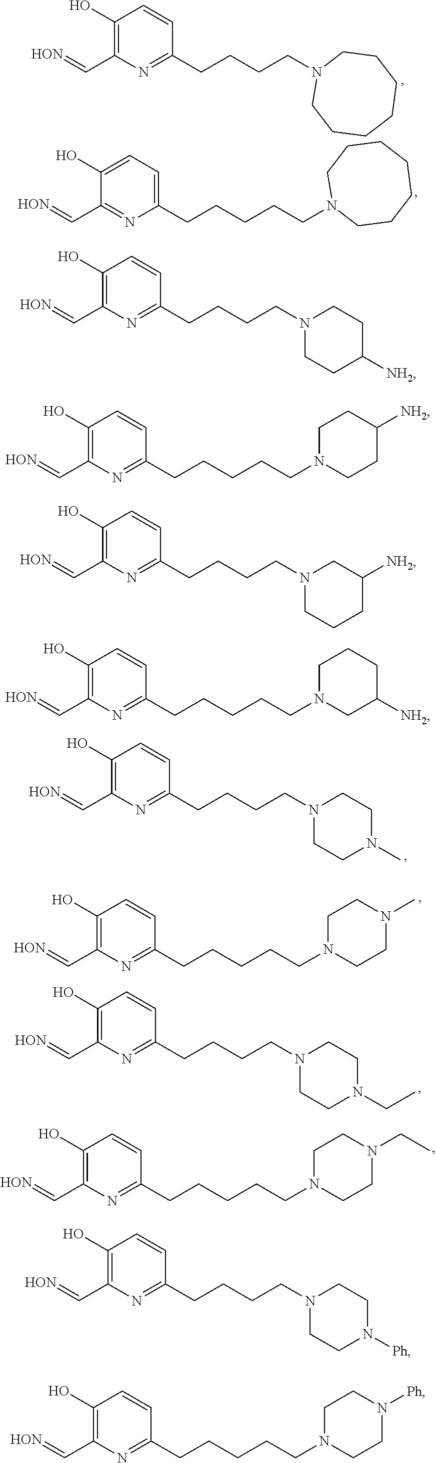

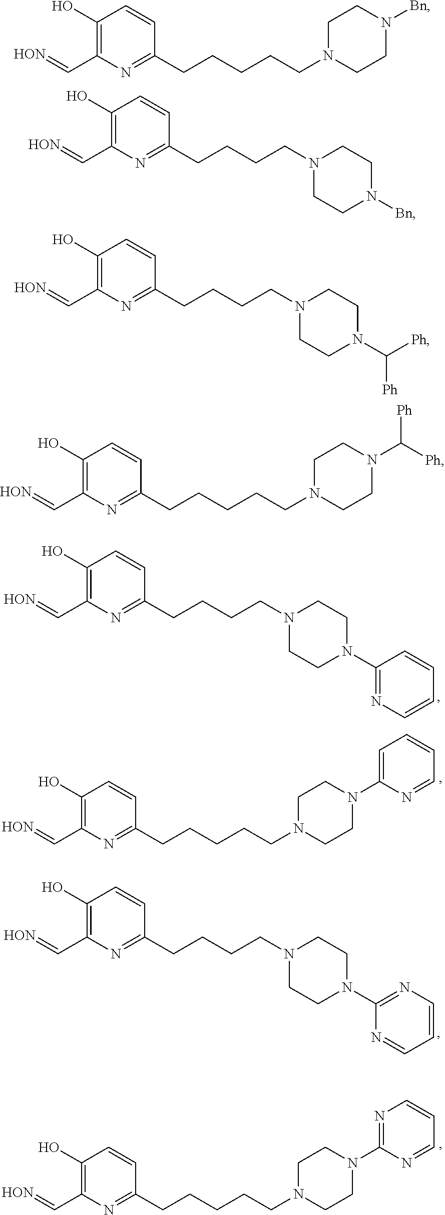

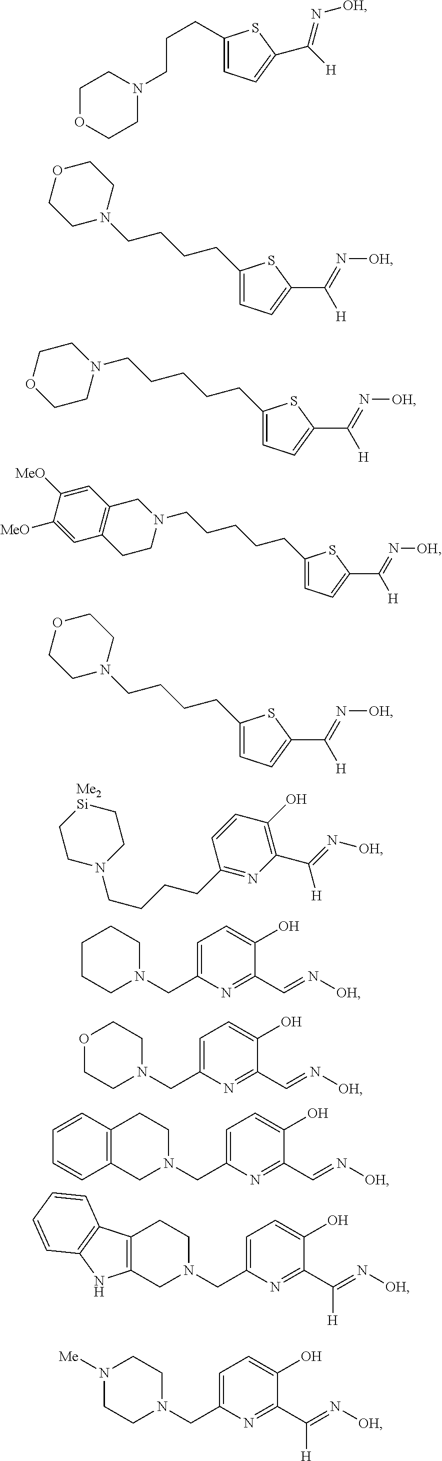

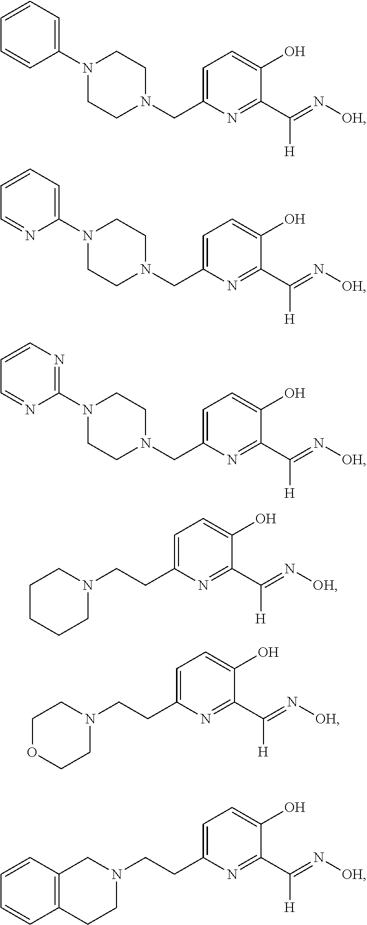

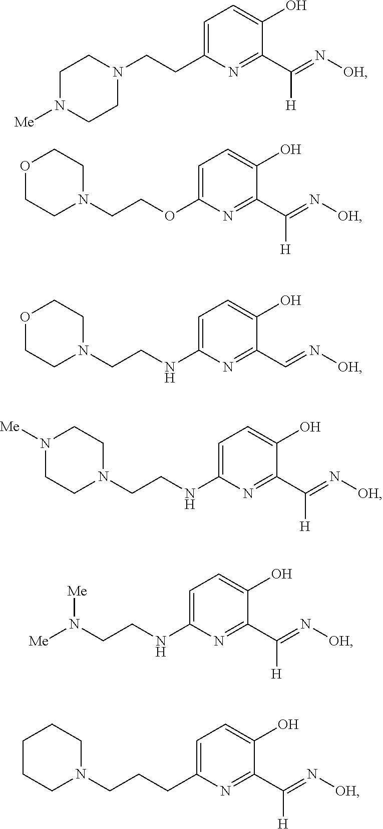

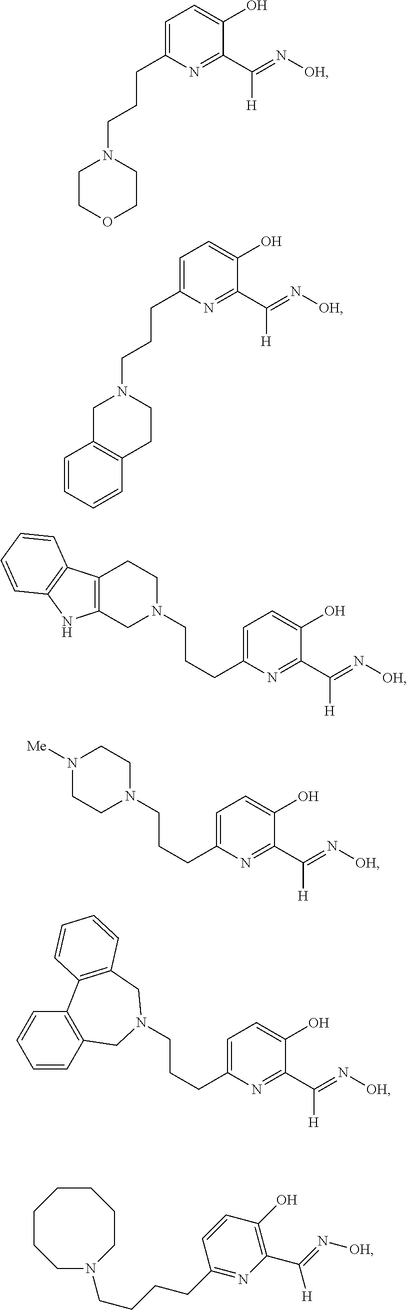 [SMILES: O/N=C/c1nc(CCCN2CCOCC2)ccc1O ; O/N=C/c1nc(CCCN2CCc3ccccc3C2)ccc1O ; O/N=C/c1nc(CCCN2CCc3c([nH]c4ccccc34)C2)ccc1O ; CN1CCN(CCCc2ccc(O)c(/C=N/O)n2)CC1 ; O/N=C/c1nc(CCCN2Cc3ccccc3-c3ccccc3C2)ccc1O ; O/N=C/c1nc(CCCCN2CCCCCCC2)ccc1O]

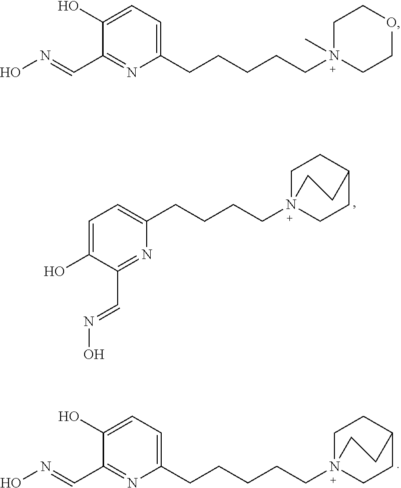

The present invention also concerns a pharmaceutical composition comprising a compound of formula (II) as defined above in admixture with at least one pharmaceutically acceptable excipient.

All embodiments mentioned above, for example related to formula (I), (Ia), (Ib), (Ic), (Id), (Ie) or (If), apply here as well, alone or in combination.

The compound of formula (III) or the pharmaceutical composition of the present invention may be administered in the form of a conventional pharmaceutical composition by any route including orally, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of the present invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavouring agents, solubilizers, lubricants, suspending agents, binders, or tablet disintegrating agents; it can also be an encapsulating material.

Tablets, powders, cachets, and capsules can be used as solid dosage forms suitable for oral administration.

Liquid form compositions include solutions, suspensions, and emulsions. For example, sterile water or propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavouring agents, stabilizers, and thickening agents as desired. Aqueous solutions for oral use can be made by dispersing the finely divided active component in water together with a viscous material such as natural synthetic gums. resins, methyl cellulose, sodium carboxymethyl cellulose, and other suspending agents known to the pharmaceutical formulation art.

Depending on the mode of administration, the pharmaceutical composition will according to one embodiment of the present invention include 0.05% to 99% weight (percent by weight), according to an alternative embodiment from 0.10 to 50% weight, of the compound of the present invention, all percentages by weight being based on total composition. A therapeutically effective amount for the practice of the present invention may be determined, by the use of known criteria including the age, weight and response of the individual patient, and interpreted within the context of the disease which is being treated or which is being prevented, by one of ordinary skills in the art.

The present invention also concerns a compound of formula (II) as defined above.

All embodiments mentioned above, for example related to formula (I), (Ia), (Ib), (Ic), (Id), (Ie) or (If), apply here as well, alone or in combination.

The present invention also concerns a kit comprising a butyrylcholinesterase, in particular a human butyrylcholinesterase, notably selected from wild-type, recombinant, or synthetic butyrylcholinesterase enzymes, and their variants, including peptidomimetics, and a compound of formula (I) or a pharmaceutical composition as defined above.

The present invention also concerns a kit comprising a butyrylcholinesterase, in particular a human butyrylcholinesterase, and a compound of formula (I) or a pharmaceutical composition as defined above, for simultaneous, sequential or separate use in the treatment or prevention of a nervous and/or respiratory failure due to intoxication with at least one organophosphorus nerve agent.

The BChE may be purified and/or concentrated from natural sources (e.g., blood) or may be expressed recombinantly. In a particular embodiment, the BChE is expressed recombinantly. In a particular embodiment, the BChE is isolated. In a particular embodiment, the BChE is purified over a procainamide or huprine affinity column.

For example, the BChE is purified according to the procedure mentioned in WO2015077317 or EP2556057.

By “variant” is in particular meant any protein that has 90, 91, 92, 93, 94, 95, 96, 97, 98, 99% or more of identity with Human BChE, as for instance described in GenBank Gene ID: 590.

Said variants can optionally include nonnatural aminoacids, being in that case peptidomimetics while still defined as variants.

### Definitions

The following terms and expressions contained herein are defined as follows:

As used herein, a range of values in the form “x-y” or “x to y”, or “x through y”, include integers x, y, and the integers therebetween. For example, the phrases “1-6”, or “1 to 6” or “1 through 6” are intended to include the integers 1, 2, 3, 4, 5, and 6. Preferred embodiments include each individual integer in the range, as well as any subcombination of integers. For example, preferred integers for “1-6” can include 1, 2, 3, 4, 5, 6, 1-2, 1-3, 1-4, 1-5, 2-3, 2-4, 2-5, 2-6, etc.

Synthesis

The compounds of the present invention may be prepared in a number of methods well known to those skilled in the art, including, but not limited to those described below, or through modifications of these methods by applying standard techniques known to those skilled in the art of organic synthesis. The appropriate modifications and substitutions will be readily apparent and well known or readily obtainable from the scientific literature to those skilled in the art. In particular, such methods can be found in R. C. Larock, Comprehensive Organic Transformations, Wiley-VCH Publishers, 1999.

All processes disclosed in association with the present invention are contemplated to be practiced on any scale, including milligram, gram, multigram, kilogram, multikilogram or commercial industrial scale.

It will be appreciated that the compounds of the present invention may contain one or more asymmetrically substituted carbon atoms, and may be isolated in optically active or racemic forms. Thus, all chiral, diastereomeric, racemic forms, isomeric forms of a structure are intended, unless the specific stereochemistry or isomeric form is specifically indicated. It is well-known in the art how to prepare and isolate such optically active forms. For example, mixtures of stereoisomers may be separated by standard techniques including, but not limited to, resolution of racemic forms, normal, reverse-phase, and chiral chromatography, preferential salt formation, recrystallization, and the like, or by chiral synthesis either from chiral starting materials or by deliberate synthesis of target chiral centers.

Compounds of the present invention may be prepared by a variety of synthetic routes. The reagents and starting materials are commercially available, or readily synthesized by well-known techniques by one of ordinary skill in the arts. All substituents, unless otherwise indicated, are as previously defined.

In the reactions described hereinafter, it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts in Protective Groups in Organic Chemistry, 3^{rd} ed., John Wiley and Sons, 1999; J. F. W. McOmie in Protective Groups in Organic Chemistry. Plenum Press, 1973.

The reagents and starting materials are commercially available, or readily synthesized by well-known techniques by one of ordinary skill in the arts.

In particular, the compounds defined above are obtained according to the following general procedure:

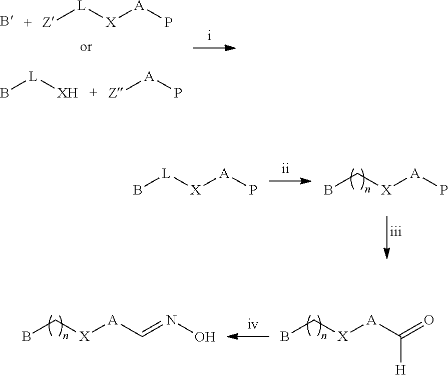

wherein:
B′ is a precursor of the —B group, B′ being for example BH;
Z′ and Z″ are independently chosen from the leaving groups known from the skilled in the art;
L is a precursor of the group that links B to X in the final compound;
P is a protective group of an aldehyde or a precursor of an aldehyde;
Step iv can be performed using AcONa and NH₂OH·HCl.

### EXAMPLES

### Example 1: Synthesis of Compounds of the Invention

General Procedure

Sonogashira Cross-Coupling Reaction—Procedure A

To an argon degassed solution of bromo aryl (1 equiv.) in dry dichloromethane/triethylamine (2/1, v/v, 0.1 M) and alkyne (0.9-1.1 equiv) were poured Pd(PPh₃)₄ (0.05 equiv.) CuI (0.1 equiv.) and the solution was stirred under argon at rt overnight in absence of light. The reaction mixture was concentrated under reduced pressure and purified by flash chromatography (Petroleum ether/EtOAc) to give the desired cross-coupling product.

Mesyl Activation—Procedure B

Primary alcohol (1 equiv.) was dissolved in dry dichloromethane (0.1 M) and the resulting mixture was cooled down to 0° C. Triethylamine (3 equiv.) followed by Mesyl chloride (1.5 equiv.) were added dropwise in this order. The resulting mixture was stirred 1 to 3 hours at rt. The crude mixture is diluted in dichloromethane, washed with water and brine. Organic layer was dried over magnesium sulfate and concentrated under reduced pressure. The mesylate was directly used in following step without further purification.

Tosyl Activation—Procedure C

Primary alcohol (1 equiv.) was dissolved in dry dichloromethane (0.1 M) under argon atmosphere and the resulting mixture was cooled down to 0° C. Triethylamine (3 equiv.) followed by tosyl chloride (1.5 equiv.) were added in this order. The resulting mixture was stirred overnight at rt. The crude mixture was concentrated under reduced pressure and purified using flash chromatography (Petroleum ether/EtOAc).

Nucleophilic Substitution—Procedure D

Activated alcohol (1.0-1.1 equiv) was dissolved in dry MeCN (0.1 M) under argon atmosphere and the resulting mixture was cooled down to 0° C. Amine (1 equiv.) followed by potassium carbonate (1.5 equiv.) were added in this order. The resulting heterogenous mixture was reflux overnight. Salt were removed by filtration and solvent was evaporated under reduced pressure. The crude product was purified using flash chromatography on silica gel.

Hydrogenation—Procedure E

Dry benzyl ether was dissolved in MeOH or EtOAc (0.05 M) and the homogenous solution was degassed 20 min using argon. Pearlman's catalyst or Pd/C (20% w/w) was added. After 10 min of degassing using argon, balloon of hydrogen gas was bubbled and the mixture was stirred under hydrogen atmosphere at rt until completion. The crude mixture was filtered over Celite®, solvents were removed under vacuum. If needed, the crude can be purified by flash chromatography on silica gel (dichloromethane/MeOH).

Aldehyde Generation (Protection—Reduction—Deprotection)—Procedure F

Unprotected 3-hydroxypyridine-2-methyl ester was dissolved in dry dichloromethane (0.1 M), then triethylamine (2.2 equiv.) and TBSOTf (1.1 equiv.) were added dropwise at 0° C. The mixture was stirred at rt until completion (30 min to 3 h). Resulting mixture was diluted in dichloromethane, washed with water, brine and dried under sodium sulfate. Dichloromethane was removed under vacuum giving the crude silyl ether that was dissolved in dry dichloromethane (0.1 M) and cooled down to −78° C. DIBAL-H (n equiv.=2+heteroatom number on ligand*0.5) was added dropwise and the mixture was stirred at −78° C. during 15 min. Methanol was added and the crude mixture was heat up to rt. After dilution with dichloromethane, the organic layer was washed using aqueous NaOH (1 M), water and brine. After drying under sodium sulfate or magnesium sulfate, dichloromethane was removed under vacuum giving the crude silyl ether aldehyde used without further purification in the next step. Crude product was dissolved in dry THF and cooled down to 0° C. TBAF (1.05-1.1 equiv., 1.0 M in THF) was added dropwise and the mixture was stirred at 0° C. during 30 min. THF was removed under vacuum and the crude product was purified by flash chromatography on silica gel.

Oxime Generation—Procedure G

Aldehyde was dissolved in dry MeOH under argon. Hydroxylamine hydrochloride (1.1-3 equiv.) and sodium acetate (1.2-3.1 equiv.) were added and the mixture was stirred at rt until completion (3-4 h). Methanol was removed under vacuum and the crude oxime was purified using either normal or reverse phase chromatography.

Analytic HPLC Methods

Method A: Analytic HPLC (Thermo Hypersil GOLD C18 column, 5 μm, 2.1×100 mm) with MeOH and trifluoroacetic acid (aq. 0.1%; pH 2.0) as eluents [100% aq. TFA (5 min), then linear gradient from 0% to 100% (45 min) MeOH, then 100% MeOH (5 min)] at a flow rate of 0.25 mL/min. UV/Vis detection (220-400 nm) was achieved with “Max Plot” (i.e., chromatogram at absorbance maximum for each compound) mode.

Method B: Analytic HPLC (Thermo Hypersil GOLD C18 column, 5 μm, 2.1×100 mm) with MeCN and trifluoroacetic acid (aq. 0.1%; pH 2.0) as eluents [100% aq. TFA (5 min), then linear gradient from 0% to 100% (45 min) MeCN, then 100% MeCN (5 min)] at a flow rate of 0.25 mL/min. UV/Vis detection (220-700 nm) was achieved with “Max Plot”.

Method C: Analytic HPLC (Thermo Hypersil GOLD C18 column, 5 μm, 2.1×100 mm) with MeCN and water as eluents [100% water (5 min), then linear gradient from 0% to 100% (45 min) MeCN, then 100% MeCN (5 min)] at a flow rate of 0.25 mL/min. UV/Vis detection (220-700 nm) was achieved with “Max Plot” (i.e., chromatogram at absorbance maximum for each compound) mode.

Preparative HPLC and SFC Methods

Method D: Automated flash purification (Biotage, Interchim—Puriflash® 430, PF-C18HP/120G) with aq. 0.1% TFA as aq. mobile phase [0% MeCN (5 min), then linear gradient from 0 to 50% (70 min) MeCN] at a flow rate of 35.0 mL/min. Dual UV-detection was achieved at 254 and 330 nm.

Method E: Semi-preparative HPLC (Thermo scientific Syncronis C18 250*21 mm, 5 μm) with isocratic mobile phase (water/MeCN [80/20]), at a flow rate of 19 ml/min. UV-detection was achieved at 290 nm

Method F: Supercritic flash chromatography (Princeton SFC-60A, 2 Ethylpyridine, 250*10 mm, 5 μm) was performed with CO₂+10% MeOH as mobile phase at 120 bars and at flow rate of 15.0 mL/min. UV detection was achieved at 271 nm.

### Methyl 3-hydroxypicolinate—8

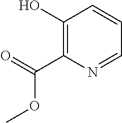

To a suspension of 3-hydroxypicolinic acid (10 g, 72 mmol, 1 equiv.) in methanol (150 mL, 0.5 M) was added dropwise at 0° C. concentrated sulfuric acid (12 mL, 216 mmol, 3 equiv.). The obtained solution was stirred at reflux 6 h. The reaction mixture was concentrated under reduced pressure. The pH was adjusted to 8.5 with an aqueous solution of saturated NaHCO₃ and solid NaHCO₃. The aqueous layer was extracted with EtOAc and the combined organic layers were washed with brine, dried over magnesium sulfate and concentrated under reduced pressure to give 8 as a white solid (10.9 g, 99%). Rf=0.3 (Petroleum ether/EtOAc 1/1, v/v). m.p.=74° C. ¹H NMR (300 MHz, CDCl₃): δ (ppm) 4.06 (s, 3H, Me), 7.34 (dd, J=3.9, 8.4 Hz, 1H), 7.38 (dd, J=1.5, 8.4 Hz, 1H), 8.38 (dd, J=1.5, 3.9 Hz, 1H), 10.64 (s, 1H). ¹³C NMR (75 MHz, CDCl₃) δ (ppm) 53.2, 126.3, 129.8, 130.2, 141.6, 158.9, 169.9. MS (ESI+): m/z (%): 154 (100) [M+H]⁺.

### Methyl 6-bromo-3-hydroxypicolinate—9a

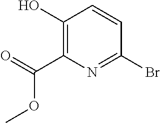

To a solution of methyl 3-hydroxypicolinate 8 (10 g, 65.36 mmol) in osmosed water (0.1 M) with crushed ice at 0° C., was added portionwise bromine (4×1.02 mL every 30 min, 78.4 mmol, 1.2 equiv.) under vigorous stirring. The mixture was vigorously stirred at 0° C. for 1-2 h. The solution was extracted by dichloromethane and the combined organic layers were washed with brine, dried over magnesium sulfate and concentrated under reduced pressure to give 9a as a white-off solid (15.08 g, 99%). Rf=0.3 (Petroleum ether/EtOAc 6/4, v/v). ¹H NMR (300 MHz, CDCl₃) δ (ppm) 4.07 (s, 3H), 7.29 (d, J=8.7 Hz, 1H), 7.58 (dd, J=0.3, 8.7 Hz, 1H), 10.72 (br s, 1H). ¹³C NMR (75 MHz, CDCl₃): δ (ppm) 53.5, 129.5, 130.0, 130.7, 134.5, 158.5, 169.1. MS (ESI+): m/z (%): 234 (85) and 232 (100) [M+H]⁺.

### Methyl 3-(benzyloxy)-6-bromopicolinate—4a

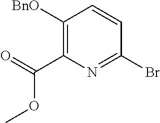 [SMILES: COC(=O)c1nc(Br)ccc1OCc1ccccc1]

To a solution of methyl 3-hydroxy-6-bromopicolinate 9a (11.6 g, 49.8 mmol), in acetone (200 mL, 0.25 M) was added successively potassium carbonate (21 g, 149 mmol, 3 equiv.) and benzyl bromide (12 mL, 100 mmol, 2.0 equiv.). The heterogeneous reaction mixture was reflux overnight. Salts were removed by filtration and the crude product was concentrated under reduced pressure. Purification by flash chromatography on silica gel (Petroleum ether/EtOAc 95/5 to 60/40, v/v) afforded the desired product 4a as a white solid (15.2 g, 95%). Rf=0.4 (Petroleum ether/EtOAc 8/2, v/v). ¹H NMR (300 MHz, CDCl₃): δ (ppm) 3.97 (s, 3H), 5.21 (s, 2H), 7.24 (d, J=8.7 Hz, 1H), 7.47-7.34 (m, 5H), 7.51 (d, J=8.7 Hz, 1H). ¹³C NMR (75 MHz, CDCl₃): δ (ppm) 52.9, 71.2, 125.0, 126.9, 128.4, 128.8, 131.2, 131.4, 135.2, 139.8, 154.0, 164.0. MS (ESI+): m/z (%): 324 (85) and 322 (100) [M+H]⁺.

### 3-(benzyloxy)-6-bromopicolinaldehyde—84

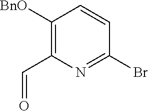 [SMILES: O=Cc1nc(Br)ccc1OCc1ccccc1]

Compound 4a (1.43 g, 4.46 mmol, 1 equiv.) was dissolved in dry dichloromethane (30 mL), the solution was cooled down to −78° C., DIBAL (8.92 mL, 8.92 mmol, 2 equiv.) was slowly added. This mixture was stirred at −78° C. for 15 min. MeOH (9 mL) was slowly added, then the mixture was allowed to warm to rt. The reaction mixture was diluted with dichloromethane, washed with aqueous NaOH (1 M). water and brine. Organic layer was dried over magnesium sulfate and evaporated under reduced pressure to give 84 as a white powder (1.3 g, quant.). ¹H NMR (300 MHz, CDCl₃) δ (ppm): 10.18 (s, 1H), 7.49 (d, J=8.8 Hz, 1H), 7.39-7.22 (m, 6H), 5.19 (s, 2H). ¹³C NMR (75 MHz, CDCl₃) δ (ppm): 189.2, 157.0, 143.6, 139.8, 134.7, 129.0, 128.8, 127.3, 126.1, 124.6, 71.1. MS (ESI+): m/z (%): 293 [M+H].

### 3-(benzyloxy)-6-bromo-2-(1,3-dioxolan-2-yl)pyridine—85

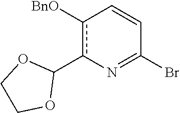

A bed of molecular sieves previously activated was put in a round bottom flask under argon. dichloromethane (80 mL) was added and 84 (1.3 g, 4.45 mmol, 1.0 equiv.) was dissolved. After cooling at 0° C., ethylene glycol (1.23 mL, 22.25 mmol, 5 equiv.), then dropwise BF₃·Et₂O (1.1 mL, 8.9 mmol. 2 equiv.) were added in this order. The mixture was allowed to warm at rt overnight. Molecular sieves were filtered off and the filtrate was washed with water. Organic layer was dried over magnesium sulfate and evaporated to give 85 as a white powder (1.42 g, 95%). Rf=0.5 (Petroleum ether/EtOAc, 8/2, v/v). ¹H NMR (300 MHz, CDCl₃): δ (ppm) 7.39-7.21 (m, 7H), 7.05 (d, J=8.7 Hz, 1H), 6.21 (s, 1H), 5.04 (s, 2H), 4.24-4.06 (m, 2H), 4.06-3.91 (m, 2H). ¹³C NMR (75 MHz, CDCl₃): δ (ppm) 65.9, 71.0, 99.9, 123.3, 127.5, 128.5, 128.8, 128.9, 131.4, 135.6, 147.5, 152.9. MS (ESI+): m/z (%): 336 (85) and 338 (100) [M+H]⁺.

### But-3-yn-1-yl 4-methylbenzenesulfonate—62b

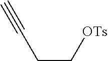

But-3-yn-1-yl 4-methylbenzenesulfonate was synthetized following Procedure B using but-3-yn-1-ol and 62b was isolated after purification by flash chromatography on silica gel (Petroleum ether/EtOAc, 90/10, v/v) as a colorless liquid (quant.). Rf=0.4 (Petroleum ether/EtOAc 9/1, v/v). ¹H NMR (300 MHz, CDCl₃) δ (ppm) 7.80 (d, J=8.3 Hz, 2H), 7.35 (d, J=8.0 Hz, 2H), 4.10 (t, J=7.1 Hz, 2H), 2.55 (td, J=7.1, 2.7 Hz, 2H), 2.45 (s, 3H), 1.97 (t, J=2.7 Hz, 1H). ¹³C NMR (75 MHz, CDCl₃) δ (ppm) 144.9, 133.0, 129.9, 128.1, 82.2, 69.6, 68.8, 27.8, 21.8, 14.8.

### 1-(but-3-yn-1-yl)-1,2,3,4-tetrahydroquinoline—95

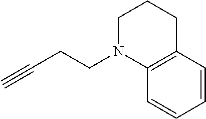

Tosylate 62b (1.12 g, 5 mmol, 1.0 equiv) was dissolved in dry MeCN (80 mL, 0.05 M) under argon atmosphere and the resulting mixture was cooled down to 0° C. 1,2,3,4-tetrahydroquinoline (660 μL, 5.05 mmol, 1.01 equiv.) followed by potassium carbonate (800 mg, 5.7 mmol, 1.2 equiv.) were added in this order. The resulting heterogenous mixture was reflux overnight. Salt were removed by filtration and solvent was evaporated under reduced pressure. The crude product was purified by flash chromatography on silica gel (Petroleum ether/EtOAc, 100/0 to 96/4, v/v) to give the 95 as a yellow oil (86 mg, 9%). ¹H NMR (300 MHz, CDCl₃): δ (ppm) 7.13 (t, J=7.1 Hz, 1H), 7.02 (d, J=6.7 Hz, 1H), 6.65 (dd, J=6.7, 7.1 Hz, 2H), 3.56 (d, J=6.2 Hz, 2H), 3.41 (m, 2H), 2.82 (m, 2H), 2.53 (m, 2H), 2.15-1.90 (m, 3H). ¹³C NMR (75 MHz, CDCl₃): δ (ppm) 144.45, 129.48, 127.30, 122.59, 116.14, 110.49, 82.52, 69.63, 50.55, 49.75, 28.20, 22.27, 16.06.

### Methyl 3-(benzyloxy)-6-(4-(3,4-dihydroquinolin-1(2H)-yl)but-1-yn-1-yl)picolinate—99

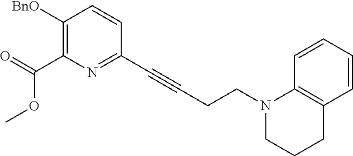 [SMILES: COC(=O)c1nc(C#CCCN2CCCc3ccccc32)ccc1OCc1ccccc1]

To an degassed solution of bromo pyridine 4a (149 mg, 0.46 mmol, 1 equiv.) in dry dichloromethane/triethylamine (2/1, v/v, 0.1 M) and 95 (86 mg, 0.46 mmol, 1 equiv.) were poured Pd(PPh₃)₄ (27 mg, 0.023 mmol, 0.05 equiv.) and CuI (9.5 mg, 0.046 mmol, 0.1 equiv.). The solution was stirred under argon at rt overnight in absence of light. The reaction mixture was concentrated under reduced pressure and purified by flash chromatography (Petroleum ether/EtOAc, 100/0 to 70/30, v/v) to give 99 as an orange oil (35 mg, 14%). Rf=0.30 (Petroleum ether/EtOAc, 80/20, v/v). ¹H NMR (300 MHz, CDCl₃): δ (ppm) 7.36 (m, 7H), 7.05 (m, 1H), 6.93 (m, 1H), 6.60 (m, 2H), 5.21 (s, 2H), 3.96 (s, 3H), 3.57 (m, 2H), 3.34 (m, 2H), 2.74 (m, 2H), 2.66 (m, 2H), 1.95 (m, 2H). MS (ESI+): m/z (%): 427 [M+H]⁺.

### Methyl 3-(benzyloxy)-6-(4-hydroxybut-1-yn-1-yl)picolinate—14b

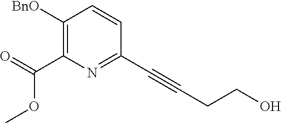 [SMILES: COC(=O)c1nc(C#CCCO)ccc1OCc1ccccc1]

To a solution of methyl 3-(benzyloxy)-6-bromopicolinate 4a (2.0 g, 6.20 mmol, 1 equiv) in dry dichloromethane (53 mL) and triethylamine (27 mL) was added 4-pentyn-1-ol (485 μL, 6.21 mmol, 1 equiv). The resulting mixture was degassed for 20 min with argon. CuI (200 mg, 0.1 equiv.) and Pd(PPh₃)₄ (330 mg, 0.05 equiv.) were then poured and the solution was stirred under argon at rt overnight. The reaction mixture was concentrated under reduced pressure. Purification by flash chromatography (Petroleum ether/EtOAc 7/3 to 3/7, v/v) afforded the desired product 14b as a yellow oil that crystallized upon standing (1.8 g, 94%). Rf=0.3 (Petroleum ether/EtOAc 4/6, v/v). ¹H NMR (300 MHz, CDCl₃) δ (ppm) 2.66 (t, J=6.6 Hz, 2H), 3.00 (br s, 1H), 3.82 (t, J=6.6 Hz, 2H), 3.96 (s, 3H), 4.29-4.35 (m, 1H), 5.18 (s, 2H), 7.28-7.44 (m, 7H). ¹³C NMR (75 MHz, CDCl₃) δ (ppm) 23.8, 52.7, 60.7, 70.9, 80.6, 87.4, 121.9, 126.9, 128.3, 128.8, 130.1, 135.0, 135.5, 139.9, 153.2, 164.8. MS (ESI+): m/z (%): 312 (100) [M+H]⁺.

### methyl 3-(benzyloxy)-6-(4-(tosyloxy)but-1-yn-1-yl)picolinate—59b

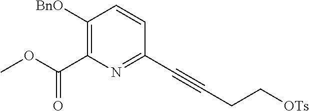 [SMILES: COC(=O)c1nc(C#CCCOS(=O)(=O)c2ccc(C)cc2)ccc1OCc1ccccc1]

Methyl 3-(benzyloxy)-6-(4-(tosyloxy)but-1-yn-1-yl)picolinate 59b was synthetized following Procedure B using 14b and was isolated after purification by flash chromatography on silica gel (Petroleum ether/EtOAc, 60/40, v/v) as a yellow oil (yield: quant.). Rf=0.42 (Petroleum ether/EtOAc, 60/40, v/v). ¹H NMR (300 MHz, CDCl₃) δ (ppm) 7.84 (d, J=8.3 Hz, 2H), 7.56-7.19 (m, 9H), 5.22 (s, 2H), 4.21 (t, J=6.9 Hz, 2H), 3.98 (s, 3H), 2.80 (t, J=6.9 Hz, 2H), 2.44 (s, 3H). ¹³C NMR (75 MHz, CDCl₃) δ (ppm) 164.5, 152.9, 152.9, 144.9, 142.8, 141.1, 139.9, 135.2, 134.4, 134.1, 132.4, 129.9, 129.7, 129.5, 128.4, 127.9, 127.6, 126.7, 125.0, 121.4, 83.6, 80.9, 70.4, 67.1, 52.4, 21.3, 20.0.

### Methyl 3-(benzyloxy)-6-(5-(3,4-dihydroquinolin-1(2H)-yl)pent-1-yn-1-yl)picolinate—100

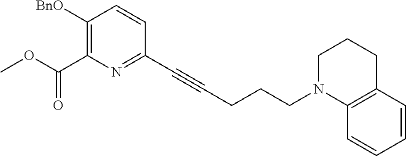 [SMILES: COC(=O)c1nc(C#CCCCN2CCCc3ccccc32)ccc1OCc1ccccc1]

Tosylate 59b (993 mg, 2.07 mmol. 1.0 equiv) was dissolved in dry MeCN (20 mL, 0.1 M) under Argon atmosphere and the resulting mixture was cooled down to 0° C. 1,2,3,4-tetrahydroquinoline (300 μL, 2.5 mmol, 1.2 equiv.) followed by potassium carbonate (575 mg, 5.7 mmol, 2 equiv.) were added in this order. The resulting heterogenous mixture was reflux overnight. Salt were removed by filtration and solvent was evaporated under reduced pressure. The crude product was purified by flash chromatography on silica gel (Petroleum ether/EtOAc, 100/0 to 50/50, v/v) to give the 95 as an orange oil (212 mg, 24%). Rf=0.5 (Petroleum ether/EtOAc, 50/50, v/v). ¹H NMR (300 MHz, CDCl₃): δ (ppm) 7.49-7.28 (m, 7H), 7.03 (td, J=8.3, 1.7 Hz, 1H), 6.94 (dd, J=7.3, 1.4 Hz, 1H), 6.62 (d, J=8.2 Hz, 1H), 6.56 (td, J=7.3, 1.0 Hz, 1H), 5.20 (s, 2H), 3.97 (s, 3H), 3.46-3.35 (m, 2H), 3.33-3.24 (m, 2H), 2.75 (t, J=6.4 Hz, 2H), 2.48 (t, J=7.0 Hz, 2H), 2.01-1.80 (m, 4H). ¹³C NMR (75 MHz, CDCl₃): δ (ppm) 164.90, 152.99, 145.26, 140.21, 135.59, 135.44, 129.99, 129.25, 128.80, 128.29, 127.14, 126.97, 122.41, 121.84, 115.63, 110.63, 89.73, 79.86, 70.89, 52.73, 50.48, 49.79, 28.20, 25.18, 22.31, 17.13. MS (ESI+): m/z (%): 441 [M+H]⁺.

### Methyl 6-(5-(3,4-dihydroquinolin-1(2H)-yl)pentyl)-3-hydroxypicolinate—101

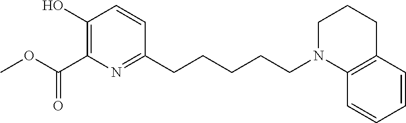 [SMILES: COC(=O)c1nc(CCCCCN2CCCc3ccccc32)ccc1O]

Benzyl ether 100 (210 mg, 0.5 mmol, 1.0 equiv.) was dissolved in MeOH (0.05 M) and the homogenous solution was degassed 20 min using argon. Pearlman's catalyst (42 mg, 20% w/w) was added. After 10 min of degassing using Argon, balloon of hydrogen gas was bubbled and the mixture was stirred under hydrogen atmosphere at rt until completion. The crude mixture was filtered over Celite®, solvents were removed under vacuum to give 101 as a red oil (171 mg, 96%). ¹H NMR (300 MHz, CDCl₃): δ (ppm) 7.34-7.25 (m, 2H), 7.04 (td. J=8.2, 1.7 Hz, 1H), 6.99-6.87 (m, 1H), 6.58-6.51 (m, 2H), 4.05 (s, 3H), 3.25 (dd, J=13.1, 6.9 Hz, 4H), 2.86-2.71 (m, 4H), 1.99-1.88 (m, 2H), 1.70 (ddt, J=23.0, 11.7, 7.6 Hz, 6H), 1.47-1.35 (m, 2H). ¹³C NMR (75 MHz, CDCl₃): δ (ppm) 170.18, 157.20, 153.95, 145.28, 129.17, 129.14, 128.84, 127.04, 126.64, 122.19, 115.28, 110.44, 53.14, 51.34, 49.47, 37.62, 29.99, 28.21, 26.92, 26.09, 22.26. MS (ESI+): m/z (%): 355 [M+H]⁺.

### 6-(5-(3,4-dihydroquinolin-1(2H)-yl)pentyl)-3-hydroxypicolinaldehyde—102

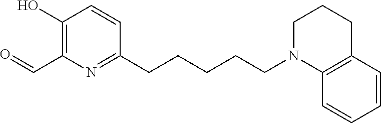 [SMILES: O=Cc1nc(CCCCCN2CCCc3ccccc32)ccc1O]

Compound 101 (169 mg, 0.48 mmol, 1.0 equiv.) was dissolved in dry dichloromethane (10 mL, 0.05 M), then triethylamine (192 μL, 1.2 mmol, 2.5 equiv.) and TBSOTf (143 μL, 0.53 mmol, 1.1 equiv.) were added dropwise at 0° C. The mixture was stirred at rt 1 h. Resulting mixture was diluted in dichloromethane, washed with water, brine and dried under sodium sulfate. Dichloromethane was removed under vacuum giving the crude silyl ether that was dissolved in dry dichloromethane (10 mL, 0.05 M) and cooled down to −78° C. DIBAL-H (1.2 mL, 1.2 mmol, 2.5 equiv.) was added dropwise and the mixture was stirred at −78° C. during 15 min. Methanol (1.2 mL) was added and the crude mixture was heat up to rt. After dilution with dichloromethane, the organic layer was washed using aqueous NaOH (1M), water and brine. After drying under sodium sulfate, dichloromethane was removed under vacuum giving the crude silyl ether aldehyde moiety that was dissolved in dry THF (20 mL) and cooled down to 0° C. TBAF (350 μL, 1 equiv.) was added dropwise and the mixture was stirred at 0° C. during 30 min. THF was removed under vacuum and the crude was purified by flash chromatography on silica gel (Petroleum ether/EtOAc, 80/20, v/v) to give the title product as a red oil (81 mg, 52% over 3 steps). Rf=0.62 (Petroleum ether/EtOAc, 80/20, v/v). ¹H NMR (300 MHz. CDCl₃): δ (ppm) 10.08 (s, 1H), 7.32 (d, J=1.9 Hz, 2H), 7.07 (t, J=7.7 Hz, 1H), 6.97 (d, J=7.7 Hz, 1H), 6.68-6.53 (m, 2H), 3.29 (dd, J=12.5, 5.5 Hz, 4H), 2.82 (dt, J=12.2, 6.8 Hz, 4H), 1.97 (dt, J=12.6, 6.2 Hz, 2H), 1.83 (dt, J=15.6, 7.9 Hz, 2H), 1.76-1.60 (m, 2H), 1.56-1.38 (m, 2H). MS (ESI+) m/z=325 [M+H]⁺.

### AB-392—93

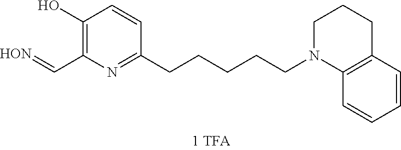

Aldehyde 102 (81 mg, 0.25 mmol, 1.0 equiv.) was dissolved in dry MeOH under argon. Hydroxylamine hydrochloride (19 mg, 0.26 mmol, 1.1 equiv.) and sodium acetate (25 mg, 0.3 mmol, 1.2 equiv.) were added and the mixture was stirred at rt during 4 h. Methanol was removed under vacuum and the crude oxime was purified using reverse phase chromatography (method D) to give the TFA salt of the title compound as a white-off solid (81.6 mg, 72%). ¹H NMR (300 MHz, CDCl₃): δ (ppm) 10.57 (br s, 3H), 8.37 (s, 1H), 8.15 (d, J=3.7 Hz, 1H), 7.47 (dd, J=11.3, 4.2 Hz, 1H), 7.14 (d, J=8.4 Hz, 1H), 6.96 (d, J=8.4 Hz, 1H), 6.67-6.55 (m, 2H), 3.65-3.50 (m, 4H), 2.77-2.61 (m, 6H), 2.51-2.39 (m, 2H), 1.76-1.53 (m, 4H), 1.42-1.28 (m, 2H). ¹³C NMR (75 MHz, CDCl₃): δ (ppm) 159.25, 153.13, 152.56, 147.82, 137.79, 135.30, 124.52, 123.68, 113.74, 107.45, 58.40, 52.63, 44.62, 37.02, 29.89, 27.04, 25.8, 22.78. HRMS (ESI+): m/z calcd for C20H26N3O2 340.2025; found: 340.2027. HPLC (method B): t_{R}=15.70 min, purity=98.34%. MS (ESI+): m/z (%): 340 [M+H]⁺.

### 2-(but-3-yn-1-yl)-1,2,3,4-tetrahydroisoquinoline—104

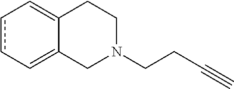

But-3-yn-1-yl methanesulfonate (740 mg, 5 mmol, 2.0 equiv.) was dissolved in dry MeCN (50 mL, 0.1 M) under argon atmosphere and the resulting mixture was cooled down to 0° C. 1,2,3,4-tetrahydroisoquinoline (330 μL, 2.5 mmol, 1 equiv.) followed by potassium carbonate (766 mg, 5.5 mmol, 2.2 equiv.) were added in this order. The resulting heterogenous mixture was reflux overnight. Salt were removed by filtration and solvent was evaporated under reduced pressure. The crude product was purified by flash chromatography (Petroleum ether/EtOAc, 80/20, v/v) to give the title compound as a yellow oil (408.4 mg, 88%). Rf=0.36 (Petroleum ether/EtOAc, 80/20, v/v). ¹H NMR (300 MHz, CDCl₃): δ (ppm) 7.24-7.06 (m, 3H), 7.06-6.97 (m, 1H), 3.69 (s, 2H), 2.91 (t, J=5.8 Hz, 2H), 2.85-2.70 (m, 4H), 2.56-2.43 (m, 2H), 2.00 (t, J=2.7 Hz, 1H). ¹³C NMR: (75 MHz, CDCl₃) δ (ppm) 134.60, 134.24, 128.80, 126.71, 126.30, 125.76, 82.85, 69.24, 56.85, 55.85, 50.82, 29.10, 17.34. MS (ESI+): m/z (%): 186 [M+H]⁺.

### Methyl 3-(benzyloxy)-6-(4-(3,4-dihydroisoquinolin-2(1H)-yl)but-1-yn-1-yl)picolinate—105

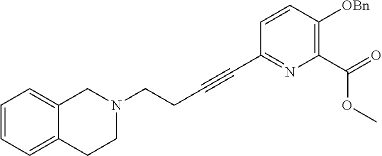 [SMILES: COC(=O)c1nc(C#CCCN2CCc3ccccc3C2)ccc1OCc1ccccc1]

To an argon degassed solution of 4a (509 mg, 1.58 mmol, 1 equiv.) in dry dichloromethane/triethylamine (2/1, v/v, 0.1M) and alkyne 104 (293 mg, 1.58 mmol, 1 equiv.) were poured Pd(PPh₃)₄ (91 mg, 0.079 mmol, 0.05 equiv.) CuI (30 mg, 0.158 mmol, 0.1 equiv.) and the solution was stirred under argon at rt overnight in absence of light. The reaction mixture was concentrated under reduced pressure and purified by flash chromatography (Petroleum ether/EtOAc, 40/60, v/v) to give the desired cross-coupling product 105 as an orange oil (300 mg, 45%, trace of PPh₃ remaining). Rf=0.25 (Petroleum ether/EtOAc, 1/1, v/v). MS (ESI+): m/z=427 [M+H]⁺. ¹H NMR (300 MHz, CDCl₃): δ (ppm) 7.51-7.31 (m, 7H), 7.18-6.97 (m, 4H), 5.20 (s, 2H), 3.96 (s, 3H), 3.70 (s, 2H), 2.95-2.76 (m, 5H), 2.76-2.63 (m, 2H), 1.26 (t, J=7.1 Hz, 1H). ¹³C NMR (75 MHz, CDCl₃): δ (ppm) 164.90, 153.06, 140.23, 135.62, 135.43, 134.57, 134.19, 130.05, 128.83, 128.76, 128.33, 127.00, 126.68, 126.26, 125.73, 121.86, 88.50, 80.16, 70.94, 56.63, 55.78, 52.76, 50.80, 29.14, 18.18.

### Methyl 6-(4-(3,4-dihydroisoquinolin-2(1H)-yl)butyl)-3-hydroxypicolinate—106

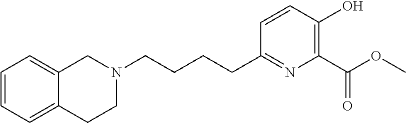 [SMILES: COC(=O)c1nc(CCCCN2CCc3ccccc3C2)ccc1O]

Compound 105 (279 mg, 0.63 mmol, 1.0 equiv.) was dissolved in MeOH (13 mL, 0.05 M) and the homogenous solution was degassed 20 min using argon. Pearlman's catalyst (56 mg, 20% w/w) was added. After 10 min of degassing using Argon, balloon of hydrogen gas was bubbled and the mixture was stirred under hydrogen atmosphere at rt overnight. The crude mixture was filtered over Celite®, solvents were removed under vacuum to give compound 106 as a brown oil (203 mg, 97%). MS (ESI+): m/z (%): 341 [M+H]⁺. ¹H NMR (300 MHz, CDCl₃): δ (ppm) 10.59 (s, 1H), 7.34-7.27 (m, 2H), 7.15-6.98 (m, 4H), 4.02 (s, 3H), 3.48 (s, 2H), 2.99-2.68 (m, 6H), 2.59 (s, 2H), 1.83-1.62 (m, 4H). ¹³C NMR (75 MHz, CDCl₃): δ (ppm) 169.69, 156.77, 153.33, 133.60, 133.54, 131.72, 131.59, 128.82, 128.43, 128.24, 126.26, 126.20, 125.92, 125.36, 57.43, 55.37, 52.70, 50.36, 36.96, 28.22, 27.46, 26.04.

### 6-(4-(3,4-dihydroisoquinolin-2(1H)-yl)butyl)-3-hydroxypicolinaldehyde—107

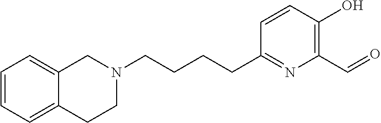 [SMILES: O=Cc1nc(CCCCN2CCc3ccccc3C2)ccc1O]

Compound 106 (690 mg, 2.03 mmol, 1 equiv.) was dissolved in dry dichloromethane (20 mL, 0.1 M), then triethylamine (850 μL, 6.09 mmol, 3 equiv.) and TBSOTf (550 μL, 3.05, 2.5 equiv.) were added dropwise at 0° C. The mixture was stirred at rt 3 h. Resulting mixture was diluted in dichloromethane, washed with water, brine and dried under sodium sulfate. Dichloromethane was removed under vacuum giving the crude silyl ether. ¹H NMR (300 MHz, CDCl₃) δ (ppm) 7.18-6.98 (m, 6H), 3.92 (s, 3H), 3.60 (s, 2H), 2.89 (t, J=5.8 Hz, 2H), 2.85-2.77 (m, 2H), 2.58-2.46 (m, 4H), 1.85-1.58 (m, 4H), 1.02-0.97 (m, 9H), 0.22-0.18 (m, 6H).

The crude silyl ether (880 mg, 2.03 mmol, 1.0 equiv.) was dissolved in dry dichloromethane (20 mL, 0.1 M) and cooled down to −78° C. DIBAL-H (5.07 mL, 5.07 mmol, 2.5 equiv.) was added dropwise and the mixture was stirred at −78° C. during 15 min. Methanol (5 mL) was added and the crude mixture was heat up to rt. After dilution with dichloromethane, the organic layer was washed using aqueous NaOH (1 M), water and brine. After drying under sodium sulfate, dichloromethane was removed under vacuum giving the crude silyl ether aldehyde moiety used without purification in the next step. ¹H NMR (300 MHz, CDCl₃): δ (ppm) 10.33 (s, 1H), 7.18 (d, J=8.5 Hz, 2H), 7.12-6.98 (m, 4H), 3.60 (s, 2H), 2.92-2.82 (m, 4H), 2.71 (t, J=5.9 Hz, 2H), 2.57-2.48 (m, 2H), 1.85-1.62 (m, 4H), 1.01 (s, 9H), 0.25 (s, 6H).

Crude product was dissolved in dry THF (20 mL, 0.1 M) and cooled down to 0° C. TBAF (2.44 mL, 2.44 mmol, 1.2 equiv.) was added dropwise and the mixture was stirred at 0° C. during 30 min. THF was removed under vacuum and the crude was purified using silica gel flash chromatography (dichloromethane/MeOH, 95/5, v/v) to give the title compound as an orange oil (180 mg, 28%). Rf=0.28 (dichloromethane/MeOH, 95/5, v/v). ¹H NMR (300 MHz, CDCl₃): δ (ppm) 10.02 (s, 1H), 7.31-7.20 (m, 2H), 7.15-6.94 (m, 4H), 3.61 (s, 2H), 2.94-2.76 (m, 4H), 2.72 (t, J=5.9 Hz, 2H), 2.61-2.50 (m, 2H), 1.87-1.60 (m, 4H). ¹³C NMR (75 MHz, CDCl₃): δ (ppm) 198.59, 156.87, 154.65, 135.63, 134.61, 134.17, 129.64, 128.54, 126.47, 126.25, 126.03, 125.51, 58.01, 56.08, 50.88, 37.06, 28.95, 27.47, 26.64. MS (ESI+): m/z (%): 311 [M+H]⁺.

### AB-558—90

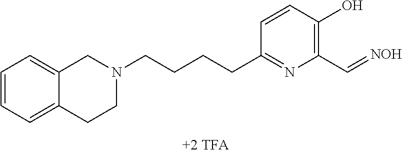

Aldehyde 107 (179 mg, 0.58 mmol, 1.0 equiv.) was dissolved in dry methanol (5.8 mL, 0.1 M). Hydroxylamine hydrochloride (84 mg, 1.2 mmol, 2 equiv.) and sodium acetate (123 mg, 1.5 mmol, 2.5 equiv.) were added and the mixture was stirred at rt during 3 h. Concentration until dryness and subsequent reverse phase purification (method D) gave TFA salt of title compound as a white-off solid (242 mg, 75%). ¹³C NMR (75 MHz, CD₃OD) δ (ppm) 161.13 (q, J=37 Hz), 154.26, 152.30, 148.51, 135.30, 132.10, 129.86, 129.46, 129.10, 128.85, 128.27, 127.85, 126.62, 56.96, 54.20, 51.14, 35.37, 27.72, 26.33, 24.64. HPLC (method B): t_{R}=18.5 min (>99%). MS (ESI+): m/z (%): 326 (100) [M+H]⁺. HRMS (ESI+): m/z calcd for C19H24N3O2 326.1869; found: 326.1863.

### Methyl 3-(benzyloxy)-6-(5-(tosyloxy)pent-1-yn-1-yl)picolinate—59a

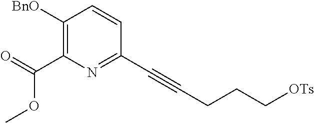 [SMILES: COC(=O)c1nc(C#CCCCOS(=O)(=O)c2ccc(C)cc2)ccc1OCc1ccccc1]

Methyl 3-(benzyloxy)-6-(5-((methylsulfonyl)oxy)pent-1-yn-1-yl)picolinate was synthetized following Procedure B using 14a and 59a was isolated after purification by flash chromatography on silica gel (Petroleum ether/EtOAc, 60/40, v/v) as a yellow oil (yield: quant.). Rf=0.4 (Petroleum ether/EtOAc, 60/40, v/v). ¹H NMR (300 MHz, CDCl₃) δ (ppm) 7.79 (d, J=8.3 Hz, 2H), 7.47-7.27 (m, 9H), 5.21 (s, 2H), 4.17 (t, J=6.1 Hz, 2H), 3.96 (s, 3H), 2.47 (t, J=7.0 Hz, 2H), 2.39 (s, 3H), 1.98-1.84 (m, 2H).

### Methyl 3-(benzyloxy)-6-(5-(3,4-dihydroisoquinolin-2(1H)-yl)pent-1-yn-1-yl)picolinate—108

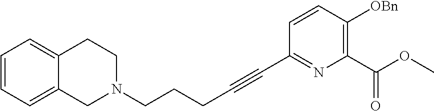 [SMILES: COC(=O)c1nc(C#CCCCN2CCc3ccccc3C2)ccc1OCc1ccccc1]

Tosylate 59a (992 mg, 2.07 mmol, 1.0 equiv) was dissolved in dry MeCN (20 mL, 0.1 M) under Argon atmosphere and the resulting mixture was cooled down to 0° C. 1,2,3,4-tetrahydroisoquinoline (300 μL, 2.28 mmol, 1.1 equiv.) followed by potassium carbonate (575 g, 4.14 mmol, 2 equiv.) were added in this order. The resulting heterogenous mixture was reflux overnight. Salt were removed by filtration and solvent was evaporated under reduced pressure. The crude product was purified by chromatography on silica gel (dichloromethane/MeOH, 98/2, v/v) to afford 108 as a yellow oil (662 mg, 72%). Rf=0.24 (dichloromethane/MeOH, 98/2, v/v). ¹H NMR (300 MHz, CDCl₃): δ (ppm) 7.45-7.24 (m, 6H), 7.14-6.99 (m, 4H), 5.20 (s, 2H), 3.96 (s, 3H), 3.64 (s, 2H), 2.90 (t, J=5.9 Hz, 2H), 2.74 (t, J=5.9 Hz, 2H), 2.67-2.59 (m, 2H), 2.51 (dd, J=12.1, 4.9 Hz, 2H), 1.96-1.84 (m, 2H). ¹³C NMR (75 MHz, CDCl₃): δ (ppm) 164.94, 152.98, 140.18, 135.65, 134.93, 134.46, 132.10, 130.06, 128.61, 127.01, 126.67, 126.17, 125.66, 121.90, 90.26, 79.58, 70.95, 57.37, 56.28, 52.76, 51.02, 29.24, 26.10, 17.50

### Methyl 6-(5-(3,4-dihydroisoquinolin-2(1H)-yl)pentyl)-3-hydroxypicolinate—109

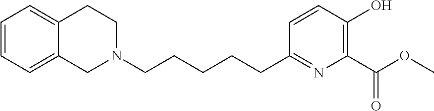 [SMILES: COC(=O)c1nc(CCCCCN2CCc3ccccc3C2)ccc1O]

Compound 108 (660 mg, 1.5 mmol, 1.0 equiv.) was dissolved in MeOH (30 mL, 0.05 M) and the homogenous solution was degassed 20 min using argon. Pearlman's catalyst (132 mg, 20% w/w) was added. After 10 min of degassing using Argon, balloon of hydrogen gas was bubbled and the mixture was stirred under hydrogen atmosphere at rt until completion. The crude mixture was filtered over Celite®, solvents were removed under vacuum to afford the product as orange oil (510 mg, 97%). ¹H NMR (300 MHz, CDCl₃): δ (ppm) 7.25 (d, J=4.8 Hz, 2H), 7.11-6.95 (m, 4H), 4.01 (s, J=6.3 Hz, 3H), 3.61 (s, 2H), 2.88 (t, J=5.8 Hz, 2H), 2.83-2.75 (m, 2H), 2.71 (t, J=5.9 Hz, 2H), 2.54-2.46 (m, 2H), 1.80-1.69 (m, 2H), 1.68-1.58 (m, 2H), 1.48-1.35 (m, 2H). ¹³C NMR (75 MHz, CDCl₃): δ (ppm) 170.01, 157.04, 153.86, 134.47, 134.08, 129.01, 128.58, 126.45, 125.99, 125.47, 58.07, 55.97, 52.97, 50.73, 37.46, 29.87, 28.83, 27.13, 26.82.

### 6-(5-(3,4-dihydroisoquinolin-2(1H)-yl)pentyl)-3-hydroxypicolinaldehyde—110

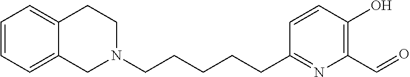 [SMILES: O=Cc1nc(CCCCCN2CCc3ccccc3C2)ccc1O]

Compound 109 (462 mg, 1.65 mmol, 1.0 equiv.) was dissolved in dry dichloromethane (0.1 M), then triethylamine (500 μL, 3.5 mmol, 2.1 equiv.) and TBSOTf (360 μL, 1.98 mmol, 1.2 equiv.) were added dropwise at 0° C. The mixture was stirred at rt during 4 h. Resulting mixture was diluted in dichloromethane, washed with water, brine and dried under sodium sulfate. dichloromethane was removed under vacuum giving the crude silyl ether that was dissolved in dry dichloromethane (0.1 M) and cooled down to −78° C. DIBAL-H (4.2 mL, 4.2 mmol, 2.5 equiv.) was added dropwise and the mixture was stirred at −78° C. during 15 min. Methanol was added and the crude mixture was heat up to rt. After dilution with dichloromethane, the organic layer was washed using aqueous NaOH (1 M), water and brine. After drying under sodium sulfate or magnesium sulfate, dichloromethane was removed under vacuum giving the crude silyl ether aldehyde moiety used without purification in the next step. Crude reduced silyl ether was dissolved in dry THF (0.1 M) and cooled down to 0° C. TBAF (1.66 mL, 1.66 mmol, 1.2 equiv.) was added dropwise and the mixture was stirred at 0° C. during 60 min. THF was removed under vacuum and the crude was purified using flash chromatography (dichloromethane/MeOH, 95/5, v/v) to afford the product as a pale-yellow oil (322 mg, 60% over 3 steps). Rf=0.43 (dichloromethane/MeOH, 95/5, v/v). ¹H NMR (300 MHz, CDCl₃): δ (ppm) 10.02 (s, 1H), 7.30-7.21 (m, 2H), 7.14-6.91 (m, 4H), 3.62 (s, 2H), 2.89 (t, J=5.8 Hz, 2H), 2.83-2.66 (m, 4H), 2.55-2.46 (m, 2H), 1.84-1.58 (m, 4H), 1.50-1.34 (m, 2H). ¹³C NMR (75 MHz, CDCl₃): δ (ppm) 198.7, 156.94, 154.91, 135.71, 134.70, 134.25, 129.71, 128.62, 126.32, 126.31, 126.10, 125.58, 58.22, 56.16, 50.91, 37.26, 29.62, 29.00, 27.19, 26.97.

### AB-746—91

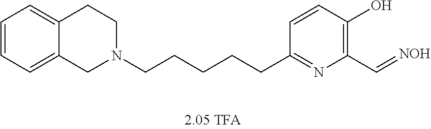

Aldehyde 110 (322 mg, 0.99 mmol, 1 equiv.) was dissolved in dry MeOH (10 mL) under argon. Hydroxylamine hydrochloride (75 mg, 1.08 mmol, 1.1 equiv.) and sodium acetate (110 mg, 1.35 mmol, 1.5 equiv.) were added and the mixture was stirred at 4 h. Methanol was removed under vacuum and the crude oxime was purified using reverse phase chromatography (method D) to give the title compound as a yellow solid (227.1 mg, 64%). ¹H NMR (300 MHz, CD₃OD): δ (ppm) 8.37 (s, 1H), 7.84 (d, J=8.8 Hz, 1H), 7.61 (d, J=8.8 Hz, 1H), 7.33-7.02 (m, 4H), 5.60 (s, 3H), 4.54 (d, J=15.1 Hz, 1H), 4.26 (d, J=15.1 Hz, 1H), 3.73 (s, 1H), 3.45-3.15 (m, 4H), 3.12-2.81 (m, 3H), 2.00-1.60 (m, 4H), 1.56-1.38 (m, 2H). ¹³C NMR (75 MHz, CD₃OD): δ (ppm) 162.74 (q, J=34.9 Hz, CF₃), 154.41, 151.33, 143.78, 133.32, 132.48, 132.01, 129.70, 129.26, 128.76, 128.10, 127.72, 123.78, 119.90, 116.02, 112.14, 57.03, 53.99, 50.95, 49.00, 33.97, 30.05, 26.86, 26.23, 24.71. HPLC (method B): t_{R}=19.07 min (95%). MS (ESI+): m/z (%): 340 (100) [M+H]⁺. HRMS (ESI+): m/z calcd for C20H26N3O2 340.2029; found: 340.2025.

### Methyl 3-(benzyloxy)-6-(5-((methylsulfonyl)oxy)pent-1-yn-1-yl)picolinate—57a

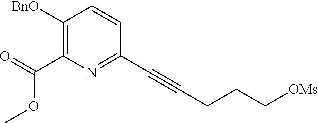 [SMILES: COC(=O)c1nc(C#CCCCOS(C)(=O)=O)ccc1OCc1ccccc1]

Methyl 3-(benzyloxy)-6-(4-((methylsulfonyl)oxy)pent-1-yn-1-yl)picolinate was synthetized following Procedure A using 14a and 57a was used without further purification in the next step (orangeous oil, yield: quant.). Caution: must be used within 12 h. ¹H NMR (300 MHz, CDCl₃) δ (ppm) 7.46-7.26 (m, 7H), 5.20 (s, 2H), 4.39 (t, J=6.1 Hz, 2H), 3.96 (s, 3H), 3.04 (s, 3H), 2.58 (t, J=6.9 Hz, 2H), 2.14-1.97 (m, 2H).

### Methyl 6-(5-(azepan-1-yl)pent-1-yn-1-yl)-3-(benzyloxy)picolinate—121

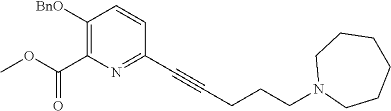 [SMILES: COC(=O)c1nc(C#CCCCN2CCCCCC2)ccc1OCc1ccccc1]

Mesylate 57a (581 mg, 1.8 mmol, 1.03 equiv) was dissolved in dry MeCN (20 mL, 0.1 M) under Argon atmosphere and the resulting mixture was cooled down to 0° C. Azepane (200 μL, 1.74 mmol, 1.0 equiv.) followed by potassium carbonate (745 mg, 5.4 mmol, 1.5 equiv.) were added in this order. The resulting heterogenous mixture was reflux overnight. Salt were removed by filtration and solvent was evaporated under reduced pressure. The crude product was purified by flash chromatography (dichloromethane/MeOH, 95/5, v/v) to afford the desired product as a brown oil (438 mg, 62%). Rf=0.27 (dichloromethane/MeOH, 95/5, v/v). ¹H NMR (300 MHz, CDCl₃): δ (ppm) 7.54-7.14 (m, 7H), 5.18 (s, 2H), 3.94 (s, 3H), 2.78-2.50 (m, 6H), 2.44 (t, J=7.2 Hz, 2H), 1.85-1.68 (m, 2H), 1.59 (s, 8H). ¹³C NMR (75 MHz, CDCl₃): δ (ppm) 164.74, 152.68, 139.88, 135.45, 129.85, 128.56, 128.05, 126.78, 121.69, 90.34, 79.20, 70.65, 57.01, 55.33, 52.48, 28.07, 26.89, 26.40, 17.18.

### Methyl 6-(5-(azepan-1-yl)pentyl)-3-hydroxypicolinate—122

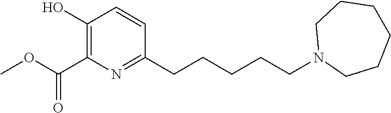 [SMILES: COC(=O)c1nc(CCCCCN2CCCCCC2)ccc1O]

Compound 121 (369 mg, 0.91 mmol, 1 equiv.) was dissolved in MeOH (18 mL, 0.05 M) and the homogenous solution was degassed 20 min using argon. Pearlman's catalyst (147 mg, 20% w/w) was added. After 10 min of degassing using Argon, balloon of hydrogen gas was bubbled and the mixture was stirred under hydrogen atmosphere at rt until completion. The crude mixture was filtered over Celite®, solvents were removed under vacuum to give 122 as an orangeous oil (291 mg, quant.). ¹H NMR (300 MHz, CDCl₃): δ (ppm) 8.97 (br s, 1H), 7.25 (s, 2H), 4.01 (s, 3H), 2.83-2.69 (m, 2H), 2.69-2.53 (m, 4H), 2.54-2.39 (m, 2H), 1.75-1.44 (m, 12H), 1.40-1.26 (m, 2H). ¹³C NMR (75 MHz, CDCl₃): δ (ppm) 170.16, 157.18, 154.00, 129.18, 128.78, 126.63, 57.94, 55.25, 53.14, 37.56, 29.91, 27.15, 27.01, 26.72.

### 6-(5-(azepan-1-yl)pentyl)-3-hydroxypicolinaldehyde—123

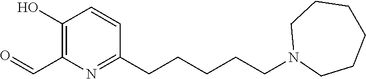 [SMILES: O=Cc1nc(CCCCCN2CCCCCC2)ccc1O]

Unprotected 3-hydroxypyridine-2-methyl ester 122 (86 mg, 0.28 mmol, 1.0 equiv.) was dissolved in dry dichloromethane (10 mL, 0.2 M), then triethylamine (118 μL, 0.84, 3 equiv.) and TBSOTf (77 μL, 0.42 mmol, 1.5 equiv.) were added dropwise at 0° C. The mixture was stirred at rt overnight. Resulting mixture was diluted in dichloromethane, washed with water, brine and dried under sodium sulfate. dichloromethane was removed under vacuum giving the crude silyl ether that was dissolved in dry dichloromethane (0.1 M) and cooled down to −78° C. DIBAL-H (685 μL, 685 μmol. 2.5 equiv.) was added dropwise and the mixture was stirred at −78° C. during 15 min. Methanol was added and the crude mixture was heat up to rt. After dilution with dichloromethane, the organic layer was washed using aqueous NaOH (1 M), water and brine. After drying under sodium sulfate or magnesium sulfate, dichloromethane was removed under vacuum giving the crude silyl ether aldehyde moiety used without purification in the next step. Crude reduced silyl ether was dissolved in dry THF and cooled down to 0° C. TBAF (1.05-1.1 equiv.) was added dropwise and the mixture was stirred at 0° C. during 30 min. THF was removed under vacuum and the crude was purified using silica gel flash chromatography (dichloromethane/MeOH, 95/5 to 90/10, v/v) to give the title compound as a brown oil (56 mg, 68% over 3 step, with trace of TBAF, 1 aldehyde for 0.4 TBAF). Rf=0.5 (dichloromethane/MeOH, 90/10, v/v). ¹H NMR (300 MHz, CDCl₃) δ (ppm) 10.04 (s, 1H), 7.36 (d, J=8.7 Hz, 1H), 7.32 (d, J=8.5 Hz, 1H), 3.58-3.42 (br s, 2H), 3.06-2.83 (m, 4H), 2.69 (t, J=7.6 Hz, 2H), 2.25-2 (m, 2H), 1.96-1.59 (m, 12H). ¹³C NMR (75 MHz, CDCl₃) δ (ppm) 198.68, 157.10, 154.29, 135.76, 130.01, 126.57, 57.48, 54.39, 36.64, 26.93, 26.30, 24.16, 23.84, 23.38. HRMS (ESI+): m/z calcd. for C17H27N2O2 291.2079; found 291.2073.

### AB-152—112 & 112a

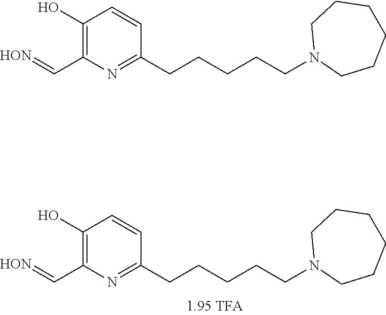

Aldehyde 123 (60 mg, 0.21 mmol, 1 equiv.) was dissolved in dry MeOH under argon. Hydroxylamine hydrochloride (15 mg, 1.1 equiv.) and sodium acetate (21 mg, 1.2 equiv) were added and the mixture was stirred at rt during 4 h. Methanol was removed under vacuum and the crude oxime was purified using normal phase purification (dichloromethane/MeOH, 90/10, v/v) to give 112 (38 mg, 60%) as a brown oil. Rf=0.26 (dichloromethane/MeOH, 90/10, v/v). ¹H NMR (300 MHz, CD₃OD): δ (ppm) 8.28 (s. 1H), 7.26 (d, J=8.5 Hz, 1H), 7.14 (d, J=8.5 Hz, 1H), 2.92-2.78 (m, 4H), 2.72 (s, 2H), 2.68-2.59 (m, 2H), 1.75-1.61 (m, 12H), 1.49-1.35 (m, 2H). ¹³C NMR (75 MHz, CD₃OD): δ (ppm) 154.36, 152.79, 136.40, 126.15, 125.33, 59.08, 56.37, 37.69, 30.98, 30.76, 27.98, 27.82, 26.97, 24.79. HPLC (method A): tR=17.9 min (purity: 97%). HRMS (ESI+): m/z calcd. for C17H28N3O2 306.2177; found 306.2182.

Aldehyde 123 (295 mg, 1.017 mmol, 1.0 equiv.) was dissolved in dry MeOH under argon. Hydroxylamine hydrochloride (87 mg, 1.2 equiv.) and sodium acetate (120 mg, 1.4 equiv.) were added and the mixture was stirred at rt during 4 h. Methanol was removed under vacuum and the crude oxime was purified using reverse phase purification (method D) to give 112 as a brown oil (98 mg, 18%). ¹H NMR (300 MHz, CDCl₃): δ (ppm) 11.67 (s, 2H), 8.35 (s, 1H), 7.16 (d, J=8.5 Hz, 1H), 6.98 (d, J=8.5 Hz, 1H), 3.58-3.42 (br s, 2H), 3.06-2.83 (m, 4H), 2.69 (t, J=7.6 Hz, 2H), 2.25-2 (m, 2H), 1.96-1.59 (m, 12H). ¹³C NMR (75 MHz, CDCl₃): δ (ppm) 162.05 (q, J=35 Hz), 153.18, 152.64, 152.52, 135.09, 124.67, 123.90, 57.48, 54.39, 36.64, 26.93, 26.30, 24.16, 23.84, 23.38. HPLC (method B): t_{R}=17.57 min (96.5%)

### Methyl 6-(4-(azocan-1-yl)but-1-yn-1-yl)-3-(benzyloxy)picolinate—125

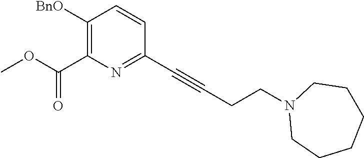 [SMILES: COC(=O)c1nc(C#CCCN2CCCCCC2)ccc1OCc1ccccc1]

Mesylate 57a (1.12 g, 2.90 mmol, 1.2 equiv.) was dissolved in dry MeCN (30 mL, 0.1 M) under Argon atmosphere and the resulting mixture was cooled down to 0° C. Azocane (315 μL, 2.40 mmol, 1.0 equiv.) followed by potassium carbonate (666 mg, 4.80 mmol, 2.0 equiv.) were added in this order. The resulting heterogenous mixture was reflux overnight. Salt were removed by filtration and solvent was evaporated under reduced pressure. The crude product was purified by flash chromatography (dichloromethane/MeOH, 100/0 to 95/5, v/v) to give the title compound as a brown oil (336 mg, 29%, with trace of PPh₃). Rf=0.31 (dichloromethane/MeOH, 96/4, v/v). TLC-MS (ESI+): m/z=407 [M+H]+, 429 [M+Na]+. ¹H NMR (300 MHz, CDCl₃): δ (ppm) 7.50-7.12 (m, 8H), 5.07 (s, 2H), 3.83 (s, 3H), 2.69 (t, J=7.4 Hz, 2H), 2.50 (br s, 3H), 2.44 (t, J=7.4 Hz, 2H), 1.49 (t, J=8.7 Hz, 11H). ¹³C NMR (75 MHz, CDCl₃): δ (ppm) 164.78 (q. J=36 Hz), 152.74, 139.92, 135.47, 135.42, 133.05, 132.00, 131.90, 131.87, 131.67, 129.81, 128.59, 128.50, 128.34, 128.08, 126.81, 121.72, 89.24, 79.69, 70.70, 57.12, 53.39, 52.50, 27.85, 27.25, 26.09, 18.59.

### Methyl 6-(4-(azocan-1-yl)butyl)-3-hydroxypicolinate—126

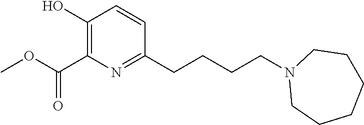 [SMILES: COC(=O)c1nc(CCCCN2CCCCCC2)ccc1O]

Compound 125 (336 mg, 0.827 mmol, 1.0 equiv.) was dissolved in MeOH (83 mL, 0.01 M) and the homogenous solution was degassed 20 min using argon. Pearlman's catalyst (150 mg, 40% w/w) was added. After 10 min of degassing using Argon, balloon of hydrogen gas was bubbled and the mixture was stirred under hydrogen atmosphere at rt overnight. The crude mixture was filtered over Celite®, solvents were removed under vacuum. The crude was purified by flash chromatography on silica gel (dichloromethane/MeOH, 90/10, v/v) to afford the title product (39 mg, 15%). Rf=0.50 (dichloromethane/MeOH, 90/10, v/v). TLC-MS (ESI+): m/z=321 [M+H]+, 344 [M+Na]+. ¹H NMR (300 MHz, CDCl₃): δ (ppm) 10.56 (s, 1H), 7.32 (s, 2H), 4.04 (s, 3H), 3.22 (br s, 4H), 3.06-2.90 (m, 2H), 2.90-2.71 (m, 2H), 2.06-1.86 (m, 6H), 1.86-1.71 (m, 3H), 1.67 (br s, 5H). ¹³C NMR (75 MHz, CDCl₃): δ (ppm) 170.46, 157.86, 153.10, 129.88, 129.35, 127.45, 55.63, 53.63, 50.80, 37.00, 27.50, 26.79, 24.87, 24.30, 22.85.

### AB-643—127

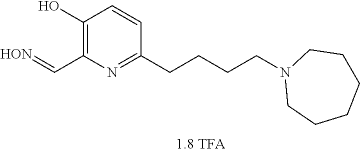

Compound 126 (39 mg, 0.122 mmol, 1.0 equiv.) was dissolved in dry dichloromethane (10 mL, 0.1 M). then triethylamine (52 μL, 0.366 mmol, 3 equiv.) and TBSOTf (43 μL, 0.188 mmol, 3 equiv.) were added dropwise at 0° C. The mixture was stirred at rt overnight. Resulting mixture was diluted in dichloromethane, washed with water, brine and dried under sodium sulfate. Dichloromethane was removed under vacuum giving the crude silyl ether that was dissolved in dry dichloromethane (10 mL, 0.1 M) and cooled down to −78° C. DIBAL-H (305 μL, 0.305 mmol, 2.5 equiv.) was added dropwise and the mixture was stirred at −78° C. during 15 min. Methanol (305 μL) was added and the crude mixture was heat up to rt. After dilution with dichloromethane, the organic layer was washed using aqueous NaOH (1 M), water and brine. After drying under sodium sulfate, dichloromethane was removed under vacuum giving the crude silyl ether aldehyde moiety used without purification in the next step. Crude reduced silyl ether was dissolved in dry THF (20 mL, 0.05 M) and cooled down to 0° C. TBAF (150 μL, 0.150 mmol, 1.2 equiv.) was added dropwise and the mixture was stirred at 0° C. during 30 min. THF was removed under vacuum and the crude was purified using silica gel flash chromatography (dichloromethane/MeOH, 100/0 to 90/10, v/v) to afford the aldehyde as a yellow oil (16.2 mg, 46% over 3 steps—trace of TBAF). TLS-MS (ESI+): m/z=291 [M+H]⁺, 313 [M+Na]⁺; Rf=0.18 (dichloromethane/MeOH, 90/10, v/v).

The crude aldehyde was dissolved in MeOH (5 mL, 0.02 M) under argon. Hydroxylamine hydrochloride (10 mg, 0.140 mmol, 2.5 equiv.) and sodium acetate (14 mg, 0.168 mmol, 3 equiv.) were added and the mixture was stirred at rt overnight. Methanol was removed under vacuum and the crude oxime was purified using reverse phase chromatography (method D) to give the title compound as brown oil (4.98 mg, 8% over 4 steps). ¹H NMR (300 MHz, CD₃OD): δ (ppm) 8.34 (s, 1H), 7.46 (d, J=8.6 Hz, 1H), 7.32 (d, J=8.6 Hz, 1H), 3.55-3.40 (m, 2H), 3.27-3.09 (m, 4H), 2.95-2.80 (m, 2H), 2.10-1.93 (m, 2H), 1.90-1.70 (m, 10H), 1.70-1.50 (br s, 3H). ¹³C NMR (75 MHz, CD₃OD): δ (ppm) 152.75, 151.32, 148.49, 134.32, 126.67, 124.77, 55.89, 51.34, 34.45, 26.35, 25.16, 24.03, 23.53, 22.38. HRMS (ESI+): m/z calcd. for C17H28N3O2 306.2182; found 306.2184. HPLC (method A): t_{R}=17.90 min (purity=95.1%).

### Methyl 6-(5-(azocan-1-yl)pent-1-yn-1-yl)-3-(benzyloxy)picolinate—128

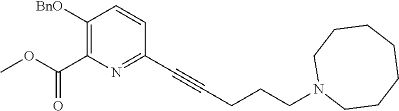 [SMILES: COC(=O)c1nc(C#CCCCN2CCCCCCC2)ccc1OCc1ccccc1]

Tosylate 59a (1.02 g, 2.13 mmol, 1.0 equiv) was dissolved in dry MeCN (0.1 M) under Argon atmosphere and the resulting mixture was cooled down to 0° C. Azocane (305 μL, 2.35 mmol, 1.1 equiv.) followed by potassium carbonate (590 mg, 4.26 mmol, 2 equiv.) were added in this order. The resulting heterogenous mixture was reflux overnight. Salt were removed by filtration and solvent was evaporated under reduced pressure. The crude product was purified using Silica gel flash chromatography (dichloromethane/MeOH, 95/5 to 90/10, v/v) to give the title compound as a brown oil (780 mg, 85%). Rf=0.43 (dichloromethane/MeOH, 90/10, v/v). ¹H NMR (300 MHz, CDCl₃): δ (ppm) 7.54-7.23 (m, 7H), 5.19 (s, 2H), 3.95 (s, 3H), 2.65 (t, J=6.6 Hz, 5H), 2.50 (t, J=7.2 Hz, 2H), 1.93-1.73 (m, 2H), 1.61 (s, 9H). ¹³C NMR (75 MHz, CDCl₃): δ (ppm) 164.66, 152.58, 139.77, 135.33, 135.19, 129.83, 128.43, 127.93, 126.68, 121.62, 89.86, 79.30, 70.52, 57.30, 53.41, 52.35, 26.89, 26.68, 26.25, 25.93, 16.96.

### Methyl 6-(5-(azocan-1-yl)pentyl)-3-hydroxypicolinate—129

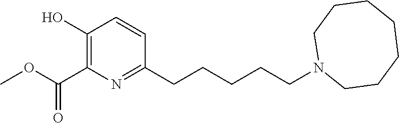 [SMILES: COC(=O)c1nc(CCCCCN2CCCCCCC2)ccc1O]

Compound 128 (780 mg, 1.86 mmol, 1 equiv.) was dissolved in MeOH (186 mL, 0.01 M) and the homogenous solution was degassed 20 min using argon. Pearlman's catalyst (150 mg, 20% w/w) was added. After 10 min of degassing using Argon, balloon of hydrogen gas was bubbled and the mixture was stirred under hydrogen atmosphere at rt overnight. The crude mixture was filtered over Celite®, solvents were removed under vacuum. The crude was purified by flash chromatography on silica gel (dichloromethane/MeOH, 90/10, v/v) to afford the title product (621 mg, 100%). Rf=0.52 (dichloromethane/MeOH, 90/10, v/v). TLC-MS (ESI+) m/z=335 [M+H]⁺, 357 [M+Na]⁺. ¹H NMR (300 MHz, CDCl₃): δ (ppm) 7.11 (d, J=8.7 Hz, 1H), 7.07 (d, J=8.4 Hz, 1H), 3.80 (s, 3H), 3.17 (br s, 2H), 3.03 (br s, 3H), 2.77 (br s, 2H), 2.56 (t, J=7.6 Hz, 2H), 1.72 (br s, 6H), 1.48 (d, J=19.1 Hz, 7H), 1.18 (br s, 3H). ¹³C NMR (75 MHz, CDCl₃): δ (ppm) 169.79, 156.91, 153.18, 129.17, 128.59, 126.53, 55.58, 52.91, 50.55, 36.82, 29.06, 26.13, 25.85, 24.24, 24.03, 22.39.

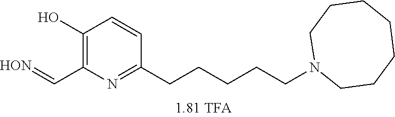

Compound 129 (621, 1.86 mmol, 1 equiv.) was dissolved in dry dichloromethane (20 mL, 0.1 M), then triethylamine (783 μL, 5.58, 3 equiv.) and TBSOTf (510 μL, 2.79 mmol, 1.5 equiv.) were added dropwise at 0° C. The mixture was stirred at rt until overnight. Resulting mixture was diluted in dichloromethane, washed with water, brine and dried under sodium sulfate. dichloromethane was removed under vacuum giving the crude silyl ether that was dissolved in dry dichloromethane (20 mL, 0.1 M) and cooled down to −78° C. DIBAL-H (3.29 mL, 3.29 mmol, 2.5 equiv.) was added dropwise and the mixture was stirred at −78° C. during 15 min. Methanol was added and the crude mixture was heat up to rt. After dilution with dichloromethane, the organic layer was washed using aqueous NaOH (1 M), water and brine. After drying under sodium sulfate or magnesium sulfate, dichloromethane was removed under vacuum giving the crude silyl ether aldehyde moiety used without purification in the next step. Crude reduced silyl ether was dissolved in dry THF (40 mL) and cooled down to 0° C. TBAF (1.9 mL, 1.9 mmol, 1.02 equiv.) was added dropwise and the mixture was stirred at 0° C. during 30 min. THF was removed under vacuum and the crude was purified through a silica gel plug (dichloromethane/MeOH, 95/5, v/v) to give the crude deprotected aldehyde (105.4 mg, 0.347 mmol, 1 equiv.) that was dissolved in dry MeOH under argon. Hydroxylamine hydrochloride (30 mg, 0.417 mmol, 1.1 equiv.) and sodium acetate (36 mg, 0.417 mmol, 1.2 equiv.) were added and the mixture was stirred overnight at rt. Methanol was removed under vacuum and the crude oxime was purified using reverse phase chromatography to afford the title compound as light orange oil (5.42 mg, 0.9% over 4 steps). ¹H NMR (300 MHz, CD₃OD): δ (ppm) 7.46 (d, J=8.7 Hz, 1H), 7.40 (d, J=8.7 Hz, 1H), 3.54-3.37 (m, 2H), 3.28-3.05 (m, 3H), 2.86-2.71 (m, 2H), 2.10-1.90 (m, 2H), 1.90-1.54 (m, 13H), 1.51-1.36 (m, 2H). ¹³C NMR (75 MHz, CD₃OD): δ (ppm) 170.40 (q, 38 Hz), 158.07, 154.94, 130.81, 130.03, 128.50, 57.61, 53.36, 52.78, 37.20, 30.48, 27.07, 26.56, 25.47, 25.29, 23.83, 9.18. HRMS (ESI+): m/z calcd. for C18H30N3O2 320.2338; found 320.2332. HPLC (method B): t_{R}=18.58 min (purity=95.2%).

### N-(tert-Butoxycarbonyl)-N-(4-piperidyl)amine—132

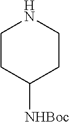 [SMILES: CC(C)(C)OC(=O)NC1CCNCC1]

1-benzylpiperidin-4-amine (4 mL, 19.05 mmol, 1.0 equiv.) and DIPEA (8.3 mL, 45.82 mmol, 2.4 equiv.) were dissolved in dry THF (40 mL) at 0° C. and Boc₂O (4.97 g, 22.86 mmol, 1.2 equiv.) in dry THF (23 mL) was added dropwise. The mixture was stirred overnight at rt. After concentration until dryness, a crude white powder was obtained and used without purification (5.33 g, 97%). The product obtained 5.33 g, 18.40 mmol, 1 equiv.) was dissolved in degassed methanol (100 mL). Pd/C (533 mg, 10% w/w) was poured into this solution. After 10 min of degassing using argon, balloon of hydrogen gas was bubbled and the mixture was stirred under hydrogen atmosphere (1 atm) at rt overnight. The crude mixture was filtered over Celite®, solvents were removed under vacuum to give the title compound as a white solid (3.68 g, 97% over 1 steps). Mp=164° C. (dec). MS (ESI+): m/z=201 [M+H]⁺, 223 [M+Na]⁺. ¹H NMR (300 MHz, CDCl₃): δ (ppm) 4.88 (br s, 1H), 3.36 (br s, 1H), 2.94 (dt, J=12.4, 3.0 Hz, 2H), 2.53 (td, J=12.4, 2.3 Hz, 2H), 1.80 (d, J=10.1 Hz, 2H), 1.63-1.44 (m, 1H), 1.28 (d, J=30.7 Hz, 9H), 1.16 (ddd, J=15.4, 11.7, 4.0 Hz, 2H). ¹³C NMR (75 MHz, CDCl₃): δ (ppm) 155.00, 78.72, 47.99, 45.25, 33.75, 28.22.

### Methyl 3-(benzyloxy)-6-(4-((methylsulfonyl)oxy)but-1-yn-1-yl)picolinate—57b

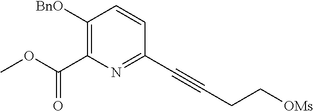 [SMILES: COC(=O)c1nc(C#CCCOS(C)(=O)=O)ccc1OCc1ccccc1]

Methyl 3-(benzyloxy)-6-(5-((methylsulfonyl)oxy)but-1-yn-1-yl)picolinate was synthetized following Procedure A using 14b and 57b was used without purification in the next step (orangeous oil, yield: quant.). Caution: must be used within 6 h. Rf=0.5 (dichloromethane/MeOH, 1/1, v/v). ¹H NMR (300 MHz, CDCl₃) δ (ppm) 7.51-7.27 (m, 7H), 5.21 (s, 2H), 3.96 (s, 3H), 3.82 (q, J=6.3 Hz, 2H), 2.69 (t, J=6.3 Hz, 2H), 1.56 (s, 3H).

### Methyl 3-(benzyloxy)-6-(4-(4-((tert-butoxycarbonyl)amino)piperidin-1-yl)but-1-yn-1-yl)picolinate—133

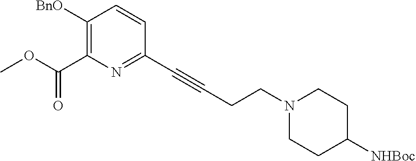 [SMILES: COC(=O)c1nc(C#CCCN2CCC(NC(=O)OC(C)(C)C)CC2)ccc1OCc1ccccc1]

Mesylate 57b (973 mg, 2.5 mmol, 1.0 equiv.) was dissolved in dry MeCN (30 mL, 0.08 M) under argon atmosphere and the resulting mixture was cooled down to 0° C. Compound 132 (500 mg, 2.5 mmol, 1.0 equiv.) followed by potassium carbonate (700 g, 5 mmol, 2.0 equiv.) were added in this order. The resulting heterogenous mixture was reflux overnight. Salt were removed by filtration and solvent was evaporated under reduced pressure. The crude product was purified by flash chromatography (dichloromethane/MeOH, 94/6 to 90/10, v/v) to give the title compound 136 as an orange oil (389 g, 32%). Rf=0.25 (dichloromethane/MeOH, 95/5, v/v). MS (ESI+): m/z=494 [M+H]⁺. ¹H NMR (300 MHz, CDCl₃): δ (ppm) 7.43-7.04 (m, 7H), 5.10 (s, 2H), 4.56 (br s, 1H), 3.85 (s, 3H), 3.45-3.26 (m, 1H), 2.76 (d, J=11.8 Hz, 2H), 2.63-2.52 (m, 2H), 2.52-2.43 (m, 2H), 2.06 (t, J=10.5 Hz, 2H), 1.83 (d, J=10.4 Hz, 2H), 1.35 (s, 10H). ¹³C NMR (75 MHz, CDCl₃): δ (ppm) 172.81, 164.68, 155.13, 152.88, 139.81, 135.38, 135.06, 132.00, 131.93, 131.90, 131.87, 129.91, 128.62, 128.52, 128.36, 128.12, 126.80, 121.71, 88.28, 79.89, 79.06, 70.65, 56.59, 52.58, 51.94, 32.29, 28.32, 17.61.

### Methyl 6-(4-(4-((tert-butoxycarbonyl)amino)piperidin-1-yl)butyl)-3-hydroxypicolinate—134

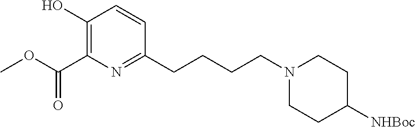 [SMILES: COC(=O)c1nc(CCCCN2CCC(NC(=O)OC(C)(C)C)CC2)ccc1O]

Compound 133 (388 mg, 0.788 mmol, 1.0 equiv.) was ether was dissolved in MeOH or EtOAc (0.05 M) and the homogenous solution was degassed 20 min using argon. Pearlman's catalyst (77 mg, 20% w/w) was added. After 10 min of degassing using Argon, balloon of hydrogen gas was bubbled and the mixture was stirred under hydrogen atmosphere at rt until completion (4 days). The crude mixture was filtered over Celite®, solvents were removed under vacuum to give the title compound as a brown solid (321 mg, quant.). MS (ESI+): m/z=409 [M+H]⁺. ¹H NMR (300 MHz, CDCl₃): δ (ppm) 7.22 (s, 2H), 4.86 (br s, 1H), 3.94 (s, 3H), 3.41 (br s, 1H), 2.88 (d, J=10.7 Hz, 2H), 2.71 (t, J=7.4 Hz, 2H), 2.49-2.30 (m, 2H), 2.12 (t, J=8.7 Hz, 2H), 1.88 (d, J=13.8 Hz, 3H), 1.73-1.42 (m, 6H), 1.34 (s, 10H). ¹³C NMR (75 MHz, CDCl₃): δ (ppm) 169.92, 157.05, 155.21, 153.45, 132.00, 131.95, 131.87, 129.14, 128.66, 128.55, 128.39, 126.62, 79.08, 57.91, 53.02, 52.15, 37.06, 31.58, 28.32, 27.67, 25.85.

### Tert-butyl (1-(4-(6-formyl-5-hydroxypyridin-2-yl)butyl)piperidin-4-yl)carbamate—135

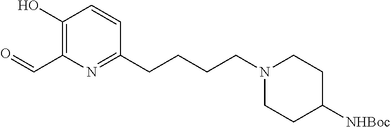 [SMILES: CC(C)(C)OC(=O)NC1CCN(CCCCc2ccc(O)c(C=O)n2)CC1]

Compound 134 (321 mg, 0.788 mmol, 1 equiv.) in dry dichloromethane (10, mL, 0.08 M), then triethylamine (280 μL, 1.97 mmol, 2.5 equiv.) and TBSOTf (220 μL. 0.946 mmol, 1.2 equiv.) were added dropwise at 0° C. The mixture was stirred at rt overnight. Resulting mixture was diluted in dichloromethane, washed with water, brine and dried under sodium sulfate. Dichloromethane was removed under vacuum giving the crude silyl ether that was dissolved in dry dichloromethane (10 mL, 0.08 M) and cooled down to −78° C. DIBAL-H (2.36 mL, 2.36 mmol, 3.0 equiv.) was added dropwise and the mixture was stirred at −78° C. during 15 min. Methanol was added and the crude mixture was heat up to rt. After dilution with dichloromethane, the organic layer was washed using aqueous NaOH (1 M), water and brine. After drying under sodium sulfate or magnesium sulfate, dichloromethane was removed under vacuum giving the crude silyl ether aldehyde moiety used without purification in the next step. Crude reduced silyl ether was dissolved in dry THF 20 mL and cooled down to 0° C. TBAF (790 μL, 0.790 mmol, 1 equiv.) was added dropwise and the mixture was stirred at 0° C. during 30 min. THF was removed under vacuum and the crude was purified using silica gel flash chromatography (dichloromethane/MeOH, 90/10, v/v) to give the title compound as a yellow oil (168 mg, 57% over 3 steps). TLC-MS (ESI+): m/z=378 [M+H]⁺; Rf=0.33 (dichloromethane/MeOH, 95/5, v/v). ¹H NMR (300 MHz, CDCl₃): δ (ppm) 9.95 (s, 1H), 7.24 (d, J=8.7 Hz, 1H), 7.20 (d, J=8.6 Hz, 1H), 4.42 (s, 1H), 3.39 (s, 1H), 2.87-2.66 (m, 4H), 2.40-2.23 (m, 2H), 2.00 (t, J=10.8 Hz, 2H), 1.86 (d, J=11.6 Hz, 2H), 1.77-1.58 (m, 2H), 1.52 (dt, J=23.2, 13.0 Hz, 4H), 1.37 (s, 9H). ¹³C NMR (75 MHz, CDCl₃): δ (ppm) 198.83, 157.08, 155.28, 154.75, 135.76, 129.86, 126.49, 77.16, 58.44, 52.55, 37.19, 32.48, 28.49, 27.69, 26.65.

### AB-740—115

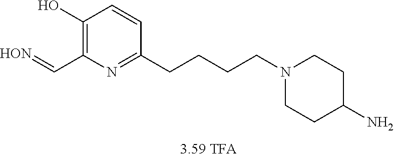

Aldehyde 135 (138 mg, 0.445 mmol, 1 equiv.) was dissolved in dry MeOH (20 mL, 0.02 M) under argon. Hydroxylamine hydrochloride (47 mg, 1.5 equiv.) was added and the mixture was stirred at rt 10 min. Then TFA (5 mL) was added at 0° C. and the mixture was stirred during 10 min. Volatiles were removed under vacuum and the crude oxime was purified using reverse phase chromatography to give the title compound as golden white off solid (177 mg, 57%). MS (ESI+): m/z: =293 (100) [M+H]⁺, 315 (15) [M+Na]⁺. ¹H NMR (300 MHz, CD₃OD): δ (ppm) 8.40 (s, 1H), 7.73 (d, J=8.7 Hz, 1H), 7.53 (d, J=8.7 Hz, 1H), 5.19 (s, 11H), 3.68 (d, J=9.9 Hz, 2H), 3.48 (s, 1H), 3.17 (s, 4H), 2.96 (d, J=6.6 Hz, 2H), 2.29 (d, J=12.7 Hz, 2H), 2.02 (dd, J=12.9, 9.8 Hz, 2H), 1.82 (t, J=11.6 Hz, 4H). ¹⁹F NMR (282 MHz, CD₃OD): δ (ppm) −76.84. ¹³C NMR (75 MHz, CD₃OD): δ (ppm) 162.95 (q, J=35.0 Hz), 154.52, 151.06, 145.03, 134.10, 131.60, 127.45, 123.79, 119.92, 116.05, 112.17, 57.51, 51.78, 49.00, 46.75, 34.02, 28.46, 27.51, 24.43. HRMS (ESI+): m/z calcd. for C15H25N4O2 293.1978; found 293.1974. HPLC (method B): t_{R}=9.70 min (purity=95.35%).

### Methyl 3-(benzyloxy)-6-(5-(4-((tert-butoxycarbonyl)amino)piperidin-1-yl)pent-1-yn-1-yl)picolinate—136

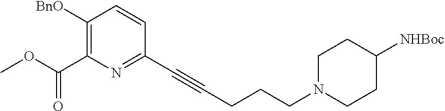 [SMILES: COC(=O)c1nc(C#CCCCN2CCC(NC(=O)OC(C)(C)C)CC2)ccc1OCc1ccccc1]

Tosylate 59a (2.0 g, 4.2 mmol, 1.0 equiv) was dissolved in dry MeCN (42 mL, 0.1 M) under Argon atmosphere and the resulting mixture was cooled down to 0° C. Compound 132 (840 mg, 4.32 mmol, 1.0 equiv.) followed by potassium carbonate (1.7 g, 4.2 mmol, 3.0 equiv.) were added in this order. The resulting heterogenous mixture was reflux overnight. Salt were removed by filtration and solvent was evaporated under reduced pressure. The crude product was purified using silica gel flash chromatography (dichloromethane/MeOH, 94/6 to 90/10, v/v) to give the title compound 136 as a yellow oil (2.1 g, 98%). ¹H NMR (300 MHz, CDCl₃): δ (ppm) 7.40-7.15 (m, 7H), 5.11 (s, 2H), 4.42 (s, 1H), 3.87 (s, 3H), 3.37 (s, 1H), 2.76 (d, J=11.5 Hz, 2H), 2.36 (dd, J=13.4, 6.6 Hz, 4H), 2.00 (t, J=11.0 Hz, 2H), 1.84 (d, J=11.1 Hz, 2H), 1.75-1.57 (m, 2H), 1.36 (s, 12H). ¹³C NMR (75 MHz, CDCl₃): δ (ppm) 164.85, 155.23, 152.90, 135.56, 135.48, 129.97, 128.74, 128.24, 126.93, 121.83, 89.94, 79.55, 77.16, 70.85, 57.44, 52.67, 52.40, 32.51, 28.44, 25.83, 17.41. TLS-MS: Rf=0.26 (dichloromethane/MeOH, 94/6, v/v) m/z=508 [M+H]+.

### Methyl 6-(5-(4-((tert-butoxycarbonyl)amino)piperidin-1-yl)pentyl)-3-hydroxypicolinate—137

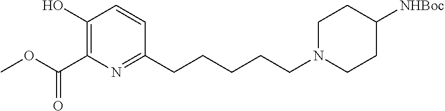 [SMILES: COC(=O)c1nc(CCCCCN2CCC(NC(=O)OC(C)(C)C)CC2)ccc1O]

Compound 136 (2.1 g, 4.15 mmol, 1.0 equiv.) was dissolved in MeOH (400 mL, 0.01 M) and the homogenous solution was degassed 20 min using argon. Pearlman's catalyst (350 mg, 20% w/w) was added. After 10 min of degassing using Argon, balloon of hydrogen gas was bubbled and the mixture was stirred under hydrogen atmosphere at rt overnight. The crude mixture was filtered over Celite®, solvents were removed under vacuum to give compound 137 as an orangeous oil (1.75 g, quant.). ¹H NMR (300 MHz, CDCl₃): δ (ppm) 10.59 (s, 1H), 7.28 (s, 2H), 4.51 (s, 1H), 4.04 (s, 3H), 3.53 (s, 1H), 3.08 (s, 2H), 2.89-2.64 (m, 2H), 2.53 (s, 2H), 2.33 (d, J=8.7 Hz, 2H), 2.01 (d, J=8.3 Hz, 3H), 1.88-1.55 (m, 6H), 1.53-1.17 (m, 13H). ¹³C NMR (75 MHz, CDCl₃): δ (ppm) 170.10, 157.18, 155.21, 153.70, 129.17, 128.80, 126.66, 125.88, 79.46, 57.95, 53.13, 52.08, 46.95, 37.38, 31.15, 29.68, 28.38, 26.91, 25.68.

### Tert-butyl (1-(5-(6-formyl-5-hydroxypyridin-2-yl)pentyl)piperidin-4-yl)carbamate—138

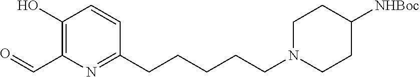 [SMILES: CC(C)(C)OC(=O)NC1CCN(CCCCCc2ccc(O)c(C=O)n2)CC1]

Compound 137 (1.75 g, 4.15 mmol, 1.0 equiv.) was dissolved in dry dichloromethane (40 mL, 0.1 M), then triethylamine (900 μL, 6.22 mmol, 1.5 equiv.) and TBSOTf (1.10 mL, 4.98 mmol, 1.2 equiv.) were added dropwise at 0° C. The mixture was stirred at rt during 3 h. Resulting mixture was diluted in dichloromethane, washed with water, brine and dried over sodium sulfate. Dichloromethane was removed under vacuum giving the crude silyl ether that was dissolved in dry dichloromethane (40 mL, 0.1 M) and cooled down to −78° C. DIBAL-H (8.2 mL, 8.2 mmol, 2.0 equiv.) was added dropwise and the mixture was stirred at −78° C. during 15 min. Methanol (8.2 mL) was added and the crude mixture was heat up to rt. After dilution with dichloromethane, the organic layer was washed using aqueous NaOH (1M), water and brine. After drying under sodium sulfate or magnesium sulfate, dichloromethane was removed under vacuum giving the crude silyl ether aldehyde moiety used without purification in the next step. Crude reduced silyl ether was dissolved in dry THF (100 mL) and cooled down to 0° C. TBAF (4.2 mL, 4.2 mmol, 1.01 equiv.) was added dropwise and the mixture was stirred at 0° C. during 30 min. THF was removed under vacuum and the crude was purified using silica gel flash chromatography (dichloromethane/MeOH, 100/0 to 90/10, v/v) to give the title product as a white off foam (316 mg, 30% over 3 steps). Rf=0.5 (dichloromethane/MeOH, 90/10). MS (ESI+): m/z (%)=392 (100) [M+H]⁺. ¹H NMR (300 MHz, CDCl₃): δ (ppm) 10.46 (br s, J=67.7 Hz, 1H), 10.05 (s, 1H), 7.32 (s, 2H), 4.63 (br s, 1H), 3.57 (br s, 1H), 3.10 (br s, 2H), 2.81 (t, J=7.7 Hz, 2H), 2.57 (br s, 2H), 2.35 (br s, 2H), 2.16-1.92 (m, 2H), 1.75 (dt, J=25.1, 12.6 Hz, 5H), 1.60-1.05 (m, 11H). ¹³C NMR (75 MHz, CDCl₃): δ (ppm) 198.85, 157.10, 155.30, 154.72, 135.82, 129.87, 126.50, 79.64, 58.12, 52.26, 47.11, 37.15, 31.31, 29.79, 29.40, 28.49, 26.98, 25.82.

### AB-737—116

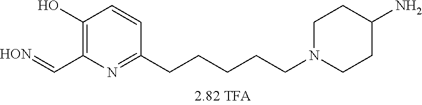

Same procedure as for AB-740. Yellow solid (298 mg, 65%). ¹H NMR (300 MHz, CD₃OD): δ (ppm) 8.42 (s, 1H), 7.84 (d, J=8.8 Hz, 1H), 7.61 (d, J=8.8 Hz, 1H), 5.48 (br s, 7H), 3.70 (d, J=10.8 Hz, 2H), 3.49 (s, 1H), 3.12 (s, 4H), 3.01-2.82 (m, 2H), 2.31 (d, J=12.9 Hz, 2H), 2.16-1.90 (m, 2H), 1.77 (t, J=10.8 Hz, 4H), 1.59-1.29 (m, 2H). ¹⁹F NMR (282 MHz, CD₃OD): δ (ppm) −76.66. ¹³C NMR (75 MHz, CD₃OD): δ (ppm) 162.87 (q, J=35.0 Hz), 154.52, 151.17, 143.34, 133.18, 132.92, 127.85, 123.76, 119.88, 116.01, 112.13, 57.73, 51.74, 46.81, 33.75, 29.96, 28.48, 26.79, 24.60. HRMS (ESI+): m/z calcd. for C16H27N4O2 307.2134; found 307.2133. HPLC (method B): t_{R}=14.51 min (97.37%).

### Methyl 3-(benzyloxy)-6-(4-(3-((tert-butoxycarbonyl)amino)piperidin-1-yl)but-1-yn-1-yl)picolinate—140

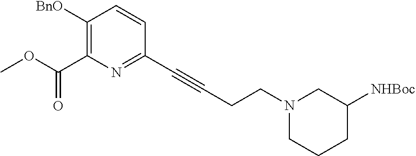 [SMILES: COC(=O)c1nc(C#CCCN2CCCC(NC(=O)OC(C)(C)C)C2)ccc1OCc1ccccc1]

Same procedure as 133, using commercially available 3-(Boc-amino)piperidine and methyl 3-(benzyloxy)-6-(4-((methylsulfonyl)oxy)but-1-yn-1-yl)picolinate as starting material. Yellow oil (461 mg, 38%). Rf=0.25 (dichloromethane/MeOH, 95/5). MS (ESI+): m/z=494 [M+H]⁺. ¹H NMR (300 MHz, CDCl₃): δ (ppm) 7.47-7.05 (m, 7H), 5.10 (s, 2H), 4.55 (s, 1H), 3.85 (s, 3H), 3.36 (s, 1H), 2.76 (d, J=11.8 Hz, 2H), 2.52 (qd, J=6.6, 3.7 Hz, 4H), 2.06 (t, J=10.5 Hz, 2H), 1.93-1.68 (m, 3H), 1.35 (s, 13H). ¹³C NMR (75 MHz, CDCl₃): δ (ppm) 172.81, 164.68, 155.13, 152.88, 139.81, 135.38, 135.06, 132.00, 131.93, 131.90, 131.87, 129.91, 129.04, 128.62, 128.52, 128.36, 128.12, 126.80, 121.71, 88.28, 79.89, 79.06, 70.65, 56.59, 52.58, 51.94, 47.51, 32.29, 28.32, 17.61.

### Methyl 6-(4-(3-((tert-butoxycarbonyl)amino)piperidin-1-yl)butyl)-3-hydroxypicolinate—141

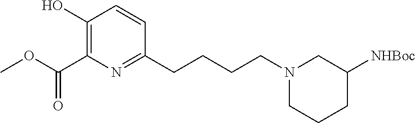 [SMILES: COC(=O)c1nc(CCCCN2CCCC(NC(=O)OC(C)(C)C)C2)ccc1O]

Same procedure as for compound 137, using compound 140 as starting material. Light orange oil (374 mg, 98%). ¹H NMR (300 MHz, CDCl₃): δ (ppm) 10.51 (br s, 1H), 7.23 (s, 2H), 5.05 (br s, 1H), 3.97 (s, 3H), 3.68 (br s, 1H), 2.86-2.66 (m, 2H), 2.55-2.07 (m, 5H), 1.62 (dd, J=15.7, 7.9 Hz, 3H), 1.56-1.41 (m, 4H), 1.36 (s, 10H). ¹³C NMR (75 MHz, CDCl₃): δ (ppm) 170.16, 157.24, 155.22, 153.86, 129.21, 128.80, 126.72, 79.05, 58.54, 58.35, 53.75, 53.19, 46.22, 37.40, 29.68, 28.47, 27.87, 26.19, 22.12. MS (ESI+): m/z=408 [M+H]⁺.

### Tert-butyl (1-(4-(6-formyl-5-hydroxypyridin-2-yl)butyl)piperidin-3-yl)carbamate—142

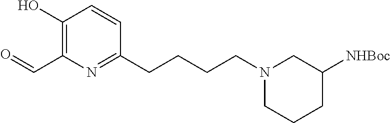 [SMILES: CC(C)(C)OC(=O)NC1CCCN(CCCCc2ccc(O)c(C=O)n2)C1]

Same procedure as for 138, using 141 as starting material. Yellow oil (189 mg, 55% over 3 steps). Rf=0.5 (dichloromethane/MeOH, 90/10). MS (ESI+): m/z=378 [M+H]⁺. ¹H NMR (300 MHz, CDCl₃): δ (ppm) 9.96 (s, 1H), 7.24 (s, 2H), 4.98 (s, 1H), 3.66 (s, 1H), 3.46-3.16 (m, 1H), 2.81-2.60 (m, 2H), 2.44 (s, 1H), 2.39-2.16 (m, 4H), 1.63 (dt, J=15.6, 8.0 Hz, 4H), 1.53-1.41 (m, 4H), 1.36 (s, 10H). ¹³C NMR (75 MHz, CDCl₃): δ (ppm) 198.64, 156.98, 155.16, 154.75, 135.70, 129.74, 126.41, 78.94, 58.98, 58.62, 58.29, 53.75, 46.30, 37.09, 29.67, 28.43, 27.49, 26.27, 24.12, 22.27, 19.76, 13.69.

### AB-738—117

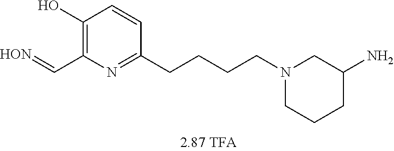

Same procedure as for 136, using 142 as starting material. White off solid (199 mg, 65%). ¹H NMR (300 MHz, CD₃OD): δ (ppm) 8.41 (s, 1H), 7.87-7.74 (m, 1H), 7.61 (t, J=8.9 Hz, 1H), 5.10 (br s, 9H), 3.78 (d, J=10.2 Hz, 1H), 3.70-3.41 (m, 2H), 3.20 (dd, J=9.7, 6.5 Hz, 2H), 3.14-2.80 (m, 4H), 2.34-2.00 (m, 2H), 2.00-1.58 (m, 4H), 1.46 (dt, J=15.0, 7.7 Hz, 2H). ¹⁹F NMR (282 MHz, CD₃OD): δ (ppm) −76.80. ¹³C NMR (75 MHz, CD₃OD): δ (ppm) 162.93 (q, J=35.0 Hz), 154.51, 151.31, 143.64, 133.46, 132.60, 129.07, 127.78, 123.76, 119.89, 116.01, 112.14, 58.25, 53.67, 52.96, 46.45, 33.95, 30.02, 27.30, 26.78, 24.53. HRMS (ESI+): m/z calcd. for C15H25N4O2 293.1978; found 293.1972. HPLC (method B): t_{R}=12.87 min (95%).

### Methyl 3-(benzyloxy)-6-(5-(3-((tert-butoxycarbonyl)amino)piperidin-1-yl)pent-1-yn-1-yl)picolinate—143

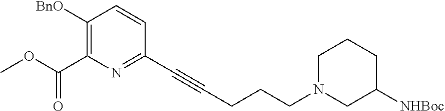 [SMILES: COC(=O)c1nc(C#CCCCN2CCCC(NC(=O)OC(C)(C)C)C2)ccc1OCc1ccccc1]

Same procedure as for compound 136, using commercially available 3-(Boc-amino)piperidine and methyl 3-(benzyloxy)-6-(5-(tosyloxy)pent-1-yn-1-yl)picolinate 59a as starting materials. Yellow oil (873 mg, 83%). Rf=0.36 (dichloromethane/MeOH, 90/10, v/v). MS (ESI+): m/z=508 [M+H]⁺. ¹H NMR (300 MHz, CDCl₃): δ (ppm) 7.53-7.11 (m, 7H), 5.16 (s, 2H), 5.01 (br s, 1H), 3.92 (s, 3H), 3.70 (br s, 1H), 2.48 (d, J=7.9 Hz, 1H), 2.39 (dd, J=14.7, 7.4 Hz, 5H), 2.25 (d, J=4.6 Hz, 2H), 1.81-1.59 (m, 3H), 1.42 (s, 12H). ¹³C NMR (75 MHz, CDCl₃): δ (ppm) 164.74, 155.07, 152.73, 139.97, 135.46, 135.37, 129.87, 128.59, 128.36, 128.30, 128.08, 127.04, 126.81, 121.71, 89.96, 79.39, 78.81, 70.69, 58.67, 57.29, 53.59, 52.50, 46.28, 29.65, 28.36, 25.52, 22.25, 17.18.

### Tert-butyl (1-(4-(6-formyl-5-hydroxypyridin-2-yl)butyl)piperidin-3-yl)carbamate—144

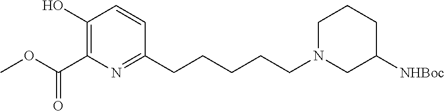 [SMILES: COC(=O)c1nc(CCCCCN2CCCC(NC(=O)OC(C)(C)C)C2)ccc1O]

Same procedure as for compound 137 using compound 143 as starting material. Yellow oil (666 mg, 92%). MS (ESI+): m/z=422 [M+H]⁺. ¹H NMR (300 MHz, CDCl₃): δ (ppm) 10.14 (s, 1H), 7.18 (s, 2H), 5.08 (s, 1H), 3.91 (s, 3H), 3.61 (s, 1H), 3.31 (s, 1H), 2.84-2.53 (m, 2H), 2.42 (s, 1H), 2.33-2.05 (m, 4H), 1.57 (d, J=7.4 Hz, 4H), 1.45-1.17 (m, 15H). ¹³C NMR (75 MHz, CDCl₃): δ (ppm) 169.86, 156.90, 155.00, 153.77, 128.94., 128.49, 126.40, 78.63, 58.41, 58.29, 53.49, 52.85, 49.85, 46.10, 37.30, 29.73, 29.59, 28.20, 26.95, 26.30, 22.12.

### Tert-butyl (1-(5-(6-formyl-5-hydroxypyridin-2-yl)pentyl)piperidin-3-yl)carbamate—145

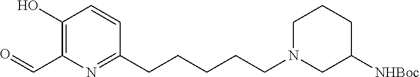 [SMILES: CC(C)(C)OC(=O)NC1CCCN(CCCCCc2ccc(O)c(C=O)n2)C1]

Same procedure as for compound 138, using compound 144 as starting material Yellow oil (230 mg, 76%). Rf=0.5 (dichloromethane/MeOH, 90/10, v/v). MS (ESI+): m/z=392 [M+H]⁺; ¹H NMR (300 MHz, CDCl₃): δ (ppm) 10.25 (s, 1H), 9.95 (s, 1H), 7.21 (s, 2H), 4.95 (s, 1H), 3.64 (s, 1H), 2.79-2.63 (m, 2H), 2.42 (s, 1H), 2.23 (dd, J=21.7, 14.6 Hz, 5H), 1.63 (dd, J=15.3, 7.6 Hz, 4H), 1.50-1.21 (m, 16H). ¹³C NMR (75 MHz, CDCl₃): δ (ppm) 198.66, 157.00, 155.18, 154.77, 135.72, 129.77, 126.43, 77.16, 59.00, 58.64, 58.31, 53.77, 46.32, 37.11, 28.45, 27.51, 26.29, 24.14, 19.78, 13.71.

### AB-739—118

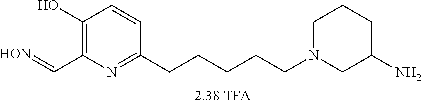

Same procedure as for AB-737 using compound 145 as starting material. Yellow solid (259 mg, 76%). ¹H NMR (300 MHz, CD₃OD) δ (ppm): 8.31 (s, 1H), 7.66 (d, J=8.7 Hz, 1H), 7.47 (d, J=8.7 Hz, 1H), 5.12 (br s, 7H), 3.68 (d, J=11.1 Hz, 1H), 3.53 (dd, J=24.5, 12.3 Hz, 2H), 3.28-3.08 (m, 2H), 3.08-2.77 (m, 4H), 2.21-1.92 (m, 2H), 1.92-1.44 (m, 7H). ¹³C NMR (75 MHz, CD₃OD) δ (ppm): 162.89 (q, J=35.3 Hz), 154.55, 150.90, 144.62, 134.04, 131.85, 127.54, 123.74, 119.87, 116.00, 112.13, 57.99, 53.72, 53.00, 46.45, 33.89, 27.44, 27.29, 24.31. HPLC (method B): t_{R}=14.97 (97.31%). HRMS (ESI+): m/z calcd. for C16H27N4O2 307.2134; found 307.2129.

### Methyl 3-(benzyloxy)-6-(4-(4-methylpiperazin-1-yl)but-1-yn-1-yl)picolinate—160

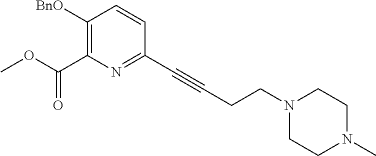

Mesylate 57b (1.28 g, 3.28 mmol, 1.1 equiv) was dissolved in dry MeCN (33 mL, 0.1 M) under Argon atmosphere and the resulting mixture was cooled down to 0° C. 1-methylpiperazine (340 μL, 2.98 mmol, 1.0 equiv.) followed by potassium carbonate (820 mg, 5.96 mmol, 2.0 equiv.) were added in this order. The resulting heterogenous mixture was reflux overnight. Salt were removed by filtration and solvent was evaporated under reduced pressure. The crude product was purified using Silica gel flash chromatography (dichloromethane/MeOH, 95/5, v/v) to give the title compound as a brown oil (509 mg, 43%). Rf=0.2 (dichloromethane/MeOH, 95/5, v/v). MS (ESI+): m/z=394 [M+H]⁺, 416 [M+Na]+, 426 [M+K]+. ¹H NMR (300 MHz, CDCl₃): δ (ppm) 7.53-7.24 (m, 7H), 5.20 (s, 2H), 3.97 (s, 3H), 2.81-2.66 (m, 2H), 2.67-2.41 (m, 10H), 2.34 (s, 3H). ¹³C NMR (75 MHz, CDCl₃): δ (ppm) 164.57, 152.67, 139.86, 135.30, 134.97, 129.74, 128.46, 127.96, 126.68, 121.55, 88.05, 79.83, 70.53, 56.40, 54.69, 52.39, 52.29, 45.63, 17.37.

### Methyl 3-hydroxy-6-(4-(4-methylpiperazin-1-yl)butyl)picolinate—161

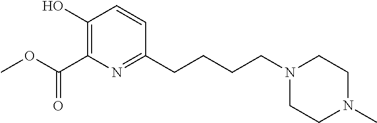

Procedure E on compound 160 (Pearlman's catalyst: 150 mg; MeOH: 200 mL: overnight; no purification). Brown oil (398 mg, quant.). Rf=0.33 (dichloromethane/MeOH, 90/10, v/v). MS (ESI+): m/z=306 [M+H]⁺, 328 [M+Na]⁺. ¹H NMR (300 MHz, CDCl₃): δ (ppm) 10.51 (s, 1H), 7.23 (s, 2H), 3.97 (s, 3H), 3.39 (s, 3H), 3.10-2.35 (br m, 10H), 1.66 (br s, 4H). ¹³C NMR (75 MHz, CDCl₃): δ (ppm) 170.08, 157.35, 129.29, 128.93, 126.89, 57.22, 53.23, 50.65, 50.24, 44.29, 36.99, 27.37.

### 3-hydroxy-6-(4-(4-methylpiperazin-1-yl)butyl)picolinaldehyde—162

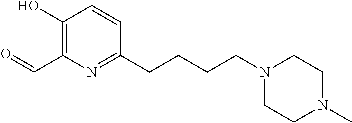 [SMILES: CN1CCN(CCCCc2ccc(O)c(C=O)n2)CC1]

Procedure F on compound 161 (DIBAL-H: 3 equiv.). Brown oil (188 mg, 58% over 3 steps). Rf=0.14 (dichloromethane/MeOH, 90/10, v/v). MS (ESI+): m/z (%)=278 (100) [M+H]⁺. ¹H NMR (300 MHz, CDCl₃): δ (ppm) 10.58 (br s, 1H), 9.96 (s, 1H), 7.22 (s, 1H), 7.19 (s, 1H), 2.82-2.64 (m, 2H), 2.43 (br s, 7H), 2.37-2.27 (m, 3H), 2.24 (s, 3H), 1.68 (dt, J=8.7, 7.3 Hz, 2H), 1.49 (dt, J=10.4, 4.9 Hz, 2H). ¹³C NMR (75 MHz, CDCl₃): δ (ppm) 198.31, 156.71, 154.37, 135.52, 129.47, 126.16, 58.03, 54.67, 52.72, 45.64, 36.84, 27.28, 26.08.

### 3-hydroxy-6-(4-(4-methylpiperazin-1-yl)butyl)picolinaldehyde oxime—AB639

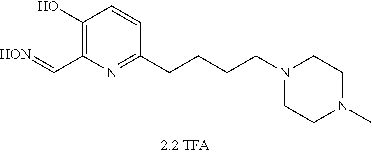

Procedure G on compound 162. Reverse phase purification (method F). White off powder (200.84 mg, 54%). ¹H NMR (300 MHz, CD₃OD) δ (ppm) 8.32 (s, 1H), 7.62 (d, J=8.7 Hz, 1H), 7.44 (d, J=8.7 Hz, 1H), 3.50 (s, 8H), 3.12 (s, 2H), 2.88 (s, 5H), 1.73 (s, 4H). ¹³C NMR (75 MHz, CD₃OD): δ (ppm) 162.64 (d, J=35.9 Hz), 154.47, 151.41, 145.67., 134.51, 131.04, 127.30, 119.69, 115.83, 57.42, 51.79, 50.03, 43.47, 34.37, 27.53, 24.58. HPLC (method A): t_{R}=8.70 (97.68%). HRMS (ESI+): m/z calcd. for C15H25N4O2 293.1978; found 293.1975.

### Methyl 3-(benzyloxy)-6-(5-(4-methylpiperazin-1-yl)pent-1-yn-1-yl)picolinate—163

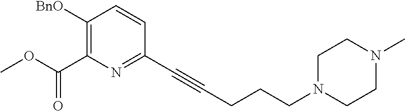

Procedure C using methyl 3-(benzyloxy)-6-(5-((methylsulfonyl)oxy)pent-1-yn-1-yl)picolinate and 1-methylpiperazine as substrates. Purification: dichloromethane/MeOH+2% triethylamine, 100/0/2 to 95/5/2, v/v/v. Orange Oil (1.73 g, 76%). Rf=0.18 (dichloromethane/MeOH+2% triethylamine, 95/5/2, v/v/v). MS (ESI+): m/z (%)=408 (100). ¹H NMR (300 MHz, CDCl₃): δ (ppm) 7.65-7.20 (m, 7H), 5.23 (s, 2H), 3.98 (s, 3H), 2.84-2.40 (m, 12H), 2.35 (s, 4H), 1.91-1.72 (m, 2H). ¹³C NMR (75 MHz, CDCl₃): δ (ppm) 164.77, 152.84, 139.83, 135.43, 135.31, 129.97, 128.66, 128.16, 126.83, 121.71, 89.89, 79.39, 70.68, 57.26, 54.90, 53.47, 52.80, 52.64, 45.80, 25.50, 17.27.

### Methyl 3-hydroxy-6-(5-(4-methylpiperazin-1-yl)pentyl)picolinate—164

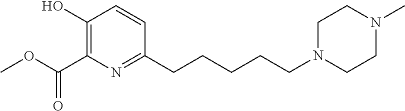

Procedure D on compound 163 (Perlman catalyst in MeOH). Brown solid (1.19 g, 97%). MS (ESI+): m/z=322 [M+H]⁺. ¹H NMR (300 MHz, CD₃OD): δ (ppm) 7.44 (d, J=8.7 Hz, 1H), 7.37 (d, J=8.6 Hz, 1H), 4.03 (s, 3H), 2.86-2.70 (m, 3H), 2.52 (d, J=24.6 Hz, 5H), 2.41 (dd, J=9.0, 6.7 Hz, 3H), 2.32 (s, 3H), 1.71 (dt, J=15.4, 7.6 Hz, 2H), 1.63-1.45 (m, 2H), 1.45-1.26 (m, 2H). ¹³C NMR (75 MHz, CD₃OD): δ (ppm) 198.84, 186.25, 183.42, 158.87, 158.20, 156.30, 87.47, 83.47, 81.61, 81.47, 77.16, 73.95, 65.83, 59.19, 56.26, 55.28.

### 3-hydroxy-6-(5-(4-methylpiperazin-1-yl)pentyl)picolinaldehyde—165

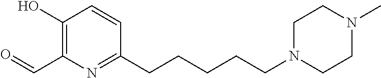 [SMILES: CN1CCN(CCCCCc2ccc(O)c(C=O)n2)CC1]

Procedure E on compound 164: 2,6-lutidine as base, DIBAL-H=3.0 equiv. Purification=silica gel, dichloromethane/MeOH/triethylamine, 92.5/2.5/2 (v/v/v). Brown reddish oil (577 mg, 61% over 3 steps). Rf=0.33 (dichloromethane/MeOH/triethylamine, 95/5/2, v/v/v). MS (ESI+): m/z=295 [M+H]⁺. ¹H NMR (300 MHz, CDCl₃): δ (ppm) 10.34 (s, 1H), 10.01 (s, 1H), 7.30 (d, J=8.7 Hz, 1H), 7.26 (d, J=8.8 Hz, 1H), 2.85-2.66 (m, 2H), 2.49 (s, 8H), 2.41-2.31 (m, 2H), 2.29 (s, 3H), 1.73 (dt, J=15.4, 7.6 Hz, 2H), 1.63-1.44 (m, 2H), 1.35 (td, J=14.8, 7.6 Hz, 2H). ¹³C NMR (75 MHz, CDCl₃): δ (ppm) 198.02, 156.51, 154.36, 135.41, 129.27, 125.98, 58.01, 54.52, 52.67, 52.57, 45.62, 45.51, 36.81, 29.16, 26.75, 26.19.

### AB-069—147

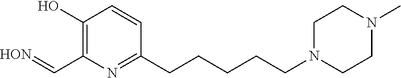

Compound 165 (422 mg, 1.09 mmol, 1.0 equiv.) was dissolved in dried MeOH (11 mL). Then, sodium acetate (369 mg, 4.46 mmol, 4.1 equiv.) was introduced followed by hydroxylamine hydrochloride (304 mg, 4.36 mmol, 4.0 equiv.). The mixture was stirred at room temperature overnight. Concentration under reduced pressure and purification by flash chromatography on silica gel (dichloromethane/MeOH/30% aq NH₄OH, 98:2:1 v/v/v) gave AB-069 as a white-off solid (201 mg, 46%). Rf=0.21 (dichloromethane/MeOH/triethylamine, 97.5/2.5/2, v/v/v). ¹H NMR (300 MHz, CD₃OD): δ (ppm) 8.28 (s, 1H), 7.26 (d, J=8.5 Hz, 1H), 7.14 (d, J=8.5 Hz, 1H, H), 2.79-2.52 (br m, 8H), 2.46 (dd, J=9.0, 6.7 Hz, 2H), 2.38 (s, 3H), 1.79-1.64 (m, 2H), 1.64-1.47 (m, 2H), 1.46-1.26 (m, 4H). ¹³C NMR (75 MHz, CD₃OD): δ (ppm) 154.6, 153.8, 152.9, 136.2, 126.0, 125.3, 59.0, 55.0, 53.1, 45.5, 37.7, 30.9, 27.9, 26.9. MS (ESI+): m/z=307 (100%) [M+H]⁺. HPLC (method A): t_{R}=19.32 min (purity: 96%).

### AB-069 HCl

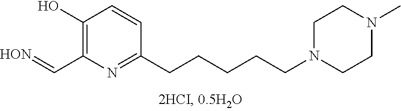

AB-069 (57.1 mg, 0.186 mmol, 1.0 equiv.) was dissolved in methanol (2.2 mL). Aqueous HCl (650 μL, 0.650 mmol, 3.5 equiv., 1.01 N) was added at rt. After 2 h, concentration to dryness and storage in vacuum desiccator gave the title compound as white off solid (53 mg, 75%). Elemental analysis calculated: N % 15.92, C % 54.61, H % 8.02; found N % 17.03, C % 58.78 H % 6.85. HPLC (method A): t_{R}=14.27 (purity: 96.68%).

### Methyl 6-bromo-3-(methoxymethoxy)picolinate—4b

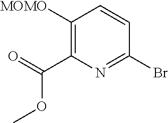

To a solution of 9a (4.84 g, 20.87 mmol) in acetone (100 mL) was slowly added potassium carbonate (5.8 g, 2 equiv.) and MOMCl (20 mL, 41.74 mmol, 2 equiv.) under argon at 0° C. The mixture was stirred heated to reflux overnight. Salts were removed by filtration and the crude was concentrated under vacuum. The desired product was obtained after purification by flash chromatography on silica gel (Petroleum ether/EtOAc, 60/40, v/v) as a colourless oil (1.70 g, 94%).

To a solution of 9a (1 g, 6.5 mmol) in DMF (65 mL) was slowly added NaH (230 mg, 9.75 mmol, 1.2 equiv.) and MOMCl (3 mL, 6.5 mmol, 1.0 equiv.) under argon at 0° C. The mixture was stirred 1 h and quenched with aq. NH₄Cl. The cooled reaction mixture was washed extracted with dichloromethane, washed with water, dried under magnesium sulfate and concentrated under reduced pressure. The desired product was obtained without further purification as a colourless oil (5.18 g, 95%). Rf=0.25 (Petroleum ether/EtOAc 60/40, v/v). ¹H NMR (300 MHz, CDCl₃) δ (ppm) 7.53 (d, J=8.8 Hz, 1H), 7.49 (d, J=8.8 Hz, 1H), 5.25 (s, 2H), 3.95 (s, 3H), 3.50 (s, 3H). ¹³C NMR (75 MHz, CDCl₃) δ (ppm) 52.7, 56.6, 95.1, 127.1, 131.3, 132.1, 140.1, 152.4, 163.9. MS (ESI+): m/z (%): 276 (85) [M+H]⁺ and 278 (100).

### Methyl 6-(4-hydroxybut-1-yn-1-yl)-3-(methoxymethoxy)picolinate—23b

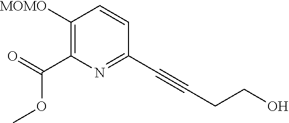

To a degassed solution of 4b (1.25 g, 4.55 mmol, 1 equiv.) in dry dichloromethane/triethylamine (2/1, v/v, 0.1M) and but-3-yn-1-ol (345 μL, 4.55 mmol, 1.0 equiv) were added Pd(PPh₃)₄ (263 mg, 0.23 mmol 0.05 equiv.) CuI (87 mg, 0.46 mmol, 0.1 equiv.) and the solution was stirred under argon at rt overnight in absence of light. The reaction mixture was concentrated under reduced pressure and purified by flash chromatography on silica gel (Petroleum ether/EtOAc, 60/40 to 30/70, v/v) to give the desired product as a yellow solid (1.13 g. 94%). Rf=0.25 (Petroleum ether/EtOAc, 40/60, v/v). ¹H NMR (300 MHz, CDCl₃) δ (ppm) 7.54 (d, J=8.8 Hz, 1H), 7.46 (d, J=8.7 Hz, 1H), 5.26 (s, 2H), 3.96 (s, 3H), 3.83 (dd, J=11.8, 5.9 Hz, 2H), 3.51 (s, 3H), 2.70 (t, J=6.3 Hz, 2H), 1.92 (s, 1H), ¹³C NMR (75 MHz, CDCl₃) δ (ppm) 164.9, 151.8, 140.5, 136.1, 130.2, 124.2, 95.1, 87.1, 81.0, 77.1, 60.9, 56.7, 52.9, 23.9. MS (ESI+): m/z (%): 266 (100) [M+H]⁺.

### Methyl 6-(4-hydroxybutyl)-3-(methoxymethoxy)picolinate—24b

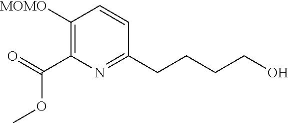

To a solution of 23b (550 mg, 1.97 mmol) in dry DMF (20 mL) were added imidazole (410 mg, 7.03 mmol, 3.5 equiv.) and TBDMSCl (460 mg, 3 mmol, 1.5 equiv.). The mixture was stirred at rt overnight. The DMF was removed and the product was dissolved in EtOAc. The organic layer was washed with brine and dried over magnesium sulfate. Concentration under reduced pressure furnished the desired product as orange oil (775 mg, quant.). ¹H NMR (300 MHz, CDCl₃) δ (ppm) 7.52 (d, J=8.8 Hz, 1H), 7.42 (d, J=8.7 Hz, 1H), 5.26 (s, 2H), 3.95 (s, 3H), 3.72 (t, J=6.0 Hz, 2H), 3.50 (s, 3H), 2.50 (t, J=7.2 Hz, 2H), 1.92-1.68 (m, 2H), 0.90 (s, 9H), 0.06 (s, 6H).

To a solution of silyl ether in deglazed MeOH was added Perlman's catalyst (155 mg, 20% w/w). The homogeneous solution was deglazed with argon, and hydrogen gas was bubbled into the solution. The reaction was stirred at rt, under hydrogen gas atm (1 atm) during 2 h. Subsequent filtration on Celite® gave the crude product as an orangeous oil (658 mg, 84%) that was used without further purification in the next step. To a solution of crude product in dry THF was added TBAF (2.2 mL, 1M in dichloromethane, 1.1 equiv) at 0° C. The mixture was stirred at rt overnight. After concentration under vacuum, the crude product was purified using flash chromatography on silica gel (EtOAc/PE, 60/40 to 90/10, v/v) to give the desired compound as yellow oil that crystallized upon standing (413 mg, 78% over 3 steps). Rf=0.5 (EtOAc/PE, 90/10, v/v). ¹H NMR (300 MHz, CDCl₃) δ (ppm) 7.50 (d, J=8.7 Hz, 1H), 7.22 (d, J=8.7 Hz, 1H), 5.22 (s, 2H), 3.95 (s, 3H), 3.65 (t, J=6.3 Hz, 2H), 3.50 (s, 3H), 2.89-2.76 (m, 2H), 1.91 (s, 1H), 1.86-1.71 (m, 2H), 1.70-1.53 (m, 2H). ¹³C NMR (75 MHz, CDCl₃) δ (ppm) 165.8, 155.1, 151.0, 139.5, 126.0, 125.1, 95.3, 62.5, 56.5, 52.7, 36.8, 32.2, 25.9. MS (ESI+): m/z (%): 270 (100) [M+H]⁺.

### Methyl 6-(4-(4-ethylpiperazin-1-yl)butyl)-3-(methoxymethoxy)picolinate—166

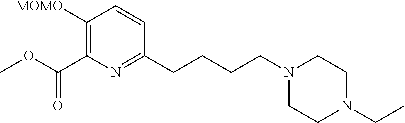

Primary alcohol 24b (1.2 g, 2.23 mmol, 1.0 equiv.) was dissolved in dry dichloromethane (0.1 M) and the resulting mixture was cooled down to 0° C. triethylamine (675 μL, 4.69 mmol, 2.1 equiv.) followed by mesyl chloride (190 μL, 2.45 mmol, 1.1 equiv.) were added dropwise in this order. The resulting mixture was stirred 3 h at rt. The crude mixture is diluted in dichloromethane, washed with water and brine. Organic layer was dried over magnesium sulfate and evaporated under reduced pressure. The residue was dissolved in dry MeCN (0.1M). N-ethylpiperazine (314 μL, 2.45 mmol, 1.1 equiv.) and potassium carbonate (619 mg, 4.46 mmol, 2 equiv.) were added in this order. The resulting heterogenous mixture was reflux overnight. Salt were removed by filtration and solvent was evaporated under reduced pressure. The crude product was purified (silica gel, dichloromethane/MeOH, 95/5, v/v) to give the title compound as a brown oil (253 mg, 31%). Rf=0.21 (dichloromethane/MeOH, 95/5, v/v). MS (ESI+): m/z=366 [M+H]⁺, 388 [M+Na]⁺. ¹H NMR (300 MHz, CDCl₃): δ (ppm) 7.43 (d, J=8.6 Hz, 1H), 7.15 (d, J=8.6 Hz, 1H), 5.15 (s, 2H), 3.89 (s, 3H), 3.43 (s, 3H), 2.79-2.60 (m, 2H), 2.59-2.19 (m, 10H), 1.76-1.55 (m, 2H), 1.47 (dt, J=15.1, 7.4 Hz, 2H), 1.01 (t, J=7.2 Hz, 3H). ¹³C NMR (75 MHz, CDCl₃): δ (ppm) 165.81, 155.12, 150.74, 139.64, 125.73, 124.89, 95.23, 58.38, 56.41, 53.03, 52.64, 52.62, 52.26, 37.20, 27.90, 26.38, 11.83.

### Methyl 6-(4-(4-ethylpiperazin-1-yl)butyl)-3-hydroxypicolinate—167

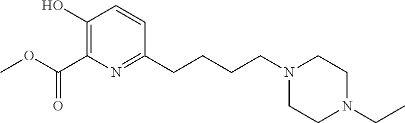 [SMILES: CCN1CCN(CCCCc2ccc(O)c(C(=O)OC)n2)CC1]

Compound 166 (252 mg, 0.69 mmol, 1 equiv.) in dry dichloromethane (14 mL, 0.05 M) and TFA (3.8 mL, 49 mmol, 72 equiv.) was added at 0° C. and the reaction was stirred at this temperature during 2 h. The crude was evaporated until dryness and purified on silica gel (dichloromethane/MeOH, 100/0 to 90/10, v/v) to give the title product as an orange oil (47 mg, 22%). Rf=0.5 (dichloromethane/MeOH, 90/10, v/v). MS (ESI+): m/z=322 [M+H]⁺, 344 [M+Na]⁺. ¹H NMR (300 MHz, CDCl₃): δ (ppm) 10.50 (s, 1H), 7.22 (s, 2H), 3.97 (s, 3H), 2.93-2.60 (m, 2H), 2.60-2.08 (m, 12H), 1.73-1.57 (m, 2H), 1.57-1.40 (m, 2H), 1.04 (t, J=7.2 Hz, 3H). ¹³C NMR (75 MHz, CDCl₃): δ (ppm) 170.18, 157.26, 153.82, 129.20, 128.86, 126.71, 58.36, 53.50, 53.19, 52.86, 52.53, 52.30, 37.46, 27.94, 26.36, 11.73.

### AB-588—148

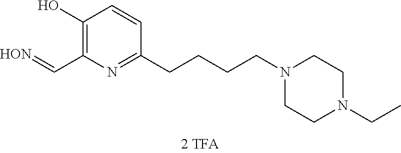

AB-588 oxime was synthetized using procedure G on compound 167 and was purified using reverse phase purification (method D) to afford the title compound as white off powder (45.4 mg, 75%). ¹H NMR (300 MHz, CD₃OD): δ (ppm) 8.32 (s, 1H), 7.58 (d, J=8.7 Hz, 1H), 7.41 (d, J=8.7 Hz, 1H), 3.53 (br s, 8H), 3.24-3.10 (m, 4H), 2.86 (br s, 2H), 1.74 (br s, 4H), 1.30 (t, J=7.3 Hz, 3H). ¹³C NMR (75 MHz, CD₃OD): δ (ppm) 162.86 (q, J=35.3 Hz). 154.40, 151.71, 146.73, 134.74, 130.37, 127.04, 123.71, 119.84, 115.98, 112.11, 57.47, 53.10, 49.99, 49.62, 34.70, 27.56, 24.55, 9.49. HPLC (method B): t_{R}=24.31 min (purity: 98.90%). HRMS (ESI+): m/z calcd. for C16H27N4O2 307.2134; found 307.2132.

### Methyl 3-(benzyloxy)-6-(5-(4-ethylpiperazin-1-yl)pent-1-yn-1-yl)picolinate—168

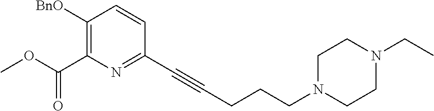 [SMILES: CCN1CCN(CCCC#Cc2ccc(OCc3ccccc3)c(C(=O)OC)n2)CC1]

Procedure D was used on methyl 3-(benzyloxy)-6-(5-(tosyloxy)pent-1-yn-1-yl)picolinate using 1-ethylpiperazine (purification: silica gel, dichloromethane/MeOH/triethylamine, 95/5/1, v/v/v) to give the title compound as a brown oil (1.12 g, 92%). Rf=0.29 (dichloromethane/MeOH/triethylamine, 95/5/1, v/v/v). MS (ESI+): m/z=422 [M+H]⁺. ¹H NMR (300 MHz, CDCl₃): δ (ppm) 7.55-7.25 (m, 7H), 5.23 (s, 2H), 3.98 (s, 3H), 2.68-2.39 (br m, 12H), 1.92-1.73 (m, 2H), 1.33 (t, J=7.3 Hz, 2H), 1.15 (t, J=7.2 Hz, 1H). ¹³C NMR (75 MHz, CDCl₃): δ (ppm) 164.93, 152.98, 140.19, 135.63, 135.58, 130.03, 128.82, 128.32, 127.01, 126.00, 121.90, 90.05, 79.59, 70.95, 57.46, 52.85, 52.75, 52.72, 52.39, 46.16, 25.67, 17.46, 11.75, 9.29.

### Methyl 6-(5-(4-ethylpiperazin-1-yl)pentyl)-3-hydroxypicolinate—169

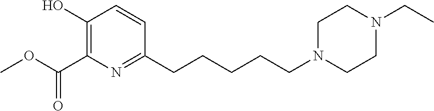 [SMILES: CCN1CCN(CCCCCc2ccc(O)c(C(=O)OC)n2)CC1]

Procedure E (Pearlman's catalyst, MeOH) was used on compound 168 and the crude product was purified using silica gel (dichloromethane/MeOH, 95/5 to 90/10 v/v) to give the title compound as an orange oil (446 mg, 49%). Rf=0.38 (dichloromethane/MeOH, 90/10 v/v). MS (ESI+): m/z=336 [M+H]⁺. ¹H NMR (300 MHz, CDCl₃): δ (ppm) 10.52 (s, 1H), 7.22 (s, 2H), 3.97 (s, 3H), 2.98-2.53 (m, 12H), 2.53-2.32 (m, 2H), 1.79-1.42 (m, 4H), 1.42-1.23 (m, 2H), 1.23-1.04 (m, 3H). ¹³C NMR (75 MHz, CDCl₃): δ (ppm) 170.65, 157.74, 154.30, 129.69, 129.34, 129.30, 127.22, 126.45, 58.28, 53.69, 52.59, 51.89, 37.98, 30.28, 27.42.

### AB-557—149

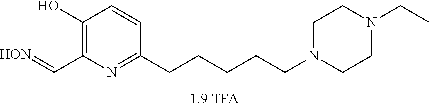

Procedure F (DIBAL=3 equiv.) and procedure G (Reverse phase purification, method D) were used on compound 169 to give the title compound as a brown solid (69.9 mg, 10% over 4 steps). ¹H NMR (300 MHz, CD₃OD): δ (ppm) 8.31 (s, 1H), 7.58 (d, J=8.6 Hz, 1H), 7.40 (d, J=8.4 Hz, 1H), 3.25-3.08 (m, 8H), 2.91-2.73 (m, 2H), 1.71 (s, 8H), 1.38 (s, 2H), 1.28 (t, J=7.0 Hz, 3H). ¹³C NMR (75 MHz, CD₃OD): δ (ppm) 161.63 (q, J=35.4 Hz), 154.31, 134.52, 130.51, 127.08, 119.91, 116.06, 57.68, 53.08, 35.08, 32.83, 30.22, 29.55, 26.90, 26.72, 24.84. HPLC (method B): t_{R}=14.52 min (96.1%). HRMS (ESI+): m/z calcd. for C17H29N4O2 321.2291; found 321.2287.

### 1-(but-3-yn-1-yl)-4-phenylpiperazine—170

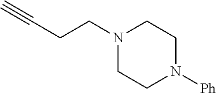 [SMILES: C#CCCN1CCN(c2ccccc2)CC1]

Procedure D was used on but-3-yn-1-yl 4-methanesulfonate (1 equiv.) with N-phenylpiperazine (1 equiv.) and potassium carbonate (1.1 equiv.), normal phase purification (Petroleum ether/EtOAc, 100/0 to 1/1, v/v) gave the title compound as a yellow oil (829 mg, 78%). Rf=0.28 (Petroleum ether/EtOAc, 80/40, v/v). ¹H NMR (300 MHz, CD₂Cl₂): δ (ppm) 7.23 (dd, J=8.7, 7.3 Hz, 2H), 6.90 (d, J=8.0 Hz, 2H), 6.82 (t, J=7.3 Hz, 1H), 3.24-3.08 (m, 4H), 2.70-2.58 (m, 6H), 2.41 (td, J=7.4, 2.4 Hz, 2H), 2.01 (t, J=2.7 Hz, 1H). ¹³C NMR (75 MHz, CD₂Cl₂): δ (ppm) 152.06, 129.55, 119.91, 116.40, 83.40, 71.09, 69.25, 68.06, 57.50, 53.49, 49.58, 38.06, 20.26, 17.37.

### Methyl 3-(benzyloxy)-6-(4-(4-phenylpiperazin-1-yl)but-1-yn-1-yl)picolinate—171

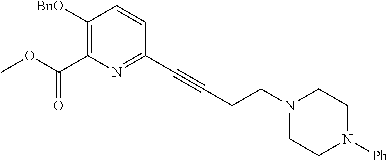 [SMILES: COC(=O)c1nc(C#CCCN2CCN(c3ccccc3)CC2)ccc1OCc1ccccc1]

Procedure A was used with compounds 170 and 4a (purification on silica gel, Petroleum ether/EtOAc/triethylamine, 70/30/1 to 50/50/1, v/v/v), affording the title product as an orange oil (505 mg, 54%, trace of PPh₃). Rf=0.28 (Petroleum ether/EtOAc/triethylamine, 50/50/1, v/v/v). ¹H NMR (300 MHz, CD₂Cl₂): δ (ppm) 7.31-7.05 (m, 9H), 6.76 (d, J=8.6 Hz, 2H), 6.67 (t, J=7.3 Hz, 1H), 4.98 (s, 2H), 3.76 (s, 3H), 3.11-2.94 (m, 4H), 2.49-2.40 (m, 4H), 2.36 (t, J=7.1 Hz, 4H), 1.75-1.60 (m, 2H). ¹³C NMR (75 MHz, CD₂Cl₂): δ (ppm) 165.35, 153.25, 152.02, 140.62, 136.28, 135.69, 132.46, 132.38, 132.33, 130.31, 129.46, 129.10, 129.07, 128.91, 128.66, 127.59, 122.10, 119.68, 116.21, 90.23, 79.89, 71.19, 60.67, 57.68, 53.68, 52.82, 49.48, 26.33, 21.27, 17.56, 14.53.

### Methyl 3-hydroxy-6-(4-(4-phenylpiperazin-1-yl)butyl)picolinate—172

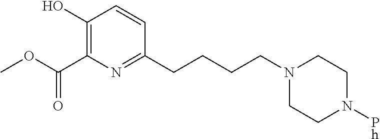 [SMILES: COC(=O)c1nc(CCCCN2CCN(c3ccccc3)CC2)ccc1O]

Procedure E (Perlmaan 0.4 equiv. in methanol, 0.005M) was used on compound 172 to give the title compound without purification needed as a light orange oil (178 mg, 99%). ¹H NMR (300 MHz, CDCl₃): δ (ppm) 10.53 (s, 1H), 7.19 (dd, J=11.0, 4.9 Hz, 4H), 6.91-6.67 (m, 3H), 3.96 (s, 3H), 3.23-3.05 (m, 4H), 2.79-2.69 (m, 2H), 2.66-2.53 (m, 4H), 2.50-2.36 (m, 2H), 1.76-1.59 (m, 2H), 1.60-1.41 (m, 2H). ¹³C NMR (75 MHz, CDCl₃): δ (ppm) 170.15, 157.29, 153.67, 151.07, 129.26, 129.21, 128.87, 128.68, 128.52, 126.80, 120.07, 116.28, 58.26, 53.20, 53.06, 48.81, 37.32, 29.75, 27.83, 25.94, 3-hydroxy-6-(4-(4-phenylpiperazin-1-yl)butyl)picolinaldehyde—173

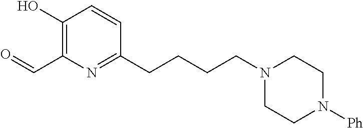 [SMILES: O=Cc1nc(CCCCN2CCN(c3ccccc3)CC2)ccc1O]

Procedure F (DIBAL=3 equiv.) was used on compound 172. The title compound was isolated as an orange oil (67 mg, 38% over 3 steps) using normal phase purification (dichloromethane/MeOH, 100/0 to 85/15, v/v). Rf=0.62 (dichloromethane/MeOH, 90/10). ¹H NMR (300 MHz, CDCl₃): δ (ppm) 10.08 (s, 1H), 7.42-7.19 (m, 4H), 6.97 (d, J=8.1 Hz, 2H), 6.89 (t, J=7.3 Hz, 1H), 3.37-3.14 (m, 4H), 2.87 (t, J=7.6 Hz, 2H), 2.77-2.57 (m, 4H), 2.57-2.42 (m, 2H), 1.96-1.72 (m, 2H), 1.72-1.55 (m, 2H). ¹³C NMR (75 MHz, CDCl₃): δ (ppm) 198.81, 157.07, 154.78, 151.36, 135.78, 129.81, 129.16, 126.45, 119.75, 116.09, 58.49, 53.35, 49.15, 37.22, 27.65, 26.49.

### AB-277—150

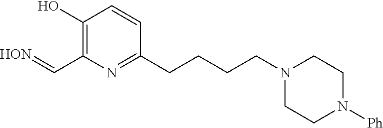

Procedure G was used on compound 173; purification using silica gel (dichloromethane/MeOH, 95/5 to 90/10, v/v) gave the title product as light orange solid (59 mg, 92%). Rf=0.5 (dichloromethane/MeOH, 90/10). ¹H NMR (300 MHz, CD₂Cl₂): δ (ppm) 9.19 (br s, 2H), 8.27 (s, 1H), 7.25 (t, J=8.0 Hz, 2H), 7.14 (d, J=8.5 Hz, 1H), 7.02 (d, J=8.5 Hz, 1H), 6.96-6.82 (m, 3H), 3.43-3.24 (m, 4H), 3.04-2.83 (m, 3H). 2.75 (t, J=6.1 Hz, 4H), 1.85-1.65 (m, 4H), 1.26 (t, J=6.0 Hz, 3H). ¹³C NMR (75 MHz, CD₂Cl₂): δ (ppm) 154.05, 153.09, 151.15, 135.61, 129.71, 125.43, 124.85, 124.49, 120.91, 116.97, 58.22, 52.94, 48.24, 36.77, 27.58, 24.86, 21.52. HPLC (method A): t_{R}=18.85 (purity=96.45%). MS (ESI+): m/z=355 [M+H]⁺

### AB-745—150a

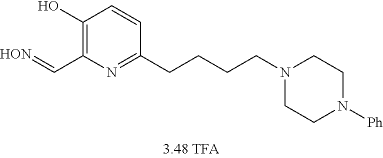

AB-277 (58 mg, 0.164 mmol, 1.0 equiv.) was dissolved in dry methanol (1 mL, 0.1 M), and TFA (1 mL) was added. Concentration until dryness and reverse phase purification gave the title compound as golden white off solid (57.25 mg, 46%). ¹H NMR (300 MHz, CD₃OD): δ (ppm) 8.42 (s, 1H), 7.75 (d, J=8.7 Hz, 1H), 7.56 (d, J=8.7 Hz, 1H), 7.35-7.23 (m, 2H), 7.01 (d, J=7.9 Hz, 2H), 6.93 (t, J=7.3 Hz, 1H), 3.98-3.48 (m, 4H), 3.29-2.86 (m, 8H). ¹³C NMR (75 MHz, CD₃OD): δ (ppm) 162.48 (d, J=35.9 Hz), 154.46, 151.30, 151.12, 145.60, 134.44, 131.11, 130.33, 127.32, 122.36, 119.80, 117.98, 115.94, 57.44, 53.06, 48.02, 34.32, 27.59, 24.36. HPLC (method B): t_{R}=19.42 min (purity: 96.91%). MS (ESI+): m/z=355 [M+H]⁺, 386 [M+K]⁺. HRMS (ESI+): m/z calcd. for C20H27N4O2 355.2134; found 355.2148.

### 1-(pent-4-yn-1-yl)-4-phenylpiperazine—174

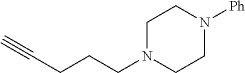 [SMILES: C#CCCCN1CCN(c2ccccc2)CC1]

Procedure D was used on but-3-yn-1-yl 4-methanesulfonate (1 equiv.) with N-phenylpiperazine (1 equiv.) and potassium carbonate (1.1 equiv.); normal phase purification (Petroleum ether/EtOAc, 100/0 to 1/1, v/v) gave the title compound as a yellow oil (989 mg, 87%). Rf=0.25 (Petroleum ether/EtOAc, 80/40, v/v). ¹H NMR (300 MHz, CD₂Cl₂): δ (ppm) 7.23 (dd, J=8.7, 7.3 Hz, 2H), 6.90 (d, J=8.0 Hz, 2H), 6.81 (t, J=7.3 Hz, 1H), 3.22-3.07 (m, 4H), 2.62-2.51 (m, 4H), 2.46 (t, J=7.1 Hz, 2H), 2.26 (td, J=7.1, 2.6 Hz, 2H), 1.98 (t, J=2.7 Hz, 1H), 1.72 (p, J=7.1 Hz, 2H). ¹³C NMR (75 MHz, CD₂Cl₂): δ (ppm) 152.15, 129.53, 119.79, 116.32, 84.89, 69.95, 69.11, 68.67, 57.61, 53.80, 49.63, 37.68, 28.44, 26.47, 16.75.

### Methyl 3-(benzyloxy)-6-(5-(4-phenylpiperazin-1-yl)pent-1-yn-1-yl)picolinate—175

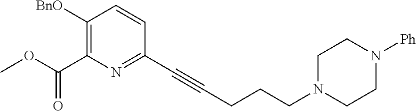 [SMILES: COC(=O)c1nc(C#CCCCN2CCN(c3ccccc3)CC2)ccc1OCc1ccccc1]

Procedure A was used with compounds 174 and 4a, purification on silica gel (Petroleum ether/EtOAc/triethylamine, 70/30/1 to 50/50/1, v/v/v) afforded the title product as an orange oil (283 mg, 32%, trace of PPh₃). Rf=0.28 (Petroleum ether/EtOAc/triethylamine, 50/50/1, v/v/v). ¹H NMR (300 MHz, CD₂Cl₂): δ (ppm) 7.52-7.19 (m, 9H), 7.00-6.79 (m, 3H), 5.15 (s, 2H), 3.94 (s, 3H), 3.32-3.15 (m, 4H), 2.71-2.57 (m, 4H), 2.53 (t, J=7.1 Hz, 4H), 1.85 (p, J=7.2 Hz, 2H), 1.38-1.17 (m, 1H). ¹³C NMR (75 MHz, CD₂Cl₂): δ (ppm) 165.35, 153.25, 152.02, 140.62, 136.28, 135.69, 132.46, 132.38, 132.33, 130.31, 129.46, 129.10, 129.07, 128.91, 128.66, 127.59, 122.10, 119.68, 116.21, 90.23, 79.89, 71.19, 57.68, 53.68, 52.82, 49.48, 26.33, 17.56.

### Methyl 3-hydroxy-6-(5-(4-phenylpiperazin-1-yl)pentyl)picolinate—176

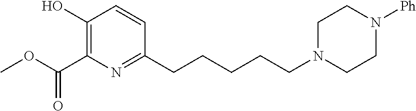 [SMILES: COC(=O)c1nc(CCCCCN2CCN(c3ccccc3)CC2)ccc1O]

Procedure E (Pearlman's catalyst: 0.4 equiv., methanol as solvent, 0.006 M) was used on compound 175 to give the title compound without purification needed as an orange oil (173 mg, quant.). ¹H NMR (300 MHz, CDCl₃): δ (ppm) 7.17 (dd, J=8.3, 7.6 Hz, 4H), 6.84 (d, J=8.2 Hz, 2H), 6.76 (t, J=7.2 Hz, 1H), 3.94 (s, 3H), 3.25-3.02 (m, 4H), 2.82-2.62 (m, 2H), 2.62-2.42 (m, 4H), 2.42-2.20 (m, 2H), 1.65 (dt, J=15.5, 7.7 Hz, 2H), 1.49 (dt, J=15.0, 7.4 Hz, 2H), 1.32 (dt, J=14.6, 7.4 Hz, 2H). ¹³C NMR (75 MHz, CDCl₃): δ (ppm) 170.18, 157.45, 153.95, 151.41, 132.21, 132.08, 129.13, 128.63, 128.47, 126.79, 119.66, 116.04, 58.67, 53.35, 53.18, 49.18, 37.68, 30.10, 27.38, 26.84.

### 3-hydroxy-6-(5-(4-phenylpiperazin-1-yl)pentyl)picolinaldehyde—177

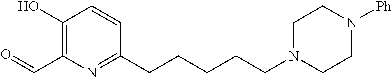 [SMILES: O=Cc1nc(CCCCCN2CCN(c3ccccc3)CC2)ccc1O]

Procedure F (2,6-lutidine, DIBAL=3 equiv.) was used on compound 176 that was isolated using normal phase purification (dichloromethane/MeOH, 100/0 to 90/10, v/v) to give the title compound as a white off solid (40 mg, 50% over 3 steps). Rf=0.55 (dichloromethane/MeOH, 90/10). ¹H NMR (300 MHz, CDCl₃): δ (ppm) 10.38 (s, 1H), 10.00 (s, 1H), 7.31-7.15 (m, 4H), 6.93-6.86 (m, 2H), 6.82 (dd, J=11.4, 4.1 Hz, 1H), 3.33-3.08 (m, 4H), 2.85-2.67 (m, 2H), 2.67-2.46 (m, 4H), 2.37 (dd, J=8.7, 6.6 Hz, 2H), 1.73 (dt, J=15.5, 7.6 Hz, 2H), 1.57 (dt, J=15.2, 7.3 Hz, 2H), 1.46-1.30 (m, 2H). ¹³C NMR (75 MHz, CDCl₃): δ (ppm) 198.89, 157.09, 155.04, 151.44, 135.82, 132.28, 132.15, 129.84, 129.20, 128.69, 128.53, 126.47, 119.77, 116.12, 58.71, 53.40, 49.21, 37.41, 29.77, 27.35, 26.83.

### AB-269—151

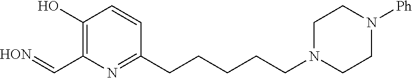

Procedure G was used for compound 177 and the title product was purified by flash chromatography on silica gel (dichloromethane/MeOH, 100/0 to 90/10, v/v) as a a white off solid (29 mg, 70%). Rf=0.2 (dichloromethane/MeOH, 95/5, v/v). ¹H NMR (300 MHz, CD₂Cl₂): δ (ppm) 10.60 (s, 2H), 8.24 (s, 1H), 7.15 (dd, J=8.5, 7.4 Hz, 2H), 7.06 (d, J=8.4 Hz, 1H), 6.91 (d, J=8.4 Hz, 1H), 6.82 (d, J=8.0 Hz, 2H), 6.75 (t, J=7.3 Hz, 1H), 3.28-3.07 (m, 4H), 2.80-2.55 (m, 6H), 2.53-2.36 (m, 2H), 1.72-1.43 (m, 4H), 1.37-1.24 (m, 2H). ¹³C NMR (75 MHz, CD₂Cl₂): δ (ppm) 153.79, 153.73, 153.04, 151.54, 135.78, 129.63, 124.86, 124.26, 120.48, 116.73, 58.70, 53.32, 48.81, 37.40, 30.24, 27.42, 26.14. HPLC (method A): t_{R} 19.63 (98.60%)

### AB-744—151a

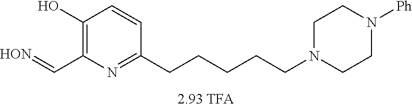

Same procedure used for AB-745 on AB-269; white off solid (57.4 mg, 49%). ¹H NMR (300 MHz, CD₃OD): δ (ppm) 8.29 (s, 1H), 7.72 (d, J=8.8 Hz, 1H), 7.51 (d, J=8.8 Hz, 1H), 7.13 (t, J=8.0 Hz, 2H), 6.86 (d, J=8.0 Hz, 2H), 6.77 (t, J=7.3 Hz, 1H), 3.66 (d, J=12.1 Hz, 2H), 3.51 (d, J=9.7 Hz, 2H), 3.13-2.73 (m, 8H), 1.78-1.54 (m, 4H), 1.45-1.22 (m, 2H). ¹³C NMR (75 MHz, CD₃OD): δ (ppm) 160.5 (q, J=36 Hz), 153.17, 149.84, 149.75, 141.68, 132.15, 131.51, 128.95, 126.59, 121.01, 116.62, 56.27, 51.67, 46.68, 32.48, 28.75, 25.55, 23.24. HPLC (method B): t_{R}=20.00 (95.30%). MS (ESI+): m/z=369 (100) [M+H], 401 (5) [M+K]+. HRMS (ESI+): m/z calcd. for C21H29N4O2 369.2291; found 369.2291.

### Methyl 3-(benzyloxy)-6-(5-(4-benzylpiperazin-1-yl)pent-1-yn-1-yl)picolinate—173

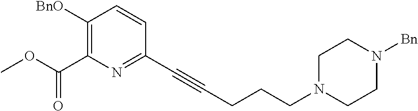 [SMILES: COC(=O)c1nc(C#CCCCN2CCN(Cc3ccccc3)CC2)ccc1OCc1ccccc1]

Procedure D was used for mesylate 57a and N-benzylpiperazine, purification by flash chromatography on silica gel (dichloromethane/MeOH/triethylamine, 100/0/1 to 95/5/1, v/v/v) to give the title compound as an orange oil (870 mg, 68%). Rf=0.68 (dichloromethane/MeOH/triethylamine, 90/10/1, v/v/v). ¹H NMR (300 MHz, CDCl₃): δ (ppm) 7.53-7.10 (m, 13H), 5.13 (s, 2H), 3.92 (s, 3H), 3.49 (s, 2H), 2.53-2.31 (m, 12H), 1.91-1.67 (m, 2H). ¹³C NMR (75 MHz, CDCl₃): δ (ppm) 164.46, 152.44, 139.59, 137.83, 135.19, 135.01, 129.63, 128.78, 128.32, 127.82, 126.62, 126.51, 121.36, 89.69, 79.13, 70.28, 62.69, 57.11, 52.84, 52.75, 52.25, 25.36, 17.00. MS (ESI+): m/z=484 (100) [M+H]⁺.

### Methyl 6-(5-hydroxypent-1-yn-1-yl)-3-(methoxymethoxy)picolinate—23a

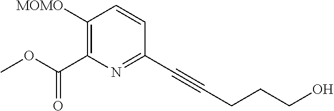

Procedure A was used on 4-pentyn-1-ol and 4b. Purification by flash chromatography on silica gel (Petroleum ether/EtOAc, 75/25 to 20/80, v/v) gave the title compound as a yellow solid (1.13 mg, 80%). Rf=0.15 (Petroleum ether/EtOAc, 60/40, v/v). ¹H NMR (300 MHz, CDCl₃): δ (ppm) 7.52 (d, J=8.7 Hz, 1H), 7.43 (d, J=8.8 Hz, 1H), 5.26 (s, 2H), 3.95 (s, 3H), 3.80 (dd, J=11.6, 6.0 Hz, 2H), 3.50 (s, 3H), 2.55 (t, J=7.0 Hz, 2H), 1.98-1.77 (m, 2H), 1.56 (s, 1H), 1.43 (t, J=5.3 Hz, 1), 1.27 (d, J=8.5 Hz, 1H), 0.84 (d, J=7.1 Hz, 1H). ¹³C NMR (75 MHz, CDCl₃): δ (ppm) 164.91, 151.89, 140.51, 136.11, 130.22, 124.20, 95.13, 87.10, 81.07, 77.36, 60.95, 56.76, 52.90, 23.96. MS (ESI+) m/z=280 [M+H]⁺.

### Methyl 6-(5-hydroxypentyl)-3-(methoxymethoxy)picolinate—24a

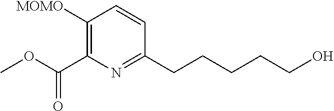

To a solution of 23b (562 mg, 2.01 mmol, 1 equiv.) in dry DMF (20 mL, 0.1 M) were added imidazole (411 mg, 7.05 mmol, 3.5 equiv.) and TBDMSCl (463 mg, 3.02 mmol, 1.5 equiv.). The mixture was stirred at rt overnight. The DMF was removed and the product was dissolved in EtOAc. The organic layer was washed with brine and dried over magnesium sulfate. Concentration under reduced pressure furnished the intermediate silyl ether as orange oil that was used without further purification.

To a solution of silyl ether in degassed MeOH was added Perlman's catalyst (158 mg, 20% w/w). The heterogeneous solution was degassed with argon, and hydrogen gas was bubbled into the solution. The reaction was stirred at rt, under hydrogen gas atm (1 atm) during 2 h. Subsequent filtration on Celite® gave the crude product reduced product as an orange oil that was used without further purification in the next step.

To a solution of reduced silyl ether in dry THF was added TBAF (2.2 mL, 1 M in dichloromethane, 1.1 equiv.) at 0° C. The mixture was stirred at rt overnight. After concentration under vacuum, the crude product was purified using flash chromatography on silica gel (EtOAc/PE, 60/40 to 90/10, v/v) to give the desired compound as light-yellow oil (342 mg, 60% over 3 steps). Rf=0.25 (Petroleum ether/EtOAc, 40/60, v/v). ¹H NMR (300 MHz, CDCl₃): δ (ppm) 7.49 (d, J=8.7 Hz, 1H), 7.21 (d, J=8.7 Hz, 1H), 5.20 (s, 2H), 3.92 (s, 3H), 3.58 (t, J=6.5 Hz, 2H), 3.47 (s, 3H), 2.87-2.69 (m, 2H), 1.81-1.61 (m, 2H), 1.61-1.49 (m, 2H), 1.49-1.32 (m, 2H). ¹³C NMR (75 MHz, CDCl₃): δ (ppm) 165.47, 154.89, 150.21, 139.17, 125.42, 124.52, 94.77, 61.76, 55.95, 52.16, 36.81, 32.06, 29.39, 25.11. MS (ESI+): m/z=284 [M+H]+.

### Methyl 6-(5-(4-benzylpiperazin-1-yl)pentyl)-3-(methoxymethoxy)picolinate—179

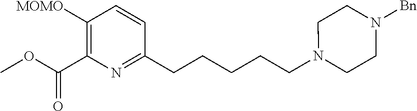

Procedure B was used on 24a to generate the mesylate that was used without purification on the next steps (procedure D) with N-benzyl morpholine (1.2 equiv.) to give the title compound after purification by flash chromatography on silica gel (dichloromethane/MeOH/triethylamine, 100/0/2 to 90/10/2, v/v/v) as an orangeous oil (446 mg, 86%). Rf=0.55 (dichloromethane/MeOH/triethylamine, 90/10/2, v/v/v). ¹H NMR (300 MHz, CDCl₃): δ (ppm) 7.54 (d, J=8.6 Hz, 1H), 7.36-7.23 (m, 6H), 5.25 (s, 2H), 3.98 (s, 3H), 3.53 (s, 3H), 2.86-2.73 (m, 3H), 2.53 (br s, 10H), 2.44-2.31 (m, 2H), 1.86-1.66 (m, 2H), 1.63-1.52 (m, 2H). ¹³C NMR (75 MHz, CDCl₃): δ (ppm) 166.29, 155.50, 150.57, 140.52, 138.56, 132.31, 129.46, 128.99, 128.54, 127.40, 125.82, 125.16, 95.71, 62.96, 58.37, 56.59, 53.22, 52.63, 46.23, 37.45, 30.00, 27.30, 26.28. MS (ESI+): m/z=442 (100%) [M+H]⁺.

### Methyl 6-(5-(4-benzylpiperazin-1-yl)pentyl)-3-hydroxypicolinate—180

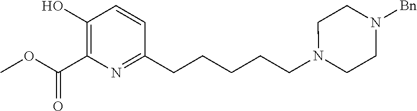 [SMILES: COC(=O)c1nc(CCCCCN2CCN(Cc3ccccc3)CC2)ccc1O]

Compound 179 (404 mg, 0.915 mmol, 1 equiv.) was dissolved in dry dichloromethane and TFA was added at 0° C. The mixture was stirred at rt during 2 h, then excess of TFA was removed under vacuum. The residue was dissolved in dichloromethane and washed with saturated aqueous Na₂CO₃. The organic phase was dried with magnesium sulfate and removing of volatiles gave the desired product as an orange oil (233 mg, 64%). Rf=0.57 (dichloromethane/MeOH/triethylamine, 95/5/2, v/v/v). ¹H NMR (300 MHz, CDCl₃): δ (ppm) 7.47-7.20 (m, 7H), 4.07 (s, 2H), 3.56 (s, 3H), 2.96-2.71 (m, 2H), 2.71-2.26 (m, 10H), 1.92-1.69 (m, 2H), 1.69-1.51 (m, 2H), 1.51-1.34 (m, 2H), 1.06-0.81 (m, 1H). MS (ESI+): 398 [M+H]⁺.

### 6-(5-(4-benzylpiperazin-1-yl)pentyl)-3-hydroxypicolinaldehyde—181

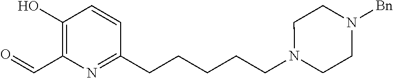 [SMILES: O=Cc1nc(CCCCCN2CCN(Cc3ccccc3)CC2)ccc1O]

Procedure F was used on compound 180 (2,6-lutidine as base; DIBAL-H=3 equiv.) and after a normal phase purification (dichloromethane/MeOH, 100/0 to 95/5, v/v/v) the title compound was obtained as yellow oil (94 mg, 43% over 3 steps). ¹H NMR (300 MHz, CDCl₃): δ (ppm) 9.85 (s, 1H), 7.13 (dd, J=7.0, 3.0 Hz, 7H), 3.39 (s, 2H), 2.71-2.22 (m, 12H), 1.62-1.42 (m, 4H), 1.26-1.15 (m, 3H). ¹³C NMR (75 MHz, CDCl₃) δ (ppm) 198.74, 157.00, 154.71, 137.36, 135.71, 129.83, 129.28, 128.35, 127.35, 126.42, 62.64, 58.11, 52.76, 51.78, 37.09, 29.36, 26.90, 25.63. HRMS (ESI+): m/z calcd. for C22H30N3O2 368.2338; found 368.2338.

### AB-153—153

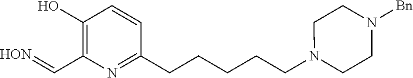

Procedure G was used on compound 181 to give after normal phase purification (dichloromethane/MeOH, 90/10, v/v) the title product as a yellow oil that crystalize upon standing (78.2 mg, 82%). ¹H NMR (300 MHz, CD₃OD): δ (ppm) 8.08 (s, 1H), 7.16-7.03 (m, 6H), 6.94 (d, J=8.5 Hz, 1H), 3.45 (s, 2H), 2.94 (s, 4H), 2.74 (dd, J=9.5, 6.8 Hz, 2H), 2.64-2.48 (m, 6H), 1.75 (s, 4H), 1.60-1.45 (m, 4H), 1.26-1.13 (m, 2H). ¹³C NMR (75 MHz, CD₃OD): δ (ppm) 154.28, 153.77, 152.75, 137.43, 136.27, 130.49, 129.53, 128.83, 126.04, 125.37, 62.73, 57.91, 52.90, 51.11, 37.43, 30.56, 27.22, 25.15, 22.38. MS (ESI+): m/z (%)=383 (100) [M+H]⁺. HPLC (method A): t_{R}=17.87 min (98.4%).

### Methyl 3-(benzyloxy)-6-(4-(4-benzylpiperazin-1-yl)but-1-yn-1-yl)picolinate—152

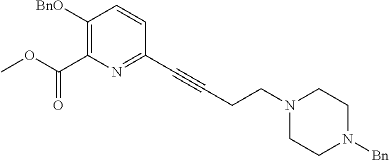 [SMILES: COC(=O)c1nc(C#CCCN2CCN(Cc3ccccc3)CC2)ccc1OCc1ccccc1]

Procedure D was used on mesylate 57b (1.1 equiv.), potassium carbonate (2.2 equiv.) and N-benzylpiperazine (1 equiv.), purification by chromatography on silica gel (dichloromethane/MeOH, 100/0 to 95/5, v/v) gave the title product as an orange oil (714 mg, 46%). Rf=0.28 (dichloromethane/MeOH, 96/4, v/v). ¹H NMR (300 MHz, CDCl₃): δ (ppm) 7.45-7.19 (m, 12H), 5.20 (s, 2H), 3.95 (s, 3H), 3.51 (s, 2H), 2.74-2.64 (m, 2H), 2.64-2.27 (m, 10H). ¹³C NMR (75 MHz, CDCl₃): δ (ppm) 164.67, 152.80, 139.97, 137.98, 135.41, 135.14, 132.00, 131.87, 131.82, 129.83, 129.05, 128.59, 128.48, 128.32, 128.09, 126.91, 126.78, 121.66, 88.31, 79.92, 70.66, 62.89, 56.65, 52.85, 52.69, 52.52, 17.46. MS (ESI+): m/z=470 [M+H]⁺, 492 [M+Na]⁺, 502 [M+K]⁺.

### Methyl 6-(4-(4-benzylpiperazin-1-yl)butyl)-3-hydroxypicolinate—183

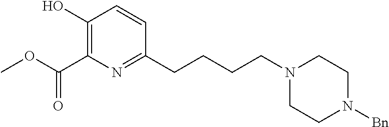 [SMILES: COC(=O)c1nc(CCCCN2CCN(Cc3ccccc3)CC2)ccc1O]

Procedure E was used on compound 182. After normal phase purification, the title compound was obtained as a brown oil (335 mg, 58%, trace of toluene). Rf=0.5 (dichloromethane/MeOH, 90/10, v/v). ¹H NMR (300 MHz, CDCl₃): δ (ppm) 7.32-7.20 (m, 7H), 4.00 (s, 3H), 3.49 (s, 3H), 2.88-2.67 (m, 2H), 2.62-2.23 (m, 10H), 1.79-1.63 (m, 2H), 1.60-1.45 (m, 2H). ¹³C NMR (75 MHz, CDCl₃): δ (ppm) 170.17, 157.26, 153.80, 137.94, 132.21, 129.28, 128.84, 128.64, 128.27, 127.15, 63.00, 58.37, 53.18, 53.13, 52.75, 37.42, 27.94, 26.23. MS (ESI+): m/z=384 [M+H]⁺.

### 6-(4-(4-benzylpiperazin-1-yl)butyl)-3-hydroxypicolinaldehyde—184

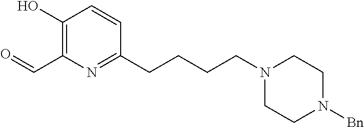 [SMILES: O=Cc1nc(CCCCN2CCN(Cc3ccccc3)CC2)ccc1O]

Procedure F (triethylamine as base, DIBAL-H=3 equiv.) was used on compound 183 and the title compound was isolated after normal phase purification (dichloromethane/MeOH, 100/0 to 90/10, v/v) as a brown oil (216 mg, 70% over 3 steps. TLC-MS (ESI+): m/z=354 [M+H]⁺, 376 [M+Na]⁺; Rf=0.33. ¹H NMR (300 MHz, CDCl₃): δ (ppm) 10.49 (br s, 1H), 9.95 (s, 1H), 7.37-7.03 (m, 7H), 3.44 (s, 2H), 2.86-2.62 (m, 2H), 2.60-2.18 (m, 8H), 1.78-1.57 (m, 2H), 1.57-1.36 (m, 2H). ¹³C NMR (75 MHz, CDCl₃): δ (ppm) 198.53, 156.86, 154.53, 137.82, 135.61, 129.62, 129.11, 128.10, 126.96, 126.26, 62.87, 58.21, 53.02, 52.70, 36.97, 27.44, 26.14.

### AB-641—152

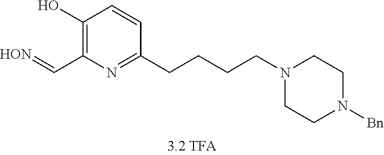

Procedure G was used on compound 184, reverse phase purification (method D) gave the title compound as white off powder (189.84 mg, 42%). ¹H NMR (300 MHz, CD₃OD): δ (ppm) 8.29 (s, 1H), 7.66 (d, J=8.7 Hz, 1H), 7.46 (d, J=8.8 Hz, 1H), 7.41-7.25 (m, 5H), 4.17 (s, 2H), 3.44 (s, 4H), 3.35 (d, J=4.1 Hz, 4H), 3.10 (t, J=7.2 Hz, 2H), 2.85 (t, J=6.9 Hz, 2H), 1.81-1.56 (m, 4H). ¹³C NMR (75 MHz, CD₃OD): δ (ppm) 162.33 (q, J=36.5 Hz), 154.59, 150.88, 144.19, 134.08, 131.99, 131.25, 130.96, 130.28, 127.67, 61.55, 57.33, 50.47, 49.71, 33.81, 27.42, 24.37. HPLC (method B): t_{R}=17.28 min (purity: 97%). HRMS (ESI+): m/z calcd. for C21H19N4O3 369.2291; found 329.2287.

### Methyl 6-(4-(4-benzhydrylpiperazin-1-yl)but-1-yn-1-yl)-3-(benzyloxy)picolinate—185

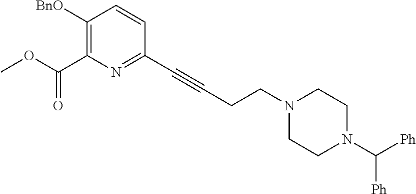 [SMILES: COC(=O)c1nc(C#CCCN2CCN(C(c3ccccc3)c3ccccc3)CC2)ccc1OCc1ccccc1]

Procedure D was used on mesylate 57b (1.1 equiv.), potassium carbonate (2 equiv) and N-benzhydrylpiperazine (1 equiv.); purification by chromatography on silica gel (dichloromethane/MeOH, 100/0 to 95/5, v/v) gave the title product as brown foaming solid (767 mg, 43%). TLC-MS (ESI+): m/z=546 [M+H]⁺; Rf=0.4 (dichloromethane/MeOH, 96/4, v/v); ¹H NMR (300 MHz, CDCl₃): δ (ppm) 7.44-7.13 (m, 17H), 5.19 (s, 2H), 4.21 (s, 1H), 3.95 (s, 3H), 2.75-2.63 (br m, 2H), 2.63-2.22 (m, 10H). ¹³C NMR (75 MHz, CDCl₃): δ (ppm) 164.60, 152.70, 142.53, 139.88, 135.35, 135.02, 133.04, 131.91, 131.78, 131.75, 131.66, 129.76, 128.50, 128.41, 128.29, 128.25, 127.99, 127.71, 126.73, 126.70, 121.56, 88.26, 79.87, 75.99, 70.52, 56.55, 52.89, 52.42, 51.61, 17.41.

### Methyl 6-(4-(4-benzhydrylpiperazin-1-yl)butyl)-3-hydroxypicolinate—186

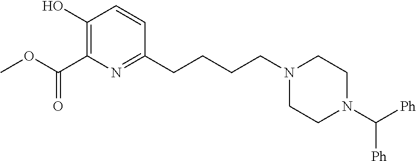 [SMILES: COC(=O)c1nc(CCCCN2CCN(C(c3ccccc3)c3ccccc3)CC2)ccc1O]

Procedure E (Pearlman s catalyst, Me H) was use on compound 185 to give after normal phase purification (dichloromethane/MeOH, 100/0 to 90/10) the title compound as a brown oil (574 mg, 89%). Rf=0.52 (dichloromethane/MeOH, 90/10, v/v). ¹H NMR (300 MHz, CDCl₃): δ (ppm) 7.36-7.03 (m, 12H), 4.12 (s, 1H), 3.90 (s, 3H), 2.76-2.58 (m, 2H), 2.58-2.06 (m, 10H), 1.72-1.52 (m, 2H), 1.52-1.31 (m, 2H). ¹³C NMR (75 MHz, CDCl₃): δ (ppm) 170.07, 157.14, 153.77, 142.73, 132.11, 131.98, 131.92, 131.89., 129.12, 128.74, 128.60, 128.55, 128.41, 127.88, 126.91, 126.85, 126.58, 76.22, 70.96, 58.37, 53.45, 53.07, 51.78, 37.38, 37.08, 27.94, 26.33.

### 6-(4-(4-benzhydrylpiperazin-1-yl)butyl)-3-hydroxypicolinaldehyde—187

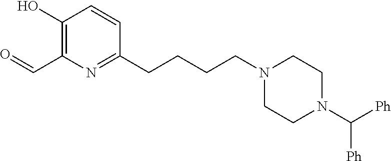 [SMILES: O=Cc1nc(CCCCN2CCN(C(c3ccccc3)c3ccccc3)CC2)ccc1O]

Procedure F (triethylamine as base, DIBAL-H=3 equiv.) was used on compound 186, normal phase purification (dichloromethane/MeOH, 100/0 to 90/10, v/v, gave the title compound as an orange oil (499.7 mg, 93% over 3 steps). TLC-MS (ESI+): m/z=430 [M+H]⁺; Rf=0.5 (dichloromethane/MeOH, 90/10, v/v). ¹H NMR (300 MHz, CDCl₃): δ (ppm) 10.52 (s, 1H), 9.92 (s, 1H), 7.35-7.02 (m, 12H), 4.12 (s, 1H), 2.77-2.58 (m, 2H), 2.51-1.89 (m, 10H), 1.73-1.53 (m, 2H), 1.53-1.35 (m, 2H). ¹³C NMR (75 MHz, CDCl₃): δ (ppm) 198.43, 156.80, 154.47, 142.57, 131.97, 131.84, 131.79, 129.56, 128.61, 128.44, 128.31, 128.28, 127.75, 126.90, 126.76, 126.18, 76.04, 58.17, 53.35, 53.30, 51.56, 36.91, 27.40, 26.12.

### AB-643—154

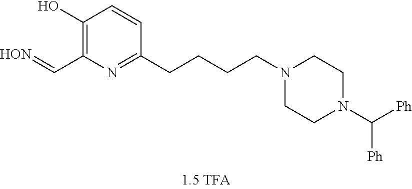

Procedure G was used on compound 187, reverse phase purification (method A) gave the title compound as a white off powder (444.1 mg, 62%). ¹H NMR (300 MHz, CD₃OD): δ (ppm) 8.42 (s, 1H), 7.78 (t, J=10.6 Hz, 1H), 7.58 (t, J=8.9 Hz, 1H), 7.53-7.39 (m, 5H), 7.33 (t, J=7.3 Hz, 4H), 7.24 (t, J=7.3 Hz, 2H), 4.52 (d, J=8.9 Hz, 1H), 3.51-3.25 br (m, 6H), 3.19 (br s, 3H), 2.98 (br s, 3H), 2.78 (br s, 4H). ¹³C NMR (75 MHz, CD₃OD): δ (ppm) 161.65 (q, J=38.1 Hz), 154.59, 150.88, 144.21, 142.20, 134.07, 132.04, 129.93, 128.89, 128.77, 127.65, 119.37, 115.52, 76.37, 57.26, 53.08, 49.87, 33.87, 27.52, 24.38. HPLC (method B): t_{R}=24.55 min (purity: 97.99%). HRMS (ESI+): m/z calcd. for C27H33N4O2 445.2604; found 445.2611.

### Methyl 6-(5-(4-benzhydrylpiperazin-1-yl)pent-1-yn-1-yl)-3-(benzyloxy)picolinate—188

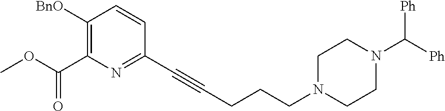 [SMILES: COC(=O)c1nc(C#CCCCN2CCN(C(c3ccccc3)c3ccccc3)CC2)ccc1OCc1ccccc1]

Procedure D was used on mesylate 57a (1.1 equiv.), potassium carbonate (2 equiv.) and N-benzhydrylpiperazine (1.0 equiv.), purification on silica gel (dichloromethane/MeOH, 100/0 to 95/5, v/v) gave the title product as yellow foaming solid (2.21 g, 60%). TLC-MS (ESI+): m/z=560 [M+H]⁺, 582 [M+Na]⁺. Rf=0.30 (dichloromethane/MeOH, 98/2, v/v). ¹H NMR (300 MHz, CDCl₃): δ (ppm) 7.73-7.16 (m, 17H), 5.24 (s, 2H), 4.36 (s, 1H), 4.04 (s, 3H), 2.77-2.43 (m, 12H), 1.98-1.83 (m, 2H). ¹³C NMR (75 MHz, CDCl₃): δ (ppm) 164.66, 152.63, 142.67, 139.92, 135.41, 135.27, 131.94, 131.80, 131.75, 129.76, 128.52, 128.42, 128.29, 128.01, 127.74, 126.73, 121.58, 89.90, 79.34, 76.08, 70.57, 57.32, 53.37, 53.30, 52.42, 51.76, 25.55, 17.24.

### Methyl 6-(5-(4-benzhydrylpiperazin-1-yl)pentyl)-3-hydroxypicolinate—189

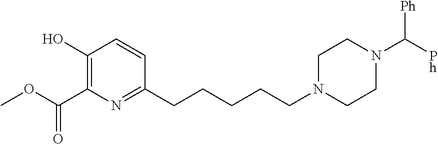 [SMILES: COC(=O)c1nc(CCCCCN2CCN(C(c3ccccc3)c3ccccc3)CC2)ccc1O]

Procedure E (Pearlman's catalyst, MeOH) was used for compound 188 to give the title compound as a yellowish oil (1.69 g, quant.). ¹H NMR (300 MHz, CDCl₃): δ (ppm) 7.34, 7.34, 7.32, 7.22, 7.21, 7.19, 7.19, 7.17, 7.14, 7.13, 7.12, 7.12, 7.10, 4.19, 3.96, 2.73, 2.71, 2.68, 2.60, 2.48, 2.46, 2.43, 2.40, 1.69, 1.66, 1.64, 1.61, 1.59, 1.56, 1.54, 1.51, 1.48, 1.46, 1.34, 1.32, 1.29, 1.27, 1.24, 1.18, 13C NMR (75 MHz, CDCl₃): δ (ppm) 170.21, 157.28, 153.90, 142.50, 141.18, 132.23, 132.10, 129.26, 129.00, 128.87, 128.64, 128.52, 127.91, 127.14, 126.74, 126.13, 76.03, 58.21, 53.21, 50.96, 42.01, 37.52, 29.85, 27.11, 25.83.

### 6-(5-(4-benzhydrylpiperazin-1-yl)pentyl)-3-hydroxypicolinaldehyde—190

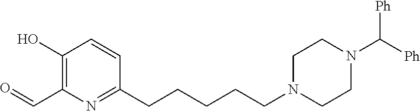 [SMILES: O=Cc1nc(CCCCCN2CCN(C(c3ccccc3)c3ccccc3)CC2)ccc1O]

Procedure F (triethylamine as base, DIBAL-H=3.0 equiv.) was used on compound 189 to give after normal phase purification (dichloromethane/MeOH, 100/0 to 90/10, v/v) the desired product as brown oil (763 mg, 66% over 3 steps). TLC-MS (ESI+): m/z=444 [M+H]⁺. Rf=0.55 (dichloromethane/MeOH, 90/10, v/v); ¹H NMR (300 MHz, CDCl₃): δ (ppm) 9.94 (s, 1H), 7.95 (br s, 1H), 7.41-7.27 (m, 51H), 7.22-6.99 (m, 7H), 4.16 (s, 1H), 2.74-2.59 (m, 2H), 2.59-2.12 (br m, 10H), 1.73-1.56 (m, 2H), 1.56-1.36 (m, 3H), 1.36-1.16 (m, 2H). ¹³C NMR (75 MHz, CDCl₃): δ (ppm) 198.31, 156.68, 154.50, 142.48, 135.48, 131.89, 131.76, 129.41, 128.35, 128.22, 127.66, 126.66, 126.09, 75.97, 58.23, 53.17, 51.41, 36.91, 29.30, 26.96, 26.19, 25.65.

### AB-555—155

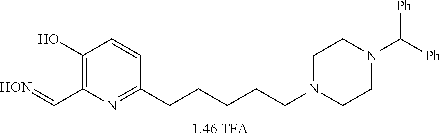

Procedure G was used on compound 190, reverse phase purification (method A) gave the title compound as a yellow solid (416 mg, 39%). ¹H NMR (300 MHz, CD₃OD): a (ppm) 8.36 (s, 1H), 7.70 (d, J=8.7 Hz, 1H), 7.50 (d, J=8.7 Hz, 1H), 7.43 (dd, J=5.2, 3.4 Hz, 4H), 7.32-7.22 (m, 4H), 7.22-7.12 (m, 2H), 4.41 (s, 1H), 3.44-3.22 (m, 8H), 3.13-3.04 (m, 2H), 2.92-2.85 (m, 2H), 1.80-1.62 (m, 4H), 1.51-1.31 (m, 2H). ¹³C NMR (75 MHz, CD₃OD): δ (ppm) 161.92 (q, J=37.3 Hz), 154.45, 151.74, 144.70, 142.54, 134.04, 131.75, 129.88, 128.88, 128.67, 127.53, 123.40, 119.54, 115.70, 76.34, 57.56, 53.24, 49.89, 49.00, 34.45, 30.16, 26.95, 24.67. HPLC (method B): t_{R} 24.75 (98.08%). HRMS (ESI+): m/z calcd. for C28H35N4O4 459.2760; found 459.2774.

### Methyl 3-(methoxymethoxy)-6-(4-(4-(pyridin-2-yl)piperazin-1-yl)butyl)picolinate—191

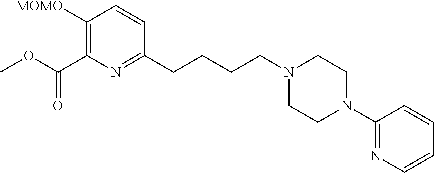

Procedure B was applied to compound 24b to generate the activated mesylate quantitatively that was used without purification. Then, procedure D was used on the mesylate (1 equiv.) and the 1-(pyridin-2-yl)piperazine (1.1 equiv.) with potassium carbonate (2 equiv.). Normal phase purification (dichloromethane/MeOH, 100/0 to 90/10, v/v) gave the title product as brown oil (428 mg, 46% over 2 steps). TLC-MS (ESI+): m/z=415 [M+H]⁺, 437 [M+Na]⁺. Rf=0.37 (dichloromethane/MeOH, 90/10, v/v). ¹H NMR (300 MHz, CDCl₃) δ (ppm) 8.12 (dd, J=4.9, 1.1 Hz, 1H), 7.47-7.35 (m, 2H), 7.18 (d, J=8.7 Hz, 1H), 6.63-6.51 (m, 2H), 5.17 (s, 2H), 3.90 (s, 3H), 3.49 (dd, J=10.2, 5.3 Hz, 4H), 3.45 (s, 2H), 2.81-2.70 (m, 2H), 2.48 (dd, J=9.3, 4.3 Hz, 4H), 2.42-2.30 (m, 2H), 1.78-1.60 (m, 2H), 1.60-1.43 (m, 2H). ¹³C NMR (75 MHz, CDCl₃) δ (ppm) 165.82, 159.53, 155.08, 150.70, 147.90, 139.70, 137.42, 128.43, 125.69, 124.87, 113.22, 95.21, 58.48, 56.40, 54.83, 53.47, 53.03, 52.63, 45.12, 37.20, 27.89, 26.38.

### Methyl 3-hydroxy-6-(4-(4-(pyridin-2-yl)piperazin-1-yl)butyl)picolinate—192

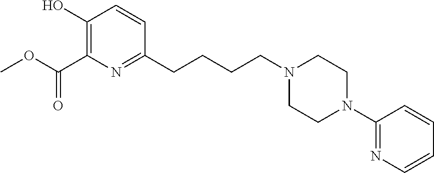 [SMILES: COC(=O)c1nc(CCCCN2CCN(c3ccccn3)CC2)ccc1O]

Compound 191 (428 mg, 1.03 mmol, 1.0 equiv.) was dissolved in dry dichloromethane (20 mL, 0.05 M). TFA (5 mL) was added dropwise at 0° C. and the mixture was stirred for 2 h at rt. Concentration to dryness and subsequent normal phase purification (dichloromethane/MeOH, 100/0 to 90/10, v/v) gave the title compound as a brown oil (116 mg, 31%). TLC-MS (ESI+): m/z=371 [M+H]⁺, 393[M+Na]⁺. Rf=0.59 (dichloromethane/MeOH, 90/10, v/v). ¹H NMR (300 MHz, CDCl₃): δ (ppm) 10.46 (br s, 1H), 8.10 (dd, J=4.8, 1.2 Hz, 1H), 7.49-7.30 (m, 1H), 7.22 (s, 2H), 6.63-6.41 (m, 2H), 3.95 (s, 3H), 3.53-3.40 (m, 4H), 2.83-2.66 (m, 2H), 2.56-2.42 (m, 4H), 2.42-2.22 (m, 2H), 1.77-1.60 (m, 2H), 1.60-1.42 (m, 2H). ¹³C NMR (75 MHz, CDCl₃): δ (ppm) 170.14, 159.51, 157.23, 153.80, 147.96, 137.46, 129.19, 128.83, 126.69, 113.32, 107.07, 58.47, 53.16, 53.03, 45.09, 37.43, 27.93, 26.33.

### AB-589—156

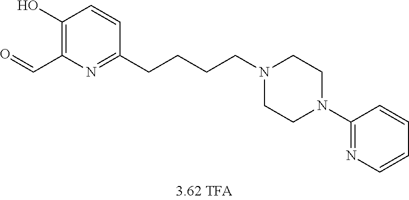 [SMILES: O=Cc1nc(CCCCN2CCN(c3ccccn3)CC2)ccc1O]

Procedure F (triethylamine, DIBAL-H=4 equiv.) was applied to compound 192 to generate the 3-hydroxy-6-(4-(4-(pyridin-2-yl)piperazin-1-yl)butyl)picolinaldehyde that was isolated with impurities using normal phase purification. TLC-MS(ESI+): m/z=341 [M+H]⁺, 363 [M+Na]⁺. Rf=0.33 (dichloromethane/MeOH, 90/10, v/v). The crude aldehyde was used without further purification in the oxime generation (procedure G). Subsequent reverse phase purification (method A) gave the title product as a tan solid (71.21 mg, 27% over 4 steps). ¹H NMR (300 MHz, CD₃OD): δ (ppm) 8.42 (s, 1H), 8.13 (dd, J=5.9, 1.2 Hz, 1H), 8.02 (ddd, J=9.0, 7.2, 1.8 Hz, 1H), 7.73 (d, J=8.7 Hz, 1H), 7.55 (d, J=8.7 Hz, 1H), 7.34 (d, J=9.1 Hz, 1H), 7.07 (dd, J=9.6, 3.4 Hz, 1H), 4.01 (s, 4H), 3.52 (s, 4H), 3.32-3.24 (m, 2H), 2.99 (t, J=7.0 Hz, 2H), 1.98-1.73 (m, 4H). ¹³C NMR (75 MHz, CD₃OD): δ (ppm) 161.13 (q, J=37.6 Hz), 154.10, 153.12, 149.87, 144.15, 142.94, 140.03, 133.03, 129.87, 125.98, 114.50, 111.55, 56.20, 50.63, 43.00, 32.87, 26.13, 22.90. HRMS (ESI+): m/z calcd. for C19H26N5O2 356.2087; found 356.2088. HPLC (method B): t_{R}=15.58 min (purity: 97.09%).

### Methyl 3-(benzyloxy)-6-(5-(4-(pyridin-2-yl)piperazin-1-yl)pent-1-yn-1-yl)picolinate—193

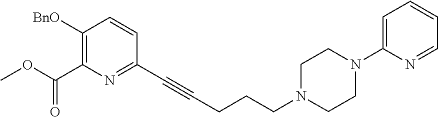 [SMILES: COC(=O)c1nc(C#CCCCN2CCN(c3ccccn3)CC2)ccc1OCc1ccccc1]

Procedure D was used on tosylate 59a (1.0 equiv.) and the 1-(pyridin-2-yl)piperazine (1.1 equiv.) with potassium carbonate (2.0 equiv.). Normal phase purification (dichloromethane/MeOH, 99/1 to 85/15, v/v) gave the title compound as a brown oil (1.04 g, 72%). TLC-MS (ESI+): m/z=471 [M+H]⁺, 493 [M+Na]⁺; Rf=0.29 (dichloromethane/MeOH, 95/5, v/v). ¹H NMR (300 MHz, CDCl₃): δ (ppm) 8.25-8.13 (m, 1H), 7.54-7.19 (m, 8H), 6.71-6.51 (m, 2H), 5.18 (s, 2H), 3.95 (s, 3H), 3.59-3.48 (m, 4H), 2.63-2.52 (m, 4H), 2.52-2.44 (m, 4H), 1.97-1.71 (m, 2H).

### Methyl 3-hydroxy-6-(5-(4-(pyridin-2-yl)piperazin-1-yl)pentyl)picolinate—194

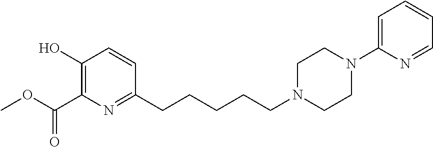 [SMILES: COC(=O)c1nc(CCCCCN2CCN(c3ccccn3)CC2)ccc1O]

Procedure E (Pearlman's catalyst, MeOH) was used on compound 193 to give the title compound without purification as a brown oil (796 mg, 95%). ¹H NMR (300 MHz, CDCl₃): δ (ppm) 10.36 (br s, 1H), 8.18 (d, J=3.7 Hz, 1H), 7.52 (t, J=7.3 Hz, 1H), 7.31 (s, 2H), 6.82-6.53 (m, 2H), 4.04 (s, 3H), 3.91 (br s, 4H), 3.06 (br s, 2H), 2.92-2.69 (m, 4H), 2.62 (br s, 2H), 1.87 (br s, 2H), 1.83-1.71 (m, 2H), 1.48-1.33 (m, 2H). ¹³C NMR (75 MHz, CDCl₃): δ (ppm) 169.85, 160.30, 158.13, 156.96, 153.24, 147.74, 137.67, 129.06, 126.48, 114.18, 107.24, 57.42, 52.92, 51.56, 43.00, 36.98, 29.20, 26.32, 24.06, 20.22, 18.78.

### 3-hydroxy-6-(5-(4-(pyridin-2-yl)piperazin-1-yl)pentyl)picolinaldehyde—195

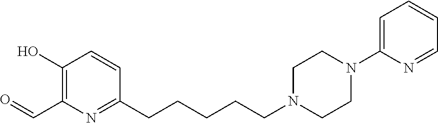 [SMILES: O=Cc1nc(CCCCCN2CCN(c3ccccn3)CC2)ccc1O]

Procedure F (triethylamine as base and DIBAL=4 equiv.) was applied to compound 194 to give after normal phase purification (dichloromethane/MeOH, 100/0 to 90/10, v/v) the title compound as a brown oil (311 mg, 44% over 3 steps, traces of TBAF remaining). Rf=0.56 (dichloromethane/MeOH, 90/10, v/v). ¹H NMR (300 MHz, CDCl₃): δ (ppm) 9.96 (s, 1H), 8.08 (dd, J=4.9, 1.3 Hz, 1H), 7.37 (ddd, J=7.2, 5.7, 2.0 Hz, 1H), 7.31-7.12 (m, 2H), 6.66-6.41 (m, 2H), 3.57-3.36 (m, 4H), 2.81-2.62 (m, 2H), 2.60-2.40 (m, 4H), 2.40-2.23 (m, 2H), 1.65-1.52 (m, 2H+TBAF), 1.37 (dt, J=14.7, 7.4 Hz, 2H+TBAF). ¹³C NMR (75 MHz, CDCl₃): δ (ppm) 197.72, 159.13, 156.69, 154.40, 147.54, 137.16, 135.58, 129.39, 126.20, 112.99, 106.80, 58.22, 52.63, 44.69, 36.92, 29.28, 26.84, 26.16, 19.43.

### AB-451—157

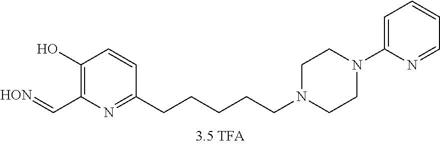

Procedure G was used on compound 195 and reverse phase purification gave the title compound as a brown oil (6.51 mg, 1%). ¹H NMR (300 MHz, CD₃OD): δ (ppm) 8.38 (s, 1H), 8.14 (dd, J=5.5, 1.1 Hz, 1H), 8.00-7.82 (m, 1H), 7.65 (d, J=8.7 Hz, 1H), 7.48 (d, J=8.7 Hz, 1H), 7.19 (d, J=8.9 Hz, 1H), 7.06-6.85 (m, 1H), 3.46 (s, 4H), 3.22-3.15 (m, 2H), 2.97-2.80 (m, 2H), 1.89-1.74 (m, 4H), 1.57-1.39 (m, 2H), 1.39-1.21 (m, 4H). ¹³C NMR (75 MHz, CD₃OD) δ (ppm) 161.13 (q, J=37.8 Hz), 157.00, 154.34, 152.29, 146.40, 144.10, 142.66, 134.56, 130.60, 127.12, 116.04, 111.71, 57.88, 52.28, 49.00, 44.26, 35.08, 30.25, 26.95, 24.67. HRMS (ESI+): m/z calcd. for C20H28N5O2 370.2235; found 370.2243. HPLC (method B): t_{R}=16.15 min (purity=95.5%).

### Methyl 3-(benzyloxy)-6-(4-(4-(pyrimidin-2-yl)piperazin-1-yl)but-1-yn-1-yl)picolinate—196

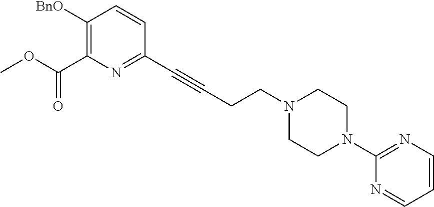 [SMILES: COC(=O)c1nc(C#CCCN2CCN(c3ncccn3)CC2)ccc1OCc1ccccc1]

Procedure D was used with mesylate 57b (1.2 equiv.) and 2-(piperazin-1-yl)pyrimidine (1.0 equiv.) with potassium carbonate (2.0 equiv.). Normal phase purification (dichloromethane/MeOH, 100/0 to 90/10, v/v) gave the title compound as a brown oil (658 mg, 48%). TLC-MS (ESI+): m/z=458 [M+H]⁺, 480 [M+Na]⁺. Rf=0.5 (dichloromethane/MeOH, 90/10, v/v). ¹H NMR (300 MHz, CDCl₃): δ (ppm) 8.34 (d, J=4.7 Hz, 2H), 7.52-7.23 (m, 8H), 6.51 (t, J=4.7 Hz, 1H), 5.22 (s, 2H), 3.99 (s, 3H), 3.92-3.80 (m, 4H), 2.80-2.72 (m, 2H), 2.72-2.64 (m, 2H), 2.64-2.50 (m, 4H). ¹³C NMR (75 MHz, CDCl₃): δ (ppm) 164.62, 161.43, 157.51, 152.77, 139.91, 135.35, 135.02, 131.79, 129.81, 128.53, 128.44, 128.28, 128.04, 126.74, 121.62, 109.70, 88.12, 79.96, 70.60, 56.65, 52.52, 52.46, 43.42, 17.48.

### Methyl 3-hydroxy-6-(4-(4-(pyrimidin-2-yl)piperazin-1-yl)butyl)picolinate—197

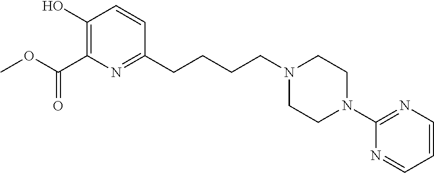 [SMILES: COC(=O)c1nc(CCCCN2CCN(c3ncccn3)CC2)ccc1O]

Procedure E (Pearlman's catalyst, MeOH) was applied to compound 196 that was isolated using normal phase purification (dichloromethane/MeOH, 100/0 to 90/10, v/v) to obtain the title compound as a brown oil (104 mg, 19%). TLC-MS (ESI+): m/z=372 [M+H]⁺, 394 [M+Na]⁺Rf=0.5 (dichloromethane/MeOH, 90/10, v/v). ¹H NMR (300 MHz, CDCl₃): δ (ppm) 8.32 (d, J=4.7 Hz, 2H), 7.33 (s, 2H), 6.49 (t, J=4.7 Hz, 1H), 4.06 (s, 3H), 3.93-3.76 (m, 4H), 2.91-2.78 (m, 2H), 2.56-2.49 (m, 4H), 2.49-2.36 (m, 2H), 1.87-1.69 (m, 2H), 1.69-1.51 (m, 2H). ¹³C NMR (75 MHz, CDCl₃): δ (ppm) 170.10, 161.64, 157.68, 157.19, 153.77, 129.13, 126.65, 109.79, 58.46, 53.13, 53.06, 43.57, 37.41, 27.91, 26.34.

### 3-Hydroxy-6-(4-(4-(pyrimidin-2-yl)piperazin-1-yl)butyl)picolinaldehyde—198

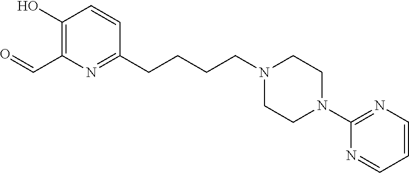 [SMILES: O=Cc1nc(CCCCN2CCN(c3ncccn3)CC2)ccc1O]

Procedure F (triethylamine as base, DIBAL-H=3 equiv.) was used on compound 197. Purification by flash chromatography on silica gel (dichloromethane/MeOH, 100/0 to 90/10, v/v) gave the title compound as light-yellow solid (93 mg, 98% over 3 steps). TLC-MS (ESI+): m/z=342 [M+H]⁺, 364 [M+Na]⁺; Rf=0.5 (dichloromethane/MeOH, 90/10, v/v). ¹H NMR (300 MHz, CDCl₃): δ (ppm) 10.33 (s, 1H), 9.96 (s, 1H), 8.22 (d, J=4.7 Hz, 2H), 7.24 (d, J=8.7 Hz, 1H), 7.21 (d, J=8.8 Hz, 1H), 6.40 (t, J=4.7 Hz, 1H), 3.90-3.65 (m, 4H), 2.75 (t, J=7.6 Hz, 2H), 2.56-2.41 (m, 4H), 2.41-2.27 (m, 2H), 1.81-1.63 (m, 2H), 1.54 (dt, J=9.7, 6.9 Hz, 2H). ¹³C NMR (75 MHz, CDCl₃): δ (ppm) 198.80, 161.71, 157.78, 157.08, 154.74, 135.80, 129.82, 126.48, 109.95, 58.55, 53.17, 43.61, 37.19, 27.61, 26.37.

### AB-647—158

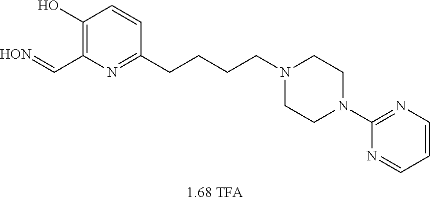

Procedure G was applied to compound 198, reverse phase purification met od A) gave the title product as a white off solid (67.31 mg, 45%). ¹H NMR (300 MHz, CD₃OD): δ (ppm) 8.16 (s, 1H), 8.12 (d, J=4.8 Hz, 2H), 7.59 (d, J=8.8 Hz, 1H), 7.38 (d, J=8.8 Hz, 1H), 6.44 (t, J=4.8 Hz, 1H), 4.61 (br s, 2H), 3.34 (br s, 4H), 3.00-2.85 (m, 3H), 2.83-2.64 (m, 3H), 1.73-1.42 (m, 4H). ¹³C NMR (75 MHz, CD₃OD): δ (ppm) 162.12, 159.24, 154.59, 150.89, 144.36, 133.98, 132.01, 127.66, 112.71, 57.56, 52.70, 42.02, 33.84, 27.51, 24.29. HRMS (ESI+): m/z calcd. for C18H25N6O2 357.2039; found 357.2040. HPLC (method B): t_{R}=17.23 min (purity=95.6%).

### Methyl 3-(benzyloxy)-6-(5-(4-(pyrimidin-2-yl)piperazin-1-yl)pent-1-yn-1-yl)picolinate—199

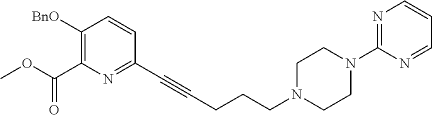 [SMILES: COC(=O)c1nc(C#CCCCN2CCN(c3ncccn3)CC2)ccc1OCc1ccccc1]

Procedure D was used with mesylate 57a (1.0 equiv.) and 2-(piperazin-1-yl)pyrimidine (1.0 equiv.) with potassium carbonate (2 equiv.). Normal phase purification (dichloromethane/MeOH, 100/0 to 90/10, v/v) gave the title compound as a brown oil (4.15 g, 78%). Rf=0.4 (dichloromethane/MeOH, 95/5, v/v). ¹H NMR (300 MHz, CD₂Cl₂): δ (ppm) 8.28 (d, J=4.7 Hz, 2H), 7.53-7.22 (m, 7H), 6.46 (t, J=4.7 Hz, 1H), 5.16 (s, 2H), 3.92 (s, 3H), 3.86-3.68 (m, 4H), 2.56-2.42 (m, 8H), 1.92-1.73 (m, 2H). ¹³C NMR (75 MHz, CD₂Cl₂): δ (ppm) 165.39, 162.36, 158.14, 153.33, 140.68, 136.32, 135.79, 130.34, 129.17, 128.74, 127.67, 122.17, 110.22, 90.25, 79.86, 71.34, 57.82, 53.60, 52.89, 44.23, 26.32, 17.59.

### Methyl 3-hydroxy-6-(5-(4-(pyrimidin-2-yl)piperazin-1-yl)pentyl)picolinate—200

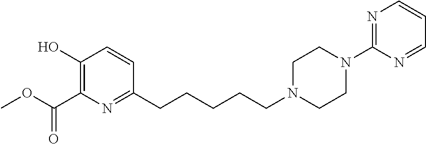 [SMILES: COC(=O)c1nc(CCCCCN2CCN(c3ncccn3)CC2)ccc1O]

Procedure E was applied on compound 199 to give without purification the title compound as a brown oil (3.4 g, quant.). ¹H NMR (300 MHz, CD₂C₂): δ (ppm) 8.30 (d, J=4.7 Hz, 2H), 7.33 (d, J=8.6 Hz, 1H), 7.29 (d, J=8.6 Hz, 1H), 6.49 (br s), 6.49 (t, J=4.7 Hz, 2H), 4.03 (s, 3H), 3.87-3.73 (m, 4H), 2.84-2.72 (m, 2H), 2.49-2.44 (m, 4H), 2.40-2.33 (m, 2H), 1.80-1.62 (m, 3H), 1.62-1.48 (m, 2H), 1.48-1.23 (m, 3H). ¹³C NMR (75 MHz, CD₂Cl₂): δ (ppm) 170.80, 162.39, 158.16, 157.71, 154.35, 129.57, 129.51, 126.91, 110.20, 59.07, 53.68, 53.37, 44.26, 37.98, 30.49, 27.72, 27.24.

### 3-Hydroxy-6-(5-(4-_(pyrimidin-2-yl)piperazin-1-yl)pentyl)picolinaldehyde—201

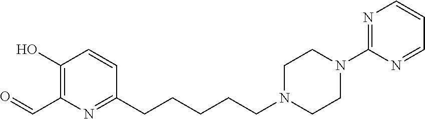 [SMILES: O=Cc1nc(CCCCCN2CCN(c3ncccn3)CC2)ccc1O]

Procedure F (triethylamine as base, DIBAL-H=4 equiv.) was applied to compound 200. Normal phase purification (dichloromethane/MeOH, 100/0 to 90/10, v/v) gave the desired product as a yellow solid (1.46 g, 40% over 3 steps). ¹H NMR (300 MHz, CDCl₃): δ (ppm) 10.04 (s, 1H), 9.73 (s, 1H), 8.30 (d, J=4.7 Hz, 2H), 7.32 (d, J=8.7 Hz, 1H), 7.28 (d, J=8.8 Hz, 1H), 6.47 (t, J=4.7 Hz, 1H), 3.94-3.76 (m, 4H), 2.90-2.72 (m, 2H), 2.63-2.44 (m, 4H), 2.44-2.31 (m, 2H), 1.87-1.69 (m, 2H), 1.69-1.50 (m, 2H), 1.50-1.31 (m, 2H). ¹³C NMR (75 MHz, CDCl₃): δ (ppm) 198.47, 161.42, 157.46, 156.68, 154.62, 135.51, 129.46, 126.12, 109.58, 58.44, 52.92, 43.40, 37.02, 29.38, 26.98, 26.43.

### AB-508—159

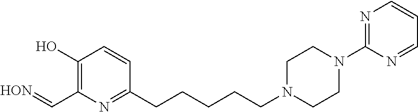

Procedure G was applied to compound 201. Normal phase purification (dichloromethane/MeOH, 100/0 to 90/10, v/v) gave the title product as a white off powder (1.01 g, 66%). Rf=0.5 (dichloromethane/MeOH, 90/10, v/v). ¹H NMR (300 MHz, CDCl₃): δ (ppm) 9.82 (s, 2H), 8.42 (s, 1H), 8.34 (d, J=4.8 Hz, 2H), 7.29 (s, 1H), 7.20 (d, J=8.5 Hz, 1H), 7.03 (d, J=8.5 Hz, 1H), 6.53 (t, J=4.8 Hz, 1H), 4.02-3.79 (m, 4H), 2.83-2.69 (m, 2H), 2.69-2.53 (m, 4H), 2.53-2.36 (m, 2H), 1.85-1.53 (m, 4H), 1.53-1.29 (m, 2H). ¹³C NMR (75 MHz, CDCl₃): δ (ppm) 161.67, 157.89, 153.89, 153.44, 152.68, 135.20, 124.61, 123.98, 110.17, 58.73, 53.05, 43.34, 37.22, 30.03, 27.19, 26.30. HRMS (ESI+): m/z calcd. for C₁₉H₂₇N₆O₂ 371.2195; found 371.2191. HPLC (method B): t_{R}=17.88 min (purity=97.25%).

### 2-(5-bromothiophen-2-yl)-1,3-dioxolane—202

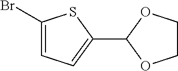 [SMILES: Brc1ccc(C2OCCO2)s1]

5-Bromo-2-thiophene-carboxaldehyde (3.76 g, 19.68 mmol, 1.0 equiv.), ethylene glycol (2.75 mL, 49.2 mmol, 2.5 equiv.), and a catalytic amount of p-toluenesulfonic acid (19 mg, 0.1 mmol, 0.005 equiv.) were dissolved in 28 mL of toluene in a 100 mL round-bottom flask equipped with a Dean-Stark apparatus. The reaction mixture was refluxed at 140° C. for 20 h. The reaction mixture was extracted by ethyl ether and followed by washing with saturated Na₂CO₃, H₂O, and saturated brine solution. The combined organic phase was dried with anhydrous Na₂SO₄. After solvent removal under reduced pressure, the residue was further purified by distillation to give the title compound as a yellow liquid (4.07 g, 82%). (Under reduced pressure (20-50 mbar, 210° C.). ¹H NMR (300 MHz, CDCl₃): δ (ppm) 6.94 (d, J=3.8 Hz, 1H), 6.90 (dd, J=3.8, 0.7 Hz, 1H), 6.02 (d, J=0.7 Hz, 1H), 4.15-4.04 (m, 2H), 4.04-3.94 (m, 2H).

### 4-(3-(5-(1,3-dioxolan-2-yl)thiophen-2-yl)prop-2-yn-1-yl)morpholine—203

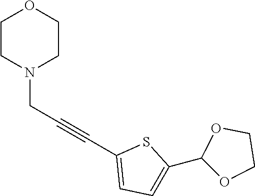 [SMILES: C(#Cc1ccc(C2OCCO2)s1)CN1CCOCC1]

Bromo compound 202 (0.57 g, 2.43 mmol, 1.0 equiv.) and 4-(prop-2-yn-1-yl)morpholine (336 mg, 2.68 mmol, 1.1 eq.) was dissolved in 12 mL of triethylamine and 24 mL of dichloromethane. This mixture was degassed with argon for 5 min, then Pd(PPh₃)₄ (141 mg, 0.05 equiv.) and CuI (46 mg, 0.1 equiv.) were added and the mixture was stirred for 18 hours under argon in absence of light at rt. Solvents were removed; the crude was purified by flash chromatography on silica gel (0 to 5% MeOH in dichloromethane over 40 min, 24G, 30 micron, 20 mL/min) to afford an orange oil (442 mg, 65%). Rf=0.36 (DCM/MeOH 95/5, v/v, vanillin). ¹H NMR (300 MHz, CDCl₃): δ (ppm) 7.11 (d, J=3.7 Hz, 1H), 7.04 (dd, J=3.7, 0.7 Hz, 1H), 6.10 (s, 1H), 4.21-4.10 (m, 2H), 4.10-3.98 (m, 2H), 3.83-3.77 (m, 4H), 3.56 (s, 2H), 2.69-2.64 (m, 4H). ¹³C NMR (75 MHz, CDCl₃): δ (ppm) 143.1, 131.7, 125.9, 123.8, 100.0, 88.7, 78.7, 66.9, 65.3, 52.5, 48.3. HRMS (ESI+): m/z calcd for [C₁₄H₁₈NO₃S]⁺ 280.1007, found 280.1005.

### 4-(4-(5-(1,3-dioxolan-2-yl)thiophen-2-yl)but-3-yn-1-yl)morpholine—204

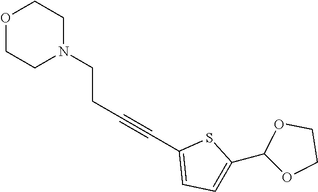 [SMILES: C(#Cc1ccc(C2OCCO2)s1)CCN1CCOCC1]

Bromo compound 202 (0.50 g, 2.13 mmol, 1.0 equiv.) and 4-(but-3-yn-1-yl)morpholine (326 mg, 2.34 mmol, 1.1 equiv.) was dissolved in 11 mL of Triethylamine and 21 mL of dichloromethane. This mixture was degassed with argon for 5 min, then Pd(PPh₃)₄ (246 mg, 0.213 mmol, 0.1 equiv.) and CuI (41 mg, 0.213 mmol, 0.1 equiv.) were added and the mixture was stirred for 18 hours under argon in absence of light at rt. Solvents were removed; the crude was purified by flash chromatography on silica gel (0 to 5% MeOH in dichloromethane over 40 min, 40G, 30 micron, 30 mL/min) to afford an orange oil (346 mg, 56%). Rf=0.22 (cyclo/EtOAc 6/4, v/v, vanillin). ¹H NMR (300 MHz, CDCl₃), δ (ppm) 7.02 (d, J=3.7 Hz, 1H), 7.00 (d, J=3.8 Hz, 1H), 6.07 (s, 1H), 4.17-4.08 (m, 2H), 4.08-4.00 (m, 2H), 3.75 (td, J=4.7, 1.8 Hz, 5H), 2.73-2.57 (m, 5H), 2.57-2.49 (m, 5H). HRMS (ESI+): m/z calcd for [C₁₅H₂₀NO₃S]⁺ 294.1164, found 294.1161.

### 5-(5-(1,3-dioxolan-2-yl)thiophen-2-yl)pent-4-yn-1-ol—205

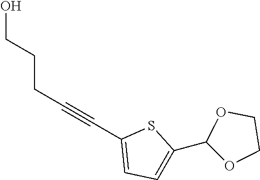 [SMILES: OCCCC#Cc1ccc(C2OCCO2)s1]

Bromo compound 202 (1 g, 4.25 mmol, 1.0 equiv.) and 4-pentynol (0.44 mL, 4.67 mmol 1.1 equiv.) was dissolved in 21 mL of triethylamine and 42 mL of dichloromethane (0.07 M). This mixture was degassed with argon for 5 min, then Pd(PPh₃)₄ (246 mg, 0.21 mmol, 0.05 equiv.) and CuI (81 mg, 0.43 mmol, 0.1 equiv.) were added and the mixture was stirred overnight under argon. and in absence of light. Solvents were removed; the crude was purified by flash chromatography on silica gel (10 to 50% EtOAc in cyclohexane over 45 min, 40G, 30 micron, 30 mL/min) to afford the alcohol (0.88 g, 87%) as an orange oil. Rf=0.16 (cyclo/EtOAc 7/3, v/v, vanillin). ¹H NMR (300 MHz, CDCl₃): δ (ppm) 6.98 (d, J=3.7 Hz, 1H), 6.96 (dd, J=3.7, 0.6 Hz, 1H), 6.03 (d, J=0.5 Hz, 1H), 4.15-4.04 (m, 2H), 4.04-3.93 (m, 2H), 3.77 (t, J=6.2 Hz, 2H), 2.53 (t, J=7.0 Hz, 2H), 1.83 (tt, J=7.0, 6.1 Hz, 2H), 1.72 (s, 1H). ¹³C NMR (75 MHz, CDCl₃): δ (ppm) 142.2, 130.8, 125.8, 124.9, 100.0, 94.1, 74.2, 65.2, 61.6, 31.2, 16.2. HRMS (ESI⁺): m/z calcd for [C₁₂H₁₅O₃S]⁺ 239.0742, found 239.0742.

### 4-(3-(5-(1,3-dioxolan-2-yl)thiophen-2-yl)propyl)morpholine—206

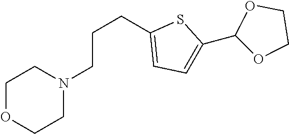 [SMILES: c1cc(C2OCCO2)sc1CCCN1CCOCC1]

Alkyne 203 (440 mg, 1.57 mmol, 1.0 equiv.) was hydrogenated with Pd on charcoal 10% (335 mg, 0.31 mmol, 0.2 equiv.) in MeOH/EtOAc (10:1, 15.7 mL, 0.1 M) for 4 h at rt then filtered on Celite®, rinsed with MeOH and concentrated. The crude was purified by flash chromatography on silica gel (0 to 5% MeOH in dichloromethane over 30 min, 24G, 30 micron, 20 mL/min) to afford the product as a yellowish solid (238 mg, 53%). Rf=0.39 (Dichloromethane/MeOH 95/5, v/v, vanillin). ¹H NMR (300 MHz, CDCl₃): δ (ppm) 7.02 (d, J=3.5 Hz, 1H), 6.74-6.67 (m, 1H), 6.07 (s, 1H), 4.26-4.11 (m, 2H), 4.10 (s, 2H), 3.81-3.71 (m, 5H), 2.92-2.83 (m, 2H), 2.52-2.45 (m, 4H), 2.45-2.39 (m, 2H), 1.89 (p, J=7.5 Hz, 3H). ¹³C NMR (75 MHz, CDCl₃): δ (ppm) 146.5, 138.9, 126.3, 123.8, 100.5, 67.0, 65.2, 57.9, 53.7, 28.4, 27.9. HRMS (ESI⁺): m/z calcd for [C₁₄H₂₂NO₃S]⁺ 284.1312, found 284.1320.

### 4-(4-(5-(1,3-dioxolan-2-yl)thiophen-2-yl)butyl)morpholine—207

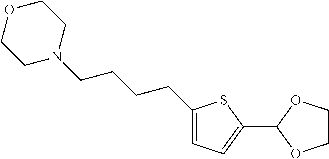 [SMILES: c1cc(C2OCCO2)sc1CCCCN1CCOCC1]

Alkyne 204 (350 mg, 1.18 mmol, 1.0 equiv.) was hydrogenated with Pd on charcoal 10% (251 mg, 0.25 mmol, 0.2 equiv.) in MeOH (11.7 mL, 0.1 M) for 2 h at rt. Filtered on Celite® and concentrated. The crude was purified by flash chromatography on silica gel (0 to 5% MeOH in DCM over 40 min, 40G, 15 micron, 30 mL/min). Isolated as a yellowish solid (165 mg, 47%). Rf=0.39 (DCM/MeOH 95/5, v/v, vanillin). ¹H NMR (300 MHz, CDCl₃): δ (ppm) 6.93 (d, J=3.5 Hz, 1H), 6.62 (d, J=3.5 Hz, 1H), 5.98 (s, 1H), 4.15-4.02 (m, 2H), 4.02-3.89 (m, 2H), 3.67 (t, J=4.7 Hz, 4H), 2.78 (t, J=7.4 Hz, 2H), 2.38 (t, J=4.6 Hz, 4H), 2.31 (t, J=7.5 Hz, 2H), 1.66 (p, J=7.2 Hz, 2H), 1.51 (tt, J=9.6, 5.5 Hz, 2H). ¹³C NMR (75 MHz, CDCl₃): δ (ppm) 146.7, 138.8, 126.2, 123.7, 100.4, 66.9, 65.1, 58.7, 53.7, 30.1, 29.4, 25.9. m.p.=43-45° C. HRMS (ESI⁺): m/z calcd for [C₁₅H₂₄NO₃S]⁺ 298.1477, found 298.1486.

### 5-(5-(1,3-dioxolan-2-yl)thiophen-2-yl)pentan-1-ol—208

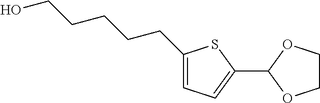 [SMILES: OCCCCCc1ccc(C2OCCO2)s1]

Alkyne 205 (910 mg, 3.81 mmol, 1.0 equiv.) was hydrogenated with Pd on charcoal 10% (227 mg, 25% wt.) in MeOH (38 mL, 0.1 M). After 2 hours, the reaction mixture was filtered and concentrated. The crude was purified by flash chromatography on silica gel (10 to 50% AcOEt in cyclohexane over 45 min, 40G, 30 micron) to afford the product as an oil (822 mg, 89%). Rf=0.54 (cyclo/EtOAc 1/1, v/v, vanillin). ¹H NMR (300 MHz, CDCl₃): δ (ppm) 7.01 (d, J=3.4 Hz, 1H), 6.70 (dt, J=3.5, 1.0 Hz, 1H), 6.07 (s, 1H), 4.22-4.10 (m, 2H), 4.10-4.00 (m, 2H), 3.67 (t, J=6.5 Hz, 2H), 2.85 (td, J=7.4, 0.9 Hz, 2H), 1.81-1.67 (m, 2H), 1.67-1.57 (m, 2H), 1.53-1.40 (m, 2H). ¹³C NMR (75 MHz, CDCl₃): δ (ppm) 147.0, 138.7, 126.3, 123.7, 100.5, 65.2, 62.8, 32.5, 31.4, 30.2, 25.2. HRMS (ESI⁺): m/z calcd for [C₁₂H₁₉O₃S]⁺ 243.1055, found 243.1047.

### 5-(5-morpholinopentyl)thiophene-2-carbaldehyde 209

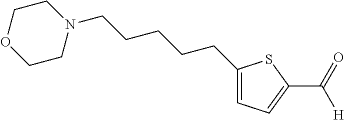 [SMILES: O=Cc1ccc(CCCCCN2CCOCC2)s1]

A solution of alcohol 208 (0.56 g, 2.3 mmol. 1.00 equiv.), and triethylamine (1.6 mL, 11.6 mmol, 5.0 equiv.) in dichloromethane (11 mL, 0.2 M) at 0° C. was treated dropwise with methanesulfonyl chloride (0.31 mL, 3.9 mmol, 1.7 equiv.), followed by stirring for 1 hour at this temperature. dichloromethane was added (10 mL), the solution was washed with water (10 mL) and brine (10 mL). Organic layer was dried over MgSO₄ and evaporated under reduced pressure. The crude mesylate was directly used in following step without further purification. The crude mesylate was dissolved in THF (11 mL) before morpholine (2.0 mL, 23 mmol, 10 equiv.) was added and the reaction mixture was refluxed overnight. Filtered, concentrated, and purified by flash chromatography (100% dichloromethane to 98% dichloromethane 2% NH₃ 7N in MeOH, 24 G, 15 micro, 20 mL/min) to afford the thiophene (410 mg, 57%). Rf=0.42 (DCM/MeOH 7N NH₃ 99/1, v/v, vanillin). ¹H NMR (300 MHz, CDCl₃): δ (ppm) 7.01 (d, J=3.5 Hz, 1H), 6.69 (dt, J=3.5, 1.0 Hz, 1H), 6.06 (s, 1H), 4.22-4.10 (m, 2H), 4.10-3.98 (m, 2H), 3.80-3.70 (m, 4H), 2.83 (t, J=7.5 Hz, 2H), 2.52-2.39 (m, 4H), 2.41-2.30 (m, 2H), 1.81-1.65 (m, 2H), 1.64-1.46 (m, 2H), 1.48-1.35 (m, 2H). ¹³C NMR (75 MHz, CDCl₃): δ (ppm) 147.0, 138.7, 126.2, 123.6, 100.5, 67.0, 65.2, 59.0, 53.8, 31.5, 30.2, 26.9, 26.3. HRMS (ESI⁺): m/z calcd for [C₁₆H₂₆NO₃S]⁺ 312.1633, found 312.1640.

The acetal (410 mg, 1.32 mmol, 1.0 equiv.) was stirred for 18 h at rt in a mixture of formic acid and water (1:1, 13 mL, 0.1 M), The reaction mixture was quenched by solid NaHCO₃ until neutral and extracted with EtOAc (3×50 mL). Dried over MgSO₄ and concentrated. Completion monitored by crude NMR. Purified by flash chromatography (0 to 2% MeOH/NH₃ in dichloromethane over 45 min, 24G, 20 mL/min) to afford an orange oil (290 mg, 82%). Rf=0.41 (dichloromethane/MeOH NH₃ 7M, 98/2, v/v). ¹H NMR (300 MHz, CDCl₃): δ (ppm) 9.81 (s, 1H), 7.60 (d, J=3.8 Hz, 1H), 6.89 (dt, J=3.8, 0.9 Hz, 1H), 3.76-3.66 (m, 4H), 2.93-2.82 (m, 2H), 2.41 (dd, J=5.7, 3.7 Hz, 4H), 2.35-2.28 (m, 2H), 1.80-1.65 (m, 2H), 1.53 (dddd, J=14.1, 9.5, 6.5, 3.1 Hz, 2H), 1.45-1.33 (m, 2H). ¹³C NMR (75 MHz, CDCl₃): δ (ppm) 182.6, 157.4, 141.7, 137.0, 125.9, 67.0, 58.9, 53.8, 31.2, 30.8, 26.9, 26.2. HRMS (ESI⁺): m/z calcd for [C₁₄H₂₂NO₂S]⁺ 268.1371, found 268.1374.

### 5-(5-(6,7-dimethoxy-3,4-dihydroisoquinolin-2(1H)-yl)pentyl)thiophene-2-carbaldehyde—210

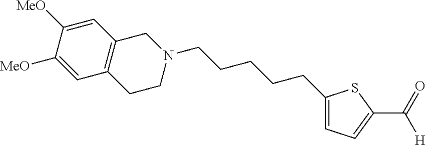 [SMILES: COc1cc2c(cc1OC)CN(CCCCCc1ccc(C=O)s1)CC2]

A solution of alcohol 208 (0.79 g, 3.3 mmol, 1.0 equiv.), and triethylamine (2.2 mL, 16.3 mmol, 5.0 equiv.) in dichloromethane (32 mL, 0.1 M) at 0° C. was treated dropwise with methanesulfonyl chloride (0.43 mL, 5.5 mmol, 1.7 equiv.), followed by stirring for 1 hour at this temperature. Dichloromethane was added (10 mL), the solution was washed with H₂O (10 mL) and brine (10 mL). Organic layer was dried over MgSO₄ and evaporated under reduced pressure. The crude mesylate was directly used in following step without further purification. Mesylate was dissolved in dry MeCN (16 mL, 0.2 M) under Argon atmosphere and quinoline.HCl (1.5 g, 6.5 mmol, 2.0 equiv.) followed by potassium carbonate (1.8 g, 13.1 mmol, 4.0 equiv.) were added in this order. The resulting heterogenous mixture was reflux overnight. Salt were removed by filtration, rinsed and solvent was evaporated under reduced pressure. Purified by flash chromatography (0 to 5% MeOH in DCM over 40 min, 24G Interchim, 20 mL/min) to afford the acetal as an orange oil (1.028 g, 78%). ¹H NMR (300 MHz, CDCl₃): δ (ppm) 6.96 (dd, J=3.5, 0.6 Hz, 1H), 6.65 (dt, J=3.5, 0.9 Hz, 1H), 6.58 (s, 1H), 6.52 (s, 1H), 6.02 (d, J=0.5 Hz, 1H), 4.16-4.09 (m, 2H), 4.02-3.96 (m, 2H), 3.83 (d, J=1.6 Hz, 8H), 3.53 (s, 3H), 2.87-2.76 (m, 5H), 2.69 (t, J=5.6 Hz, 3H), 2.53-2.42 (m, 2H), 1.77-1.65 (m, 3H), 1.65-1.54 (m, 3H), 1.48-1.38 (m, 2H). ¹³C NMR (75 MHz, CDCl₃): δ (ppm) 147.5, 147.2, 147.1, 138.7, 126.8, 126.3, 126.2, 123.6, 111.4, 109.6, 100.5, 65.2, 58.3, 56.0, 55.9, 55.9, 51.1, 31.6, 30.2, 28.7, 27.0. HRMS (ESI⁺): m/z calcd for [C₂₃H₃₂NO₄S]⁺ 418.2052, found 418.2047.

The acetal (1.028 g, 2.46 mm, 1.0 equiv.) was stirred for 18 h at rt in a mixture of formic acid and water (1:1, 33 mL, 0.1 M). Quenched by solid NaHCO₃ until neutral and extracted with AcOEt (3×150 mL). Dried over MgSO₄ and concentrated. Completion monitored by crude NMR. Purified by flash chromatography (0 to 5% MeOH in DCM over 45 min, 24G Interchim, 30 micro, 20 mL/min) to afford an orange oil (832 mg, 90%). Rf=0.64 (DCM/MeOH, 95/5, v/v). ¹H NMR (300 MHz, CDCl₃): δ (ppm) 9.80 (s, 1H), 7.59 (d, J=3.8 Hz, 1H), 6.90 (dt, J=3.8, 0.9 Hz, 1H), 6.58 (s, 1H), 6.51 (s, 1H), 3.83 (s, 3H), 3.82 (s, 3H), 3.56-3.48 (m, 2H), 2.92-2.85 (m, 2H), 2.81 (t, J=5.8 Hz, 2H), 2.68 (t, J=6.0 Hz, 2H), 2.53-2.44 (m, 2H), 1.76 (tt, J=8.4, 7.3 Hz, 2H), 1.69-1.56 (m, 2H), 1.44 (tdd, J=10.2, 7.3, 4.1 Hz, 2H). ¹³C NMR (75 MHz, CDCl₃): δ (ppm) 182.6, 157.5, 147.5, 147.2, 141.7, 137.0, 126.7, 126.2, 125.9, 111.4, 109.5, 58.1, 56.0, 55.9, 55.9, 51.1, 31.2, 30.8, 28.7, 27.0. HRMS (ESI⁺): m/z calcd for [C₂₁H₂₈NO₃S]⁺ 374.1790 found 374.1799.

### 5-(3-morpholinopropyl)thiophene-2-carbaldehyde oxime—211

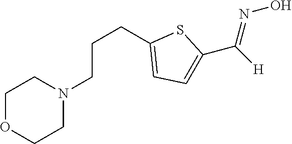 [SMILES: O/N=C/c1ccc(CCCN2CCOCC2)s1]

Acetal 206 (226 mg, 0.80 mmol, 1.0 equiv.) was heated for 18 hours at 60° C. in a mixture of formic acid/water (1:1, 16 mL, 0.05 M). The resulting solution was concentrated and quenched by saturated aqueous solution of NaHCO₃ until neutral and extracted with EtOAc (3×50 mL). Dried over MgSO₄ and concentrated. Purified by flash chromatography (0 to 5% MeOH in DCM over 20 min, 12G, 30 micron, 20 mL/min) to afford the aldehyde (145 mg, 79%). Rf=0.48 (DCM/MeOH 95/5, v/v, ninhydrin). ¹H NMR (300 MHz, CDCl₃): δ (ppm) 9.85 (s, 1H), 7.65 (d, J=3.8 Hz, 1H), 6.95 (dd, J=3.8, 0.9 Hz, 1H), 3.77-3.72 (m, 4H), 3.00-2.92 (m, 2H), 2.46 (dd, J=5.6, 3.7 Hz, 4H), 2.42 (dd, J=8.1, 6.4 Hz, 3H), 1.92 (p, J=7.4 Hz, 2H). ¹³C NMR (75 MHz, CDCl₃): δ (ppm) 182.7, 156.8, 141.8, 137.1, 126.1, 67.0, 57.5, 53.7, 28.4, 28.0. HRMS (ESI⁺): m/z calcd for [C₁₂H₁₈NO₂S]⁺ 240.1058, found 240.1052.

NaHCO₃ (155 mg, 1.84 mmol, 3.0 equiv.) and NH₂OH·HCl (107 mg, 1.53 mmol, 2.5 equiv.) added to a mixture of aldehyde (147 mg, 0.61 mmol, 1.0 equiv.) in MeOH (0.1M, 6 mL) and stirred for 24 hours. Filtered through Celite®, concentrated and purified by flash chromatography (DCM/MeOH 0 to 10% over 30 min, 24G, 30 micro, 20 mL/min) to afford the oxime (152 mg, 97%). 1M MsOH (961 mg of MsOH in 10 mL of AcOEt) was added to form the mesylate salt.

¹H NMR (300 MHz, CDCl₃): δ (ppm) 8.22 (s, 1H), 7.66 (d, J=4.1 Hz, OH), 7.19 (d, J=3.7 Hz, OH), 6.98 (d, J=3.6 Hz, 1H), 6.82 (d, J=3.7 Hz, OH), 6.74 (d, J=3.5 Hz, 1H), 3.83 (q, J=4.7 Hz, 4H), 2.91 (dt, J=19.1, 7.7 Hz, 2H), 2.69-2.54 (m, 4H), 2.54-2.40 (m, 2H), 2.03 (ddt, J=15.4, 10.4, 7.8 Hz, 2H). ¹³C NMR (75 MHz, CDCl₃): δ (ppm) 150.5, 146.9, 144.3, 134.5, 130.9, 128.9, 124.6, 123.7, 66.5, 66.4, 58.4, 58.2, 53.6, 53.6, 28.3, 28.1, 28.0, 27.9. HRMS (ESI⁺): m/z calcd for [C₁₂H₁₉N₂O₂S]⁺ 255.1167, found 255.1167. HPLC (Column Syncronis Aq, 100*2.1 mm, 1.7 m, 0.1% formic Acid H₂O/MeCN, 5 to 100% over 18 min. 0.6 mL/min): t_{R}=1.83 min (Cis), 2.00 min (Trans), purity=99.1%.

### 5-(4-morpholinobutyl)thiophene-2-carbaldehyde oxime—212

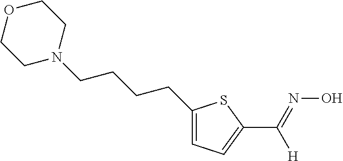 [SMILES: O/N=C/c1ccc(CCCCN2CCOCC2)s1]

Acetal 207 (157 mg, 0.53 mmol, 1.0 equiv.) was heated for 18 hours at 60° C. in a mixture of formic acid and water (1:1, 0.05 M, 10.5 mL). The resulting solution was concentrated and quenched by saturated aqueous solution of NaHCO₃ until neutral and extracted with EtOAc (4×100 mL). Dried over MgSO₄ and concentrated to give the crude material (130 mg, 97%) that was used without further purification in the next step.

¹H NMR (300 MHz, CDCl₃): δ (ppm) 9.80 (s, 1H), 7.61 (d, J=3.8 Hz, 1H), 6.91 (dt, J=3.8, 0.9 Hz, 1H), 3.76-3.62 (m, 4H), 2.98-2.81 (m, 2H), 2.42 (dd, J=5.7, 3.6 Hz, 4H), 2.39-2.30 (m, 2H), 1.82-1.65 (m, 2H), 1.64-1.48 (m, 2H).

NaOAc (126 mg, 1.534 mmol, 3.0 equiv.) and NH₂OH·HCl (89 mg, 1.28 mmol, 2.5 equiv.) added to a mixture of aldehyde (130 mg, 0.51 mmol, 1.0 equiv.) in MeOH (5 mL) and stirred for 6 hours. The crude mixture was concentrated and purified by reversed phase (0 to 30% MeCN in H₂O 0.2% AcOH over 30 min, puriflash C18, 30×250 mm, C18AQ-5 micro) as an acetate salt. This was treated with NaHCO₃ and extracted with AcOEt, concentrated and isolated as a free amine (108.1 mg, 78%). The free amine was treated with 1.0 equivalent of MeSO₃H (1.0 M in AcOEt), evaporated slowly and isolated as mesylate salt (144.4 mg, 77%) that was dried on heated vacuum and high vacuum. Mixture of two isomers: ¹H NMR (300 MHz, MeOD): δ (ppm) 8.17 (s, OH), 7.65 (s, 1H), 7.30 (d, J=3.8 Hz, 1H), 7.05 (d, J=3.6 Hz, OH), 6.95-6.86 (m, 1H), 6.86-6.78 (m, OH), 4.87 (s, 4H), 4.05 (dd, J=13.2, 3.7 Hz, 2H), 3.78 (ddd, J=13.5, 11.9, 2.2 Hz, 2H), 3.57-3.42 (m, 2H), 3.25-3.15 (m, 2H), 3.10 (dd, J=12.3, 3.7 Hz, 2H), 2.98 (d, J=6.8 Hz, 1H), 2.94-2.85 (m, 1H), 2.72 (s, 3H), 1.82 (tt, J=6.0, 3.2 Hz, 4H). ¹³C NMR (75 MHz, MeOD): δ (ppm) 150.3, 145.9, 143.8, 140.4, 134.6, 131.6, 129.5, 128.9, 124.8, 124.0, 63.6, 56.8, 51.8, 38.2, 28.9, 28.7, 28.0, 27.9, 22.6, 22.6. HRMS (ESI⁺): m/z calcd for [C₁₃H₂₁N₂O₂S]⁺ 269.1324, found 269.1313. HPLC (Column Syncronis Aq, 100*3 mm, 3 m, H₂O formic acid 0.2%/MeCN, 0 to 100% over 20 min, 0.5 mL/min): t_{R}=11.02 min (Cis), 11.33 min (Trans), purity=99.1%.

### (E)-5-(5-morpholinopentyl)thiophene-2-carbaldehyde oxime—213

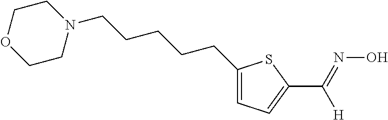 [SMILES: O/N=C/c1ccc(CCCCCN2CCOCC2)s1]

NaOAc (113 mg, 1.38 mmol, 3.0 equiv.) and NH₂OH·HCl (80 mg, 1.15 mmol, 2.5 equiv.) added to a mixture of aldehyde (123 mg, 0.46 mmol, 1.0 equiv.) in MeOH (4.6 mL, 0.1 M) and stirred for 4 hours. The resulting mixture was concentrated with Celite® and purified by flash chromatography (0 to 10 MeOH in DCM, over 40 min, 20 mL/min, 12G, 15 micro) to give the desired compound as an acetate salt (trans isomer: 46 mg, 36%, cis isomer: 53 mg, 41%).

F1 (Trans isomer, 0.69 AcOH): ¹H NMR (300 MHz, CDCl₃+MeOD-d6): δ (ppm) 8.09 (d, J=0.9 Hz, 1H), 6.89 (d, J=3.4 Hz, 1H), 6.61 (dd, J=3.5, 1.0 Hz, 1H), 3.71 (t, J=4.7 Hz, 4H), 2.71 (t, J=7.6 Hz, 2H), 2.55 (t, J=4.7 Hz, 4H), 2.45-2.33 (m, 2H), 1.95 (d, J=1.0 Hz, 2H), 1.61 (p, J=7.4 Hz, 2H), 1.51 (td, J=11.4, 9.8, 6.1 Hz, 2H), 1.30 (q, J=8.1 Hz, 2H). ¹³C NMR (75 MHz, CDCl₃+MeOD-d6): δ (ppm) 176.0, 147.5, 144.5, 133.6, 129.2, 124.4, 65.9, 58.5, 53.0, 31.1, 30.0, 26.6, 25.1, 21.8 (AcOH). HRMS (ESI⁺): m/z calcd for [C₁₄H₂₃N₂O₂S]⁺ 283.1480, found 283.1491. HPLC (Column Syncronis Aq, 100*3 mm, 3 m, Ammonium Acetate 50 mM/MeCN, 0 to 100% over 20 min, 0.5 mL/min): t_{R}=14.91 min (Cis), 15.43 min (Trans), purity=100.0%.

F2 (Cis isomer, 0.46 AcOH): ¹H NMR (300 MHz, CDCl₃+MeOD-d6): δ (ppm) 7.50 (s, 1H), 7.11 (d, J=3.7 Hz, 1H), 6.69 (dd, J=3.8, 0.9 Hz, 1H), 3.70 (t, J=4.7 Hz, 4H), 2.76 (t, J=7.5 Hz, 2H), 2.50 (t, J=4.7 Hz, 4H), 2.42-2.29 (m, 2H), 1.96 (s, 1H), 1.65 (p, J=7.6 Hz, 2H), 1.50 (tt, J=7.8, 6.2 Hz, 2H), 1.31 (qd, J=7.6, 6.6, 3.7 Hz, 2H). ¹³C NMR (75 MHz, CDCl₃+MeOD-d6): δ (ppm) 175.8, 151.5, 140.9, 131.4, 129.1, 123.6, 66.2, 58.6, 53.2, 31.2, 29.9, 26.8, 25.4, 21.8. HRMS (ESI⁺): m/z calcd for [C₁₄H₂₃N₂O₂S]⁺ 283.1480, found 283.1483. HPLC (Column Syncronis Aq, 100*3 mm, 3 mm, Ammonium Acetate 50 mM/MeCN, 0 to 100% over 20 min, 0.5 mL/min): t_{R}=15.06 min (Cis), 15.42 min (Trans), purity=99.1%.

### (E)-5-(5-(6,7-dimethoxy-3,4-dihydroisoquinolin-2(1H)-yl)pentyl)thiophene-2-carbaldehyde oxime—214

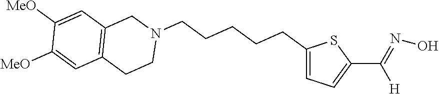 [SMILES: COc1cc2c(cc1OC)CN(CCCCCc1ccc(/C=N/O)s1)CC2]

NaOAc (86 mg, 1.04 mmol, 3.0 equiv.) and NH₂OH·HCl(60 mg, 0.887 mmol, 2.5 equiv.) added to a mixture of aldehyde (130 mg, 0.35 mmol, 1.0 equiv.) in MeOH (3.5 mL, 0.1 M) and stirred for 4 hours. The crude mixture was concentrated with Celite® and purified by flash chromatography (0 to 10% MeOH in DCM, over 40 min, 20 mL/min, 24G, 15 micro) to give the desired product as an acetate salt (trans isomer: 72 mg, 53%, cis isomer: 52 mg, 38%).

F1 (Trans isomer): ¹H NMR (300 MHz, CDCl₃+MeOD): δ (ppm) 8.06 (s, 1H), 6.84 (d, J=3.6 Hz, 1H), 6.61-6.57 (m, 1H), 6.52 (s, 1H), 6.45 (s, 1H), 3.82 (s, 2H), 3.75 (s, 31H), 3.73 (s, 3H), 2.99 (t, J=6.0 Hz, 2H), 2.91-2.82 (m, 2H), 2.69 (dq, J=7.6, 4.7, 4.1 Hz, 4H), 1.76-1.65 (m, 2H), 1.65-1.55 (m, 2H), 1.33 (h, J=7.4, 6.5 Hz, 2H). ¹³C NMR (75 MHz, CDCl₃+MeOD): δ (ppm) 148.1, 147.7, 147.2, 144.1, 134.1, 129.0, 124.5, 124.4, 122.9, 111.2, 109.5, 56.0, 55.91, 55.89, 53.6, 49.5, 31.1, 30.1, 26.7, 26.0, 25.2. HRMS (ESI⁺): m/z calcd for [C₂₁H₂₉N₂O₃S]⁺ 389.1899, found 389.1902. HPLC (Column Syncronis Aq, 100*3 mm, 3 m, Ammonium Acetate 50 mM/MeCN, 0 to 100% over 20 min, 0.5 mL/min): t_{R}=17.00 min (Cis), 17.48 min (Trans), purity=96.8%.

F2 (Cis isomer): ¹H NMR (300 MHz, CDCl₃+MeOD): δ (ppm) 7.49 (s, 1H), 7.10 (d, J=3.6 Hz, 1H), 6.68 (d, J=3.7 Hz, 1H), 6.53 (s, 1H), 6.46 (s, 1H), 3.77 (s, 2H), 3.76 (s, 3H), 3.75 (s, 3H), 2.96 (t, J=6.0 Hz, 2H), 2.84 (t, J=6.1 Hz, 2H), 2.76 (t, J=7.5 Hz. 2H), 2.71-2.61 (m, 2H), 1.71-1.56 (m, 4H), 1.40-1.23 (m, 2H). ¹³C NMR (75 MHz, CDCl₃+MeOD): δ (ppm) 151.4, 148.1, 147.7, 140.8, 131.4, 129.1, 124.6, 123.7, 123.1, 111.2, 109.4, 56.1, 55.9, 55.8, 53.7, 49.6, 31.1, 29.8, 26.7, 26.1, 25.3. HRMS (ESI⁺): m/z calcd for [C₂₁H₂₉N₂O₃S]⁺ 389.1899, found 389.1905. HPLC (Column Syncronis Aq, 100*3 mm, 3 m, Ammonium Acetate 50 mM/MeCN, 0 to 100% over 20 min, 0.5 mL/min): t_{R}=17.08 min (Cis), 17.48 min (Trans), purity=98.8%.

### Ethyl 5-bromo-1,3,4-thiadiazole-2-carboxylate—215

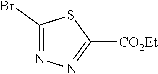 [SMILES: CCOC(=O)c1nnc(Br)s1]

To a stirred solution of ethyl 5-amino-1,3,4-thiadiazole-2—carboxylate (3.66 g, 21.1 mmol, 1.0 equiv.) in acetonitrile (70 mL, 0.6 M) at room temperature was added copper (II) bromide (9.4 g, 42.3 mmol, 2.0 equiv.) and the mixture was stirred for 20 min. Tertiary butyl nitrite (4.3 g, 42.3 mmol, 2.0 equiv.) was then added drop wise for 10 min, and the reaction mixture was heated to 60° C. for 30 min. The reaction mixture was concentrated under reduced pressure, diluted with water (300 mL), and then extracted with ethyl acetate (500 mL). The organic layer was separated and dried over anhydrous sodium sulphate, and evaporated to afford ethyl 5-bromo-1,3,4-thiadiazole-2-carboxylate as a light-yellow solid (2.43 g, 49%).

¹H NMR (300 MHz, CDCl₃): δ (ppm) 4.57 (q, J=7.1 Hz, 1H), 1.50 (t, J=7.1 Hz, 2H).

### Ethyl 5-(4-morpholinobut-1-yn-1-yl)-1,3,4-thiadiazole-2-carboxylate—216

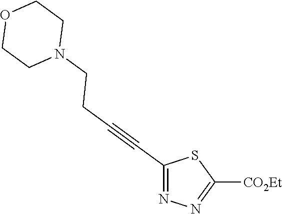 [SMILES: CCOC(=O)c1nnc(C#CCCN2CCOCC2)s1]

Bromo compound 215 (0.5 g, 2.11 mmol, 1.0 equiv.) and 4-(but-3-yn-1-yl)morpholine (323 mg, 2.32 mmol, 1.1 equiv.) was dissolved in 11 mL of triethylamine and 21 mL of dichloromethane. This mixture was degassed with argon for 5 min, then Pd(PPh₃)₄ (244 mg, 0.21 mmol, 0.1 equiv.) and CuI (40 mg, 0.21 mmol, 0.1 equiv.) were added and the mixture was stirred for 18 hours under argon in absence of light at rt. Solvents were removed; the crude was purified by flash chromatography on silica gel (dichloromethane/MeOH 0 to 5% over 45 min, 24G, 30 micron, SIHP, 30 mL/min) to afford the title product as a black oil (381 mg, 61%). Rf=0.13 (cyclo/EtOAc 6/4, v/v). 10 ¹H NMR (300 MHz, CDCl₃): δ (ppm) 4.45 (q, J=7.2 Hz, 2H), 3.71-3.58 (m, 4H), 2.65 (d, J=1.0 Hz, 4H), 2.49-2.35 (m, 4H), 1.39 (t, J=7.1 Hz, 3H). ¹³C NMR (75 MHz, CDCl₃): δ (ppm) 159.6, 158.2, 154.3, 102.2, 70.8, 66.9, 63.5, 56.2, 53.3, 18.1, 14.2. HRMS (ESI⁺): m/z calcd for [C₁₃H₁₈N₃O₃S]⁺ 296.1069, found 296.1068.

### Ethyl 5-(4-morpholinobutyl)-1,3,4-thiadiazole-2-carboxylate—217

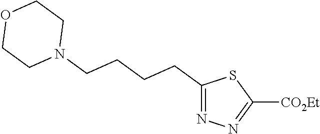 [SMILES: CCOC(=O)c1nnc(CCCCN2CCOCC2)s1]

Alkyne 216 (1.27 g, 4.28 mmol, 1.0 equiv.) was dissolved in EtOH (42 mL, 0.1 M) with 10% Pd on charcoal (456 mg, 0.43 mmol, 0.1 equiv.) under hydrogen for 4 h at rt. The crude mixture was filtered through Celite®, concentrated and purified by flash chromatography on silica gel (0 to 5% MeOH in DCM over 40 min, 40G, 15 micron, 30 mL/min) to give the desired compound as a Black oil (850 mg, 66%). ¹H NMR (300 MHz, CDCl₃): δ (ppm) 4.43 (q, J=7.1 Hz, 2H), 3.62 (t, J=4.7 Hz, 4H), 3.14 (t, J=7.6 Hz, 2H), 2.39-2.25 (m, 6H), 1.81 (p, J=7.6 Hz, 2H), 1.60-1.47 (m, 2H), 1.38 (t, J=7.1 Hz, 3H). ¹³C NMR (75 MHz, CDCl₃): δ (ppm) 175.0, 160.2, 158.7, 66.8, 63.1, 58.1, 53.6, 30.1, 27.8, 25.7, 14.1. HRMS (ESI⁺): m/z calcd for [C₁₃H₂₂N₃O₃S]⁺ 300.1382, found 300.1388.

### 5-(4-morpholinobutyl)-1,3,4-thiadiazole-2-carbaldehyde—218

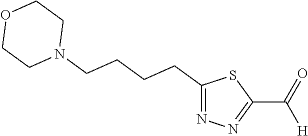 [SMILES: O=Cc1nnc(CCCCN2CCOCC2)s1]

Methyl ester 217 (0.71 g, 2.37 mmol, 1.0 equiv.) was dissolved in dry DCM (15 mL, 0.15 M), the solution was cooled down to −78° C., DIBAL-H [1 M in DCM] (4.74 mL, 2.60 mmol, 1.1 equiv.) was slowly added. This mixture was stirred at −78° C. for 45 min. Extra 0.9 equiv. of DIBAL-H added and stirred for 15 min. MeOH (10 mL) was slowly added, then the mixture was allowed to warm to rt. The solvent was evaporated and the residue dissolved by dichloromethane (100 mL), washed with saturated solution of NaCl, filtered through Celite® and extracted twice by DCM. Combined organic layers were dried over MgSO₄ and evaporated under reduced pressure to afford the aldehyde (769 mg, 92%). The crude material was used for the next step without purification. ¹H NMR (300 MHz, CDCl₃): δ (ppm) 10.23 (s, 1H), 3.83-3.62 (m, 4H), 3.28 (t, J=7.6 Hz, 2H), 2.52-2.44 (m, 4H), 2.42 (d, J=7.4 Hz, 2H), 2.02-1.88 (m, 2H), 1.74-1.59 (m, 2H). ¹³C NMR (75 MHz, CDCl₃): δ (ppm) 183.4, 175.5, 167.6, 67.0, 58.2, 53.7, 27.8, 25.8. HRMS (ESI⁺): m/z calcd for [C₁₁H₁₈N₃O₂S]⁺ 256.1120, found 256.1120.

### (E)-5-(4-morpholinobutyl)-1,3,4-thiadiazole-2-carbaldehyde oxime—219

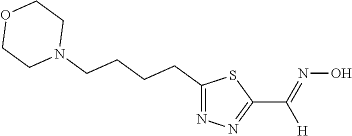 [SMILES: O/N=C/c1nnc(CCCCN2CCOCC2)s1]

NH₂OH·HCl (406 mg, 5.85 mmol, 2.5 equiv.) and NaHCO₃ (589 mg, 7.01 mmol, 3.0 equiv.) added to a mixture of aldehyde 218 (597 mg, 2.34 mmol, 1.0 equiv.) in MeOH (24 mL, 1.2 M) and stirred for 2 hours. Concentrated under reduced pressure, water was added and extracted with DCM (3×20 mL), dried over MgSO₄ and finally concentrated. The crude mixture was purified by flash chromatography (0 to 10% MeOH in DCM over 30 min, 12G, 15 microns, 20 mL/min) to afford the title oxime (538 mg, 85%). ¹H NMR (300 MHz, CDCl₃+MeOH): δ (ppm) 8.34 (s, OH), 8.02 (s, 1H), 3.71 (t, J=4.7 Hz, 4H), 3.14 (t, J=7.5 Hz, 1H), 3.08 (t, J=7.5 Hz, 1H), 2.48 (t, J=4.7 Hz, 3H), 2.45-2.31 (m, 2H), 1.82 (ddt, J=15.1, 10.1, 7.2 Hz, 2H), 1.63 (ddt, J=15.3, 10.1, 5.8 Hz, 2H). ¹³C NMR (75 MHz, CDCl₃+MeOH): δ (ppm) 172.5, 170.1, 164.1, 154.8, 141.9, 138.3, 66.3, 58.3, 58.2, 53.4, 29.8, 29.3, 27.7, 27.6, 25.3. HRMS (ESI⁺): m/z calcd for [C₁₁H₁₉N₄O₂S]≥271.1229, found 271.1232.

Dissolved in DCM/Methanol, 1 equiv. of MsOH 1M in DCM was added, concentrated and dried in high vacuum. Product crystallized. HPLC (Column Syncronis Aq, 100*3 mm, 3 m, H₂O formic acid 0.1%/MeCN, 0 to 100% over 20 min, 0.5 mL/min): t_{R}=9.29 min (purity=95.2%).

### 4-(5-(benzyloxy)-6-(1,3-dioxolan-2-yl)pyridin-2-yl)but-3-yn-1-ol—220

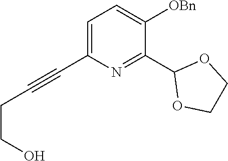 [SMILES: OCCC#Cc1ccc(OCc2ccccc2)c(C2OCCO2)n1]

Bromo compound 85 (5 g, 14.87 mmol, 1.0 equiv.) and 3-butyn-1-ol (1.13 mL, 14.97 mmol, 1.0 equiv.) was dissolved in 73 mL of TEA and 148 mL of DCM. This mixture was degassed 15 min, then Pd(PPh₃)₄ (0.86 g, 0.74 mmol, 0.05 equiv.) and CuI (283 mg, 1.49 mmol, 0.1 equiv.) were added and the mixture was stirred overnight under argon. Solvents were removed; the crude was purified by flash chromatography on silica gel (Cyclohexane/AcOEt 50:50 to 100% AcOEt over 1 hour, 120G) to afford the title compound (4.53 g, 94%) as an orange oil. Rf=0.45 (cyclo/EtOAc 2/8, v/v). ¹H NMR (300 MHz, CDCl₃) δ (ppm) 7.47-7.35 (m, 5H, Ph), 7.22 (d, J=8.6 Hz, 1H), 6.29 (s, 1H), 5.16 (s, 2H), 4.29-4.18 (m, 2H), 4.13-3.99 (m, 2H), 3.86 (t, J=6.3 Hz, 2H), 2.93 (s, 1H), 2.71 (t, J=6.3 Hz, 2H). ¹³C NMR (75 MHz, CDCl₃): δ (ppm) 152.7, 146.5, 135.7, 134.6, 128.7, 128.36, 128.35, 127.4, 120.2, 100.7, 86.5, 81.5, 70.6, 65.8, 60.8, 23.9. HRMS (ESI⁺): m/z calcd for [C₁₉H₂₀NO₄]⁺ 326.1392, found 326.1391.

### 4-(5-(benzyloxy)-6-(1,3-dioxolan-2-yl)pyridin-2-yl)butan-1-ol—221

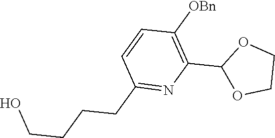 [SMILES: OCCCCc1ccc(OCc2ccccc2)c(C2OCCO2)n1]

Alkyne 220 (0.95 g, 2.94 mmol) and 2,2′-dipyridyl (181 mg, 1.16 mmol, 0.4 equiv.) was dissolved in MeOH (19 mL, 0.15 M), the solution was degassed with Argon. Pd/C 10% (618 mg, 0.58 mmol, 0.2 equiv.) was added, the mixture was stirred for 8 h at rt under hydrogen at 20 bars. The crude was purified by flash chromatography on silica gel (DCM/MeOH 0 to 7% over 45 min, 40G interchim SIHP, 15 micro, 30 mL/min) to afford a colourless oil (557 mg, 58%). Rf=0.38 (DCM/MeOH 95/5, v/v, vanillin). ¹H NMR (300 MHz, CDCl₃) δ (ppm) 7.51-7.32 (m, 5H), 7.22 (d, J=8.5 Hz, 1H), 7.10 (d, J=8.5 Hz, 1H), 6.37 (s, 1H), 5.14 (s, 2H), 4.35-4.21 (m, 2H), 4.15-4.00 (m, 2H), 3.69 (t, J=6.3 Hz, 2H), 2.85 (t, J=7.4 Hz, 2H), 2.31 (s, 1H), 1.92-1.76 (m, 2H), 1.75-1.54 (m, 2H). ¹³C NMR (75 MHz, CDCl₃): δ (ppm) 153.4, 151.6, 145.2, 136.3, 128.6, 128.1, 127.4, 123.7, 121.0, 100.4, 70.7, 65.7, 62.3, 36.3, 32.0, 25.6. HRMS (ESI⁺): m/z calcd for [C₁₉H₂₄NO₄]⁺ 330.1705, found 330.1705.

### 1-(4-(5-(benzyloxy)-6-(1,3-dioxolan-2-yl)pyridin-2-yl)butyl)-4,4-dimethyl-1,4-azasilinane—222

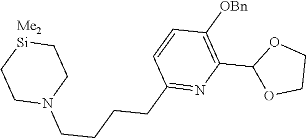

Alcohol 221 (467 mg, 1.42 mmol, 1.0 equiv.) and Et₃N (0.23 mL, 2.84 mmol, 2 equiv.) was dissolved in DCM (5 mL), then MsCl (0.165 mL, 2.12 mmol, 1.5 equiv.) was added at 0° C. The resulting mixture was stirred for 30 min at rt. After completion, the mixture was quenched with water and extracted with dichloromethane (3×20 mL). Dried over MgSO₄ and concentrated in vacuo. The obtained crude product was used directly for the next reaction.

4,4-dimethyl-1,4-azasilinane·HCl (470 mg, 2.84 mmol, 2 equiv.) was basified by an aq sat sol of K₂CO₃ and extracted with DCM, dried over MgSO4 and concentrated. The crude mesylate was dissolved in MeCN (10 mL) before 4,4-dimethyl-1,4-azasilinane and K₂CO₃ (392 mg, 2.84 mmol, 2.0 equiv.) were added and the reaction mixture was heated at 60° for 18 hours. The reaction mixture was concentrated and the crude material was purified by flash chromatography on silica gel (MeOH in DCM 0 to 10% over 40 min, 25G, SIHP, 30 microns, 25 mL/min). Rf=0.2 (95/5 DCM/MeOH, v/v, ninhydrin). ¹H NMR (300 MHz, CDCl₃) δ (ppm) 7.39-7.25 (m, 5H), 7.09 (d, J=8.5 Hz, 1H), 6.97 (d, J=8.4 Hz, 1H), 6.23 (s, 1H), 5.02 (s, 2H), 4.22-4.06 (m, 2H), 4.02-3.85 (m, 2H), 2.76 (t, J=6.4 Hz, 4H), 2.71-2.65 (m, 2H), 2.48 (t, J=7.6 Hz, 2H), 1.61 (dd., J=14.9, 6.9 Hz, 2H), 1.55 (dd, J=6.8, 3.9 Hz, 2H), 0.78 (t, J=6.3 Hz, 4H), 0.00 (s, 6H). ¹³C NMR (75 MHz, CDCl₃) δ (ppm) 153.2, 151.5, 145.5, 136.4, 128.6, 128.1, 127.4, 123.5, 120.8, 100.8, 70.7, 65.7, 57.4, 52.3, 40.0, 36.9, 27.7, 25.8, 12.6, −3.3. HRMS (ESI⁺): m/z calcd for [C₂₅H₃₁N₂O₃Si]⁺ 441.2573, found 441.2585.

### (E)-6-(4-(4,4-dimethyl-1,4-azasilinan-1-yl)butyl)-3-hydroxypicolinaldehyde oxime—223

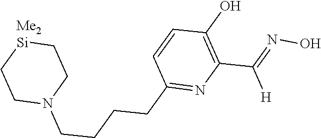

Alkane 222 (360 mg, 0.82 mmol, 1.0 equiv.) was dissolved in MeOH (8 mL) and the solution was degassed with Argon. Pd(OH)₂/C 20% (88 mg, 0.16 mmol, 0.2 equiv.) was added, the mixture was stirred for 2 h at rt under hydrogen. The reaction mixture was filtered over celite, solvents were removed under vacuum. The crude material was used for the next step. A mixture of formic acid and water (16 mL 1:1) was added to acetal and heated for 18 hours at 60° C. The reaction mixture was neutralized by a saturated aqueous solution of NaHCO₃ until neutral and extracted with DCM (4×30 mL), dried over MgSO4 and concentrated. The crude material was used for the next step. NaHCO₃ (277 mg, 3.29 mmol, 4 equiv.) and NH₂OH·HCl (143 mg, 2.06 mmol, 2.5 equiv.) were added to a mixture of crude aldehyde in MeOH and stirred for 18 hours. The reaction mixture was concentrated, and washed with water and extracted with DCM (3×25 mL), dried over MgSO₄ and concentrated. Purified by flash chromatography (10 to 40% MeOH in DCM over 50 min, 12G SiHP, 30 microns, 20 mL/min) to afford the title compound as an oil (155 mg, 58%). Dissolved in AcOEt, 0.47 mL of 1M MsOH in AcOEt (961 mg of MsOH in 10 mL of AcOEt) was added, concentrated and dried in high vacuum to afford the mesylate salt.

¹H NMR (300 MHz, MeOD) δ 8.11 (s, 1H), 7.14 (d, J=8.5 Hz, 1H), 7.03 (d, J=8.5 Hz, 1H), 3.45 (q, J=9.6, 8.1 Hz, 2H), 2.94 (t, J=7.7 Hz, 2H), 2.63 (td, J=8.4, 7.0, 5.0 Hz, 2H), 2.51 (s, 3H), 1.66-1.47 (m, 4H), 0.90 (t, J=5.5 Hz, 4H), 0.16-0.16 (m, 6H). BC NMR (75 MHz, MeOD) δ 152.6, 152.1, 150.8, 135.0, 125.0, 124.2, 55.6, 52.2, 38.1, 35.3, 26.5, 23.3, 10.0, −4.8, −6.1. HRMS (ESI⁺): m/z calcd for [C₁₆H₂₈N₃O₂Si]⁺ 322.1951, found 322.1949. HPLC (Column BEH C18 100*2.1 mm, 1.7 m, H₂O formic acid 0.1%/MeCN, 5 to 100% over 10 min, 0.5 mL/min): t_{R}=3.80 min (purity=90.5%).

### Example 2: Biological Activity of the Compounds of the Invention

Materials and Methods

1. hBChE and hAChE Preparation.

Recombinant hBChE and hAChE were produced and purified as previously described (Zueva et al., Neuropharmacology, 2019, 155: p. 131-141; Brazzolotto et al., FEBS J, 2012, 279(16): p. 2905-16).

2. Chemicals.

VX, sarin and tabun have been supplied by DGA maîtrise NRBC (Vert le Petit, France). 2-PAM and HI6 were from Pharmacie Centrale des Armées (Orléans, France) and other chemicals were purchased from Sigma-Aldrich.

3. Inhibition of hBChE and hAChE.

Stock solutions of OPNA at 5 mM in isopropanol were used to inhibit the purified hBChE and hAChE as previously described (Carletti et al., J Am Chem Soc, 2008, 130(47): p. 16011-20). Briefly, a ten-fold excess of OPNA was used to perform the inhibition of enzymes in a sodium phosphate buffer (100 mM, pH 7.4, 0.1% BSA) at 25° C. Complete inhibition of enzymes was monitored by measuring the residual activity with a modified Ellman assay as previously described (Ellman et al., Biochem Pharmacol, 1961, 7: p. 88-95) and excess of OPNA were removed by using a desalting PD-10 column (GE Healthcare).

4. IC50 Measurements.

Oximes were dissolved in water to prepare 40 mM stock solutions. Recombinant hBChE and hAChE activity was measured spectrophotometrically at 25° C., monitoring the absorbance at 412 nm. in 1 mL of Ellman's buffer (0.5 mM DTNB, 0.1% BSA, 0.1 M phosphate, pH 7.4), in the presence of appropriate oxime concentrations. Measurements were performed at least in duplicate for each concentration tested. The oxime concentration producing 50% inhibition was determined by nonlinear fitting with ProFit (Quantumsoft) using the standard IC 50 equation: % activity=100×IC50/(IC50+[Ox]).

5. Oxime reactivation of OP-Inhibited hBChE/hAChE.

The ability of oximes to reactivate OP-inhibited hBChE and optionally hAChE were assessed with a modified Ellman assay using a microplate reader (SPARK 10M, Tecan) described previously (Kitz et al., Biochem Pharmacol, 1965, 14(10): p. 1471-7; Worek et al., Biochem Pharmacol, 2004, 68(11): p. 2237-48) with minor modifications. Briefly, the desired oximes concentrations to be tested were dispensed in a 96-well flat-bottomed polystyrene microplate containing 0.1% BSA phosphate buffer and DTNB. At t=0, OP-inhibited hAChE and ATCh or OP-inhibited hBChE and BTCh diluted in 0.1% BSA phosphate buffer were injected in each well containing oximes using the built-in injectors of the microplate reader to a final volume of 200 μL. ATCh or BTCh hydrolysis was continuously monitored over 30 minutes and the increase of absorbance at 412 nm recorded every 10 seconds at 25° C. Activities were individually corrected for oxime-induced hydrolysis of ATCh or BTCh.

Reactivation of OP-inhibited enzyme by oximes proceeds according to scheme 1 and kinetics of oximes reactivation were determined as previously described (ibid.). For each oxime concentration, the apparent reactivation rate, k_{obs}, the dissociation constant, K_{D} and the reactivation rate constant, kᵣ, were calculated by nonlinear fitting with ProFit (Quantumsoft) using the following standard oxime-concentration-dependent reactivation equation (1):

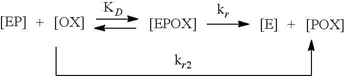

and the following equation:

$${k}_{obs} = \frac{{k}_{r} [ OX ]}{{K}_{D} + [ OX ]}$$

When [OX]<<K_{D}, Eq (1) simplifies to Eq (2):

$${k}_{obs} = ( \frac{{k}_{r}}{{K}_{D}} ) [ OX ]$$

The second order reactivation rate constant kᵣ₂, describing the specific reactivity can be derived from Eq (2):

$${k}_{r 2} = \frac{{k}_{r}}{{K}_{D}}$$

For the continuous method of recording OP-inhibited hAChE/hBChE reactivation by oximes, the velocity of substrate hydrolysis (v) is proportional to the concentration of the reactivated hAChE/hBChE and is expressed and derived as equation 4 and 5 respectively. vₜ is the velocity at time t and v₀ represents the maximum velocity. Equation 5 was used to determine the k_{obs} by non-linear regression analysis for each individual oxime concentration with ProFit (Quantumsoft).

$${v}_{t} = {v}_{0} ( 1 - {e}^{- {k}_{obs} t} ) \qquad Eq ( 4 )$$

$$- d [ S ] = {\int }_{0}^{t} v dt = {v}_{0} t + \frac{{v}_{0}}{{k}_{obs}} ( {e}^{- {k}_{obs} t} - 1 ) \qquad Eq ( 5 )$$

6. Reactivation Kinetics for Newly Synthesize Compounds

Results are presented in the following tables 1 and 2. HI-6 is asoxime chloride and 2-PAM is pralidoxime, both references outside the invention.

TABLE 1
Reactivation of OP-inhibited hBChE by oximes

| OP | Oxime | Kᵣ (min⁻¹) | K_{D} (μM) | Kᵣ₂ (mM⁻¹ · min⁻¹) |
|---|---|---|---|---|
| VX | HI-6 | 0.03 ± 0.002 | 307 ± 41 | 0.1 |
| | AB-746 | 0.065 ± 0.0009 | 0.4 ± 0.05 | 175 |
| | AB-739 | 0.09 ± 0.0013 | 81 ± 7 | 1 |
| | AB-639 | 0.14 ± 0.01 | 117 ± 22 | 1 |
| | AB-745 | 0.6 ± 0.06 | 253 ± 36 | 2.4 |
| Sarin | HI-6 | 0.3 ± 0.02 | 409 ± 71 | 0.7 |
| | AB-746 | 0.07 ± 0.009 | 0.27 ± 0.04 | 259 |
| | AB-745 | 0.16 ± 0.003 | 30 ± 2 | 5.3 |
| Paraoxon | 2-PAM | 0.35 ± 0.02 | 65 ± 22 | 5.4 |
| | HI-6 | ∅ | ∅ | ∅ |
| | AB-746 | 0.045 ± 0.0006 | 0.15 ± 0.03 | 300 |
| | AB-639 | 0.07 ± 0.001 | 8 ± 1 | 9 |

TABLE 2
IC50 of oxime on hBChE

| Oxime | IC₅₀ (μM) |
|---|---|
| 2-PAM | 10% inhibition at 5 mM |
| HI-6 | 6345 ± 515 |
| AB-746 | 19 ± 2 |
| AB-739 | 1297 ± 131 |
| AB-639 | 1776 ± 137 |
| AB-745 | 80 ± 8 |

### Example 3: Synthesis of Other Compounds of the Invention

UPLC Methods

Method A: Analytic UPLC (Waters Acquity BEH C18 column, 1.7 μm, 2.1×100 mm) with MeCN and analytical water with 0.1% formic acid as eluents (gradient from 5% to 100% MeCN over 10 min) at a flow rate of 0.4 mL/min. UV detection at 254 nm.

Method B: Analytic UPLC (Waters Acquity BEH Shield column, 1.7 m. 2.1×100 mm) with MeCN and analytical water with 0.1% formic acid as eluents (0% MeCN for 1 min, gradient from 0% to 100% MeCN over 9 min) at a flow rate of 0.4 mL/min. UV detection at 254 nm.

Method C: Analytic UPLC (Waters Acquity BEH C18 column, 1.7 μm, 2.1×100 mm) with MeCN and analytical water with 0.1% formic acid as eluents (gradient from 5% to 100% MeCN over 10 min) at a flow rate of 0.4 mL/min. UV detection at 254 nm.

General Procedure

General Procedure a for the Formation of Potassium Trifluoroborane Salts

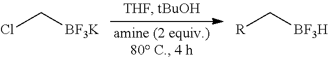

Adapted from Molander et al. (J. Org. Chem. 2011, 76, 2762-2769): A round bottom flask equipped with a stirrer bar was charged with chloromethyltrifluoroborate (1 equiv.). The vial was then evacuated under vacuum and purged with N₂. A mixture of anhydrous THF and t-BuOH (3:1, 1 M), and amine (2 equiv.) were added via syringes. The reaction mixture was stirred and heated to 80° C. for 2 to 4 hours. At this point the reaction mixture was transferred to a flask, and the volatiles were removed in vacuo. The solid was dried under high vacuum overnight before being dissolved in a solution of hot HPLC acetone and the solution filtered to remove KCl. The filtrate was concentrated in vacuo, dissolved in a minimal amount of hot acetone, and precipitated by the dropwise addition of Et₂O. Additional Et₂O was added to facilitate filtering.

General Procedure B for Suzuki-Miyaura Cross-Coupling

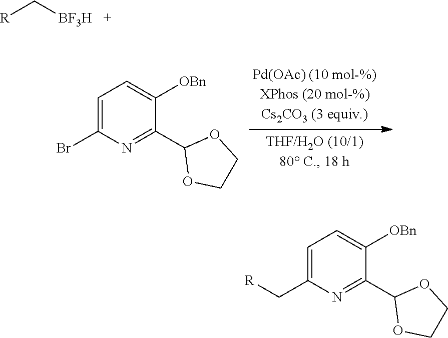

Adapted from Molander et al. (ibid.): Trifluoroborane (1.5 equiv.), Cs₂CO₃ (3.0 equiv.), 3-(benzyloxy)-6-bromo-2-(1,3-dioxolan-2-yl)pyridine (ACS Chem. Neurosci. 2020, 11, 1072-1084, 1.0 equiv.), Pd(OAc)₂ (10 mol-%), and XPhos (20 mol-%) were poured in a flask then evacuated under vacuum and purged with Ar. A mixture of THF and H₂O (10/1, 0.25 M) were added by syringe and the reaction mixture was stirred and heated at 80° C. for 18 h and then cooled to room temperature. The reaction mixture is filtered through Celite, rinsed with a large quantity of EtOAc and concentrated in vacuo. The crude material was purified by flash chromatography on silica gel to afford the desired product.

General Procedure C for Hydrogenation

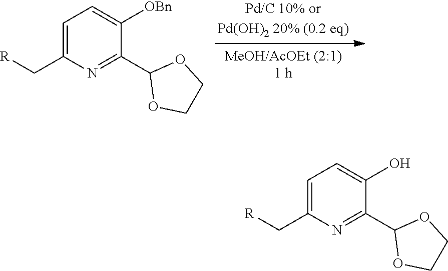

Benzyloxypyridine (1.0 equiv.) was dissolved in MeOH/AcOEt (2/1, 0.1 M), the solution was degassed with Argon. Pd/C 10% (0.2 equiv.) or Pd(OH)₂/C 20% (0.2 equiv.) was added, the mixture was stirred for 1 hour at rt under H₂. Palladium was filtered over Celite and the solvent was removed under vacuum. The crude material was purified by flash chromatography on silica gel.

General Procedure D for Acetal Hydrolysis and Oxime Formation

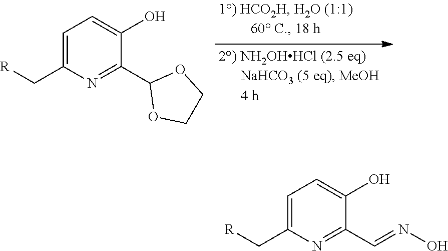

Acetal (1.0 equiv.) in HCO₂H/H₂O (1/1, 0.1 M) was heated for 18 hours at 60° C. The crude mixture was concentrated in vacuo. NaHCO₃ (5.0 equiv.) and NH₂OH·HCl (2.5 equiv.) were added to a mixture of crude aldehyde (1.0 equiv.) in MeOH (0.1 M) and stirred for 4 hours. The crude material was filtered through celite, concentrated and purified by flash chromatography on silica gel resulting in the isolation of formate salt.

General Procedure E for Acetal Hydrolysis (with Aqueous Work-Up) and Oxime Formation

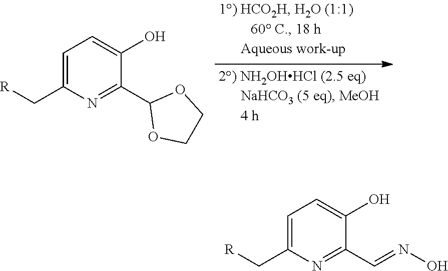

Acetal (1.0 equiv.) in HCO₂H/H₂O (1/1, 0.1 M) was heated for 18 hours at 60° C. The crude mixture was concentrated in vacuo and quenched by an aqueous saturated solution of NaHCO₃ until neutral and extracted with AcOEt (3×50 mL). Dried over MgSO₄ and concentrated, the crude material was used for the next step. NaHCO₃ (5.0 equiv.) and NH₂OH·HCl (2.5 equiv.) were added to a mixture of crude aldehyde (1.0 equiv.) in MeOH (0.1 M) and stirred for 4 hours. The reaction mixture was filtered through celite, concentrated and purified by flash chromatography on silica gel.

If needed, the mesylate salt was formed by adding 1 equivalent of 1 M MsOH in AcOEt to a solution of oxime in a mixture of AcOEt/MeOH.

General Procedure F for Conjugate Addition to Vinyl Pyridine

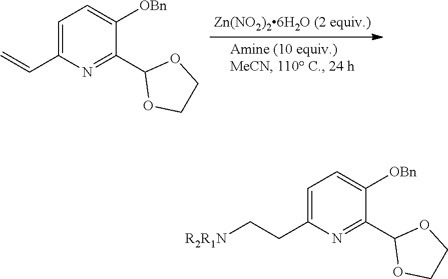

Adapted from Williams et al. (ChemSusChem 2015, 8, 1083-1087): Zn(NO₃)₂·6H₂O (2 equiv.) was added to a sealed microwave tube containing vinylpyridine 30 (1 equiv.) in acetonitrile HPLC (1 M) and amine (10 equiv.). The tube was sealed before the reaction mixture was heated to 110° C. for 24 hours. After being allowed to cool to room temperature, the reaction was filtered through celite rinsed with MeOH and concentrated. The residue was washed with an aqueous saturated solution of NaHCO₃ and extracted with DCM, dried over MgSO₄, and concentrated. The crude material was purified by flash chromatography on silica gel.

General Procedure G for Buchwald-Hartwig Cross-Coupling

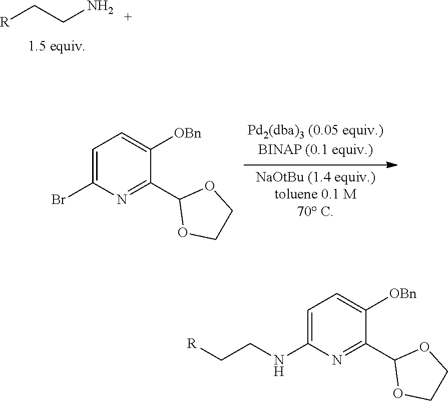

Adapted from Buchwald et al. (J. Org. Chem. 1996, 61, 7240-7241): 3-(benzyloxy)-6-bromo-2-(1,3-dioxolan-2-yl)pyridineErreur ! Signet non défini. (1 equiv.), Pd₂(dba)₃ (5 mol-%), BINAP (10 mol-%), NaO-t-Bu (1.4 equiv.), were added to an oven-dried flask, the flask was purged with argon. Amine (1.5 equiv.) and toluene (0.1 M with 2-bromopyridine) were added and the reaction mixture was then heated to 70° C. under argon for 4 to 24 hours. The reaction mixture was then allowed to cool to room temperature, taken up in diethyl ether, washed three times with saturated brine, dried over MgSO₄, and concentrated in vacuo to give the crude product. The crude material was purified by flash chromatography on silica gel.

General Procedure H for Sonogashira Cross-Coupling

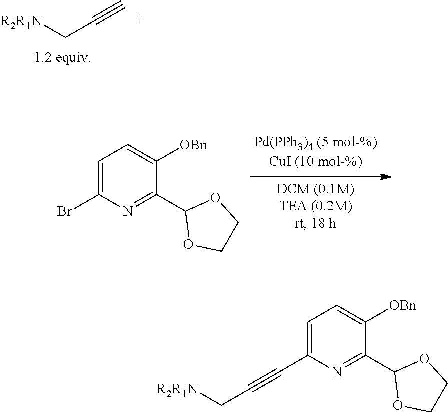

3-(benzyloxy)-6-bromo-2-(1,3-dioxolan-2-yl)pyridineErreur ! Signet non défini. (1 equiv.) and alkyne (1.2 equiv.) were added to a mixture of triethylamine (0.2 M) and dichloromethane (0.1 M). This mixture was degassed 5 min with argon, then Pd(PPh₃)₄ (5 mol-%) and CuI (10 mol-%) were added and the mixture was stirred 18 hours under argon. Solvents were removed and the crude material was purified by flash chromatography on silica gel.

Product Descriptions and Synthetic Protocols

### Trifluoro((1,2,3,4-tetrahydroisoquinolin-2-ium-2-yl)methyl)borate 1′

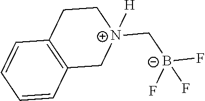 [SMILES: F[B-](F)(F)C[NH+]1CCc2ccccc2C1]

General procedure A was used with tetrahydroisoquinoline (511 mg, 3.84 mmol. 2 equiv.) to afford a white solid (212 mg, 51%). ¹H NMR (300 MHz, CD₃CN) δ 7.21 (m, 4H), 4.53-4.13 (m, 2H), 3.68 (s, 1H), 3.27 (s, 1H), 3.20-2.98 (m, 2H), 2.22 (s, 2H). ¹⁹F NMR (282 MHz, CD₃CN) δ −142.12 (dd, J=99.2, 46.8 Hz).

### Trifluoro((2,3,4,9-tetrahydro-1H-pyrido[3,4-b]indol-2-ium-2-yl)methyl)borate 2′

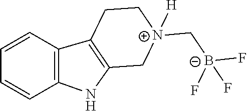 [SMILES: F[B-](F)(F)C[NH+]1CCc2c([nH]c3ccccc23)C1]

General procedure A was used with triptoline (551 mg, 3.19 mmol, 2 equiv.) to afford a white solid (0.6 g, 74%). ¹H NMR (300 MHz, DMSO) δ 11.08 (s, 1H), 7.52-7.37 (m, 2H), 7.20-7.01 (m, 2H), 4.41 (s, 2H), 3.49 (s, 2H), 2.97 (d, J=6.2 Hz, 2H), 2.25 (q, J=5.2 Hz, 2H). ¹³C NMR (75 MHz, DMSO) δ 136.7, 127.5, 126.3, 121.9, 119.4, 118.3, 111.8, 105.4, 67.5, 52.7, 50.4, 18.7. ¹⁹F NMR (282 MHz, DMSO) δ −137.41.

### Trifluoro((4-phenylpiperazin-1-ium-1-yl)methyl)borate 3′

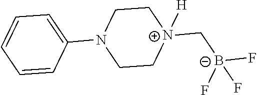 [SMILES: F[B-](F)(F)C[NH+]1CCN(c2ccccc2)CC1]

General procedure A was used with N-phenylpiperazine (2.11 g, 13.05 mmol, 2 equiv.) to afford a white solid (1.3 g, 76%). ¹H NMR (300 MHz, DMSO) δ 8.81 (s, 1H), 7.24 (dd, J=8.6, 7.3 Hz, 2H), 6.96 (d, J=7.9 Hz, 2H), 6.84 (t, J=7.3 Hz, 11H), 3.91-2.74 (m, 10H), 2.04 (dd, J=10.1, 5.0 Hz, 2H). ¹³C NMR (75 MHz, DMSO) δ 149.77, 129.03, 119.73, 115.77, 53.06, 45.46. ¹⁹F NMR (282 MHz, DMSO) δ −137.11 (s). HRMS (ESI⁺): m/z calcd for [C₁₁H₂₀BN₃F₃]⁺ 262,1702, found 262,1690.

### Trifluoro((4-(pyridin-2-yl)piperazin-1-ium-1-yl)methyl)borate 4′

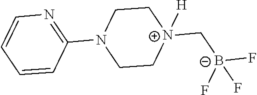 [SMILES: F[B-](F)(F)C[NH+]1CCN(c2ccccn2)CC1]

General procedure A was used with 1-(pyridin-2-yl)piperazine (1.08 g, 6.65 mmol, 2 equiv.) to afford a white solid (513 mg, 63%). 1H NMR (300 MHz, DMSO) δ 8.15 (dd, J=5.2, 1.9 Hz, 1H), 7.59 (ddd, J=8.9, 7.1, 2.0 Hz, 1H), 6.90 (d, J=8.5 Hz, 1H), 6.73 (dd, J=7.1, 4.9 Hz, 1H), 4.21-2.94 (m, 8H), 2.03 (q, J=5.2 Hz, 2H). ¹³C NMR (75 MHz, DMSO) δ 158.60, 148.04, 138.31, 114.46, 108.05, 53.34, 42.37. ¹⁹F NMR (282 MHz, DMSO) δ −137.16. HRMS (ESI⁺): m/z calcd for [C₁₀H₁₄BN₃F₃]⁺ 244.1233, found 244.1238.

### Trifluoro((4-(pyrimidin-2-yl)piperazin-1-ium-1-yl)methyl)borate 5′

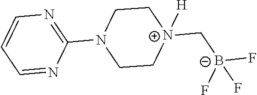 [SMILES: F[B-](F)(F)C[NH+]1CCN(c2ncccn2)CC1]

General procedure A was used with 2-(piperazin-1-yl)pyrimidine (2.14 g, 13.06 mmol, 2 equiv.) to afford a white solid (1.2 g, 70%). ¹H NMR (300 MHz, DMSO) δ 8.93 (s, 1H), 8.45-8.25 (m, 2H), 6.73 (t, J=4.8 Hz, 1H), 4.56 (s, 2H), 3.78-2.67 (m, 6H), 2.02 (q, J=5.1 Hz, 21H). ¹³C NMR (75 MHz, DMSO) δ 160.71, 158.07, 111.09, 52.84, 40.39. ¹⁹F NMR (282 MHz, DMSO) δ −137.19 (s). HRMS (ESI⁺): m/z calcd for [C₉H₁₈BN₅F₃]⁺ 264,1607, found 264,1616.

### 3-(benzyloxy)-2-(1,3-dioxolan-2-yl)-6-(piperidin-1-ylmethyl)pyridine 6′

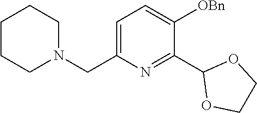 [SMILES: c1ccc(COc2ccc(CN3CCCCC3)nc2C2OCCO2)cc1]

General procedure B was used with commercially available trifluoro(piperidin-1-ium-1-ylmethyl)borate (170 mg, 0.51 mmol, 1.3 equiv.) and 3-(benzyloxy)-6-bromo-2-(1,3-dioxolan-2-yl)pyridine (110 mg, 0.66 mmol, 1.0 equiv.) to afford a white solid (90 mg, 50%). ¹H NMR (300 MHz, MeOD) δ 7.44-7.11 (m, 10H), 6.06 (s, 1H), 5.01 (s, 2H), 4.82 (s, 2H), 4.01 (dd, J=8.4, 5.0 Hz, 2H), 3.84 (dd, J=8.4, 5.0 Hz, 3H), 3.59 (s, 2H), 2.47 (s, 4H), 1.60-1.43 (m, 5H), 1.36 (d, J=4.8 Hz, 2H). ¹³C NMR (75 MHz, MeOD) δ 154.3, 148.1, 146.3, 137.6, 129.6, 129.2, 128.7, 126.7, 122.4, 102.0, 71.6, 66.7, 64.0, 55.1, 26.0, 24.6. HRMS (ESI⁺): m/z calcd for [C₂₁H₂₇N₂O₃]⁺ 355.2022, found 355.2022.

### 4-((5-(benzyloxy)-6-(1,3-dioxolan-2-yl)pyridin-2-yl)methyl)morpholine 7′

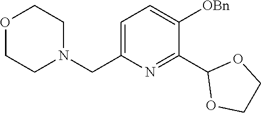 [SMILES: c1ccc(COc2ccc(CN3CCOCC3)nc2C2OCCO2)cc1]

General procedure B was used with commercially available trifluoro(morpholino-4-iummethyl)borate (1.09 g, 6.47 mmol, 1.5 equiv.) and 3-(benzyloxy)-6-bromo-2-(1,3-dioxolan-2-yl)pyridine (1.45 g, 4.31 mmol, 1.0 equiv.) to afford a white solid (1.29 g, 84%). ¹H NMR (300 MHz, CDCl₃) δ 7.51-7.36 (m, 6H), 7.27 (d, J=8.5 Hz, 1H), 6.34 (s, 1H), 5.15 (s, 2H), 4.31-4.17 (m, 2H), 4.17-3.98 (m, 2H), 3.75 (t, J=4.7 Hz, 4H), 3.68 (s, 2H), 2.54 (dd, J=5.7, 3.6 Hz, 4H). ¹³C NMR (75 MHz, CDCl₃) δ 152.4, 149.4, 145.4, 136.2, 128.7, 128.2, 127.4, 124.1, 120.7, 100.9, 70.6, 67.0, 65.7, 64.1, 53.7. HRMS (ESI⁺): m/z calcd for [C₂₀H₂₅N₂O₄]⁺ 357.1814, found 357.1802.

### 2-((5-(benzyloxy)-6-(1,3-dioxolan-2-yl)pyridin-2-yl)methyl)-1,2,3,4-tetrahydroisoquinoline 8′

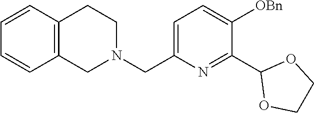 [SMILES: c1ccc(COc2ccc(CN3CCc4ccccc4C3)nc2C2OCCO2)cc1]

General procedure B was used with trifluoro((1,2,3,4-tetrahydroisoquinolin-2-ium-2-yl)methyl)borate 1′ (448 mg, 2.1 mmol. 1.4 equiv.) and 3-(benzyloxy)-6-bromo-2-(1,3-dioxolan-2-yl)pyridine (500 mg, 1.49 mmol, 1.0 equiv.) to afford a solid (329 mg, 55%). ¹H NMR (300 MHz, CDCl₃) δ 7.37-7.31 (m, 1H), 7.31-7.13 (m, 6H), 7.10 (d, J=8.5 Hz, 1H), 7.03-6.88 (m, 4H), 6.86-6.81 (m, 1H), 6.23 (s, 1H), 4.95 (s, 2H), 4.18-4.01 (m, 2H), 3.97-3.81 (m, 2H), 3.70 (s, 2H), 3.56 (s, 2H), 2.77 (t, J=5.8 Hz, 2H), 2.65 (dd, J=6.0, 4.9 Hz, 2H). ¹³C NMR (75 MHz, CDCl₃) δ 152.4, 150.2, 145.3, 136.4, 135.0, 134.4, 128.8, 128.7, 128.2, 127.5, 126.6, 126.2, 125.7, 124.0, 120.9, 101.0, 70.7, 65.8, 63.6, 56.2, 50.9, 29.3. HRMS (ESI⁺): m/z calcd for [C₂₅H₂₇N₂O₃]⁺ 403.2022, found 403.2022.

### 2-((5-(benzyloxy)-6-(1,3-dioxolan-2-yl)pyridin-2-yl)methyl)-2,3,4,9-tetrahydro-1H-pyrido[3,4-b]indole 9′

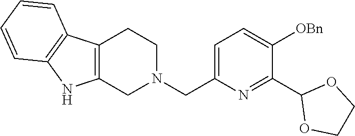 [SMILES: c1ccc(COc2ccc(CN3CCc4c([nH]c5ccccc45)C3)nc2C2OCCO2)cc1]

General procedure B was used with trifluoro((2,3,4,9-tetrahydro-1H-pyrido[3,4-b]indol-2-ium-2-yl)methyl)borate 2′ (1.64 g, 6.47 mmol, 1.5 equiv.) and 3-(benzyloxy)-6-bromo-2-(1,3-dioxolan-2-yl)pyridine (1.45 g, 4.31 mmol, 1.0 equiv.) to afford a solid (481 mg, 25%). ¹H NMR (300 MHz, CDCl₃) δ 8.57 (s, 1H), 7.27 (d, J=3.5 Hz, 1H), 7.26-7.18 (m, 6H), 7.02-6.96 (m, 2H), 6.93-6.86 (m, 2H), 6.21 (s, 1H), 4.86 (s, 2H), 4.09-3.93 (m, 2H), 3.88-3.76 (m, 2H), 3.64 (s, 2H), 3.28 (s, 2H), 2.68 (t, J=5.5 Hz, 2H), 2.62 (d, J=5.1 Hz, 2H). ¹³C NMR (75 MHz, CDCl₃) δ 152.6, 150.2, 144.9, 136.3, 136.2, 132.3., 128.8, 128.3, 127.6, 127.2, 124.6, 121.1, 121.0, 119.1, 117.9, 111.1, 107.7, 100.7, 70.6. 65.8, 62.9, 53.6, 51.1, 50.3, 21.5. HRMS (ESI⁺): m/z calcd for [C₂₇H₂₈N₃O₃]⁺ 442.2131, found 442.2120.

### 1-((5-(benzyloxy)-6-(1,3-dioxolan-2-yl)pyridin-2-yl)methyl)-4-methylpiperazine 10′

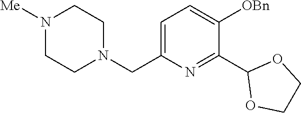 [SMILES: CN1CCN(Cc2ccc(OCc3ccccc3)c(C3OCCO3)n2)CC1]

General procedure B was used with commercially available trifluoro((4-methylpiperazin-1-ium-1-yl)methyl)borate (1.18 g, 6.47 mmol, 1.5 equiv.) and 3-(benzyloxy)-6-bromo-2-(1,3-dioxolan-2-yl)pyridine (1.45 g, 4.31 mmol, 1.0 equiv.) to afford a white solid (734 mg, 46%). ¹H NMR (300 MHz, CDCl₃) δ 7.51-7.35 (m, 6H), 7.26 (d, J=8.5 Hz, 1H), 6.35 (s, 1H), 5.15 (s, 2H), 4.34-4.17 (m, 2H), 4.15-3.98 (m, 2H), 3.68 (s, 2H), 2.58 (s, 4H), 2.50 (s, 4H), 2.33 (s, 3H). ¹³C NMR (75 MHz, CDCl₃) δ 152.3, 149.8, 145.3, 136.3, 128.7, 128.2, 127.4, 124.1, 120.7, 100.9, 70.6, 65.7, 63.6, 55.1, 53.1, 46.0. HRMS (ESI⁺): m/z calcd for [C₂₁H₂₈N₃O₃]⁺ 370.2131 found 370.2133.

### 1-((5-(benzyloxy)-6-(1,3-dioxolan-2-yl)pyridin-2-yl)methyl)-4-phenylpiperazine 11′

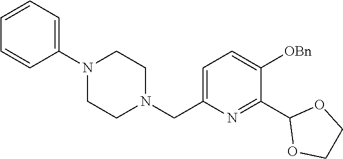 [SMILES: c1ccc(COc2ccc(CN3CCN(c4ccccc4)CC3)nc2C2OCCO2)cc1]

General procedure B was used with trifluoro((4-phenylpiperazin-1-ium-1-yl)methyl)borate 3′ (601 mg, 2.46 mmol, 1.2 equiv.) and 3-(benzyloxy)-6-bromo-2-(1,3-dioxolan-2-yl)pyridine (690 mg, 2.05 mmol, 1.0 equiv.) to afford a solid (683 mg, 77%). ¹H NMR (300 MHz, CDCl₃) δ 7.51-7.32 (m, 7H), 7.32-7.22 (m, 31H), 6.98-6.91 (m, 2H), 6.87 (tt, J=7.3, 1.1 Hz, 1H), 6.35 (s, 1H), 5.15 (s, 2H), 4.34-4.17 (m, 2H), 4.16-3.98 (m, 2H), 3.74 (s, 2H), 3.27-3.18 (m, 4H), 2.74-2.64 (m, 4H). ¹³C NMR (75 MHz, CDCl₃) δ 152.31, 151.39, 149.76, 145.28, 136.24, 129.09, 128.19, 124.01, 120.73, 119.59, 116.01, 100.98, 70.60, 65.75, 63.69, 53.20. HRMS (ESI⁺): m/z calcd for [C₂₆H₃₀N₃O₃]⁺ 432.2287 found 432.2289.

### 1-((5-(benzyloxy)-6-(1,3-dioxolan-2-yl)pyridin-2-yl)methyl)-4-(pyridin-2-yl)piperazine 12′

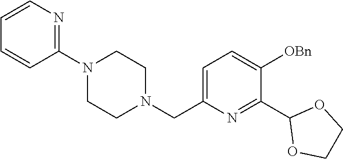 [SMILES: c1ccc(COc2ccc(CN3CCN(c4ccccn4)CC3)nc2C2OCCO2)cc1]

General procedure B was used with trifluoro((4-(pyridin-2-yl)piperazin-1-ium-1-yl)methyl)borate 4′ (513 mg, 2.09 mmol, 1.5 equiv.) and 3-(benzyloxy)-6-bromo-2-(1,3-dioxolan-2-yl)pyridine (469 mg, 1.39 mmol, 1.0 equiv.) to afford a solid (493 mg, 82%). ¹H NMR (300 MHz, CDCl₃) δ 8.23 (ddd. J=4.9, 2.0, 0.9 Hz, 1H), 7.59-7.19 (m, 7H), 6.72-6.60 (m, 2H), 6.37 (s, 1H), 5.34 (s, 2H), 4.35-4.18 (m, 2H), 4.17-4.03 (m, 2H), 3.73 (d, J=14.1 Hz, 2H), 3.64-3.55 (m, 4H), 2.66 (q, J=4.9 Hz, 4H). HRMS (ESI⁺): m/z calcd for [C₂₅H₂₉N₄O₃]⁺ 433.2240, found 433.2232.

### 2-(4-((5-(benzyloxy)-6-(1,3-dioxolan-2-yl)pyridin-2-yl)methyl)piperazin-1-yl)pyrimidine 13′

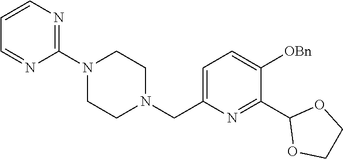 [SMILES: c1ccc(COc2ccc(CN3CCN(c4ncccn4)CC3)nc2C2OCCO2)cc1]

General procedure B was used with trifluoro((4-(pyrimidin-2-yl)piperazin-1-ium-1-yl)methyl)borate 5′ (632 mg, 2.99 mmol, 1.5 equiv.) and 3-(benzyloxy)-6-bromo-2-(1,3-dioxolan-2-yl)pyridine (670 mg, 1.99 mmol, 1.0 equiv.) to afford a solid (409 mg, 48%). ¹H NMR (300 MHz, CDCl₃) δ 8.30 (d, J=4.8 Hz, 2H), 7.47-7.36 (m, 6H), 7.27 (d, J=8.7 Hz, 1H), 6.48 (t, J=4.7 Hz, 1H), 6.33 (s, 1H), 5.14 (s, 2H), 4.31-4.15 (m, 2H), 4.13-3.96 (m, 2H), 3.84 (t, J=5.1 Hz, 4H), 3.71 (s, 2H), 2.57 (d, J=10.2 Hz, 2H). ¹³C NMR (75 MHz, CDCl₃) δ 161.67, 157.69, 152.30, 149.69, 145.33, 136.22, 128.65, 128.18, 127.42, 123.98, 120.71, 109.75, 100.91, 70.59, 65.72, 63.76, 53.45, 53.03, 43.71. HRMS (ESI⁺): m/z calcd for [C₂₄H₂₅N₅O₃]⁺ 434.2192, found 434.2204.

### 2-(1,3-dioxolan-2-yl)-6-(piperidin-1-ylmethyl)pyridin-3-ol 14′

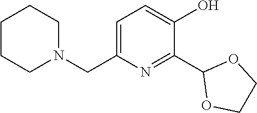 [SMILES: Oc1ccc(CN2CCCCC2)nc1C1OCCO1]

General procedure C was used with starting material 6′ (420 mg, 1.19 mmol, 1.0 equiv.) and Pd(OH)₂/C 20% (166 mg, 0.24 mmol, 0.2 equiv.) to afford an oil (281 mg, 90%). ¹H NMR (300 MHz, CDCl₃) δ 7.52 (s, 1H), 7.29 (d, J=8.5 Hz, 1H), 7.09 (d, J=8.5 Hz, 1H), 5.87 (s, 1H), 4.23-4.08 (m, 2H), 4.08-3.93 (m, 2H), 3.55 (s, 2H), 2.41 (t, J=5.4 Hz, 4H), 1.53 (q, J=5.6 Hz, 4H), 1.44-1.30 (m, 2H). ¹³C NMR (75 MHz, CDCl₃) δ 151.7, 148.7, 138.5, 125.5, 125.4, 104.9, 65.2, 64.1, 54.4, 25.5, 24.0. HRMS (ESI⁺): m/z calcd for [C₁₄H₂₁N₂O₃]⁺ 265.1552, found 265.1514.

### 2-(1,3-dioxolan-2-yl)-6-(morpholinomethyl)pyridin-3-ol 15′

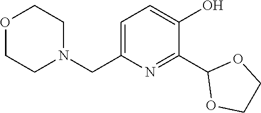 [SMILES: Oc1ccc(CN2CCOCC2)nc1C1OCCO1]

General procedure C was used with starting material 7′ (1.13 g, 3.18 mmol, 1.0 equiv.) Pd(OH)₂/C and 20% (892 mg, 0.64 mmol, 0.2 equiv.) to afford an oil (705 mg, 83%). ¹H NMR (300 MHz, CDCl₃) δ 8.03 (s, 1H), 7.21 (dd, J=8.4, 2.3 Hz, 1H), 7.09 (d, J=8.3 Hz, 1H), 5.88 (s, 1H), 4.10 (tt, J=5.0, 2.2 Hz, 2H), 4.02-3.86 (m, 2H), 3.66-3.52 (m, 4H), 3.52-3.43 (m, 2H), 2.36 (d, J=3.8 Hz, 4H). ¹³C NMR (75 MHz, CDCl₃) δ 151.6, 148.6, 139.1, 125.4, 125.0, 104.0, 66.7, 65.1, 63.7, 53.5. HRMS (ESI⁺): m/z calcd for [C₁₃H₁₉N₂O₄]⁺ 267.1345, found 267.1347.

### 6-((3,4-dihydroisoquinolin-2(1H)-yl)methyl)-2-(1,3-dioxolan-2-yl)pyridin-3-ol 16′

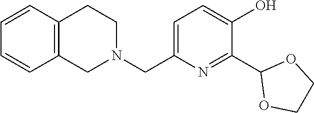 [SMILES: Oc1ccc(CN2CCc3ccccc3C2)nc1C1OCCO1]

General procedure C was used with starting material 8′ (330 mg, 0.82 mmol, 1.0 equiv.) and Pd(OH)₂/C 20% (115 mg, 0.16 mmol, 0.2 equiv.) to afford an oil (90 mg, 35%). ¹H NMR (300 MHz, CDCl₃) δ 7.46 (d, J=8.5 Hz, 1H), 7.24 (d, J=8.5 Hz, 1H), 7.20-7.07 (m, 3H), 7.07-6.97 (m, 1H), 5.99 (s, 1H), 4.34-4.19 (m, 2H), 4.19-4.04 (m, 2H), 3.83 (s, 2H), 3.72 (s, 2H), 2.94 (t, J=5.8 Hz, 2H), 2.83 (dd, J=6.1, 5.0 Hz, 2H). ¹³C NMR (75 MHz, CDCl₃) δ 151.6, 149.7, 138.1, 134.7, 134.3, 128.7, 126.6, 126.2, 125.8, 125.6, 125.1, 105.3, 65.2, 63.4, 56.1, 50.8, 29.1. HRMS (ESI⁺): m/z calcd for [C₁₈H₂₁N₂O₃]⁺ 313.1552, found 313.1553.

### 2-(1,3-dioxolan-2-yl)-6-((1,3,4,9-tetrahydro-2H-pyrido[3,4-b]indol-2-yl)methyl)pyridin-3-ol 17′

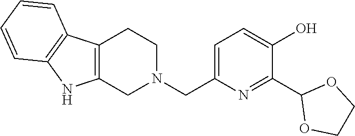 [SMILES: Oc1ccc(CN2CCc3c([nH]c4ccccc34)C2)nc1C1OCCO1]

General procedure C was used with starting material 9′ (360 mg, 0.82 mmol, 1.0 equiv.) and Pd/C 10% (230 mg, 0.16 mmol, 0.2 equiv.) to afford an oil (199 mg, 69%). ¹H NMR (300 MHz, DMSO) δ 10.67 (s, 1H), 9.85 (s, 1H), 7.35 (dd, J=8.0, 5.5 Hz, 2H), 7.30-7.19 (m, 2H), 7.01 (td, J=7.5, 1.4 Hz, 1H), 6.94 (td, J=7.3, 1.2 Hz, 1H), 6.08 (s, 1H), 4.23-4.09 (m, 2H), 4.02-3.86 (m, 2H), 3.75 (s, 2H), 3.60 (s, 2H), 2.82 (t, J=5.6 Hz. 2H), 2.71 (d, J=5.5 Hz, 2H). ¹³C NMR (75 MHz, DMSO) δ 151.2, 148.7, 143.0, 136.3, 133.3, 127.2, 124.5, 124.5, 120.7, 118.7, 117.8, 111.3, 106.8, 100.7, 65.4, 62.9, 51.1, 50.3, 21.6. HRMS (ESI⁺): m/z calcd for [C₂₀H₂₂N₃O₃]⁺ 352.1661, found 352.1670.

### 2-(1,3-dioxolan-2-yl)-6-((4-methylpiperazin-1-yl)methyl)pyridin-3-ol 18′

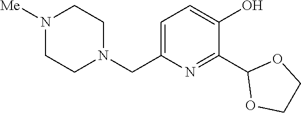 [SMILES: CN1CCN(Cc2ccc(O)c(C3OCCO3)n2)CC1]

General procedure C was used with starting material 10′ (700 mg, 1.89 mmol, 1.0 equiv.) and Pd/C 10% (530 mg, 0.38 mmol, 0.2 equiv.) to afford an oil (441 mg, 84%). ¹H NMR (300 MHz, CDCl₃) δ 8.44-8.15 (m, 1H), 7.26 (d, J=8.5 Hz, 1H), 7.10 (d, J=8.5 Hz, 1H), 5.93 (s, 1H), 4.27-4.11 (m, 2H), 4.11-3.95 (m, 2H), 3.57 (s, 2H), 2.63-2.34 (m, 7H), 2.26 (s, 3H). ¹³C NMR (75 MHz, CDCl₃) δ 151.5, 148.8, 125.3, 124.9, 104.1, 65.2, 63.3, 54.8, 52.6, 45.7. HRMS (ESI⁺): m/z calcd for [C₁₄H₂₂N₃O₃]⁺ 280.1661, found 280.1651.

### 2-(1,3-dioxolan-2-yl)-6-((4-phenylpiperazin-1-yl)methyl)pyridin-3-ol 19′

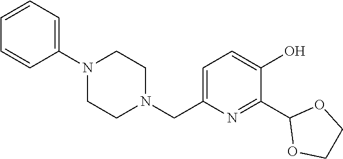 [SMILES: Oc1ccc(CN2CCN(c3ccccc3)CC2)nc1C1OCCO1]

General procedure C was used with starting material 11′ (1.11 mg, 2.57 mmol, 1.0 equiv.) and Pd/C 10% (722 mg, 0.51 mmol, 0.2 equiv.) to afford an oil (417 mg, 48%). ¹H NMR (300 MHz, CDCl₃) δ 7.33 (d, J=8.5 Hz, 1H), 7.25-7.10 (m, 4H), 6.84 (dt, J=7.9, 1.1 Hz, 2H), 6.77 (tt, J=7.3, 1.1 Hz, 1H), 5.83 (s, 1H), 4.24-3.95 (m, 4H), 3.60 (s, 2H), 3.18-3.03 (m, 4H), 2.64-2.54 (m, 4H). HRMS (ESI⁺): m/z calcd for [C₁₉H₂₄N₃O₃]⁺ 342.1818, found 342.1812.

### 2-(1,3-dioxolan-2-yl)-6-((4-(pyridin-2-yl)piperazin-1-yl)methyl)pyridin-3-ol 20′

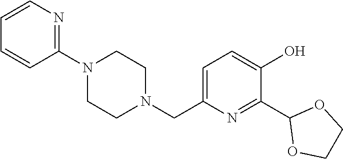 [SMILES: Oc1ccc(CN2CCN(c3ccccn3)CC2)nc1C1OCCO1]

General procedure C was used with starting material 12′ (490 mg, 1.14 mmol, 1.0 equiv.) and Pd/C 10% (160 mg, 0.16 mmol, 0.2 equiv.) to afford an oil (200 mg, 51%). ¹H NMR (300 MHz, CDCl₃) δ 8.14-8.06 (m, 1H), 7.45-7.28 (m, 2H), 7.18 (d, J=9.0 Hz, 1H), 6.60-6.49 (m, 2H), 5.84 (s, 1H), 4.25-3.96 (m, 4H), 3.58 (s, 2H), 3.47 (t, J=5.1 Hz, 4H), 2.53 (d, J=10.2 Hz, 2H). HRMS (ESI⁺): m/z calcd for [C₁₈H₂₃N₄O₃]⁺ 343.1770, found 343.1757.

### 2-(1,3-dioxolan-2-yl)-6-((4-(pyrimidin-2-yl)piperazin-1-yl)methyl)pyridin-3-ol 21′

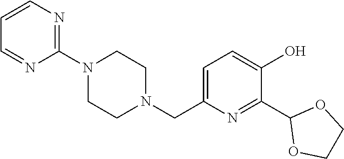 [SMILES: Oc1ccc(CN2CCN(c3ncccn3)CC2)nc1C1OCCO1]

General procedure C was used with starting material 13′ (880 mg, 2.03 mmol, 1.0 equiv.) and Pd/C 10% (569 mg, 0.41 mmol, 0.2 equiv.) to afford an oil (230 mg, 33%). ¹H NMR (300 MHz, CDCl₃) δ 8.35 (d, J=4.7 Hz, 2H), 8.17 (s, 1H), 7.46 (d, J=8.5 Hz, 1H), 7.28 (d, J=8.5 Hz, 1H), 6.52 (t, J=4.7 Hz, 1H), 5.95 (s, 1H), 4.35-4.11 (m, 4H), 3.93-3.84 (m, 4H), 3.70 (s, 2H), 2.65-2.55 (m, 4H). ¹³C NMR (75 MHz, CDCl₃) δ 157.72, 151.63, 137.67, 125.79, 109.79, 105.92, 65.16, 63.80, 53.07, 43.70. HRMS (ESI⁺): m/z calcd for [C₁₄H₂₂N₃O₃]⁺ 344,1723, found 344,1712.

### (E)-3-hydroxy-6-(piperidin-1-ylmethyl)picolinaldehyde oxime 22′

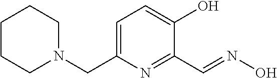 [SMILES: O/N=C/c1nc(CN2CCCCC2)ccc1O]

General procedure D was used with starting material 14′ (281 mg, 1.11 mmol, 1.0 equiv.) to afford the desired product as a formate salt (142 mg, 46%). ¹H NMR (300 MHz. MeOD) δ 8.54 (s, 1H), 8.38 (s, 1H), 7.39 (s, 2H), 4.29 (s, 2H), 3.21 (t, J=5.7 Hz, 4H), 1.87 (p, J=5.8 Hz, 4H), 1.69 (q, J=6.0 Hz, 2H). ¹³C NMR (75 MHz, MeOD) δ 168.5, 154.1, 150.4, 141.2, 136.8, 125.7, 124.6, 60.0, 53.1, 22.8, 21.5. HRMS (ESI⁺): m/z calcd for [C₁₂H₁₈N₃O₂]⁺ 236.1399, found 236.1394. HPLC (method A): t_{R}=1.95 min (97.67%).

### (E)-3-hydroxy-6-(morpholinomethyl)picolinaldehyde oxime 23′

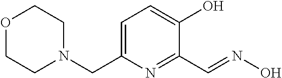 [SMILES: O/N=C/c1nc(CN2CCOCC2)ccc1O]

General procedure D was used with starting material 15′ (705 mg, 2.65 mmol, 1.0 equiv.) to afford the desired product as a 0.85.formate salt (675 mg, 92%). ¹H NMR (300 MHz, MeOD) δ 8.39 (s, 1H), 8.35 (s, 1H), 7.40 (d, J=8.4 Hz, 1H), 7.36 (d, J=8.5 Hz, 1H), 4.02 (s, 2H), 3.89-3.78 (m, 4H), 2.95 (t, J=4.8 Hz, 4H). ¹³C NMR (75 MHz, MeOD) δ 166.7, 153.8, 151.0, 144.3, 136.1, 125.4, 124.5, 64.9, 61.5, 52.6. HRMS (ESI⁺): m/z calcd for [C₁₁H₁₆N₃O₃]⁺ 238.1192, found 238.1185. HPLC (method A): t_{R}=1.00 min (99.48%).

### (E)-6-((3,4-dihydroisoquinolin-2(1H)-yl)methyl)-3-hydroxypicolinaldehyde oxime 24′

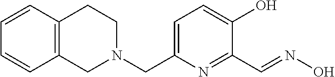 [SMILES: O/N=C/c1nc(CN2CCc3ccccc3C2)ccc1O]

General procedure E was used with starting material 16′ (90 mg, 0.29 mmol, 1.0 equiv.) to afford the desired product as a mesylate salt (54 mg, 49%). ¹H NMR (300 MHz, MeOD) δ 8.43 (s, 1H), 7.54-7.40 (m, 2H), 7.39-7.09 (m, 4H), 4.59 (s, 2H), 4.55 (s, 2H), 3.87-3.43 (m, 2H), 3.25 (s, 2H), 2.68 (s, 3H). ¹³C NMR (75 MHz, MeOD) δ 154.2, 150.0, 140.4, 137.0, 130.8, 128.5, 128.1, 127.5, 126.9, 126.5, 125.6, 124.9, 58.5, 53.1, 49.7, 38.1, 24.9. HRMS (ESI⁺): m/z calcd for [C₁₆H₁₈N₃O₂]⁺ 284.1399, found 284.1393. HPLC (method B): t_{R}=6.81 min (92.64%).

### (E)-3-hydroxy-6-((1,3,4,9-tetrahydro-2H-pyrido[3,4-b]indol-2-yl)methyl)picolinaldehyde oxime 25′

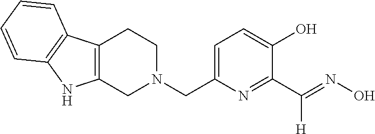 [SMILES: O/N=C/c1nc(CN2CCc3c([nH]c4ccccc34)C2)ccc1O]

General procedure E was used with starting material 17′ (172 mg, 0.49 mmol, 1.0 equiv.) to afford the desired product as a mesylate salt (147 mg, 72%). ¹H NMR (300 MHz, DMSO) δ 12.02 (s, 1H), 11.03 (s, 1H), 10.53 (s, 2H), 8.37 (s, 1H), 7.56 (d, J=8.5 Hz, 1H), 7.53-7.43 (m, 2H), 7.37 (d, J=8.0 Hz, 1H), 7.12 (ddd, J=8.1, 7.0, 1.3 Hz, 1H), 7.07-6.97 (m, 1H), 4.61 (s, 2H), 4.55 (s, 2H), 3.92-3.43 (m, 2H), 3.04 (t, J=6.0 Hz, 2H), 2.36 (s, 4H). ¹³C NMR (75 MHz, DMSO) δ 153.8, 149.6, 141.7, 137.5, 136.7, 127.1, 126.5, 126.1, 125.1, 122.2, 119.5, 118.5, 111.9, 105.6, 58.4, 50.5, 48.9, 40.2, 18.5. HRMS (ESI⁺): m/z calcd for [C₁₈H₁₉N₄O₂]⁺ 323.1508, found 323.1511. HPLC (method A): t_{R}=7.17 min (97.22%).

### (E)-3-hydroxy-6-((4-methylpiperazin-1-yl)methyl)picolinaldehyde oxime 26′

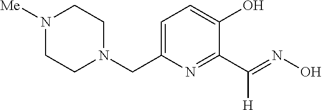 [SMILES: CN1CCN(Cc2ccc(O)c(/C=N/O)n2)CC1]

General procedure D was used with starting material 18′ (437 mg, 1.56 mmol, 1.0 equiv.) to afford the desired product as a 0.85.formate salt (404 mg, 89%). ¹H NMR (300 MHz, CDCl₃) δ 8.52 (s, 1H), 8.29 (s, 1H), 7.36 (d, J=8.4 Hz, 1H), 7.32 (d, J=8.5 Hz, 1H), 3.69 (s, 2H), 3.07 (t, J=5.3 Hz, 4H), 2.75 (s, 4H), 2.70 (s, 3H). ¹³C NMR (75 MHz, CDCl₃) δ 170.0, 154.8, 152.7, 149.3, 136.8, 126.1, 125.9, 63.2, 54.8, 51.6, 44.1. HRMS (ESI⁺): m/z calcd for [C₁₂H₁₉N₄O₂]⁺ 251.1508, found 251.1512. HPLC (method A): t_{R}=0.97 min (98.31%).

### (E)-3-hydroxy-6-((4-phenylpiperazin-1-yl)methyl)picolinaldehyde oxime 27′

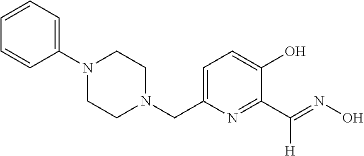 [SMILES: O/N=C/c1nc(CN2CCN(c3ccccc3)CC2)ccc1O]

General procedure E was used with starting material 19′ (417 mg, 1.22 mmol, 1.0 equiv.) to afford the desired product as a dimesylate salt (205 mg, 33%). ¹H NMR (300 MHz, MeOD) δ 8.32 (s, 1H), 7.36 (d, J=1.1 Hz, 2H), 7.28-7.15 (m, 2H), 7.05-6.95 (m, 2H), 6.95-6.83 (m, 1H), 4.40 (s, 2H), 3.44 (s, 8H), 3.25 (s, 2H), 2.61 (s, 4H), 1.22-1.12 (m, 2H). ¹³C NMR (75 MHz, MeOD) δ 149.74, 148.90, 129.10, 125.89, 125.12, 121.91, 117.03, 59.18, 51.68, 47.04, 38.13. HRMS (ESI⁺): m/z calcd for [C₁₇H₂₁N₄O₂]⁺ 313.1655, found 313.1655. HPLC (method C): t_{R}=3.59 min (98.3%).

### (E)-3-hydroxy-6-((4-(pyridin-2-yl)piperazin-1-yl)methyl)picolinaldehyde oxime 28′

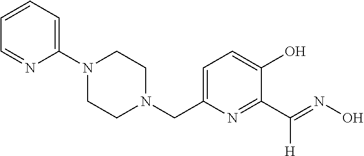 [SMILES: O/N=C/c1nc(CN2CCN(c3ccccn3)CC2)ccc1O]

General procedure E was used with starting material 20 (230 mg, 0.77 mmol, 1.0 equiv.) to afford the desired product as a dimesylate salt (159 mg, 67%). ¹H NMR (300 MHz, MeOD) δ 8.45 (s, 1H), 8.19-8.09 (m, 2H), 7.60-7.32 (m, 3H), 7.14 (ddd, J=7.0, 6.2, 0.9 Hz, 1H), 4.55 (s, 2H), 4.08 (s, 4H), 3.70-3.57 (m, 4H), 2.72 (s, 6H). ¹³C NMR (75 MHz, MeOD) δ 154.3, 152.8, 150.5, 144.5, 140.0, 137.7, 137.0, 125.8, 124.8, 114.4, 112.7, 59.5, 50.6, 43.0, 38.2. HRMS (ESI+): m/z calcd for [C₁₆H₂₀N₅O₂]⁺ 314.1617, found 314.1609. HPLC (method C): t_{R}=1.72 min (99.44%).

### (E)-3-hydroxy-6-((4-(pyrimidin-2-yl)piperazin-1-yl)methyl)picolinaldehyde oxime 29′

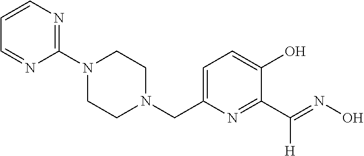 [SMILES: O/N=C/c1nc(CN2CCN(c3ncccn3)CC2)ccc1O]

General procedure E was used with starting material 21′ (230 mg, 0.77 mmol, 1.0 equiv.) to afford the desired product as a dimesylate salt (160 mg, 47%). ¹H NMR (300 MHz, MeOD) δ 8.32 (d, J=1.9 Hz, 2H), 8.30 (s, 1H), 7.46-7.30 (m, 2H), 6.58 (t, J=4.8 Hz, 1H), 3.81 (q, J=5.4 Hz, 5H), 3.66 (s, 2H), 2.62-2.53 (m, 4H), 1.31 (d, J=12.2 Hz, 1H). ¹³C NMR (75 MHz, MeOD) δ161.4, 157.6, 153.4, 151.6, 148.5, 135.3, 124.8, 124.5, 109.9, 62.9, 52.7, 43.2. HRMS (ESI⁺): m/z calcd for [C₁₈H₁₉N₆O₂]⁺ 315.1569, found 315.1574. HPLC (method C): t_{R}=2.64 min (98.49%).

### 3-(benzyloxy)-2-(1,3-dioxolan-2-yl)-6-vinylpyridine 30′

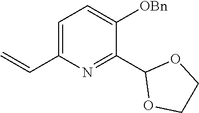 [SMILES: C=Cc1ccc(OCc2ccccc2)c(C2OCCO2)n1]

Procedure adapted from Molander et al. (Org. Lett. 2002, 4, 107-109): A solution of potassium vinyltrifluoroborate (478 mg, 3.57 mmol), PdCl₂(dppf)CH₂Cl₂ (121 mg, 0.149 mmol, 5 mol-%), 3-(benzyloxy)-6-bromo-2-(1,3-dioxolan-2-yl)pyridine (1.00 g, 2.98 mmol) and Et₃N (0.415 mL, 2.98 mmol) in n-PrOH (29 mL) was heated at reflux under a N₂ atmosphere. The reaction mixture was stirred at reflux for 4 h, then cooled to room temperature and evaporated. Purified by flash chromatography (0 to 50% AcOEt in Cyclo over 30 min) to afford the vinylated product as a sticky oil (660 mg, 78%). ¹H NMR (300 MHz, CDCl₃) δ 7.38-7.24 (m, 5H), 7.19 (d, J=8.6 Hz, 1H), 7.11 (d, J=8.6 Hz, 1H), 6.70 (dd, J=17.5, 10.8 Hz, 1H), 6.31 (s, 1H), 5.95 (dd, J=17.5, 1.4 Hz, 1H), 5.27 (dd, J=10.8, 1.4 Hz, 1H), 5.05 (s, 2H), 4.29-4.10 (m, 2H), 4.06-3.87 (m, 2H). ¹³C NMR (75 MHz, CDCl₃) δ 152.4, 147.9, 146.2, 136.3, 136.1, 128.7, 128.2, 127.4, 121.9, 120.4, 116.2, 100.5, 70.6, 65.7. HRMS (ESI⁺): m/z calcd for [C₁₇H₁₈NO₃]⁺ 284.1287, found 284.1278.

### 3-(benzyloxy)-2-(1,3-dioxolan-2-yl)-6-(2-(piperidin-1-yl)ethyl)pyridine 31′

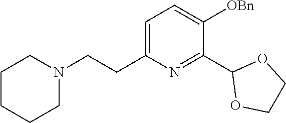 [SMILES: c1ccc(COc2ccc(CCN3CCCCC3)nc2C2OCCO2)cc1]

General procedure F was used with starting material 30′ (230 mg, 0.77 mmol, 1.0 equiv.) and piperidine (1.76 mL, 17.8 mmol, 10 equiv.) to afford the desired product (496 mg, 76%). ¹H NMR (300 MHz, CDCl₃) δ 7.37-7.21 (m, 5H), 7.08 (d, J=8.5 Hz, 1H), 7.01 (d, J=8.5 Hz, 1H), 6.25 (s, 1H), 5.00 (s, 2H), 4.23-4.06 (m, 2H), 4.03-3.86 (m, 2H), 2.89 (dd, J=9.8, 6.1 Hz, 2H), 2.65 (dd, J=10.0, 6.0 Hz, 2H), 2.41 (t, J=5.4 Hz, 4H), 1.53 (q, J=5.6 Hz, 4H), 1.37 (q, J=6.0 Hz, 2H). ¹³C NMR (75 MHz, CDCl₃) δ 151.7, 151.6, 145.6, 136.3, 128.6, 128.1, 127.4, 123.9, 120.8, 100.7, 70.6, 65.7, 59.1, 54.4, 34.5, 25.8, 24.3. HRMS (ESI⁺): m/z calcd for [C₂₂H₂₉N₂O₃]⁺ 369.2178, found 369.2192.

### 4-(2-(5-(benzyloxy)-6-(1,3-dioxolan-2-yl)pyridin-2-yl)ethyl)morpholine 32′

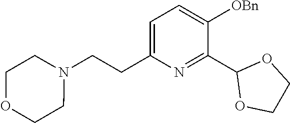 [SMILES: c1ccc(COc2ccc(CCN3CCOCC3)nc2C2OCCO2)cc1]

General procedure F was used with starting material 30′ (160 mg, 0.55 mmol, 1.0 equiv.) and morpholine (0.48 mL, 5.4 mmol, 10 equiv.) to afford the desired product (183 mg, 90%). ¹H NMR (300 MHz, CDCl₃) δ 7.40 (dddd, J=17.2, 8.7, 6.6, 3.0 Hz, 6H), 7.19 (d, J=8.5 Hz, 1H), 7.11 (d, J=8.5 Hz, 1H), 6.35 (s, 1H), 5.11 (s, 2H), 4.33-4.18 (m, 2H), 4.14-3.99 (m, 2H), 3.84-3.67 (m, 4H), 2.97 (dd, J=9.1, 6.4 Hz, 2H), 2.81-2.66 (m, 2H), 2.60-2.44 (m, 4H). ¹³C NMR (75 MHz, CDCl₃) δ 151.7, 151.4, 145.6, 136.3, 128.6, 128.2, 127.4, 123.9, 120.8, 100.7, 70.6, 66.9, 65.7, 58.7, 53.6, 34.4. HRMS (ESI⁺): m/z calcd for [C₂₁H₂₇N₂O₄]⁺ 371.1971, found 371.1958.

### 2-(2-(5-(benzyloxy)-6-(1,3-dioxolan-2-yl)pyridin-2-yl)ethyl)-1,2,3,4-tetrahydroisoquinoline 33′

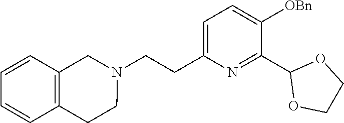 [SMILES: c1ccc(COc2ccc(CCN3CCc4ccccc4C3)nc2C2OCCO2)cc1]

General procedure F was used with starting material 30′ (100 mg, 0.36 mmol, 1.0 equiv.) and tertrahydroisoquinoline (0.45 mL, 3.57 mmol, 10 equiv.) to afford the desired product (96 mg, 65%). ¹H NMR (300 MHz, CDCl₃) δ 7.52-7.37 (m, 5H), 7.24-7.13 (m, 5H), 7.10-7.02 (m, 1H), 6.40 (s, 1H), 5.15 (s, 2H), 4.38-4.21 (m, 2H), 4.19-4.01 (m, 2H), 3.77 (s, 2H), 3.17-3.06 (m, 2H), 3.02-2.91 (m, 4H), 2.86 (td, J=5.8, 1.2 Hz, 2H). ¹³C NMR (75 MHz, CDCl₃) δ 151.7, 151.6, 145.7, 136.4, 134.8, 134.3, 128.7, 128.7, 128.2, 127.4, 126.6, 126.1, 125.6, 124.0, 120.8, 100.7, 70.7, 65.7, 58.2, 56.0, 50.9, 35.1, 29.1. HRMS (ESI⁺): m/z calcd for [C₂₆H₂₉N₂O₃]⁺ 417.2178, found 417.2171.

### 1-(2-(5-(benzyloxy)-6-(1,3-dioxolan-2-yl)pyridin-2-yl)ethyl)-4-methylpiperazine 34′

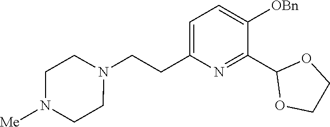 [SMILES: CN1CCN(CCc2ccc(OCc3ccccc3)c(C3OCCO3)n2)CC1]

General procedure F was used with starting material 30′ (510 mg, 1.79 mmol, 1.0 equiv.) and N-methylpiperazine (1.98 mL, 17.9 mmol, 10 equiv.) to afford the desired product (561 mg, 82%).

¹H NMR (300 MHz, CDCl₃) δ 7.50-7.28 (m, 5H), 7.20 (d, J=8.5 Hz, 1H), 7.12 (d, J=8.5 Hz, 1H), 6.37 (s, 1H), 5.13 (s, 2H), 4.35-4.19 (m, 2H), 4.16-3.99 (m, 2H), 2.98 (dd, J=9.6, 6.2 Hz, 2H), 2.83-2.72 (m, 2H), 2.59 (s, 3H), 2.51 (s, 3H), 2.33 (s, 3H). ¹³C NMR (75 MHz, CDCl₃) δ 151.6, 151.6, 145.7, 136.3, 128.6, 128.1, 127.4, 123.8, 120.8, 100.7, 70.6, 65.7, 58.3, 55.1, 53.0, 46.0, 34.7. HRMS (ESI⁺): m/z calcd for [C₂₂H₃₀N₃O₃]⁺ 384.2287, found 384.2285.

### 2-(1,3-dioxolan-2-yl)-6-(2-(piperidin-1-yl)ethyl)pyridin-3-ol 35′

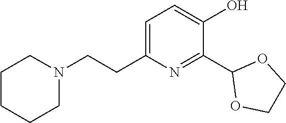 [SMILES: Oc1ccc(CCN2CCCCC2)nc1C1OCCO1]

General procedure C was used with starting material 31′ (580 mg, 1.57 mmol, 1.0 equiv.) and Pd/C 10% (441 mg, 0.31 mmol, 0.2 equiv.) to afford a foam (350 mg, 80%). ¹H NMR (300 MHz, CDCl₃) δ 7.85 (s, 1H), 7.15 (d, J=8.7 Hz, 1H), 7.09 (d, J=8.6 Hz, 1H), 5.94 (s, 1H), 4.23 (s, 2H), 4.09 (s, 2H), 3.09-2.91 (m, 2H), 2.82 (t, J=7.9 Hz, 2H), 2.60 (s, 4H), 1.68 (s, 4H), 1.48 (s, 2H). ¹³C NMR (75 MHz, CDCl₃) δ 151.1, 150.5, 138.6, 125.7, 124.9, 104.8, 65.2, 58.8, 54.2, 33.8, 25.3, 23.9. HRMS (ESI⁺): m/z calcd for [C₁₅H₂₃N₂O₃]⁺ 279.1709, found 279.1703.

### 2-(1,3-dioxolan-2-yl)-6-(2-morpholinoethyl)pyridin-3-ol 36′

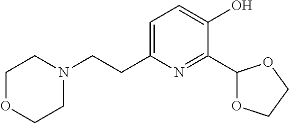 [SMILES: Oc1ccc(CCN2CCOCC2)nc1C1OCCO1]

General procedure C was used with starting material 32′ (450 mg, 1.22 mmol, 1.0 equiv.) and Pd/C 10% (342 mg, 0.24 mmol, 0.2 equiv.) to afford a foam (189 mg, 55%). ¹H NMR (300 MHz, CDCl₃) δ 7.19 (d, J=8.4 Hz, 1H), 7.14 (d, J=8.4 Hz, 1H), 5.92 (s, 1H), 4.35-4.21 (m, 2H), 4.21-4.07 (m, 2H), 3.81-3.72 (m, 4H), 3.02-2.91 (m, 2H), 2.81-2.69 (m, 2H), 2.60-2.51 (m, 4H). ¹³C NMR (75 MHz, CDCl₃) δ 151.3, 150.9, 137.8, 125.7, 124.9, 105.9, 67.0, 65.2, 58.9, 53.6, 34.5. HRMS (ESI⁺): m/z calcd for [C₁₄H₂₁N₂O₄]⁺ 281.1501, found 281.1500.

### 6-(2-(3,4-dihydroisoquinolin-2(1H)-yl)ethyl)-2-(1,3-dioxolan-2-yl)pyridin-3-ol 37′

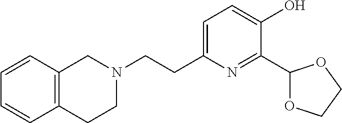 [SMILES: Oc1ccc(CCN2CCc3ccccc3C2)nc1C1OCCO1]

General procedure C was used with starting material 33′ (620 mg, 1.48 mmol, 1.0 equiv.) and Pd/C 10% (416 mg, 0.30 mmol, 0.2 equiv.) to afford a foam (256 mg, 53%). ¹H NMR (300 MHz, CDCl₃) δ 7.22-7.10 (m, 5H), 7.10-7.00 (m, 1H), 5.97 (s, 1H), 4.32-4.18 (m, 2H), 4.18-4.03 (m, 2H), 3.76 (s, 2H), 3.13-3.02 (m, 2H), 3.02-2.88 (m, 4H), 2.85 (td, J=5.8, 1.1 Hz, 2H). ¹³C NMR (75 MHz, CDCl₃) δ 151.2, 151.0, 138.3, 134.7, 134.3, 128.7, 126.6, 126.2, 125.7, 125.6, 124.9, 105.3, 65.2, 58.3, 56.0, 50.9, 34.9, 29.1. HRMS (ESI⁺): m/z calcd for [C₁₉H₂₃N₂O₃]⁺ 327.1709, found 327.1717.

### 2-(1,3-dioxolan-2-yl)-6-(2-(4-methylpiperazin-1-yl)ethyl)pyridin-3-ol 38′

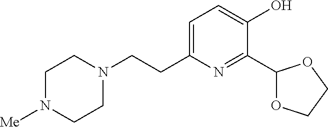 [SMILES: CN1CCN(CCc2ccc(O)c(C3OCCO3)n2)CC1]

General procedure C was used with starting material 34′ (590 mg, 1.54 mmol, 1.0 equiv.) and Pd/C 10% (431 mg, 0.31 mmol, 0.2 equiv.) to afford a foam (365 mg, 81%). ¹H NMR (300 MHz, CDCl₃) δ 9.12 (s, 1H), 7.09 (d, J=8.4 Hz, 1H), 6.93 (d, J=8.4 Hz, 1H), 5.87 (s, 1H), 4.17-4.01 (m, 2H), 4.01-3.84 (m, 2H), 2.82 (d, J=6.6 Hz, 2H), 2.80-2.73 (m, 2H), 2.68 (s, 8H), 2.36 (s, 3H). ¹³C NMR (75 MHz, CDCl₃) δ 151.0, 149.4, 139.8, 125.4, 124.6, 103.2, 65.2, 57.3, 53.6, 51.2, 44.7, 33.4. HRMS (ESI⁺): m/z calcd for [C₁₅H₂₄N₃O₃]⁺ 294.1818, found 294.1812.

### (E)-3-hydroxy-6-(2-(piperidin-1-yl)ethyl)picolinaldehyde oxime 39′

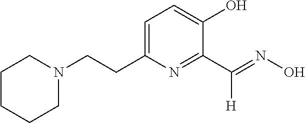 [SMILES: O/N=C/c1nc(CCN2CCCCC2)ccc1O]

General procedure D was used with starting material 35′ (350 mg, 1.26 mmol, 1.0 equiv.) to afford the desired product as a 0.24.formate salt (206 mg, 63%). ¹H NMR (300 MHz, DMSO) δ 8.27 (s, 1H), 7.77 (s, OH), 7.33 (d, J=8.4 Hz, 1H), 7.24 (d, J=8.5 Hz, 1H), 3.55-3.39 (m, 2H), 3.34 (t, J=7.6 Hz, 2H), 3.09 (t, J=7.6 Hz, 2H), 2.89 (s, 2H), 1.74 (d, J=19.2 Hz, 5H), 1.52-1.24 (m, 1H). ¹³C NMR (75 MHz, DMSO) δ 157.5, 152.3, 149.6, 148.6, 136.4, 125.1, 125.0, 55.5, 52.4, 30.3, 22.9, 21.9. HRMS (ESI⁺): m/z calcd for [C₁₃H₂₀N₃O₂]⁺ 250.1563, found 250.1556. HPLC (method A): t_{R}=3.81 min (97.88%).

### (E)-3-hydroxy-6-(2-morpholinoethyl)picolinaldehyde oxime 40′

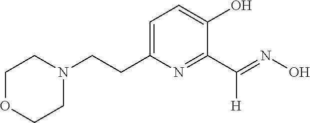 [SMILES: O/N=C/c1nc(CCN2CCOCC2)ccc1O]

General procedure D was used with starting material 36′ (185 mg, 0.66 mmol, 1.0 equiv.) to afford the desired product as a 0.8.formate salt (165 mg, 75%). ¹H NMR (300 MHz, MeOD) δ 8.47 (s, 1H), 8.30 (s, 1H), 7.29 (d, J=8.4 Hz, 1H), 7.22 (d, J=8.6 Hz, 1H), 3.98-3.86 (m, 4H), 3.40 (t, J=7.4 Hz, 2H), 3.24 (t, J=4.8 Hz, 4H), 3.16 (t, J=7.4 Hz, 2H). ¹³C NMR (75 MHz, MeOD) δ 167.8, 152.7, 150.3, 148.4, 135.7, 124.9, 124.5, 64.3, 56.6, 51.9, 29.8. HRMS (ESI⁺): m/z calcd for [C₁₂H₁₈N₃O₃]⁺ 252.1348, found 252.1349. HPLC (method A): t_{R}=2.74 min (95.54%).

### (E)-6-(2-(3,4-dihydroisoquinolin-2(1H)-yl)ethyl)-3-hydroxypicolinaldehyde oxime 41′

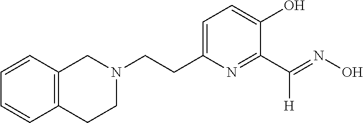 [SMILES: O/N=C/c1nc(CCN2CCc3ccccc3C2)ccc1O]

General procedure D was used with starting material 37′ (247 mg, 0.76 mmol, 1.0 equiv.) to afford the desired product as a 0.6.formate salt (183 mg, 74%). ¹H NMR (300 MHz, MeOD) δ 8.46 (s, 1H), 8.28 (s, 1H), 7.26 (tt, J=12.3, 5.2 Hz, 6H), 4.39 (s, 2H), 3.53 (dt, J=8.6, 4.8 Hz, 4H), 3.26 (t, J=7.3 Hz, 2H), 3.18 (t, J=6.2 Hz, 2H). ¹³C NMR (75 MHz, MeOD) δ 168.2, 152.9, 151.1, 148.3, 135.6, 131.4, 128.9, 128.5, 127.6, 126.6, 126.5, 124.8, 124.4, 55.4, 53.3, 49.7, 30.7, 25.5. HRMS (ESI⁺): m/z calcd for [C₁₇H₂₀N₃O₂]⁺ 298.1556, found 298.1548. HPLC (method B): t_{R}=4.67 min (99.35%).

### (E)-3-hydroxy-6-(2-(4-methylpiperazin-1-yl)ethyl)picolinaldehyde oxime 42′

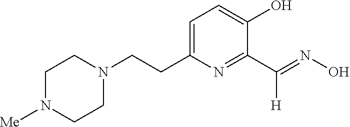 [SMILES: CN1CCN(CCc2ccc(O)c(/C=N/O)n2)CC1]

General procedure D was used with starting material 38′ (365 mg, 1.24 mmol, 1.0 equiv.) to afford the desired product as a 1.07.formate salt (157 mg, 40%). ¹H NMR (300 MHz, DMSO) δ 8.31 (s, 1H), 8.24 (s, 1H), 7.28 (d, J=8.5 Hz, 1H), 7.19 (d, J=8.5 Hz, 1H), 2.97 (s, 4H), 2.86-2.81 (m, 2H), 2.78 (s, 2H), 2.75 (s, 4H), 2.53-2.48 (m, 4H). ¹³C NMR (75 MHz, DMSO) δ 164.4, 152.1, 151.1, 151.0, 136.0, 124.7, 124.6, 57.6, 53.8, 51.5, 44.6, 34.0. HRMS (ESI⁺): m/z calcd for [C₃H₂₁N₄O₂]⁺ 265.1665, found 265.1664. HPLC (method A): t_{R}=2.34 min (91.31%).

### 4-(2-((5-(benzyloxy)-6-(1,3-dioxolan-2-yl)pyridin-2-yl)oxy)ethyl)morpholine 43′

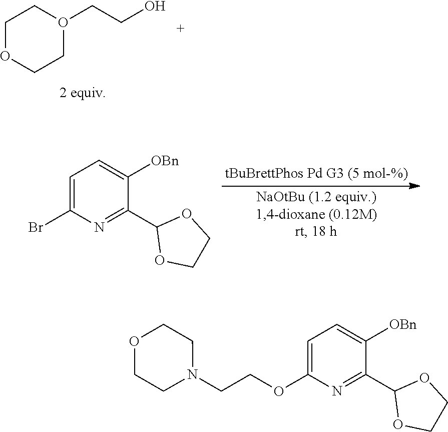

Adapted from Buchwald et al. (Org. Lett. 2018, 20, 1580-1583): A flask was charged with 3-(benzyloxy)-6-bromo-2-(1,3-dioxolan-2-yl)pyridine (400 mg, 1.19 mmol, 1.0 equiv.), 2-morpholinoethan-1-ol (0.29 mL, 2.38 mmol, 2.0 equiv), tBuBrettPhos Pd G3 (51 mg, 5 mol-%) and NaOt-Bu (137 mg, 1.4 mmol, 1.2 equiv). then evacuated and backfilled with argon (this process was repeated a total of three times). 1,4-dioxane (10 mL, 0.12 M) were added via syringe. The reaction mixture was sonicated until there were no visible chunks of NaOt-Bu. The reaction was stirred for 18 h at room temperature. EtOAc was added and the reaction slurry was then filtered through a pad of Celite® and rinsed with EtOAc. The crude material was concentrated in vacuo and purified by chromatography on silica gel (0 to 5% MeOH in DCM over 40 min) to afford a yellowish oil (261 mg, 57%).

### 2-(1,3-dioxolan-2-yl)-6-(2-morpholinoethoxy)pyridin-3-ol 44′

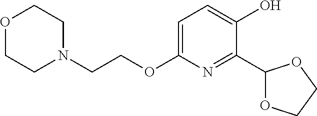 [SMILES: Oc1ccc(OCCN2CCOCC2)nc1C1OCCO1]

General procedure C was used with starting material 43′ (370 mg, 0.95 mmol, 1.0 equiv.) and Pd/C 10% (266 mg, 0.19 mmol, 0.2 equiv.) to afford a foam (277 mg, 99%). ¹H NMR (300 MHz, CDCl₃) δ 7.42 (s, 1H), 7.11 (d, J=8.8 Hz, 1H), 6.62 (d, J=8.9 Hz, 1H), 5.81 (s, 1H), 4.34 (t, J=5.7 Hz, 2H), 4.25-4.10 (m, 2H), 4.10-3.94 (m, 2H), 3.72-3.62 (m, 4H), 2.72 (t, J=5.8 Hz, 2H), 2.57-2.45 (m, 4H). ¹³C NMR (75 MHz, CDCl₃) δ 156.6, 147.1, 134.3, 129.4, 112.9, 104.7, 66.7, 65.1, 63.0, 57.6, 53.9. HRMS (ESI⁺): m/z calcd for [C₁₄H₂₁N₂O₅]⁺ 297.1450, found 297.1452.

### (E)-3-hydroxy-6-(2-morpholinoethoxy)picolinaldehyde oxime 45′

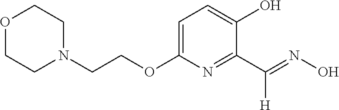 [SMILES: O/N=C/c1nc(OCCN2CCOCC2)ccc1O]

General procedure D was used with starting material 44′ (253 mg, 0.85 mmol, 1.0 equiv.) to afford the desired product (95 mg, 42%). ¹H NMR (300 MHz, MeOD) δ 8.16 (s, 1H), 7.26 (d, J=8.8 Hz, 1H), 6.69 (d, J=8.8 Hz, 1H), 4.40 (t, J=5.7 Hz, 2H), 3.75-3.66 (m, 4H), 2.77 (t, J=5.7 Hz, 2H), 2.59 (dd, J=5.7, 3.5 Hz, 4H). ¹³C NMR (75 MHz, MeOD) δ 157.8, 153.1, 150.5, 132.1, 129.3, 113.5, 67.5, 63.8, 58.6, 55.0. HRMS (ESI⁺): m/z calcd for [C₁₂H₁₈N₃O₄]⁺ 268.1297, found 268.1299. HPLC (method A): t_{R}=1.83 min (98.20%).

### 5-(benzyloxy)-6-(1,3-dioxolan-2-yl)-N-(2-(piperidin-1-yl)ethyl)pyridin-2-amine 46′

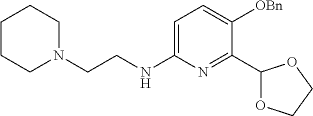 [SMILES: c1ccc(COc2ccc(NCCN3CCCCC3)nc2C2OCCO2)cc1]

General procedure G was used with 3-(benzyloxy)-6-bromo-2-(1,3-dioxolan-2-yl)pyridine (750 mg, 2.23 mmol, 1.0 equiv.) and 1-(2-Aminoethyl)piperidine (0.48 mL, 3.35 mmol, 1.5 equiv.) to afford an oil (788 mg, 92%). ¹H NMR (300 MHz, CDCl₃) δ 7.50-7.37 (m, 5H), 7.18 (d, J=8.9 Hz, 1H), 6.38 (d, J=8.8 Hz, 1H), 6.32 (s, 1H), 5.04 (s, 2H), 4.99 (s, 1H), 4.38-4.24 (m, 2H), 4.13-4.02 (m, 2H), 3.31 (q, J=5.6 Hz, 2H), 2.58 (t, J=6.2 Hz, 2H), 2.44 (t, J=5.4 Hz, 4H), 1.61 (q, J=5.5 Hz, 4H), 1.49 (q, J=5.9 Hz, 2H). ¹³C NMR (75 MHz, CDCl₃) δ 154.0, 145.8, 144.5, 137.2, 128.5, 127.9., 127.6, 126.4, 107.5, 99.6, 73.0, 65.6, 57.8, 54.5, 39.6, 26.0, 24.5. HRMS (ESI⁺): m/z calcd for [C₂₂H₃₀N₃O₃]⁺ 384.2287, found 384.2289.

### 5-(benzyloxy)-6-(1,3-dioxolan-2-yl)-N-(2-morpholinoethyl)pyridin-2-amine 47′

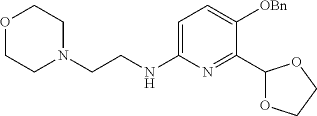 [SMILES: c1ccc(COc2ccc(NCCN3CCOCC3)nc2C2OCCO2)cc1]

General procedure G was used with 3-(benzyloxy)-6-bromo-2-(1,3-dioxolan-2-yl)pyridine (500 mg, 1.49 mmol, 1.0 equiv.) and 2-morpholinoethan-1-amine (0.59 mL, 4.46 mmol, 3 equiv.) to afford an oil (303 mg, 53%). ¹H NMR (300 MHz, CDCl₃) δ 7.52-7.26 (m, 6H), 7.19 (d, J=8.9 Hz, 1H), 6.38 (d, J=8.9 Hz, 1H), 6.31 (s, 1H), 5.04 (s, 2H), 4.40-4.18 (m, 2H), 4.15-4.00 (m, 2H), 3.78-3.68 (m, 4H), 3.31 (d, J=6.7 Hz, 2H), 2.63 (dd, J=6.6, 5.4 Hz, 2H), 2.58-2.41 (m, 4H). ¹³C NMR (75 MHz, CDCl₃) δ 153.8, 145.9, 144.4, 137.1, 128.5, 128.0, 127.6, 126.4, 107.5, 99.6, 72.9, 66.9, 65.7, 57.5, 53.5, 39.0. HRMS (ESI⁺): m/z calcd for [C₂₁H₂₈N₃O₄]⁺ 386.2080, found 386.2984.

### 5-(benzyloxy)-N-(2-(3,4-dihydroisoquinolin-2(1H)-yl)ethyl)-6-(1,3-dioxolan-2-yl)pyridin-2-amine 48′

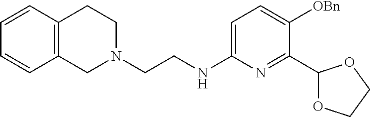 [SMILES: c1ccc(COc2ccc(NCCN3CCc4ccccc4C3)nc2C2OCCO2)cc1]

General procedure G was used with 3-(benzyloxy)-6-bromo-2-(1,3-dioxolan-2-yl)pyridine (750 mg, 2.23 mmol, 1.0 equiv.) and 2-(3,4-dihydroisoquinolin-2(1H)-yl)ethanamine (590 mg, 3.35 mmol, 1.5 equiv.) to afford an oil (1.03 mg, 100%). ¹H NMR (300 MHz, CDCl₃) δ 7.53-7.36 (m, 5H), 7.18 (dq, J=4.2, 2.2 Hz, 4H), 7.07 (dd, J=6.7, 2.4 Hz, 1H), 6.40 (d, J=9.0 Hz, 1H), 6.35 (s, 1H), 5.05 (s, 2H), 5.03 (s, 1H), 4.39-4.25 (m, 2H), 4.17-4.01 (m, 2H), 3.72 (s, 2H), 3.46 (q, J=5.4 Hz, 2H), 2.97 (t, J=5.9 Hz, 2H), 2.82 (t, J=6.1 Hz, 4H). ¹³C NMR (75 MHz, CDCl₃) δ 153.9, 145.8, 144.6, 137.2, 134.7, 134.4, 128.7, 128.5, 128.0, 127.6, 126.6, 126.4, 126.2, 125.7, 108.0, 99.6, 73.0, 65.7, 56.8, 55.8, 50.9, 39.5, 29.1. HRMS (ESI⁺): m/z calcd for [C₂₆H₃₀N₃O₃]⁺ 432.2287, found 432.2283.

### 5-(benzyloxy)-6-(1,3-dioxolan-2-yl)-N-(2-(4-methylpiperazin-1-yl)ethyl)pyridin-2-amine 49′

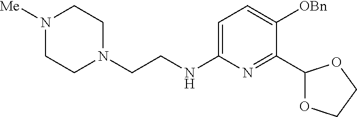 [SMILES: CN1CCN(CCNc2ccc(OCc3ccccc3)c(C3OCCO3)n2)CC1]

General procedure G was used with 3-(benzyloxy)-6-bromo-2-(1,3-dioxolan-2-yl)pyridine (750 mg, 2.23 mmol, 1.0 equiv.) and 2-(4-methyl-piperazin-1-yl)-ethylamine (0.5 mL, 3.35 mmol, 1.5 equiv.) to afford an oil (912 mg, 100%). ¹H NMR (300 MHz, CDCl₃) δ 7.50-7.28 (m, 5H), 7.18 (d, J=8.9 Hz, 1H), 6.37 (d, J=8.9 Hz, 1H), 6.31 (s, 1H), 5.04 (s, 2H), 4.95 (t, J=4.9 Hz, 1H), 4.37-4.23 (m, 2H), 4.16-3.99 (m, 2H), 3.31 (q, J=5.5 Hz, 2H), 2.63 (d, J=12.1 Hz, 2H), 2.53 (d, J=14.3 Hz, 8H), 2.33 (s, 3H). ¹³C NMR (75 MHz, CDCl₃) δ 153.9, 145.8, 144.5, 137.1, 128.5, 128.0, 127.6, 126.4, 107.5, 99.6, 73.0, 65.7, 57.0, 55.1, 53.0, 46.1, 39.4. HRMS (ESI⁺): m/z calcd for [C₂₂H₃₁N₄O₃]⁺ 399.2396, found 399.2140.

### N¹-(5-(benzyloxy)-6-(1,3-dioxolan-2-yl)pyridin-2-yl)-N²,N²-dimethylethane-1,2-diamine 50′

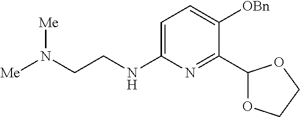 [SMILES: CN(C)CCNc1ccc(OCc2ccccc2)c(C2OCCO2)n1]

General procedure G was used with 3-(benzyloxy)-6-bromo-2-(1,3-dioxolan-2-yl)pyridine (750 mg, 2.23 mmol, 1.0 equiv.) and N,N-dimethylethylenediamine (0.37 mL, 3.35 mmol, 1.5 equiv.) to afford an oil (661 mg, 86%). ¹H NMR (300 MHz, CDCl₃) δ 7.50-7.34 (m, 5H), 7.17 (d, J=8.9 Hz, 1H), 6.38 (d, J=8.9 Hz, 1H), 6.34 (s, 1H), 5.04 (s, 2H), 4.92 (s, 1H), 4.38-4.23 (m, 2H), 4.16-4.00 (m, 2H), 3.33 (q, J=4.8, 4.0 Hz, 2H), 2.56 (t, J=6.1 Hz, 2H), 2.29 (s, 6H). ¹³C NMR (75 MHz, CDCl₃) δ 153.9, 145.7, 144.6, 137.2, 128.5, 128.0, 127.6, 126.4, 107.8, 99.6, 73.0, 65.7, 58.3, 45.3, 40.1. HRMS (ESI⁺): m/z calcd for [C₁₉H₂₆N₃O₃]⁺ 344.1974, found 344.1970.

### 2-(1,3-dioxolan-2-yl)-6-((2-(piperidin-1-yl)ethyl)amino)pyridin-3-ol 51′

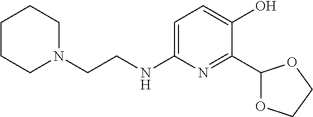 [SMILES: Oc1ccc(NCCN2CCCCC2)nc1C1OCCO1]

General procedure C was used with starting material 46′ (750 mg, 1.95 mmol, 1.0 equiv.) and Pd/C 10% (548 mg, 0.39 mmol, 0.2 equiv.) to afford an oil (360 mg, 63%). ¹H NMR (300 MHz, CDCl₃) δ 9.91 (s, 1H), 7.11 (d, J=8.9 Hz, 1H), 6.47 (d, J=8.8 Hz, 1H), 5.82 (s, 1H), 5.16 (s, 1H), 4.33-4.18 (m, 2H), 4.18-4.03 (m, 2H), 3.41 (t, J=6.0 Hz, 2H), 2.70 (t, J=6.1 Hz, 2H), 2.57 (s, 4H), 1.69 (p, J=6.1, 5.6 Hz, 4H), 1.55-1.47 (m, 2H). ¹³C NMR (75 MHz, CDCl₃) δ 152.6, 144.5, 134.9, 128.3, 109.8, 105.7, 65.1, 63.8, 57.6, 54.4, 54.3, 53.4, 39.0, 25.2, 25.1, 24.0. HRMS (ESI⁺): m/z calcd for [C₁₅H₂₄N₃O₃]+294.1818, found 294.1823.

### 2-(1,3-dioxolan-2-yl)-6-((2-morpholinoethyl)amino)pyridin-3-ol 52′

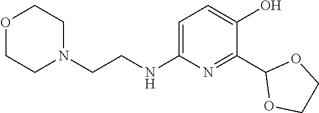 [SMILES: Oc1ccc(NCCN2CCOCC2)nc1C1OCCO1]

General procedure C was used with starting material 47′ (300 mg, 0.79 mmol, 1.0 equiv.) and Pd(OH)₂/C 20% (110 mg, 0.16 mmol, 0.2 equiv.) to afford an oil (136 mg, 59%). ¹H NMR (300 MHz, CDCl₃) δ 7.06 (d, J=8.8 Hz, 1H), 6.37 (d, J=8.8 Hz, 1H), 5.78 (s, 1H), 5.08-4.54 (m, 1H), 4.25-4.10 (m, 2H), 4.10-3.95 (m, 2H), 3.75-3.64 (m, 4H), 3.27 (dd, J=6.6, 5.3 Hz, 2H), 2.56 (dd, J=6.6, 5.4 Hz, 2H), 2.45 (dd, J=5.7, 3.8 Hz, 4H). ¹³C NMR (75 MHz, CDCl₃) δ 152.7, 144.4, 135.5, 128.2, 109.2, 105.1, 66.8, 65.1, 57.4, 53.4, 38.9. HRMS (ESI⁺): m/z calcd for [C₁₄H₂₂N₃O₂]⁺ 296.1610, found 296.1616.

### 6-((2-(3,4-dihydroisoquinolin-2(1H)-yl)ethyl)amino)-2-(1,3-dioxolan-2-yl)pyridin-3-ol 53′

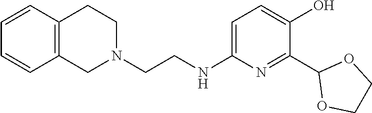 [SMILES: Oc1ccc(NCCN2CCc3ccccc3C2)nc1C1OCCO1]

General procedure C was used with starting material 48′ (960 mg, 2.29 mmol, 1.0 equiv.) and Pd/C 10% (626 mg, 0.45 mmol, 0.2 equiv.) to afford an oil (477 mg, 63%). ¹H NMR (300 MHz, CDCl₃) δ 7.31 (s, 1H), 7.18 (dd, J=5.6, 2.4 Hz, 3H), 7.12 (d, J=8.8 Hz, 1H), 7.06 (d, J=5.2 Hz, 1H), 6.45 (d, J=8.8 Hz, 1H), 5.82 (s, 1H), 4.94 (s, 1H), 4.33-4.18 (m, 2H), 4.18-4.04 (m, 2H), 3.71 (s, 2H), 3.45 (s, 2H), 2.96 (t, J=6.1 Hz, 2H), 2.84-2.76 (m, 4H). ¹³C NMR (75 MHz, CDCl₃) δ 152.8, 144.5, 134.9, 134.7, 134.4, 128.7, 128.2, 126.6, 126.2, 125.7, 109.7, 105.9, 65.1, 56.8, 55.8, 50.8, 39.5, 29.1. HRMS (ESI⁺): m/z calcd for [C₁₄H₂₂N₃O₃]⁺ 342.1818, found 342.1822.

### 2-(1,3-dioxolan-2-yl)-6-((2-(4-methylpiperazin-1-yl)ethyl)amino)pyridin-3-ol 54′

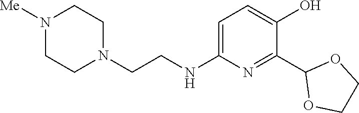 [SMILES: CN1CCN(CCNc2ccc(O)c(C3OCCO3)n2)CC1]

General procedure C was used with starting material 49′ (850 mg, 2.14 mmol, 1.0 equiv.) and Pd/C 10% (601 mg, 0.43 mmol, 0.2 equiv.) to afford an oil (518 mg, 79%). ¹H NMR (300 MHz, CDCl₃) δ 7.12 (d, J=8.8 Hz, 1H), 6.43 (d, J=8.8 Hz, 1H), 5.83 (s, 1H), 4.86 (s, 1H), 4.32-4.18 (m, 2H), 4.18-4.04 (m, 2H), 3.31 (t, J=6.0 Hz, 2H), 2.63 (t, J=6.0 Hz, 2H), 2.52 (s, 8H), 2.34 (s, 3H). ¹³C NMR (75 MHz, CDCl₃) δ 152.8, 144.4, 135.2, 128.3, 109.1, 105.6, 65.1, 56.9, 55.0, 52.8, 46.0, 39.4. HRMS (ESI⁺): m/z calcd for [C₁₅H₂₅N₄O₃]⁺ 309.1927, found 309.1927.

### ((2-(dimethylamino)ethyl)amino)-2-(1,3-dioxolan-2-yl)pyridin-3-ol 55′

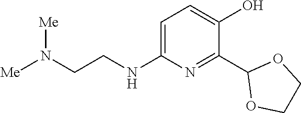 [SMILES: CN(C)CCNc1ccc(O)c(C2OCCO2)n1]

General procedure C was used with starting material 50′ (640 mg, 1.85 mmol, 1.0 equiv.) and Pd/C 10% (519 mg, 0.37 mmol, 0.2 equiv.) to afford an oil (336 mg, 72%). ¹H NMR (300 MHz, CDCl₃) δ 7.10 (d, J=8.8 Hz, 1H), 6.43 (d, J=8.8 Hz, 1H), 5.82 (s, 1H), 4.32-4.17 (m, 2H), 4.17-4.03 (m, 2H), 3.35-3.30 (m, 2H), 2.58 (t, J=6.0 Hz, 2H), 2.31 (s, 6H). ¹³C NMR (75 MHz, CDCl₃) δ 152.8, 144.4, 135.1, 128.2, 109.7, 105.7, 65.1, 58.2, 45.2, 40.0. HRMS (ESI⁺): m/z calcd for [C₁₂H₂₀N₃O₃]⁺ 254.1505, found 254.1515.

### (E)-3-hydroxy-6-((2-morpholinoethyl)amino)picolinaldehyde oxime 56′

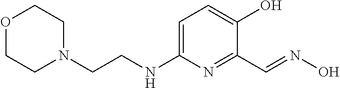 [SMILES: O/N=C/c1nc(NCCN2CCOCC2)ccc1O]

General procedure E was used with starting material 52′ (136 mg, 0.46 mmol, 1.0 equiv.) to afford the desired product (67 mg, 55%). ¹H NMR (300 MHz, MeOD) δ 8.15 (s, 1H), 7.12 (d, J=8.9 Hz, 1H), 6.50 (d, J=9.0 Hz, 1H), 3.76-3.67 (m, 4H), 3.41 (t, J=6.6 Hz, 2H), 2.60 (t, J=6.6 Hz, 2H), 2.56-2.49 (m, 4H). ¹³C NMR (75 MHz, MeOD) δ 152.9, 152.2, 146.5, 131.5, 126.7, 110.5, 66.3, 57.5, 53.4, 38.3. HRMS (ESI⁺): m/z calcd for [C₁₂H₁₉N₄O₃]⁺ 267.1473, found 267.1458. HPLC (method A): t_{R}=0.66, 1.01 min (96.01%).

### (E)-3-hydroxy-6-((2-(4-methylpiperazin-1-yl)ethyl)amino)picolinaldehyde oxime 57′

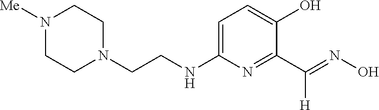 [SMILES: CN1CCN(CCNc2ccc(O)c(/C=N/O)n2)CC1]

General procedure E was used with starting material 54′ (500 mg, 1.62 mmol, 1.0 equiv.) to afford the desired product as a 1.1.formate salt (206 mg, 39%). ¹H NMR (300 MHz, MeOD) δ 8.55 (s, 1H), 8.21 (s, 1H), 7.15 (d, J=8.9 Hz, 1H), 6.57 (d, J=8.9 Hz, 1H), 3.48 (t, J=5.9 Hz, 2H), 3.04 (s, 8H), 2.92 (t, J=5.9 Hz, 2H), 2.67 (s, 3H). ¹³C NMR (75 MHz, MeOD) δ 169.2, 153.1, 150.7, 146.8, 131.8, 127.9, 111.9, 56.6, 52.3, 50.4, 43.4, 38.8. HRMS (ESI⁺): m/z calcd for [C₁₃H₂₂N₅O₂]⁺ 280.1774, found 280.1776. HPLC (method A): t_{R}=2.20 min (94.19%).

### (E)-6-((2-(dimethylamino)ethyl)amino)-3-hydroxypicolinaldehyde oxime 58′

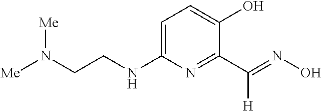 [SMILES: CN(C)CCNc1ccc(O)c(/C=N/O)n1]

General procedure E was used with starting material 55′ (301 mg, 1.19 mmol, 1.0 equiv.) to afford the desired product (58 mg, 22%). ¹H NMR (300 MHz, MeOD) δ 8.38 (s, 1H), 7.20 (d, J=9.0 Hz, 1H), 6.67 (d, J=9.0 Hz, 1H), 3.57-3.48 (m, 2H), 3.32-3.23 (m, 3H), 2.94 (s, 6H). ¹³C NMR (75 MHz, MeOD) δ 152.6, 145.7, 144.9, 132.1, 128.9, 113.4, 61.3, 42.1, 38.5. HRMS (ESI⁺): m/z calcd for [C₁₀H₁₇N₄O₂]⁺ 225.1352 found 225.1350. HPLC (method A): t_{R}=1.41 min (98.67%).

### 3-(benzyloxy)-2-(1,3-dioxolan-2-yl)-6-(3-(piperidin-1-yl)prop-1-yn-1-yl)pyridine 59′

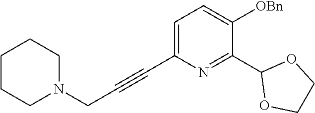 [SMILES: C(#Cc1ccc(OCc2ccccc2)c(C2OCCO2)n1)CN1CCCCC1]

General procedure H was used with 3-(benzyloxy)-6-bromo-2-(1,3-dioxolan-2-yl)pyridine (1.0 g, 2.98 mmol, 1.0 equiv.) and 1-(prop-2-yn-1-yl)piperidine (440 mg, 3.57 mmol, 1.2 equiv.) to afford an oil (989 mg, 88%). ¹H NMR (300 MHz, CDCl₃) δ 7.40 (tdd, J=10.0, 5.7, 4.0 Hz, 6H), 7.20 (d, J=8.6 Hz, 1H), 6.29 (s, 1H), 5.15 (s, 2H), 4.30-4.13 (m, 2H), 4.12-3.96 (m, 2H), 3.55 (s, 2H), 2.69-2.60 (m, 4H), 1.73-1.66 (m, 4H), 1.47 (s, 2H). ¹³C NMR (75 MHz, CDCl₃) δ 152.8, 146.8, 135.7, 134.3, 128.7, 128.6, 128.3, 127.4, 120.0, 101.1, 84.8, 83.3, 70.6, 65.8, 53.4, 48.4, 25.7, 23.7. HRMS (ESI⁺): m/z calcd for [C₂₃H₂₇N₂O₃]⁺ 379.2022, found 379.2019.

### 4-(3-(5-(benzyloxy)-6-(1,3-dioxolan-2-yl)pyridin-2-yl)prop-2-yn-1-yl)morpholine 60′

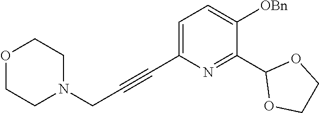 [SMILES: C(#Cc1ccc(OCc2ccccc2)c(C2OCCO2)n1)CN1CCOCC1]

General procedure H was used with 3-(benzyloxy)-6-bromo-2-(1,3-dioxolan-2-yl)pyridine (1.0 g, 2.98 mmol, 1.0 equiv.) and 1-(prop-2-yn-1-yl)morpholine (447 mg, 3.57 mmol, 1.2 equiv.) to afford an oil (1.13 g. 100%). ¹H NMR (300 MHz, CDCl₃) δ 7.51-7.32 (m, 6H), 7.22 (d, J=8.5 Hz, 1H), 6.31 (s, 1H), 5.18 (s, 2H), 4.32-4.13 (m, 2H), 4.13-3.94 (m, 2H), 3.80 (t, J=4.9 Hz, 4H), 3.55 (s, 2H), 2.68 (t, J=4.7 Hz, 4H). ¹³C NMR (75 MHz, CDCl₃) δ 152.9, 146.8, 135.7, 134.3, 128.7, 128.6, 128.4, 127.5, 119.9, 101.0, 84.8, 83.1, 70.6, 66.9, 65.8, 52.5, 48.1. HRMS (ESI⁺): m/z calcd for [C₂₂H₂₅N₂O₄]⁺ 381.1814, found 381.1817.

### 2-(3-(5-(benzyloxy)-6-(1,3-dioxolan-2-yl)pyridin-2-yl)prop-2-yn-1-yl)-1,2,3,4-tetrahydroisoquinoline 61′

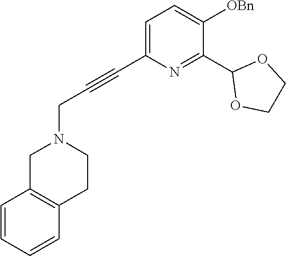 [SMILES: C(#Cc1ccc(OCc2ccccc2)c(C2OCCO2)n1)CN1CCc2ccccc2C1]

General procedure H was used with 3-(benzyloxy)-6-bromo-2-(1,3-dioxolan-2-yl)pyridine (1.0 g, 2.98 mmol, 1.0 equiv.) and 2-(prop-2-yn-1-yl)-1,2,3,4-tetrahydroisoquinoline (611 mg, 3.57 mmol, 1.2 equiv.) to afford an oil (1.11 g, 88%). ¹H NMR (300 MHz, CDCl₃) δ 7.50-7.34 (m, 6H), 7.21-7.03 (m, 5H), 6.32 (s, 1H), 5.18 (s, 2H), 4.33-4.15 (m, 2H), 4.15-3.98 (m, 2H), 3.89 (s, 2H), 3.77 (s, 2H), 3.00 (d, J=4.8 Hz, 2H), 2.98-2.88 (m, 2H). ¹³C NMR (75 MHz, CDCl₃) δ 152.8, 146.8, 135.7, 134.6, 134.4, 133.8, 128.7, 128.7, 128.6, 128.4, 127.5, 126.6, 126.2, 125.7, 120.0, 101.0, 83.5, 70.6, 65.8, 54.7, 50.0, 47.7, 29.3. HRMS (ESI⁺): m/z calcd for [C₂₇H₂₇N₂O₃]⁺ 427.2022, found 427.2019.

### 2-(3-(5-(benzyloxy)-6-(1,3-dioxolan-2-yl)pyridin-2-yl)prop-2-yn-1-yl)-2,3,4,9-tetrahydro-1H-pyrido[3,4-b]indole 62′

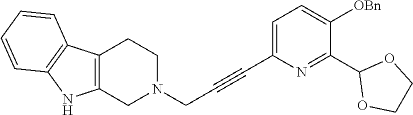 [SMILES: C(#Cc1ccc(OCc2ccccc2)c(C2OCCO2)n1)CN1CCc2c([nH]c3ccccc23)C1]

General procedure H was used with 3-(benzyloxy)-6-bromo-2-(1,3-dioxolan-2-yl)pyridine (1.0 g, 2.98 mmol, 1.0 equiv.) and 2-(prop-2-yn-1-yl)-2,3,4,9-tetrahydro-1H-pyrido[3,4-b]indole (751 mg, 3.57 mmol, 1.2 equiv.) to afford an oil (1.03 g, 74%). ¹H NMR (300 MHz, CDCl₃) δ 9.14 (s, 11H), 7.53-7.47 (m, 1H), 7.47-7.33 (m, 7H), 7.22 (d, J=8.5 Hz, 1H), 7.14 (d, J=6.3 Hz, 1H), 7.10 (d, J=6.9 Hz, 1H), 6.33 (s, 1H), 5.17 (s, 2H), 4.27-4.09 (m, 2H), 4.09-3.94 (m, 2H), 3.69 (s, 4H), 2.94 (s, 2H), 2.85 (d, J=5.2 Hz, 2H). ¹³C NMR (75 MHz, CDCl₃) δ 153.0, 146.6, 136.3, 135.7, 134.3, 131.9, 128.8, 128.7, 128.4, 127.5, 127.1, 121.0, 120.1, 118.9, 117.8, 111.1, 107.4, 100.5, 84.8, 83.8, 70.6, 65.8, 50.5, 48.8, 47.3, 21.6. HRMS (ESI⁺): m/z calcd for [C₂₉H₂₈N₃O₃]⁺ 466.2131, found 466.2112.

### 1-(3-(5-(benzyloxy)-6-(1,3-dioxolan-2-yl)pyridin-2-yl)prop-2-yn-1-yl)-4-methylpiperazine 63′

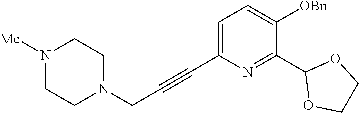 [SMILES: CN1CCN(CC#Cc2ccc(OCc3ccccc3)c(C3OCCO3)n2)CC1]

General procedure H was used with 3-(benzyloxy)-6-bromo-2-(1,3-dioxolan-2-yl)pyridine (1.0 g, 2.98 mmol, 1.0 equiv.) and 1-methyl-4-(prop-2-yn-1-yl)piperazine (493 mg, 3.57 mmol, 1.2 equiv.) to afford an oil (804 mg, 69%). ¹H NMR (300 MHz, CDCl₃) δ 7.49-7.28 (m, 6H), 7.21 (d, J=8.6 Hz, 1H), 6.26 (s, 1H), 5.14 (s, 2H), 4.28-4.11 (m, 2H), 4.11-3.94 (m, 2H), 3.55 (s, 2H), 2.84 (d, J=4.5 Hz, 4H), 2.78 (s, 4H), 2.49 (s, 3H). ¹³C NMR (75 MHz, CDCl₃) δ 152.9, 146.7, 135.7, 134.1, 128.8, 128.7, 128.3, 127.5, 120.0, 101.0, 85.0, 82.7, 70.6, 65.8, 54.5, 50.8, 47.5, 45.1. HRMS (ESI⁺): m/z calcd for [C₂₃H₂₈N₃O₃]⁺ 394.2131, found 394.2115.

### 6-(3-(5-(benzyloxy)-6-(1,3-dioxolan-2-yl)pyridin-2-yl)prop-2-yn-1-yl)-6,7-dihydro-5H-dibenzo[c,e]azepine 64′

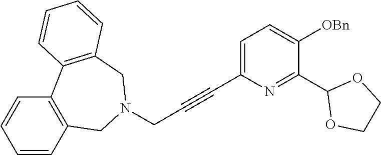 [SMILES: C(#Cc1ccc(OCc2ccccc2)c(C2OCCO2)n1)CN1Cc2ccccc2-c2ccccc2C1]

General procedure H was used with 3-(benzyloxy)-6-bromo-2-(1,3-dioxolan-2-yl)pyridine (1.44 g, 4.28 mmol, 1.0 equiv.) and 6-(prop-2-yn-1-yl)-6,7-dihydro-5H-dibenzo[c,e]azepine (Org. Biomol. Chem. 2018, 16, 555-565) (1.19 g, 5.14 mmol, 1.2 equiv.) to afford an oil (1.73 g, 83%). ¹H NMR (300 MHz, CDCl₃) δ 7.56-7.38 (m, 13H), 7.34 (d, J=8.6 Hz, 1H), 7.21 (d, J=8.6 Hz, 1H), 6.34 (s, 1H), 5.16 (s, 2H), 4.34-4.21 (m, 2H), 4.12-3.98 (m, 2H), 3.65 (s, 2H), 3.62 (s, 4H). ¹³C NMR (75 MHz, CDCl₃) δ 152.8, 146.8, 141.1, 135.8, 134.3, 130.1, 128.7, 128.6, 128.4, 128.2, 127.8, 127.7, 127.5, 119.9, 101.0, 85.2, 83.9, 70.6, 65.8, 55.0, 45.0. HRMS (ESI⁺): m/z calcd for [C₃₂H₂₉N₂O₃]⁺ 489.2178, found 489.2163.

### 2-(1,3-dioxolan-2-yl)-6-(3-(piperidin-1-yl)propyl)pyridin-3-ol 65′

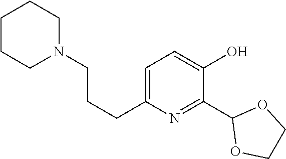 [SMILES: Oc1ccc(CCCN2CCCCC2)nc1C1OCCO1]

General procedure C was used with starting material 59′ (1.15 g, 3.03 mmol, 1.0 equiv.) and Pd(OH)₂/C 20% (425 mg, 0.61 mmol, 0.2 equiv.) to afford an oil (196 mg, 22%). ¹H NMR (300 MHz, CDCl₃) δ 7.33 (s, 1H), 7.08 (d, J=8.4 Hz, 1H), 7.01 (d, J=8.3 Hz, 1H), 5.95 (s, 1H), 4.30-4.14 (m, 2H), 4.14-3.98 (m, 2H), 2.70 (t, J=7.7 Hz, 2H), 2.53-2.45 (m, 4H), 2.43 (dd, J=6.8, 3.7 Hz, 2H), 2.01-1.84 (m, 2H), 1.62 (q, J=5.6 Hz, 4H), 1.43 (p. J=5.9 Hz, 2H). ¹³C NMR (75 MHz, CDCl₃) δ 152.2, 150.8, 138.9, 125.4, 124.3, 104.5, 65.2, 58.3, 54.2, 34.9, 26.4, 25.2, 24.0. HRMS (ESI⁺): m/z calcd for [C₁₆H₂₅N₂O₃]⁺ 293.1865, found 293.1860.

### 2-(1,3-dioxolan-2-yl)-6-(3-morpholinopropyl)pyridin-3-ol 66′

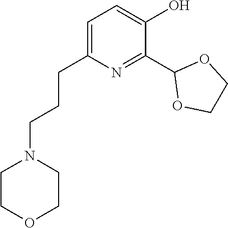 [SMILES: Oc1ccc(CCCN2CCOCC2)nc1C1OCCO1]

General procedure C was used with starting material 60′ (1.21 g, 3.17 mmol, 1.0 equiv.) and Pd(OH)₂/C 20% (446 mg, 0.64 mmol, 0.2 equiv.) to afford an oil (436 mg, 47%). ¹H NMR (300 MHz, CDCl₃) δ 7.18 (d, J=8.4 Hz, 1H), 7.10 (d, J=8.4 Hz, 1H), 5.91 (s, 1H), 4.40-4.21 (m, 2H), 4.21-4.05 (m, 2H), 3.77-3.71 (m, 4H), 2.89-2.69 (m, 2H), 2.56-2.27 (m, 6H), 2.03-1.76 (m, 2H). ¹³C NMR (75 MHz, CDCl₃) δ 152.9, 150.7, 137.7, 125.6, 124.5, 105.9, 67.0, 65.1, 58.4, 53.7, 35.0, 26.8. HRMS (ESI⁺): m/z calcd for [C₁₅H₂₃N₂O₄]⁺ 295.1658, found 295.1652.

### 6-(3-(3,4-dihydroisoquinolin-2(1H)-yl)propyl)-2-(1,3-dioxolan-2-yl)pyridin-3-ol 67′

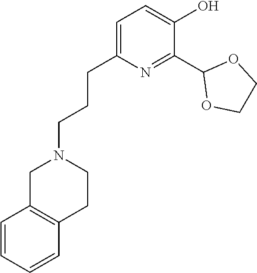 [SMILES: Oc1ccc(CCCN2CCc3ccccc3C2)nc1C1OCCO1]

General procedure C was used with starting material 61′ (1.08 g, 2.53 mmol, 1.0 equiv.) and Pd(OH)₂/C 20% (355 mg, 0.51 mmol, 0.2 equiv.) to afford an oil (431 mg, 50%). ¹H NMR (300 MHz, CDCl₃) δ 7.23-6.98 (m, 6H), 5.94 (s, 1H), 4.34-4.21 (m, 2H), 4.21-4.09 (m, 2H), 3.70-3.61 (m, 2H), 2.94 (t, J=5.9 Hz, 2H), 2.83 (dd, J=8.5, 7.0 Hz, 2H), 2.77 (t, J=5.9 Hz, 2H), 2.64-2.54 (m, 2H), 2.16-1.93 (m, 2H). ¹³C NMR (75 MHz, CDCl₃) δ 153.1, 150.7, 137.8, 134.9, 134.4, 128.7, 126.6, 126.1, 125.6, 125.6, 124.5, 105.9, 65.1, 57.8, 56.2, 51.0, 35.1, 29.2, 27.5. HRMS (ESI⁺): m/z calcd for [C₂₀H₂₅N₂O₃]⁺ 341.1863, found 341.1873.

### 2-(1,3-dioxolan-2-yl)-6-(3-(1,3,4,9-tetrahydro-2H-pyrido[3,4-b]indol-2-yl)propyl)pyridin-3-ol 68′

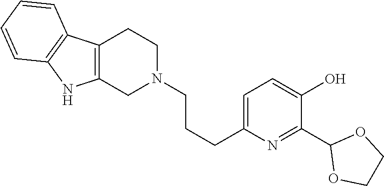 [SMILES: Oc1ccc(CCCN2CCc3c([nH]c4ccccc34)C2)nc1C1OCCO1]

General procedure C was used with starting material 62′ (0.99 g, 2.12 mmol, 1.0 equiv.) and Pd(OH)₂/C 20% (298 mg, 0.43 mmol, 0.2 equiv.) to afford an oil (362 mg, 45%). ¹H NMR (300 MHz, CDCl₃) δ 9.08 (s, 1H), 7.46 (dd. J=6.6, 2.2 Hz, 1H), 7.29-7.22 (m, 1H), 7.17-7.07 (m, 3H), 6.99 (d, J=8.4 Hz, 1H), 6.03 (s, 1H), 4.25-4.09 (m, 2H), 4.09-3.93 (m, 2H), 3.46 (s, 2H), 2.83 (q, J=5.9, 4.8 Hz, 4H), 2.75 (t, J=7.8 Hz, 2H), 2.56 (t, J=7.4 Hz, 2H), 1.96 (q, J=8.2, 7.7 Hz, 2H). ¹³C NMR (75 MHz, CDCl₃) δ 152.5, 151.0, 139.0, 136.3, 131.7, 127.1, 125.8, 124.7, 121.2, 119.1, 117.9, 111.0, 107.6, 104.1, 65.2, 57.2, 51.3, 49.8, 34.9, 27.3, 21.1. HRMS (ESI⁺): m/z calcd for [C₂₂H₂₆N₃O₃]⁺ 380.1974, found 280.1973.

### 2-(1,3-dioxolan-2-yl)-6-(3-(4-methylpiperazin-1-yl)propyl)pyridin-3-ol 69′

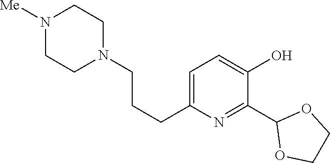 [SMILES: CN1CCN(CCCc2ccc(O)c(C3OCCO3)n2)CC1]

General procedure C was used with starting material 63′ (1.27 g, 3.19 mmol, 1.0 equiv.) and Pd(OH)₂/C 20% (448 mg, 0.64 mmol, 0.2 equiv.) to afford an oil (386 mg, 39%). ¹H NMR (300 MHz, CDCl₃) δ 8.11 (s, 1H), 7.02 (d, J=8.3 Hz, 1H), 6.93 (d, J=8.3 Hz, 1H), 5.87 (s, 1H), 4.21-4.08 (m, 2H), 4.08-3.88 (m, 2H), 2.75-2.54 (m, 8H), 2.39 (dd, J=9.0, 6.2 Hz, 2H), 2.29 (s, 3H), 1.83 (h, J=6.8 Hz, 2H). ¹³C NMR (75 MHz, CDCl₃) δ 152.0, 150.6, 139.3, 125.3, 124.3, 104.0, 65.2, 57.3, 54.0, 51.8, 45.2, 34.6, 26.3. HRMS (ESI⁺): m/z calcd for [C₁₆H₂₆N₃O₃]⁺ 308.1974, found 308.1972.

### 6-(3-(5,7-dihydro-6H-dibenzo[c,e]azepin-6-yl)propyl)-2-(1,3-dioxolan-2-yl)pyridin-3-ol 70′

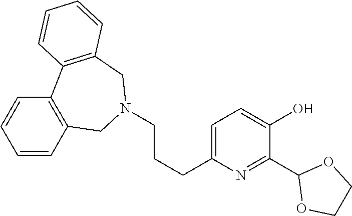 [SMILES: Oc1ccc(CCCN2Cc3ccccc3-c3ccccc3C2)nc1C1OCCO1]

General procedure C was used with starting material 64′ (1.66 g, 3.39 mmol, 1.0 equiv.) and Pd(OH)₂/C 20% (476 mg, 0.68 mmol, 0.2 equiv.) to afford an oil (246 mg, 18%). 1H NMR (300 MHz, CDCl₃) δ 7.68-7.59 (m, 1H), 7.51 (dd, J=7.3, 1.4 Hz, 2H), 7.45 (td, J=7.4, 7.0, 2.1 Hz, 2H), 7.40-7.31 (m, 4H), 7.14 (d, J=8.4 Hz, 1H), 7.09 (d, J=8.4 Hz, 1H), 6.05 (s, 1H), 4.32-4.16 (m, 2H), 4.16-3.99 (m, 2H), 3.44 (s, 4H), 2.85 (t, J=7.5 Hz, 2H), 2.65 (t, J=7.7 Hz, 2H), 2.20-2.02 (m, 2H). ¹³C NMR (75 MHz, CDCl₃) δ 152.5, 150.9, 141.1, 139.2, 134.3, 130.0, 128.2, 127.7, 127.6, 125.5, 124.5, 104.5, 65.3. 55.1, 54.5, 35.0, 28.0. HRMS (ESI⁺): m/z calcd for [C₂₅H₂₇N₂O₃]⁺ 403.2022, found 403.2038.

### (E)-3-hydroxy-6-(3-(piperidin-1-yl)propyl)picolinaldehyde oxime 71′

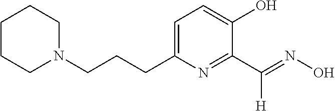 [SMILES: O/N=C/c1nc(CCCN2CCCCC2)ccc1O]

General procedure E was used with starting material 65′ (293 mg, 0.75 mmol, 1.0 equiv.) to afford the desired product as a mesylate salt (205 mg, 57%). ¹H NMR (300 MHz, MeOD) δ 8.51 (s, 1H), 8.03 (d, J=8.8 Hz, 1H), 7.86 (d, J=8.8 Hz, 1H), 3.66-3.54 (m, 2H), 3.29-3.18 (m, 2H), 3.13 (t, J=7.9 Hz, 2H), 2.99 (td, J=12.3, 3.6 Hz, 2H), 2.74 (s, 3H), 2.34-2.17 (m, 2H), 2.02-1.75 (m, 5H), 1.64-1.49 (m, 1H), ¹³C NMR (75 MHz, MeOD) δ 153.6, 147.3, 139.4, 133.3, 131.9, 127.5, 55.5, 53.0, 38.2, 28.9, 23.5, 22.8, 21.3. HRMS (ESI⁺): m/z calcd for [C₁₄H₂₂N₃O₂]⁺ 264.1712, found 264.1715. HPLC (method B): t_{R}=3.61 min (100.00%).

### (E)-3-hydroxy-6-(3-morpholinopropyl)picolinaldehyde oxime 72′

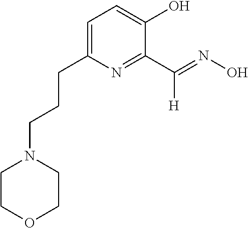 [SMILES: O/N=C/c1nc(CCCN2CCOCC2)ccc1O]

General procedure E was used with starting material 66′ (363 mg, 1.23 mmol, 1.0 equiv.) to afford the desired product as a mesylate salt (333 mg, 75%). ¹H NMR (300 MHz, MeOD) δ 8.32 (s, 1H), 7.31 (d, J=8.5 Hz, 1H), 7.22 (d, J=8.5 Hz, 1H), 3.92 (s, 4H), 3.30 (s, 4H), 3.24-3.12 (m, 3H), 2.86 (t, J=7.3 Hz, 2H), 2.72 (s, 3H), 2.28-2.09 (m, 2H). ¹³C NMR (75 MHz, MeOD) δ 152.6, 151.0, 150.8, 135.4, 124.7, 124.2, 63.7, 56.5, 51.8, 38.1, 32.9, 23.3. HRMS (ESI⁺): m/z calcd for [C₁₃H₂₀N₃O₃]⁺ 266.1505, found 266.1510. HPLC (method A): t_{R}=1.22 min (99.47%).

### (E)-6-(3-(3,4-dihydroisoquinolin-2(1H)-yl)propyl)-3-hydroxypicolinaldehyde oxime 73′

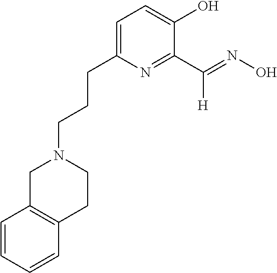 [SMILES: O/N=C/c1nc(CCCN2CCc3ccccc3C2)ccc1O]

General procedure E was used with starting material 67′ (364 mg, 1.07 mmol, 1.0 equiv.) to afford the desired product as a mesylate salt (333 mg, 76%). ¹H NMR (300 MHz. MeOD) δ 8.32 (s, 1H), 7.40-7.13 (m, 6H), 4.91 (s, 5H), 4.50 (s, 2H), 3.65 (t, J=6.5 Hz, 2H), 3.36 (dd, J=8.0, 6.6 Hz, 2H), 3.22 (t, J=6.4 Hz, 2H), 2.97 (t, J=6.9 Hz, 2H), 2.68 (s, 3H), 2.26 (p, J=7.0 Hz, 2H). ¹³C NMR (75 MHz, MeOD) δ 153.8, 152.3, 151.2, 136.9, 132.2, 129.9, 129.4, 128.8, 128.3, 128.1, 126.4, 125.9, 56.4, 53.8, 50.7, 39.5, 34.9, 25.7, 24.6. HRMS (ESI⁺): m/z calcd for [C₁₈H₂₂N₃O₂]⁺ 312.1719, found 312.1719. HPLC (method A): t_{R}=2.98 min (99.72%).

### (E)-3-hydroxy-6-(3-(1,3,4,9-tetrahydro-2H-pyrido[3,4-b]indol-2-yl)propyl)picolinaldehyde oxime 74′

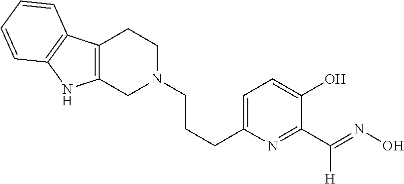 [SMILES: O/N=C/c1nc(CCCN2CCc3c([nH]c4ccccc34)C2)ccc1O]

General procedure E was used with starting material 68′ (356 mg, 0.94 mmol, 1.0 equiv.) to afford the desired product as a mesylate salt (49 mg, 19%). ¹H NMR (300 MHz, MeOD) δ 8.41 (s, 1H), 7.63 (d, J=8.7 Hz, 1H), 7.53 (d, J=8.7 Hz, 1H), 7.49 (d, J=7.8 Hz, 1H), 7.37 (d, J=8.1 Hz, 1H), 7.16 (ddd, J=8.2, 7.0, 1.3 Hz, 1H), 7.11-7.01 (m, 1H), 4.72 (s, 1H), 4.49 (s, 1H), 3.89 (s, 1H), 3.52-3.41 (m, 2H), 3.36 (s, 1H), 3.15 (s, 2H), 3.11-2.97 (m, 2H), 2.69 (s, 3H), 2.39-2.22 (m, 2H). ¹³C NMR (75 MHz, MeOD) δ152.9, 149.3, 145.1, 137.0, 134.0, 128.6, 125.8, 124.6, 122.1, 119.2, 117.6, 111.0, 105.2, 54.8, 50.7, 48.8, 38.1, 31.6, 23.6, 17.9. HRMS (ESI⁺): m/z calcd for [C₂₀H₂₃N₄O₂]⁺ 351.1821, found 351.1835. HPLC (method B): t_{R}=3.74 min (94.37%).

### (E)-3-hydroxy-6-(3-(4-methylpiperazin-1-yl)propyl)picolinaldehyde oxime 75′

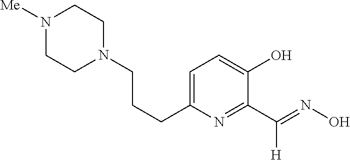 [SMILES: CN1CCN(CCCc2ccc(O)c(/C=N/O)n2)CC1]

General procedure E was used with starting material 69′ (668 mg, 2.17 mmol, 1.0 equiv.) to afford the desired product as a mesylate salt (425 mg, 84%). H NMR (300 MHz, MeOD) δ 8.37 (s, 1H), 7.41 (d, J=8.5 Hz, 1H), 7.31 (d, J=8.6 Hz, 1H), 3.55-3.43 (m, 4H), 3.38 (d, J=7.8 Hz, 4H), 3.10 (t, J=7.4 Hz, 2H), 2.92 (d, J=2.5 Hz, 5H), 2.75 (s, 3H), 2.12 (p, J=7.4 Hz, 2H). ¹³C NMR (75 MHz, MeOD) δ 152.6, 150.9, 148.7, 134.8, 126.1, 124.9, 55.6, 50.8, 48.9, 42.4, 38.2, 32.9, 24.1. HRMS (ESI⁺): m/z calcd for [C₁₄H₂₃N₄O₂]⁺ 279.1821, found 279.1826. HPLC (method A): t_{R}=1.03 min (99.06%).

### (E)-6-(3-(5,7-dihydro-6H-dibenzo[c,e]azepin-6-yl)propyl)-3-hydroxypicolinaldehyde oxime 76′

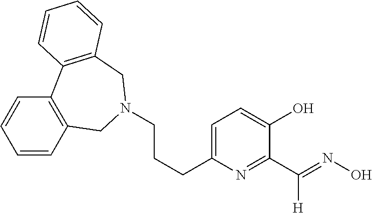 [SMILES: O/N=C/c1nc(CCCN2Cc3ccccc3-c3ccccc3C2)ccc1O]

General procedure E was used with starting material 70′ (429 mg, 1.07 mmol, 1.0 equiv.) to afford the desired product as a mesylate salt (60 mg, 18%). ¹H NMR (300 MHz, MeOD) δ 8.32 (s, 1H), 7.75-7.66 (m, 4H), 7.62-7.51 (m, 4H), 7.40 (d, J=8.5 Hz, 1H), 7.32 (d, J=8.5 Hz, 1H), 4.31-3.78 (m, 4H), 3.36 (s, 2H), 2.99 (t, J=7.0 Hz, 2H), 2.71 (s, 3H), 2.32 (h, J=7.2 Hz, 2H). ¹³C NMR (75 MHz, MeOD) δ 152.6, 150.5, 149.4, 140.8, 135.2, 131.2, 130.7, 128.7, 128.4, 127.8, 125.6, 124.8, 53.6, 53.3, 38.2, 32.9, 23.8. HRMS (ESI⁺): m/z calcd for [C₂₃H₂₄N₃O₂]⁺ 374.1869, found 374.1859. HPLC (method B): t_{R}=5.32 min (94.62%).

### 4-(5-(benzyloxy)-6-(1,3-dioxolan-2-yl)pyridin-2-yl)but-3-yn-1-ol 77′

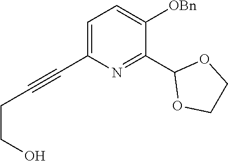 [SMILES: OCCC#Cc1ccc(OCc2ccccc2)c(C2OCCO2)n1]

General procedure H was used with 3-(benzyloxy)-6-bromo-2-(1,3-dioxolan-2-yl)pyridine (5.0 g. 14.87 mmol, 1 equiv.) and 3-butyn-1-ol (1.13 mL, 14.87 mmol, 1 equiv.) to afford an oil (4.53 g, 94%). ¹H NMR (300 MHz, CDCl₃) δ 7.47-7.36 (m, 6H), 7.22 (d, J=8.6 Hz, 1H), 6.29 (s, 1H), 5.16 (s, 2H), 4.26-4.19 (m, 2H), 4.08-4.01 (m, 2H), 3.86 (t, J=6.3 Hz, 2H), 2.93 (s, 1H), 2.71 (t, J=6.3 Hz, 2H). ¹³C NMR (75 MHz, CDCl₃) δ 152.7, 146.5, 135.7, 134.6, 128.7, 128.4, 127.4, 120.2, 100.7, 86.5, 81.5, 70.6, 65.8, 60.8, 23.9. HRMS (ESI⁺): m/z calcd for [C₁₉H₂₀NO₄]⁺ 326.1392, found 326.1391.

### 2-(1,3-dioxolan-2-yl)-6-(4-hydroxybutyl)pyridin-3-ol 78′

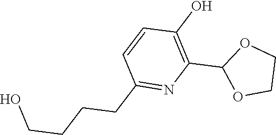 [SMILES: OCCCCc1ccc(O)c(C2OCCO2)n1]

General procedure C was used with starting material 77′ (3.89 g, 11.89 mmol, 1.0 equiv.) and Pd(OH)₂/C 20% (1.67 g, 2.38 mmol, 0.2 equiv.) to afford an oil (2.32 g, 100%). ¹H NMR (300 MHz, CDCl₃) δ 8.12 (s, 1H), 7.19 (d, J=8.4 Hz, 1H), 7.10 (d, J=8.4 Hz, 1H), 5.92 (s, 1H), 4.34-4.20 (m, 2H), 4.20-4.05 (m, 2H), 3.69 (t, J=6.3 Hz, 2H), 2.80 (t, J=7.5 Hz, 2H), 2.21 (s, 1H), 1.89-1.73 (m, 2H), 1.73-1.58 (m, 2H). ¹³C NMR (75 MHz, CDCl₃) δ 153.2, 150.7, 137.7, 125.8, 124.6, 105.7, 65.1, 62.5, 36.6, 32.2, 26.0. HRMS (ESI⁺): m/z calcd for [C₁₂H₁₈NO₄]⁺ 240.1236, found 240.1236.

### 4-(5-(benzyloxy)-6-(1,3-dioxolan-2-yl)pyridin-2-yl)butan-1-ol 79′

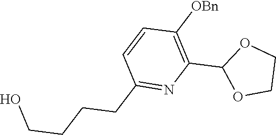 [SMILES: OCCCCc1ccc(OCc2ccccc2)c(C2OCCO2)n1]

To a solution of alcohol 78′ (2.49 g, 10.4 mmol, 1 equiv.), in acetone (100 mL, 0.1 M) was added successively Cs₂CO₃ (5.8 g, 17.7 mmol, 1.7 equiv.) and benzyl bromide (1.9 mL, 15.7 mmol, 1.5 equiv.). The heterogeneous reaction mixture was stirred at room temperature for 4 hours. Salts were removed by filtration and the crude product was concentrated under reduced pressure. Purification by flash chromatography on silica gel (0 to 6% MeOH in dichloromethane). ¹H NMR (300 MHz, CDCl₃) δ 7.49-7.28 (m, 5H), 7.19 (d, J=8.5 Hz, 1H), 7.07 (d, J=8.5 Hz, 1H), 6.34 (s, 1H), 5.11 (s, 2H), 4.33-4.16 (m, 2H), 4.13-3.96 (m, 2H), 3.66 (t, J=6.2 Hz, 2H), 2.81 (t, J=7.4 Hz, 2H), 2.52 (s, 1H), 1.90-1.74 (m, 2H), 1.70-1.55 (m, 2H). ¹³C NMR (75 MHz, CDCl₃) δ 153.4, 151.6, 145.2, 136.3, 128.6, 128.1, 127.4, 123.6, 120.9, 100.4, 70.7, 65.7, 62.3, 36.3, 32.0, 25.6. HRMS (ESI⁺): m/z calcd for [C₁₉H₂₄NO₄]⁺ 330.1697, found 330.1705.

### 1-(4-(5-(benzyloxy)-6-(1,3-dioxolan-2-yl)pyridin-2-yl)butyl)azocane 80′

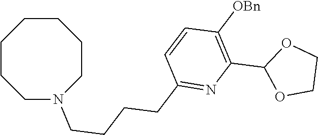 [SMILES: c1ccc(COc2ccc(CCCCN3CCCCCCC3)nc2C2OCCO2)cc1]

Alcohol 79′ (3.1 g, 9.4 mmol, 1 equiv.) and Et₃N (3.9 mL, 28.3 mmol, 3 equiv.) was dissolved in dichloromethane (63 mL, 0.15 M), then MsCl (1.8 mL, 23.6 mmol, 2.5 equiv.) was added at 0° C. The resulting mixture was allowed to warm up to room temperature and stirred for 1 hour. After completion, the mixture was quenched with water and extracted with dichloromethane (3×60 mL). Dried over MgSO₄ and concentrated in vacuo. The crude mesylated intermediate was dissolved in anhydrous acetonitrile (63 mL, 0.15 M) before azocane (5.3 mL, 47.2 mmol, 5 equiv.) and K₂CO₃ (2.6 g, 18.9 mmol, 2 equiv.) was added and the reaction mixture heated under reflux (90° C.) for 2 hours. The crude mixture was filtered though celite and concentrated before being purified by flash chromatography on silica gel (MeOH in DCM 0 to 25%) to afford an oil (2.41 g, 60%). ¹H NMR (300 MH z, CDCl₃) δ 7.52-7.35 (m, 5H), 7.21 (d, J=8.5 Hz, 1H), 7.09 (d, J=8.5 Hz, 1H), 6.38 (s, 1H), 5.14 (s, 2H), 4.41-4.20 (m, 2H), 4.14-4.01 (m, 2H), 2.86-2.74 (m, 2H), 2.62-2.53 (m, 4H), 2.52-2.44 (m, 2H), 1.83-1.70 (m, 2H), 1.70-1.46 (m, 12H). ¹³C NMR (75 MHz, CDCl₃) δ 153.9, 151.4, 145.5, 136.5, 128.6, 128.1, 127.4, 123.3, 120.8, 100.8, 70.7, 65.7, 58.8, 54.0, 37.2, 27.9, 27.9, 27.7, 27.4, 26.4. HRMS (ESI⁺): m/z calcd for [C₂₆H₃₇N₂O₃]⁺ 425.2804, found 425.2805.

### 6-(4-(azocan-1-yl)butyl)-2-(1,3-dioxolan-2-yl)pyridin-3-ol 81′

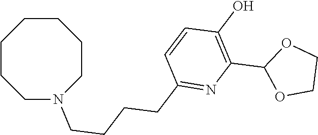 [SMILES: Oc1ccc(CCCCN2CCCCCCC2)nc1C1OCCO1]

General procedure C was used with starting material 80′ (2.38 g, 4.49 mmol, 1.0 equiv.) and Pd/C 10% (1.19 g, 1.12 mmol, 0.2 equiv.) to afford an oil (1.22 g, 65%). ¹H NMR (300 MHz, CDCl₃) δ 7.28 (s, 1H), 7.16 (dd, J=8.4, 2.5 Hz, 1H), 7.07 (dd, J=8.4, 2.9 Hz, 1H), 5.93 (s, 1H), 4.24 (tt, J=5.5, 2.5 Hz, 2H), 4.11 (tt, J=6.9, 3.2 Hz, 2H), 2.74 (td, J=7.7, 2.2 Hz, 2H), 2.55 (d, J=4.6 Hz, 4H), 2.46 (td, J=7.2, 1.7 Hz, 2H), 1.79-1.67 (m, 2H), 1.67-1.49 (m, 12H). ¹³C NMR (75 MHz, CDCl₃) δ 153.1, 150.6, 150.3, 138.4, 125.5, 124.3, 104.8, 65.1, 57.9, 53.2, 36.9, 27.9, 27.2, 26.9, 26.8, 26.4. HRMS (ESI⁺): m/z calcd for [C₁₉H₃₁N₂O₃]⁺ 335.2335, found 335.2337.

### (E)-6-(4-(azocan-1-yl)butyl)-3-hydroxypicolinaldehyde oxime 82′

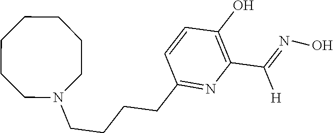 [SMILES: O/N=C/c1nc(CCCCN2CCCCCCC2)ccc1O]

General procedure E was used with starting material 81′ (1.22 g, 3.64 mmol, 1.0 equiv.) to afford the desired product as a trifluoroacetate salt (1.08 g. 71%). ¹H NMR (300 MHz, MeOD) δ 8.31 (s, 1H), 7.36 (d, J=8.5 Hz, 1H), 7.24 (d, J=8.6 Hz, 1H), 4.88 (s, 5H), 3.58-3.37 (m, 2H), 3.29-3.11 (m, 4H), 2.89-2.73 (m, 2H), 2.12-1.92 (m, 2H), 1.92-1.50 (m, 14H). ¹⁹F NMR (282 MHz, MeOD) 5-76.90. ¹³C NMR (75 MHz, MeOD): δ 161.44 (d, J=34.9 Hz), 152.6, 151.8, 150.3, 134.8, 125.4, 124.3, 116.75 (d, J=293.6 Hz), 55.9, 51.3, 35.1, 26.4, 25.2, 24.0, 23.5, 22.4. HRMS (ESI⁺): m/z calcd for [C₁₇H₂₈N₃O₂]⁺ 306.2182, found 306.2182. HPLC (method C): t_{R}=3.23 min (98.50%).

### 3-(5-(benzyloxy)-6-(1,3-dioxolan-2-yl)pyridin-2-yl)prop-2-yn-1-ol 83′

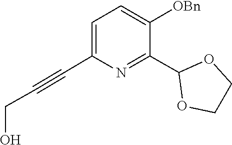 [SMILES: OCC#Cc1ccc(OCc2ccccc2)c(C2OCCO2)n1]

General procedure H was used with 3-(benzyloxy)-6-bromo-2-(1,3-dioxolan-2-yl)pyridine (1.0 g, 2.97 mmol, 1 equiv.) and propargylic alcohol (0.24 mL, 4.16 mmol, 1.4 equiv.) to afford an oil (593 g, 64%). ¹H NMR (300 MHz, CDCl₃) δ 7.49-7.34 (m, 6H), 7.23 (d, J=8.6 Hz, 1H), 6.31 (s, 1H), 5.18 (s, 2H), 4.53 (s, 2H), 4.30-4.17 (m, 2H), 4.14-4.00 (m, 2H), 2.17 (s, 1H). ¹³C NMR (75 MHz, CDCl₃): δ 153.1, 146.9, 135.6, 133.9, 128.7, 128.5, 128.4, 127.4, 120.0, 100.8, 86.4, 84.6, 70.6, 65.8, 51.5. HRMS (ESI⁺): m/z calcd for [C₁₈H₁₈NO₄]⁺ 312.1236, found 312.1232.

### 2-(1,3-dioxolan-2-yl)-6-(3-hydroxypropyl)pyridin-3-ol 84′

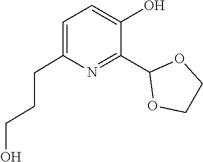 [SMILES: OCCCc1ccc(O)c(C2OCCO2)n1]

General procedure C was used with starting material 83′ (540 mg, 1.29 mmol, 1.0 equiv.) and Pd(OH)₂/C 10% (243 mg, 0.35 mmol, 0.2 equiv.) to afford an oil (283 mg, 73%). ¹H NMR (300 MHz, CDCl₃) δ 8.36 (s, 1H), 7.20 (d, J=8.4 Hz, 1H), 7.10 (d, J=8.4 Hz, 11H), 5.96 (s, 1H), 4.41 (td, J=14.4, 7.0 Hz, 1H), 4.31-4.16 (m, 2H), 4.16-4.02 (m, 2H), 3.72 (t, J=5.8 Hz, 2H), 2.91 (t, J=6.8 Hz, 2H), 1.96 (p, J=6.2 Hz, 2H). ¹³C NMR (75 MHz, CDCl₃) δ 152.4, 150.7, 138.2, 126.0, 124.9, 104.6, 104.5, 65.2, 62.4, 34.4, 31.6. HRMS (ESI⁺): m/z calcd for [C₁₁H₁₆NO₄]⁺ 226.1079, found 226.1084.

### 3-(5-(benzyloxy)-6-(13-dioxolan-2-yl)pyridin-2-yl)propan-1-ol 85′

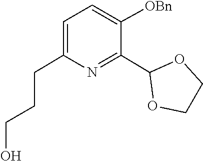 [SMILES: OCCCc1ccc(OCc2ccccc2)c(C2OCCO2)n1]

To a solution of alcohol 84′ (0.28 g, 1.24 mmol, 1 equiv.), in acetone (12 mL, 0.1 M) was added successively Cs₂CO₃ (689 mg, 2.11 mmol, 1.7 equiv.) and benzyl bromide (0.22 mL, 1.86 mmol, 1.5 equiv.). The heterogeneous reaction mixture was stirred at room temperature overnight. Salts were removed by filtration and the crude product was concentrated under reduced pressure. The crude mixture was filtered though celite and concentrated before being purified by flash chromatography on silica gel (0 to 4% MeOH in DCM) to afford an oil (380 mg, 97%). ¹H NMR (300 MHz, CDCl₃) δ 7.48-7.28 (m, 5H), 7.21 (d, J=8.5 Hz, 1H), 7.10 (d, J=8.5 Hz, 1H), 6.37 (s, 1H), 5.13 (s, 2H), 4.71 (s, 1H), 4.33-4.17 (m, 2H), 4.14-3.98 (m, 2H), 3.74 (t, J=5.6 Hz, 2H), 3.01-2.91 (m, 2H), 2.05-1.89 (m, 2H). ¹³C NMR (75 MHz, CDCl₃) δ 152.7, 151.7, 145.0, 136.2, 128.2, 127.3, 124.1, 121.3, 99.5, 70.7, 65.8, 62.7, 34.9, 31.2. HRMS (ESI⁺): m/z calcd for [C₁₈H₂₂NO₄]⁺ 316.1549, found 316.1535.

### Example 4: Biological Activity of Other Compounds of the Invention

Compounds as mentioned in example 3 have been evaluated through tests as presented in example 2.

Results are shown in the following tables.

TABLE 3
IC50 of oximes on hAChE and hBChE

IC₅₀ (μM)

| Oximes | hAChE | hBChE |
|---|---|---|
| 72′ | 734 ± 35 | 461 ± 41 |
| 73′ | 88 ± 8 | 7 ± 0.4 |
| 40′ | 576 ± 49 | 799 ± 115 |
| 41′ | 48 ± 3 | 129 ± 29 |
| 223 | 9 ± 0.8 | 18 ± 3 |
| 56′ | 262 ± 23 | 363 ± 50 |
| 23′ | 4237 ± 458 | 10302 ± 2405 |
| 26′ | 1790 ± 155 | 7356 ± 612 |
| 25′ | 373 ± 22 | 90 ± 7 |
| 24′ | 597 ± 33 | 207 ± 15 |
| 211 | 2045 ± 265 | 509 ± 28 |
| 2-PAM | 580 ± 28 | 5 mM, 90% activity |
| HI-6 | 82 ± 6 | 2480 ± 245 |

TABLE 4
Reactivation kinetics for OP-inhibited hAChE:

| OP | Molécules | kᵣ (min⁻¹) | K_{D} (μM) | kᵣ₂ (mM⁻¹ · min⁻¹) |
|---|---|---|---|---|
| VX | 72′ | 2 ± 0.2 | 861 ± 147 | 2.3 |
| | 73′ | 0.7 ± 0.03 | 41 ± 4 | 17 |
| | 40′ | 0.5 ± 0.007 | 3 ± 0.8 | 167 |
| | 41′ | 0.4 ± 0.02 | 15 ± 2 | 27 |
| | 223 | 0.2 ± 0.006 | 3 ± 0.2 | 67 |
| | 56′ | 0.2 ± 0.01 | 86 ± 15 | 2.3 |
| | 23′ | 0.1 ± 0.01 | 163 ± 41 | 0.6 |
| | 26′ | 0.7 ± 0.06 | 518 ± 122 | 1.4 |
| | 25′ | 0.1 ± 0.006 | 77 ± 14 | 1.3 |
| | 24′ | 0.2 ± 0.005 | 20 ± 3 | 10 |
| | 2-PAM | 0.2 ± 0.013 | 26 ± 7 | 7 |
| | HI-6 | 0.4 ± 0.02 | 19 ± 4 | 20 |
| Sarin | 72′ | 0.6 ± 0.03 | 51 ± 9 | 12 |
| | 73′ | 0.4 ± 0.02 | 13 ± 2 | 31 |
| | 40′ | 0.6 ± 0.02 | 39 ± 5 | 15 |
| | 41′ | 0.5 ± 0.01 | 0.3 ± 0.07 | 1667 |
| | 223 | 0.6 ± 0.03 | 9 ± 2 | 67 |
| | 23′ | 0.05 ± 0.001 | 25 ± 7 | 2 |
| | 26′ | 0.4 ± 0.03 | 421 ± 63 | 1 |
| | 25′ | 0.06 ± 0.001 | 8 ± 1 | 8 |
| | 24′ | 0.1 ± 0.001 | 9 ± 1 | 11 |
| | 2-PAM | 0.3 ± 0.02 | 25 ± 7 | 11 |
| | HI-6 | 0.8 ± 0.06 | 57 ± 11 | 13 |
| Tabun | 72′ | nd | nd | 0.3 |
| | 73′ | 0.07 ± 0.002 | 13 ± 1 | 5.4 |
| | 41′ | 0.4 ± 0.01 | 7 ± 0.8 | 57 |
| | 223 | 0.09 ± 0.004 | 1 ± 0.3 | 90 |
| | 2-PAM | 0.5 ± 0.19 | 211 ± 113 | 2 |
| | HI-6 | 0 | 0 | 0 |
| Paraoxon | 72′ | 0.4 ± 0.008 | 58 ± 6 | 7 |
| | 73′ | 0.4 ± 0.009 | 11 ± 1 | 36 |
| | 41′ | 0.07 ± 0.004 | 33 ± 5 | 2 |
| | 23′ | nd | nd | 0.025 |
| | 26′ | 0.5 ± 0.1 | 3845 ± 973 | 0.13 |
| | 25′ | 0.1 ± 0.006 | 7151 ± 22 | 0.01 |
| | 24′ | 0.5 ± 0.01 | 10 ± 1 | 50 |
| | 2-PAM | 0.07 ± 0.02 | 68 ± 16 | 1 |
| | HI-6 | 0.1 ± 0.01 | 290 ± 70 | 0.4 |

TABLE 5
Reactivation kinetics for OP-inhibited hBChE:

| OP | Molécules | kᵣ (min⁻¹) | K_{D} (μM) | kᵣ₂ (mM⁻¹ · min⁻¹) |
|---|---|---|---|---|
| VX | 72′ | 0.2 ± 0.009 | 28 ± 3 | 7 |
| | 73′ | 0.7 ± 0.03 | 47 ± 6 | 15 |
| | 40′ | 0.7 ± 0.04 | 185 ± 31 | 4 |
| | 41′ | 1 ± 0.03 | 27 ± 3 | 37 |
| | 223 | 0.3 ± 0.01 | 2 ± 0.5 | 150 |
| | 56′ | 0.4 ± 0.02 | 61 ± 12 | 7 |
| | 23′ | 1 ± 0.2 | 1242 ± 458 | 0.8 |
| | 26′ | 0.8 ± 0.05 | 321 ± 63 | 2.5 |
| | 24′ | 0.5 ± 0.01 | 13 ± 1 | 38 |
| | 2-PAM | 1 ± 0.07 | 1551 ± 155 | 0.7 |
| | HI-6 | 0.03 ± 0.002 | 307 ± 41 | 0.1 |
| Sarin | 72′ | 0.1 ± 0.03 | 22 ± 2 | 4.5 |
| | 73′ | 0.6 ± 0.02 | 19 ± 2 | 32 |
| | 40′ | 0.4 ± 0.01 | 53 ± 6 | 7.5 |
| | 41′ | 1 ± 0.02 | 33 ± 3 | 30 |
| | 23′ | 0.2 ± 0.01 | 20 ± 8 | 10 |
| | 26′ | 0.4 ± 0.02 | 173 ± 28 | 2.3 |
| | 24′ | 0.5 ± 0.02 | 33 ± 4 | 15 |
| | 2-PAM | 4 ± 0.5 | 2885 ± 487 | 1.4 |
| | HI-6 | 0.3 ± 0.02 | 409 ± 71 | 0.8 |
| Paraoxon | 72′ | 0.1 ± 0.003 | 10 ± 2 | 10 |
| | 73′ | 0.2 ± 0.007 | 5 ± 0.8 | 40 |
| | 40′ | 0.4 ± 0.01 | 46 ± 7 | 9 |
| | 41′ | 0.4 ± 0.009 | 11 ± 1 | 36 |
| | 56′ | 0.3 ± 0.009 | 9 ± 2 | 33 |
| | 23′ | 0.3 ± 0.01 | 74 ± 21 | 4 |
| | 26′ | 0.3 ± 0.005 | 59 ± 5 | 5 |
| | 24′ | 0.35 ± 0.02 | 29 ± 4 | 12 |
| | 2-PAM | 0.35 ± 0.02 | 65 ± 22 | 5.4 |
| | HI-6 | 0 | 0 | 0 |

### Example 5: Synthesis of Other Compounds of the Invention and their Biological Evaluation

Preparative HPLC Method:

Method A:

Thermo Scientific Acclaim polar advantage II column (5 m, 250×20 mm) with ACN and aq. 20 mM ammonium acetate as eluents at a flow rate of 20 mL/min: 5% ACN for 3 min, then 5% to 38% in 12 min followed by 38% ACN for 4 min and 38% to 100% ACN in 8 min. UV-detection was achieved at 230 nm and 250 nm.

HPLC Method:

Method B:

Thermo Scientific Acclaim polar advantage II column (2.2 μm, 100×2.1 mm) temperature-controlled at 25° C. with ACN and aq. 20 mM ammonium acetate as eluents at a flow rate of 0.4 mL/min: 5% ACN for 3 min, then 5% to 100% ACN in 16 min. UV-detection was achieved at 307 nm.

UHPLC-MS Method:

Method C:

Waters acquity UPLC BEH C18 column (1.7 μm, 100×2.1 mm) temperature-controlled at 40° C. with ACN and aq. 0.1% formic acid as eluents at a flow rate of 0.4 mL/min: 0% to 100% ACN in 10 min. UV-detection was achieved at 254 nm.

Method D:

Waters acquity UPLC BEH C18 column (1.7 μm, 100×2.1 mm) temperature-controlled at 40° C. with ACN and aq. 0.1% formic acid as eluents at a flow rate of 0.4 mL/min: 0% to 100% ACN in 10 min. UV-detection was achieved at 254 nm.

Method E:

Thermo Scientific Hypersil GOLD column (1.9 μm, 100×2.1 mm) temperature-controlled at 30° C. with ACN and aq. 0.2% formic acid as eluents at a flow rate of 0.5 mL/min: 5% to 100% ACN in 10 min. UV-detection was achieved at 254 nm.

### 4-Methyl-4-(pent-4-yn-1-yl)-4λ⁵-morpholino trifluoromethanesulfonate (1a)

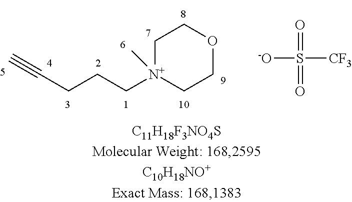

Trifluoromethanesulfonic anhydride (2.07 mL, 12.6 mmol, 1.5 equiv) was dissolved in dry DCM (63 mL) under argon and cooled to 0° C. A solution of 4-pentyn-1-ol (0.78 mL, 8.4 mmol, 1.0 equiv) and dry pyridine (0.68 mL, 8.4 mmol, 1.0 equiv) in dry DCM (21 mL) under argon was added in the previous solution dropwise at 0° C. The solution was stirred at 0° C. for 45 min with a TLC control.

The organic phase was washed twice with water and brine, dried over Na₂SO₄, filtered and concentrated under reduced pressure (temperature of the bath not exceeding 30° C.).

The crude reaction product was used immediately and dissolved in dry ACN (17 mL) under argon. N-methylmorpholine (0.83 mL, 7.56 mmol. 0.9 equiv) was added dropwise.

The solution was stirred at r.t. overnight.

The solution was concentrated under reduced pressure and was purified by flash chromatography on silica gel (DCM/MeOH 100/0→95/5 v/v) to yield the desired product 1a (1.79 g, 75%) %) as a very viscous clear yellow oil or a white solid (depending of the solvent traces and humidity).

R_{f}=0.39 (DCM/MeOH 9/1 v/v). MS (ESI+) m/z (%): 168 (100) [M]⁺. HRMS (ESI+) m/z: [M+H]⁺ calcd for [C₁₀H₁₈NO]⁺ 168.1383, found 168.1380.

¹H NMR (300 MHz, CDCl₃ (+MeOD)) δ 1.90-2.02 (m, 2H, H₂), 2.08 (t, J=2.6 Hz, 1H, H₅), 2.34 (dt, J=6.5 Hz, 2.6 Hz, 2H, H₃), 3.17 (s, 3H, H₆), 3.35-3.48 (m, 4H, H₇, H₁₀), 3.49-3.57 (m, 2H, H₁), 3.95 (br s, 3H, H₈, H₉) ppm

¹³C NMR (75 MHz, CDCl₃ (+MeOD)) δ 15.4 (C₃), 20.7 (C₂), 47.1 (C₆), 60.2 (C₇, C₁₀), 60.6 (C₈, C₉), 64.7 (C₁), 71.0 (C₅), 81.4 (C₄), 120.6 (q, J=320 Hz, CF₃) ppm

### 4-(5-(5-(Benzyloxy)-6-(1,3-dioxolan-2-yl)pyridin-2-yl)pent-4-yn-1-yl)-4-methyl-4λ⁵-morpholino trifluoromethanesulfonate (2a)

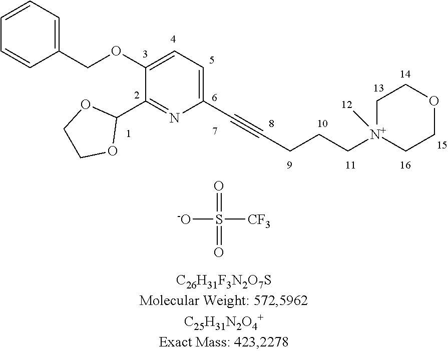

The compound 3-(benzyloxy)-6-bromo-2-(1,3-dioxolan-2-yl)pyridine (713 mg, 2.12 mmol, 1.0 equiv), K₃PO₄ (900 mg, 4.24 mmol, 2.0 equiv) and the alkyne 1a (674 mg, 2.12 mmol, 1.0 equiv) were dissolved in dry ACN (27 mL) under argon, and the solution was degassed with argon for 15 min. CuI (60 mg, 0.32 mmol, 0.15 equiv) and Pd(PPh₃)₄ (348 mg, 0.3 mmol, 0.14 equiv) were added in this order and the solution was stirred overnight at r.t. under argon, protected from light.

The solution was filtered on cotton, concentrated under reduced pressure, and was purified by flash chromatography on silica gel (DCM/MeOH 10/0-+9/1 v/v) to yield the desired product 2a (953 mg, 78%) as a foamy and gummy orange solid.

R_{f}=0.42 (DCM/MeOH 8/2 v/v). MS (ESI+) m/z (%): 423 (100) [M]⁺. HRMS (ESI+) m/z: [M]⁺ calcd for [C₂₅H₃₁N₂O₄]⁺ 423.2278, found 423.2290.

¹H NMR (300 MHz, CDCl₃) δ 2.00-2.15 (m, 2H, H₁₀), 2.57 (t, J=6.6 Hz, 2H, H₉), 3.24 (s, 3H, H₁₂), 3.40-3.54 (m, 4H, H₁₃, H₁₆), 3.59-3.68 (m, 2H, H₁₁), 3.93-3.99 (m, 4H, H₁₄, H₁₅), 3.91-4.19 (m, 4H, CH₂—O), 5.11 (s, 2H, CH₂-Ph), 6.17 (s, 1H, H₁), 7.22 (d, J=8.8 Hz, 1H, H₄), 7.29-7.43 (m, 6H, H_{Ph}, H₅) ppm

¹³C NMR (75 MHz, CDCl₃) δ 16.4 (C₉), 20.8 (C₁₀), 47.1 (C₁₂), 60.2 (C₁₃, C₁₆), 60.6 (C₁₄. C₁₅), 64.6 (C₁₁), 65.8 (CH₂—O), 70.7 (CH₂-Ph), 81.8 (C₇), 86.4 (C₈), 100.7 (C₁), 120.6 (q, J=320 Hz, CF₃), 120.6 (C₄), 127.6 (C_{Ph}), 128.5 (C_{Ph}), 128.8 (C_{Ph}), 129.0 (C₅), 134.1 (C₆), 135.7 (C_{Ph/quat.}), 146.3 (C₂), 153.1 (C₃) ppm

### 4-(5-(6-(1,3-dioxolan-2-yl)-5-hydroxypyridin-2-yl)pentyl)-4-methylmorpholin-4-ium trifluoromethanesulfonate (3a)

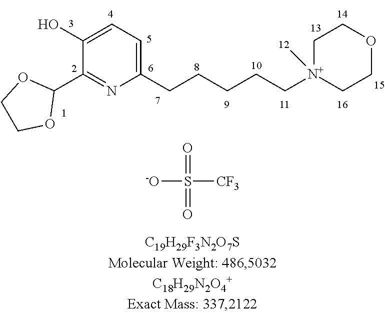

The benzylated compound 2a (1.55 g, 2.7 mmol, 1.0 equiv) was dissolved in MeOH (27 mL) and Pd(OH)₂ (20%) (379 mg, 0.54 mmol, 0.2 equiv) was added under argon. The solution was submitted to three cycles of reduced pressure followed by a H₂ filling. The solution was stirred under H₂ for 1 h10 with a regular TLC control to avoid the formation of the secondary product.

The solution was filtered on a celite pad with DCM/MeOH (1/1 v/v) and concentrated under reduced pressure. The crude reaction product was purified by flash chromatography (DCM/MeOH 10/0→9/1 v/v) to yield the phenol 3a (985 mg, 75%) as a very viscous colourless oil.

R_{f}=0.38 (DCM/MeOH 8/2 v/v). MS (ESI+) m/z (%): 337 (100) [M]⁺, 369 (10) [M+MeOH]⁺. HRMS (ESI+) m/z: [M]⁺ calcd for [C₁₈H₂₉N₂O₄]⁺ 337.2122, found 337.2125.

¹H NMR (300 MHz, CDCl₃ (+MeOD)) δ 1.30 (quint, J=7.4 Hz, 2H, H₉), 1.58-1.72 (m, 4H, H₈, H₁₀), 2.63 (t, J=7.4 Hz, 2H, H₇), 3.05 (s, 3H, H₁₂), 3.22-3.28 (m, 2H, H₁₁), 3.29-3.35 (m, 4H, H₁₃, H₁₆), 3.82-3.90 (m, 4H, H₁₄, H₁₅), 3.95-4.18 (m, 4H, CH₂—O) 5.85 (s, 1H, H₁), 7.00 (d, J=8.4 Hz, 1H, H₈), 7.09 (d, J=8.4 Hz, 1H, H₄) ppm

¹³C NMR (75 MHz, CDCl₃ (+MeOD)) δ 20.9 (C₁₀), 25.3 (C₉), 28.9 (C₈), 36.0 (C₇). 46.4 (C₁₂), 59.8 (C₁₃, C₁₆), 60.5 (C₁₄, C₁₅), 65.2 (CH₂—O), 65.8 (C₁₁), 103.5 (C₁), 120.3 (q, J=317 Hz, CF₃), 124.7 (C₅), 125.7 (C₄), 138.9 (C₂), 150.8 (C₃), 152.2 (C₆) ppm

### 4-(5-(5-hydroxy-6-((hydroxyimino)methyl)pyridin-2-yl)pentyl)-4-methylmorpholin-4-ium trifluoromethanesulfonate (4a)

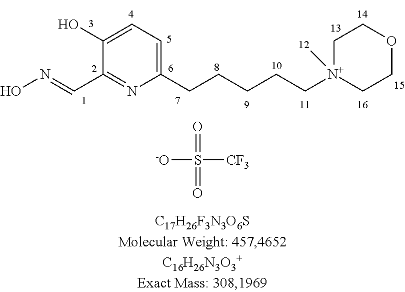

The acetal 3a (985 mg, 2.02 mmol) was dissolved in H₂O/HCO₂H (1/1, v/v) (40 mL) and stirred under argon at 60° C. overnight.

Formic acid was neutralized by a stoichiometric quantity of NaHCO₃ to reach a pH around 8. The aqueous phase was lyophilized, and the freeze-dried product was washed with DCM/MeOH (8/2, v/v). This resulting organic phase was then concentrated under reduced pressure under 30° C. to avoid the degradation of the product (a darkening could be observed). The crude material was purified by flash chromatography on silica gel (DCM/MeOH 10/0→9/1 v/v) to yield the desired aldehyde (658 mg) as a colourless viscous oil which darkens to the concentration. The NMR spectrum shows two forms of the product with the pic of the aldehyde having an integration of its area of 0.66 instead of 1.

R_{f}=0.24 (DCM/MeOH 8/2 v/v). MS (ESI+) m/z (%): 293 (100) [M]⁺. HRMS (ESI+) m/z: [M]⁺ calcd for [C₁₆H₂₅N₂O₃]⁺ 293.1865, found 293.1864.

This aldehyde (658 mg, 1.49 mmol, 1.0 equiv) was dissolved in MeOH (15 mL) and aqueous hydroxylamine (50 wt. %) (0.2 mL, 2.97 mmol, 2.0 equiv) was added. The solution was stirred at r.t. overnight.

The solution was concentrated under reduced pressure and was purified twice by flash chromatography on silica gel (DCM/MeOH 10/0→9/1 v/v) to yield the oxime 4a (265 mg, 29%) as a clear yellow solid.

R_{f}=0.35 (DCM/MeOH 8/2 v/v). MS (ESI+) m/z (%): 308 (100) [M]⁺. HRMS (ESI+) m/z: [M]⁺ calcd for [C₁₆H₂₆N₃O₃]⁺ 308.1969, found 308.1980.

¹H NMR (300 MHz, MeOD) δ 1.42 (quint, J=7.5 Hz, 2H, H₉), 1.70-1.89 (m, 4H, H₈, H₁₀), 2.75 (t, J=7.5 Hz, 2H, H₇), 3.19 (s, 3H, H₁₂), 3.40-3.54 (m, 6H, H₁₃, H₁₆, H₁₁). 3.95-4.02 (m, 4H, H₁₄, H₁₅), 7.17 (d, J=8.5 Hz, 1H, H), 7.27 (d, J=8.5 Hz, 1H, H₄), 8.28 (s, 1H, H₁) ppm

¹³C NMR (75 MHz, MeOD) δ 22.2 (C₁₀), 26.7 (C₉), 30.4 (C₈), 37.3 (C₇), 47.1 (C₁₂), 60.9 (C₁₃, C₁₆). 61.5 (C₁₄, C₁₅), 66.5 (C₁₁), 121.7 (q, J=320 Hz, CF₃), 125.4 (C₅), 126.0 (C₄), 136.2 (C₂), 152.7 (C₁), 153.7 (C₃), 154.2 (C₆) ppm

### 4-(5-(5-hydroxy-6-((hydroxyimino)methyl)pyridin-1-ium-2-yl)pentyl)-4-methylmorpholin-4-ium acetate (5a)

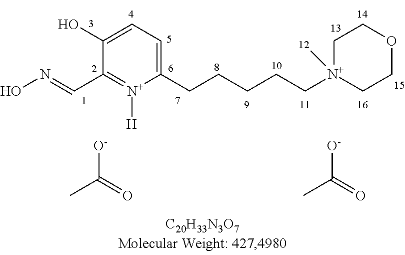

The triflate salt 4a (30 mg, 66 μmol) was purified by inverse preparative chromatography (Method A) to yield the acetate salt 5a (10 mg, 23 mol) as a hygroscopic orange solid.

HPLC (Method B) tᵣ (min), relative area (%): 9.563, 97.84.

¹H NMR (300 MHz, MeOD) δ 1.42 (quint, J=7.5 Hz, 2H, H₉), 1.70-1.89 (m, 4H, H₅, H₁₀), 1.91 (s, 6H, CH₃CO₂—), 2.77 (t, J=7.7 Hz, 2H, H₇), 3.18 (s, 3H, H₁₂), 3.40-3.54 (m, 6H, H₁₃, H₁₆, H₁₁), 3.95-4.02 (m, 4H, H₁₄, H₁₅), 7.17 (d, J=8.5 Hz, 1H, H₈), 7.28 (d, J=8.5 Hz, 1H, H₄), 8.28 (s, 1H, H₁) ppm

¹³C NMR (75 MHz, MeOD) δ 22.2 (C₁₀), 23.7 (CH₃CO₂—), 26.7 (C₉), 30.4 (C₈), 37.3 (C₇), 47.1 (C₁₂), 60.9 (C₁₃, C₁₆), 61.5 (C₁₄, C₁₅), 66.5 (C₁₁), 125.4 (C₈), 126.0 (C₄), 136.4 (C₂), 152.7 (C₁), 153.9 (C₃), 154.2 (C₆) 179.4 (CO₂⁻) ppm

### 4-(5-(5-hydroxy-6-((hydroxyimino)methyl)pyridin-2-yl)pentyl)-4-methyl-4V-morpholino trifluoromethanesulfonate methanesulfonate (6a)

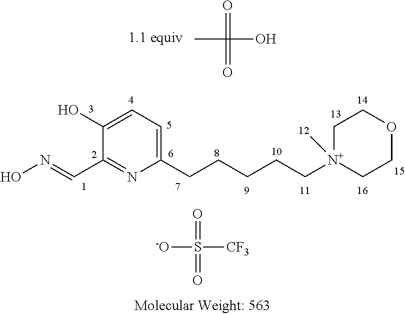

The oxime 4a (176 mg, 0.39 mmol, 1.0 equiv) was dissolved in a vial in MeOH (HPLC grade) (1.9 mL). Methanolic solution of methanesulfonic acid (0.5 M: 0.16 mL acid in 5.0 mL MeOH) (0.76 mL, 0.38 mmol, 1.0 equiv) was added, and the solution was concentrated under reduced pressure to yield the methanesulfonate salt 6a (217 mg) as a yellow solid. 1.1 equivalents of methanesulfonic acid were added according to the NMR integration and the difference of mass measured.

LC-MS (ESI+) (Method C) tᵣ (min), relative area (%), m/z: 1.957, 7.53, 308 [M]⁺; 2.130, 92.36, 308 [M]⁺. Purity: 99.89%.

¹H NMR (300 MHz, MeOD) δ 1.50 (quint, J=7.5 Hz, 2H, H₉), 1.77-1.96 (m, 4H, H., H₁₀), 2.73 (s, 3H, CH₃—S), 3.05 (t, J=7.6 Hz, 2H, H₇), 3.21 (s, 3H, H₁₂), 3.40-3.59 (m, 6H, H₁₃, H₁₆, H₁₁), 3.92-4.06 (m, 4H, H₁₄, H₁₅), 7.79 (d, J=8.9 Hz, 1H, H₈), 7.98 (d, J=8.9 Hz, 1H, H₄), 8.45 (s, 1H, H₁) ppm

¹³C NMR (75 MHz, MeOD) δ 22.0 (C₁₀), 26.4 (C₉), 29.8 (C₈), 32.9 (C₇), 39.6 (CH₃—S), 47.2 (C₁₂), 60.9 (C₁₃, C₁₆), 61.5 (C₁₄, C₁₅), 66.2 (C₁₁), 121.7 (q, J=319 Hz, CF₃), 128.8 (C₅), 132.4 (C₂), 134.7 (C₄), 140.9 (C₁), 150.6 (C₆), 154.6 (C₃) ppm.

### 1-(but-3-yn-1-yl)quinuclidin-1-ium trifluoromethanesulfonate (1b)

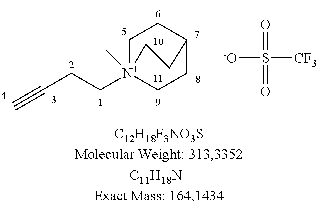

Trifluoromethanesulfonic anhydride (2.21 mL, 13.5 mmol, 1.0 equiv) was dissolved in dry DCM (100 mL) under argon and cooled to 0° C. A solution of 4-butyn-1-ol (1.02 mL, 13.5 mmol, 1.0 equiv) and dry pyridine (1.09 mL, 13.5 mmol, 1.0 equiv) in dry DCM (25 mL) under argon was added in the previous solution dropwise at 0° C. The solution was stirred at 0° C. for 45 min with a TLC control.

The organic phase was washed twice with water and brine, dried over Na₂SO₄, filtered and concentrated under reduced pressure (temperature of the bath not exceeding 30° C.).

The crude reaction product was used immediately and dissolved in dry ACN (20 mL) under argon. Quinuclidine (1.43 g, 12.8 mmol, 0.95 equiv) in dry ACN (7.0 mL) was added dropwise. The solution was stirred at r.t. overnight.

The solution was concentrated under reduced pressure and was purified by flash chromatography on silica gel (DCM/MeOH 100/0→96/4 v/v) to yield the desired product 1b (3.50 g, 83%) as a white solid.

R_{f}=0.36 (DCM/MeOH 95/5 v/v). MS (ESI+) m/z (%): 164 (100) [M]⁺. HRMS (ESI+) m/z: [M+H]⁺ calcd for [C₁₁H₁₈N]⁺ 164.1434, found 164.1430.

¹H NMR (300 MHz, MeOD) δ 1.95-2.07 (m, 6H, H₆, H₁₁, H₈), 2.17 (sept, J=3.3 Hz, 1H, H₇), 2.56 (t, J=2.6 Hz, 1H, H₄), 2.76 (td, J=7.4 Hz, 2.6 Hz, 2H, H₂), 3.36 (t, J=7.4 Hz, 2H, H₁), 3.44-3.54 (m, 6H, H₈, H₁₀, H₉) ppm

¹³C NMR (75 MHz, MeOD) δ 13.7 (C₂), 20.7 (C₇), 24.7 (C₆, C₁₁, C₈), 55.9 (C₈, C₁₀, C₉), 63.3 (C₁), 73.0 (C₄), 79.7 (C₃), 121.7 (q, J=318 Hz, CF₃) ppm

### 1-(4-(6-(1,3-dioxolan-2-yl)-5-hydroxypyridin-2-yl)butyl)quinuclidin-1-ium trifluoromethanesulfonate (2b)

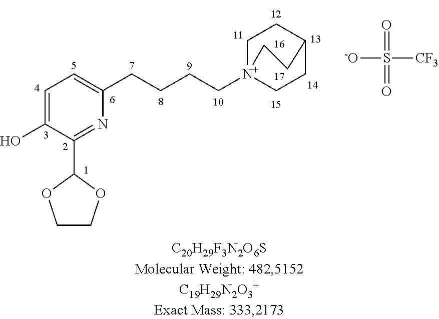

The compound 3-(benzyloxy)-6-bromo-2-(1,3-dioxolan-2-yl)pyridine (1.38 g, 4.1 mmol, 1.1 equiv), K₃PO₄ (1.57 g, 7.4 mmol, 2.0 equiv) and the alkyne 1b (1.16 g, 3.7 mmol, 1.0 equiv) were dissolved in dry ACN (29 mL) under argon, and the solution was degassed with argon for 15 min. CuI (177 mg, 0.93 mmol, 0.25 equiv) and Pd(PPh₃)₄(855 mg, 0.74 mmol, 0.2 equiv) were added in this order and the solution was stirred overnight at r.t. under argon, protected from light.

The solution was filtered on cotton, concentrated under reduced pressure, and was purified by flash chromatography on silica gel (DCM/MeOH 100/0→95/5 v/v) to yield a mixture (412 mg) of the desired product and a by-product of the quinuclidine (25% according to the NMR) as a foamy brown solid; that is around (0.31 g, 15%) of the desired product.

R_{f}=0.44 (DCM/MeOH 9/1 v/v). MS (ESI+) m/z (%): 419 (100) [M]⁺. HRMS (ESI+) m/z: [M]⁺ calcd for [C₂₆H₃₁N₂O₃]⁺ 419.2329, found 419.2332.

This mixture (505 mg) containing approximately 0.65 mmol of the benzylated compound (1.0 equiv) was dissolved in ACN (7 mL) in a Teflon vessel of a hydrogenation reactor. Pd(OH)₂ (20%) (92 mg, 0.13 mmol, 0.2 equiv) was added under argon and the reactor was closed. The reactor was subjected to three cycles vacuum-hydrogen and finally filled with hydrogen at 10 Bar. The solution was stirred under H₂ for 7 h with a TLC control.

The solution was filtered on a celite pad with DCM/MeOH (1/1 v/v) and concentrated under reduced pressure to yield after a flash chromatography (DCM/MeOH 10/0→9/1 v/v) the phenol 2b (148 mg, around 47%) as a clear yellow oil.

R_{f}=0.63 (DCM/MeOH 8/2 v/v). MS (ESI+) m/z (%): 333 (45) [M]⁺, 365 (15) [M+MeOH]⁺. HRMS (ESI+) m/z: [M]⁺ calcd for [C₁₉H₂₉N₂O₃]⁺ 333.2173, found 333.2177.

¹H NMR (300 MHz, MeOD) δ 1.60-1.79 (m, 4H, H₈, H₉), 1.88-2.02 (m, 6H, H₁₂, H₁₇, H₁₄), 2.12 (sept, J=3.2 Hz, 1H, H₁₃), 2.70-2.82 (m, 2H, H₇), 3.07-3.17 (m, 2H, H₁₀), 3.32-3.41 (m, 6H, H₁₁, H₁₆, H₁₅), 3.95-4.29 (m, 4H, CH₂—O), 6.05 (s, 1H, H₁), 7.18 (d, J=8.5 Hz, 1H, H₈), 7.22 (d, J=8.5 Hz, 1H, H₄) ppm

¹³C NMR (75 MHz, MeOD) δ 20.7 (br t, J=4 Hz, C₁₃), 22.2 (C₉), 24.7 (C₁₂, C₁₄, C₁₇), 27.8 (C₈), 36.4 (C₇), 55.6 (br s, C₁₅, C₁₆, C₁₈), 65.1 (br s, C₁₀), 66.5 (CH₂—O), 102.0 (C₁), 121.8 (q, J=318 Hz, CF₃), 125.9 (C₅), 126.3 (C₄), 142.3 (C₂), 152.3 (C₃), 152.7 (C₆) ppm

### 1-(4-(5-hydroxy-6-((hydroxyimino)methyl)pyridin-2-yl)butyl)quinuclidin-1-ium trifluoromethanesulfonate (3b)

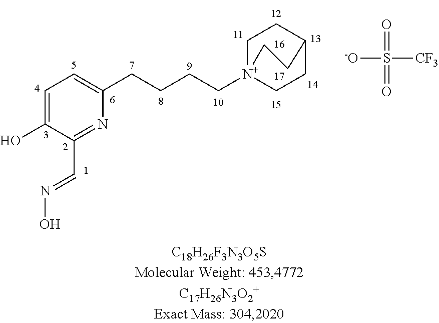

The acetal 2b (148 g, 0.3 mmol, 1.0 equiv) was dissolved in H₂O/HCO₂H (1/1, v/v) (6.0 mL) and stirred under argon at 60° C. overnight.

Formic acid was neutralized by a stoichiometric quantity of NaHCO₃ to reach a pH around 8. The aqueous phase was lyophilized, and the freeze-dried product was washed with DCM/MeOH (8/2, v/v). This resulting organic phase was then concentrated under reduced pressure under 30° C. to avoid the degradation of the product (a darkening could be observed). The crude material was purified by flash chromatography on silica gel (DCM/MeOH 100/0→9/1 v/v) to yield a mixture (95 mg) of a secondary product (33% according to the NMR) and the desired aldehyde as a very clear yellow solid.

R_{f}=0.49 (DCM/MeOH 8/2 v/v) from the reaction mixture H₂O/HCO₂H. MS (ESI+) m/z (%): 289 (100) [M]⁺. HRMS (ESI+) m/z: [M]⁺ calcd for [C₁₇H₂₅N₂O₂]⁺ 289.1911, found 289.1918.

This mixture (95 mg) containing around (60 mg, 0.14 mmol) of the aldehyde was dissolved in MeOH (2.0 mL) and aqueous hydroxylamine (50 wt. %) (0.03 mL, 0.41 mmol, 1.3 equiv) was added. The solution was stirred at r.t. for 4 hours and followed by TLC.

The solution was concentrated under reduced pressure and was purified by flash chromatography on silica gel (DCM/MeOH 10/0→9/1 v/v) to yield the oxime 3b (59 mg, 93%) as a very clear yellow crystalline solid.

R_{f}=0.51 (DCM/MeOH 8/2 v/v). MS (ESI+) m/z (%): 304 (100) [M]⁺. HRMS (ESI+) m/z: [M]⁺ calcd for [C₁₇H₂₆N₃O₂]⁺ 304.2020, found 304.2023.

¹H NMR (300 MHz, MeOD) δ 1.65-1.81 (m, 4H, H₈, H₉), 1.92-2.04 (m, 6H, H₁₂, H₁₇, H₁₄), 2.15 (sept, J=3.2 Hz, 1H, H₁₃), 2.72-2.85 (m, 2H, H₇), 3.09-3.22 (m, 2H, H₁₀), 3.36-3.45 (m, 6H, H₁₁, H₁₆, H₁₅), 7.18 (d, J=8.5 Hz, 1H, H₈), 7.28 (d, J=8.5 Hz, 1H, H₄), 8.28 (s, 1H, H₁) ppm

¹³C NMR (75 MHz, MeOD) δ 20.8 (C₁₃), 22.5 (C₉), 24.8 (C₁₂, C₁₄, C₁₇), 27.8 (C₈), 36.8 (C₇), 55.7 (C₁₁, C₁₅, C₁₆). 65.2 (C₁₀), 121.8 (q, J=320 Hz, CF₃), 125.5 (C₅), 126.1 (C₄), 136.4 (C₂), 152.8 (C₁), 153.6 (C₃), 153.9 (C₆) ppm

### 1-(4-(5-hydroxy-6-((hydroxyimino)methyl)pyridin-2-yl)butyl)-1λ⁵-azabicyclo[2.2.2]octan-1-yl trifluoromethanesulfonate methanesulfonate (4b)

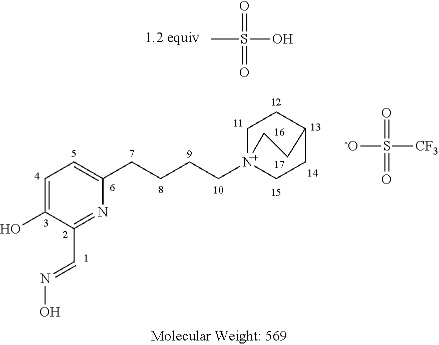

The oxime 3b (23 mg, 0.05 mmol, 1.0 equiv) was dissolved in a vial in MeOH (HPLC grade) (1.0 mL). Methanolic solution of methanesulfonic acid (0.2 M: 39 μL acid in 3.0 mL MeOH) (0.25 mL, 0.05 mmol, 1.0 equiv) was added, and the solution was concentrated under reduced pressure to yield the methanesulfonate salt 4b (28 mg) as a clear yellow solid. 1.2 equivalent of methanesulfonic acid were added according to the NMR integration and the difference of mass measured.

LC-MS (ESI+) (Method D) tᵣ (min), relative area (%), m/z: 2.290, 2.31, 304 [M]⁺; 2.487, 97.69, 304 [M]⁺. Purity: 100%.

¹H NMR (300 MHz, MeOD) δ 1.71-1.92 (m, 4H, H₈, H₉), 1.93-2.07 (m, 6H, H₁₂, H₁₇, H₁₄), 2.10-2.20 (m, 1H, H₁₃), 2.72 (s, 3H, CH₃—S), 3.08 (t, J=6.6 Hz, 2H, H₇), 3.15-3.25 (m, 2H, H₁₀), 3.39-3.49 (m, 6H, H₁₁, H₁₆, H₁₅), 7.79 (d, J=8.9 Hz, 1H, H₈), 7.98 (d, J=8.9 Hz, 1H, H₄), 8.46 (s, 1H, H₁) ppm

¹³C NMR (75 MHz, MeOD) δ 20.8 (C₁₃), 22.6 (C₉), 24.8 (C₁₂, C₁₄, C₁₇), 27.3 (C₈), 32.7 (C₇), 39.6 (CH₃—S), 55.8 (C₁₁, C₁₅, C₁₆), 64.8 (C₁₀), 121.7 (q, J=320 Hz, CF₃), 128.8 (C₅), 133.0 (C₂), 134.7 (C₄), 140.7 (C₁), 149.9 (C₆), 154.8 (C₃) ppm

### 1-(pent-4-yn-1-yl)quinuclidin-1-ium trifluoromethanesulfonate (1c)

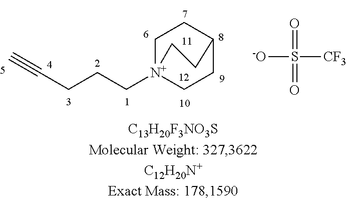

Trifluoromethanesulfonic anhydride (2.21 mL, 13.5 mmol, 1.0 equiv) was dissolved in dry DCM (100 mL) under argon and cooled to 0° C. A solution of 4-pentyn-1-ol (1.26 mL, 13.5 mmol, 1.0 equiv) and dry pyridine (1.09 mL, 13.5 mmol, 1.0 equiv) in dry DCM (25 mL) under argon was added in the previous solution dropwise at 0° C. The solution was stirred at 0° C. for 45 min with a TLC control.

The organic phase was washed twice with water and brine, dried over Na₂SO₄, filtered and concentrated under reduced pressure (temperature of the bath not exceeding 30° C.).

The crude reaction product was used immediately and dissolved in dry ACN (20 mL) under argon. Quinuclidine (1.27 g. 11.5 mmol. 0.85 equiv) in dry ACN (7.0 mL) was added dropwise. The solution was stirred at r.t. overnight.

The solution was concentrated under reduced pressure and was purified by flash chromatography on silica gel (DCM/MeOH 10/0→9/1 v/v) to yield the desired product 1c (3.55 g, 85%) as a white solid.

R_{f}=0.53 (DCM/MeOH 9/1 v/v). MS (ESI+) m/z (%): 178 (100) [M]⁺. HRMS (ESI+) m/z: [M+H] calcd for [C₁₂H₂₀N]⁺ 178.1590, found 178.1595.

¹H NMR (300 MHz, CDCl₃ (+MeOD)) δ 1.75-1.86 (m, 2H, H₂), 1.86-1.96 (m, 6H, H₇, H₁₂, H₉), 2.00 (t, J=2.5 Hz, 1H, H₅), 2.10 (sept, J=3.1 Hz, H₈), 2.21 (td, J=6.6 Hz, 2.5 Hz, 2H, H₃), 3.10-3.20 (m, 2H, H₁), 3.28-3.36 (m, 6H, H₆, H₁₁, H₁₀) ppm

¹³C NMR (75 MHz, CDCl₃ (+MeOD)) δ 15.4 (C₃), 19.2 (C₈), 20.9 (C₂), 23.6 (C₇, C₁₂, C₉), 54.9 (C₆, C₁₁, C₁₀), 63.3 (C₁), 70.6 (C₅), 81.2 (C₄), 120.3 (q, J=320 Hz, CF₃) ppm

### 1-(5-(5-(benzyloxy)-6-(1,3-dioxolan-2-yl)pyridin-2-yl)pent-4-yn-1-yl)quinuclidin-1-ium trifluoromethanesulfonate (2c)

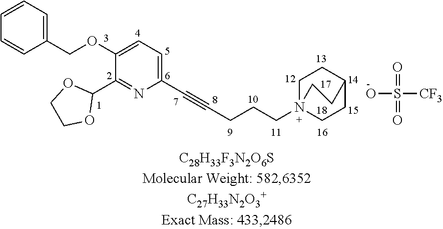

The compound 3-(benzyloxy)-6-bromo-2-(1,3-dioxolan-2-yl)pyridine (2.66 g, 7.9 mmol. 1.0 equiv), K₃PO₄ (3.35 g, 15.8 mmol, 2.0 equiv) and the alkyne 1e (2.59 g, 7.9 mmol, 1.0 equiv) were dissolved in dry ACN (100 mL) under argon, and the solution was degassed with argon for 15 min. CuI (227 mg, 1.19 mmol, 0.15 equiv) and Pd(PPh₃)₄ (913 mg, 0.79 mmol, 0.1 equiv) were added in this order and the solution was stirred overnight at r.t. under argon, protected from light.

The solution was filtered on cotton, concentrated under reduced pressure, and was purified by flash chromatography on silica gel (DCM/MeOH 10/0→8/2 v/v) to yield the desired product 2c (3.10 g, 67%) as a foamy brown solid.

R_{f}=0.55 (DCM/MeOH 8/2 v/v). MS (ESI+) m/z (%): 433 (100) [M]⁺. HRMS (ESI+) m/z: [M]⁺ calcd for [C₂₇H₃₃N₂O₃]⁺ 423.2486, found 433.2478.

¹H NMR (300 MHz, CDCl₃) δ 1.79-1.97 (m, 8H, H₁₀, H₁₃, H₁₅, H₁₈), 2.01-2.09 (m, 1H, H₁₄), 2.44 (t, J=6.3 Hz, 2H, H₉), 3.18-3.27 (m, 2H, H₁₁), 3.30-3.41 (m, 6H, H₁₂, H₁₆, H₁₇), 3.86-3.96 (m, 2H, CH₂—O), 4.01-4.10 (m, 2H, CH₂—O), 5.03 (s, 2H, CH₂-Ph), 6.11 (s, 1H, H₁), 7.20 (d, J=8.7 Hz, 1H, H₄), 7.24-7.40 (m, 6H, H₅, H_{Ph}) ppm

¹³C NMR (75 MHz, CDCl₃) δ 16.4 (C₉), 19.1 (C₁₄), 21.0 (C₁₀), 23.6 (C₁₃, C₁₅, C₁₈), 54.7 (C₁₂, C₁₆, C₁₇), 63.2 (C₁₁), 65.6 (CH₂—O), 70.4 (CH₂-Ph), 81.5 (C₇), 86.1 (C₈), 100.8 (C₁), 120.4 (C₄), 120.7 (q, J=322 Hz, CF₃), 127.4 (C_{Ph}). 128.2 (C_{Ph}), 128.6 (C_{Ph}), 128.9 (C₅), 134.0 (C₆), 135.5 (C_{Ph/quat.}), 146.1 (C₂). 152.8 (C₃) ppm

### 1-(5-(5-hydroxy-6-((hydroxyimino)methyl)pyridin-2-yl)pentyl)quinuclidin-1-ium trifluoromethanesulfonate (3c)

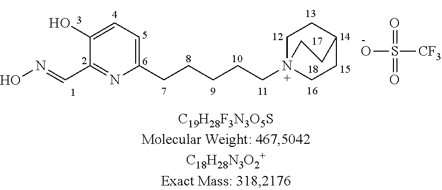

The benzylated compound 2c (1.61 g, 2.76 mmol, 1.0 equiv) was dissolved in dry ACN (28 mL) and Pd(OH)₂ (20%) (582 mg, 0.83 mmol, 0.3 equiv) was added under argon. The solution was submitted to three cycles of reduced pressure followed by a H₂ filling. The solution was stirred under H₂ for 24 h with a regular TLC control to avoid the formation of the secondary product.

The solution was filtered on a celite pad with DCM/MeOH (1/1 v/v) and concentrated under reduced pressure to yield the desired phenol product (1.24 g, 90%) as a very viscous yellow oil. Without any major impurity (according to the NMR) the crude reaction product was used in the next step without further purification

R_{f}=0.50 (DCM/MeOH 8/2 v/v). MS (ESI+) m/z (%): 347 (100) [M]⁺, 379 (20) [M+MeOH]⁺. HRMS (ESI+) m/z: [M]⁺ calcd for [C₂₀H₃₁N₂O₃]⁺ 347.2329, found 347.2341.

This compound (1.24 g, 2.50 mmol, 1.0 equiv) was dissolved in H₂O/HCO₂H (1/1, v/v) (50 mL) and stirred under argon at 60° C. overnight.

Formic acid was neutralized by a stoichiometric quantity of NaHCO₃ to reach a pH around 8. The aqueous phase was lyophilized, and the freeze-dried product was washed with DCM/MeOH (8/2, v/v). This resulting organic phase was then concentrated under reduced pressure under 30° C. to avoid the degradation of the product (a darkening could be observed). The crude material was purified by flash chromatography on silica gel (DCM/MeOH 100/0→94/6 v/v) to yield the desired aldehyde (837 mg) as a viscous yellow oil which darkens to the concentration.

R_{f}=0.37 (DCM/MeOH 8/2 v/v). MS (ESI+) m/z (%): 303 (100) [M]⁺. HRMS (ESI+) m/z: [M]⁺ calcd for [C₁₈H₂₇N₂O₂]⁺ 303.2067, found 303.2078.

This aldehyde (837 mg, 1.85 mmol, 1.0 equiv) was dissolved in MeOH (19 mL) and aqueous hydroxylamine (50 wt. %) (0.18 mL, 2.77 mmol, 1.5 equiv) was added. The solution was stirred at r.t. overnight.

The solution was concentrated under reduced pressure and was purified by flash chromatography on silica gel (DCM/MeOH 100/0→94/6 v/v) to yield the oxime 3c (578 mg, 67%) as a very clear yellow crystalline solid.

R_{f}=0.58 (DCM/MeOH 8/2 v/v). MS (ESI+) m/z (%): 318 (100) [M]⁺. HRMS (ESI+) m/z: [M]⁺ calcd for [C₁₈H₂₈N₃O₂]⁺ 318.2176, found 318.2179.

¹H NMR (300 MHz, MeOD) δ 1.37 (quint, J=7.7 Hz, 2H, H₉), 1.76 (quint, J=7.7 Hz, 4H, H₁₀, H₈), 1.92-2.05 (m, 6H, H₁₃, H₁₅, H₁₈), 2.15 (sept, J=3.3 Hz, 1H, H₁₄), 2.75 (t, J=7.7 Hz, 2H, H₇), 3.05-3.15 (m, 2H, H₁₁), 3.36-3.46 (m, 6H, H₁₂, H₁₆, H₁₇), 7.17 (d, J=8.5 Hz, 1H, H₅), 7.28 (d, J=8.5 Hz, 1H, H₄), 8.28 (s, 1H, H₁) ppm

¹³C NMR (75 MHz, MeOD) δ 20.8 (t, J=4 Hz, C₁₄), 22.8 (C₁₀), 24.8 (C₁₃, C₁₅, C₁₈), 27.0 (C₉), 30.5 (C₈), 37.4 (C₇), 55.7 (br t, J=3 Hz, C₁₂, C₁₆, C₁₇), 65.4 (t, J=3 Hz, C₁₁), 121.8 (q, J=320 Hz, CF₃), 125.4 (C₅), 126.1 (C₄), 136.3 (C₂), 152.8 (C₁), 153.8 (C₃), 154.3

### (C₆) ppm 1-(5-(5-hydroxy-6-((hydroxyimino)methyl)pyridin-2-yl)pentyl)-1λ⁵-azabicyclo[2.2.2]octan-1-yl trifluoromethanesulfonate methanesulfonate (4c)

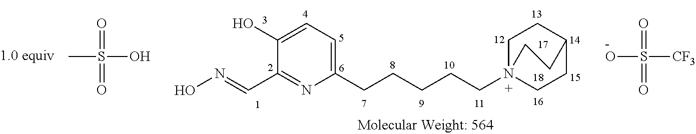

The oxime 3c (75 mg, 0.16 mmol, 1.0 equiv) was dissolved in a vial in MeOH (HPLC grade) (1.6 mL). Methanolic solution of methanesulfonic acid (0.2 M: 39 μL acid in 3.0 mL MeOH) (0.79 mL, 0.16 mmol, 1.0 equiv) was added, and the solution was concentrated under reduced pressure to yield the methanesulfonate salt 4c (86 mg) as a clear yellow solid. 1.0 equivalent of methanesulfonic acid were added according to the NMR integration and the difference of mass measured.

LC-MS (ESI+) (Method E) tᵣ (min), relative area (%), m/z: 2.107, 1.45, 318 [M]⁺; 2.197, 97.62, 318 [M]⁺. Purity: 99.07%.

¹H NMR (300 MHz, MeOD) δ 1.45 (quint, J=7.6 Hz, 2H, H₉), 1.73-1.90 (m, 4H, H₁₀, H₈), 1.92-2.05 (m, 6H, H₁₃, H₁₅, H₁₈), 2.16 (sept, J=3.2 Hz, 1H, H₁₄), 2.72 (s, 3H, CH₃—S), 3.04 (t, J=7.8 Hz, 2H, H₇), 3.10-3.20 (m, 2H, H₁₁), 3.39-3.50 (m, 6H, H₁₂, H₁₆, H₁₇), 7.78 (d, J=8.9 Hz, 1H, H₈), 7.98 (d, J=8.9 Hz, 1H, H₄), 8.45 (s, 1H, H₁) ppm

¹³C NMR (75 MHz, MeOD) δ 20.8 (br t, J=4 Hz, C₁₄), 22.7 (C₁₀), 24.8 (C₁₃, C₁₅, C₁₈), 26.8 (C₉), 30.0 (C₈), 33.0 (C₇), 39.6 (CH₃—S), 55.7 (br s, C₁₂, C₁₆, C₁₇), 65.2 (br s, C₁₁), 121.7 (q, J=320 Hz, CF₃), 128.8 (C₅), 132.8 (C₂). 134.7 (C₄), 140.7 (C₁), 150.7 (C₆), 154.7 (C₃) ppm

Compounds have been evaluated through tests as presented in example 2.

Results are shown in the following tables.

TABLE 6
Reactivation kinetics for OP-inhibited hAChE:

| OP | Oximes | kᵣ (min⁻¹) | K_{D} (μM) | kᵣ₂ (mM⁻¹ · min⁻¹) |
|---|---|---|---|---|
| VX | 5a | 0.5 ± 0.02 | 31 ± 3 | 16 |
| | 4c | 2 ± 0.3 | 259 ± 52 | 8 |
| | 4b | 1 ± 0.08 | 43 ± 6 | 23 |
| | 2-PAM | 0.2 ± 0.01 | 26 ± 7 | 7 |
| | HI-6 | 0.4 ± 0.02 | 19 ± 4 | 20 |
| Sarin | 5a | 0.25 ± 0.005 | 9 ± 0.8 | 28 |
| | 4c | 0.3 ± 0.01 | 78 ± 9 | 4 |
| | 4b | 0.2 ± 0.01 | 6 ± 1 | 33 |
| | 2-PAM | 0.3 ± 0.02 | 25 ± 7 | 11 |
| | HI-6 | 0.8 ± 0.06 | 57 ± 11 | 13 |
| Tabun | 5a | 0.06 ± 0.001 | 4 ± 0.3 | 15 |
| | 4c | 0.03 ± 0.001 | 13 ± 1.4 | 2 |
| | 4b | 0.1 ± 0.003 | 0.3 ± 0.07 | 333 |
| | 2-PAM | 0.5 ± 0.19 | 211 ± 113 | 2 |
| | HI-6 | ∅ | ∅ | ∅ |
| POX | 5a | 0.2 ± 0.007 | 6 ± 0.8 | 33 |
| | 4c | 0.6 ± 0.03 | 70 ± 12 | 9 |
| | 4b | 0.3 ± 0.009 | 0.2 ± 0.3 | 1500 |
| | 2-PAM | 0.07 ± 0.02 | 68 ± 16 | 1 |
| | HI-6 | 0.1 ± 0.01 | 290 ± 70 | 0.4 |

TABLE 7
Reactivation kinetics for OP-inhibited hBChE:

| OP | Oximes | kᵣ (min⁻¹) | K_{D} (μM) | kᵣ₂ (mM⁻¹ · min⁻¹) |
|---|---|---|---|---|
| VX | 5a | 0.2 ± 0.012 | 194 ± 13 | 1 |
| | 4c | 0.2 ± 0.01 | 23 ± 4 | 10 |
| | 4b | 0.3 ± 0.01 | 2 ± 0.3 | 150 |
| | 2-PAM | 1 ± 0.07 | 1551 ± 155 | 0.7 |
| | HI-6 | 0.03 ± 0.002 | 307 ± 41 | 0.1 |
| Sarin | 4b | 0.2 ± 0.004 | 10 ± 0.01 | 20 |
| | 2-PAM | 4 ± 0.5 | 2885 ± 487 | 1.4 |
| | HI-6 | 0.3 ± 0.02 | 409 ± 71 | 0.8 |
| POX | 5a | 0.08 ± 0.002 | 0.7 ± 0.1 | 114 |
| | 4b | 0.2 ± 0.004 | 1 ± 1 | 200 |
| | 2-PAM | 0.35 ± 0.02 | 65 ± 22 | 5.4 |
| | HI-6 | 0 | 0 | 0 |

1. A method of in vivo, in vitro, or ex vivo reactivation of human or animal butyrylcholinesterase comprising administering an effective dose of a compound of following formula (I), said butyrylcholinesterase being prior or after administering inhibited by at least one organophosphorus nerve agent: 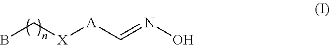
wherein:
n is an integer from 1 to 6;
at least one of the carbon atoms of 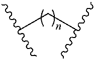
being optionally replaced by an atom chosen from nitrogen, oxygen and sulfur, said nitrogen being optionally substituted by a methyl or ethyl group;
X is a single bond or chosen from —O—, —S—, —NH—, and —NR_{c}—, with R_{c} being methyl or ethyl;
A is chosen from the group comprising arene diyles and 5 to 6 membered heteroarene diyles, said heteroarene being chosen from the group comprising pyridine, thiophene, thiadiazole, oxathiazole, in particular pyridine;
A is optionally substituted by at least one group R chosen from —OH, C₁-C₆ alkyl, —O—C₁-C₆ alkyl, -halogen, notably —Cl, —Br, —F, in particular —OH;
B is chosen:
(i) from heterocyclic groups with 4 to 10 carbon atoms comprising at least one nitrogen atom, and heteroaryl groups chosen from indole, pyrrazole, imidazole, oxazole, thiazole, oxadiazole and thiadiazole,
the heteroaryl cycle B is optionally fused with at least an arene, in particular a benzene, to form a polycycle B′;
the cycle B or B′ is optionally substituted by at least one group Z chosen from C₁-C₆ alkyl, in particular methyl or ethyl; O—C₁-C₆ alkyl, in particular —OMe; aryl, in particular phenyl; heteroaryl, in particular pyridyl, pyrimidinyl; benzyl; benzhydryl; and —NRₐR_{b} groups, wherein Rₐ and R_{b} are independently chosen from H and C₁-C₆ alkyls, Rₐ and R_{b} being in particular H;
said cycle B not being benzhydryl-piperazine, when n is 4, 5 or 6, or X is a single bond, or none of the carbon atoms of 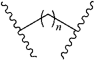
is replaced by an atom chosen from nitrogen, oxygen and sulfur, or said nitrogen of group B does not form a quaternary ammonium,
wherein said nitrogen optionally forms a quaternary ammonium by being further substituted by a C₁-C₆ alkyl or by a C₁-C₆ alkane diyl also bound to said cycle B or B′, in particular to said heterocyclic groups with 4 to 10 carbon atoms comprising at least one nitrogen atom;
(ii) from —NR_{Y}R_{Z}, wherein R_{Y} and R_{Z} are independently chosen from H and C₁-C₆ alkyl groups, in particular from C₁-C₆ alkyl groups;
and wherein said nitrogen optionally forms a quaternary ammonium by being further substituted by a C₁-C₆ alkyl;
and
(iii)) when n is an integer is 1, 2 or 3, in particular 1 or 2, or X is chosen from —O—, —S—, —NH—, and —NR_{c}—, or at least one of the carbon atoms of 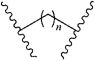
is replaced by an atom chosen from nitrogen, oxygen and sulfur, said nitrogen being optionally substituted by a methyl or ethyl group, or said nitrogen of group B forms a quaternary ammonium, from heterocyclic groups with 4 to 10 carbon atoms comprising at least one nitrogen atom,
the cycle B is fused with at least an arene, in particular a benzene, to form a polycycle B′;
the cycle B or B′ is optionally substituted by at least one group Z chosen from C₁-C₆ alkyl, in particular methyl or ethyl; O—C₁-C₆ alkyl, in particular —OMe; aryl, in particular phenyl; heteroaryl, in particular pyridyl, pyrimidinyl; benzyl; benzhydryl; and —NRₐR_{b} groups, wherein Rₐ and R_{b} are independently chosen from H and C₁-C₆ alkyls, Rₐ and R_{b} being in particular H;
wherein said nitrogen optionally forms a quaternary ammonium by being further substituted by a C₁-C₆ alkyl or by a C₁-C₆ alkane diyl also bound to said cycle B or B′, in particular to said heterocyclic groups with 4 to 10 carbon atoms comprising at least one nitrogen atom;
(iv) when n is an integer is 1 or 2, in particular 1, or X is chosen from —O—, —S—, —NH—, and —NR_{c}—, or at least one of the carbon atoms of 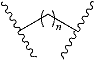
is replaced by an atom chosen from nitrogen, oxygen and sulfur, said nitrogen being optionally substituted by a methyl or ethyl group, or said nitrogen of group B forms a quaternary ammonium, from heterocyclic groups with 4 to 10 carbon atoms comprising at least one nitrogen atom, said cycle B being fused with at least a heteroarene, in particular an indole;
wherein said nitrogen optionally forms a quaternary ammonium by being further substituted by a C₁-C₆ alkyl or by a C₁-C₆ alkane diyl also bound to said cycle B or B′, in particular to said heterocyclic groups with 4 to 10 carbon atoms comprising at least one nitrogen atom;
or a stereoisomeric form, a mixture of stereoisomeric forms or a pharmaceutically acceptable salt or solvate form thereof.

2. The method according to claim 1, wherein said compound is of following formula (Ic): 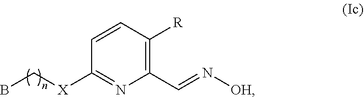
wherein B, n, X and R are as defined in claim 1, R being in particular —OH.

3. The method according to claim 1, wherein:
n is an integer from 1 to 4, in particular from 1 to 3, more particularly 1 or 2; or
at least one of the carbon atoms of 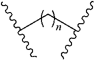
is optionally replaced by an atom chosen from nitrogen, oxygen and sulfur; or
X is chosen from —O—, —S—, —NH— and —NR_{c}—, with R_{c} being methyl or ethyl; or
or said nitrogen of group B forms a quaternary ammonium.

4. The method according to claim 1, wherein cycle B is chosen from pyrrolidine, piperidine, azepane, azocane, azonane, piperazine, thiomorpholine and morpholine, said cycle B being optionally substituted and/or fused as defined in claim 1, in particular substituted as defined in claim 1.

5. The method according to claim 1, wherein cycle B is chosen from indole, pyrrazole, imidazole, oxazole, thiazole, oxadiazole and thiadiazole, said cycle B being optionally substituted and/or fused as defined in claim 1.

6. The method according to claim 1, wherein cycle B is fused with a benzene, to form in particular a tetrahydroisoquinoline, more particularly an unsubstituted tetrahydroisoquinoline.

7. The method according to claim 1, wherein B represents —NR₁R₂ being a residue chosen from: 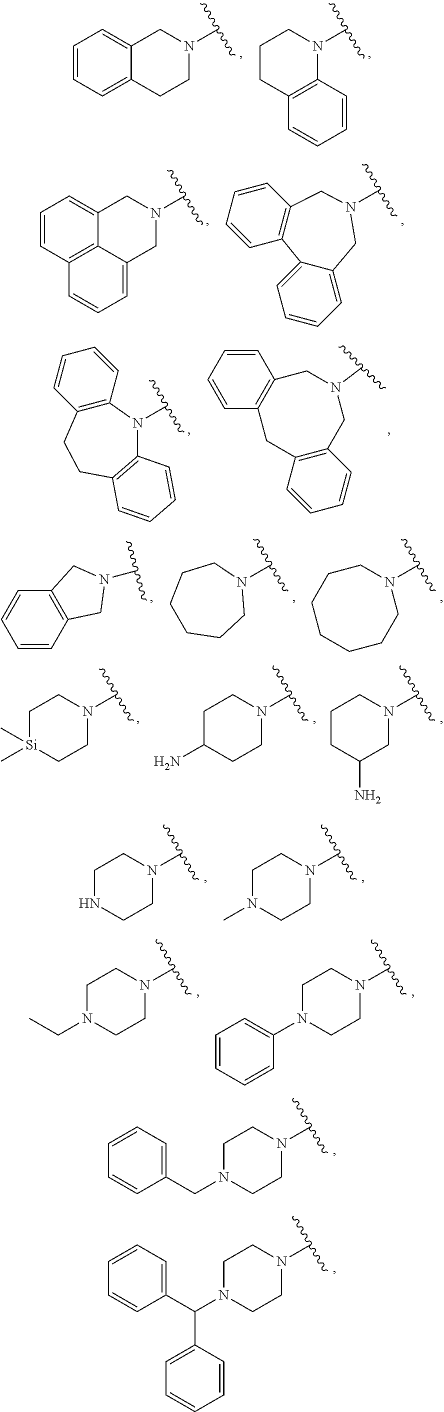 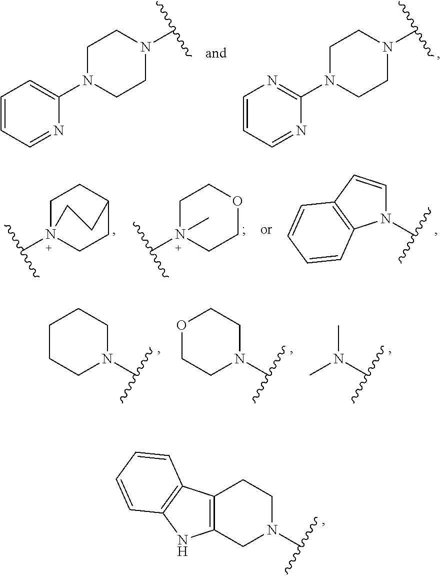
in particular when n is an integer is 1, 2 or 3, in particular 1 or 2, or X is chosen from —O—, —S—, —NH—, and —NR_{c}—, or at least one of the carbon atoms of 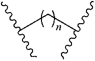
is replaced by an atom chosen from nitrogen, oxygen and sulfur, said nitrogen being optionally substituted by a methyl or ethyl group, from heterocyclic groups with 4 to 10 carbon atoms comprising at least one nitrogen atom.

8. The method according to claim 1, wherein said compound is chosen from:

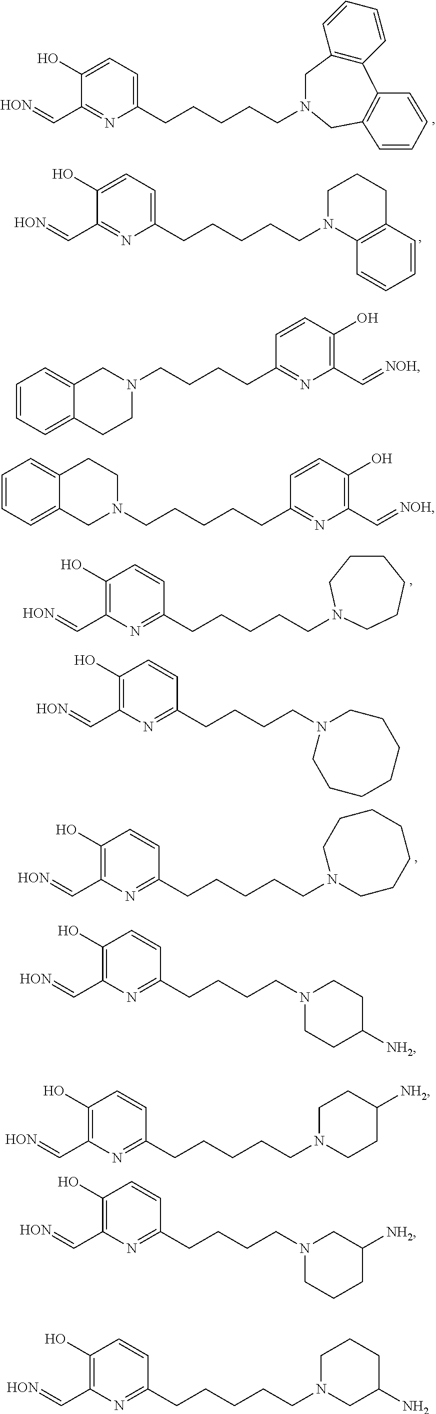

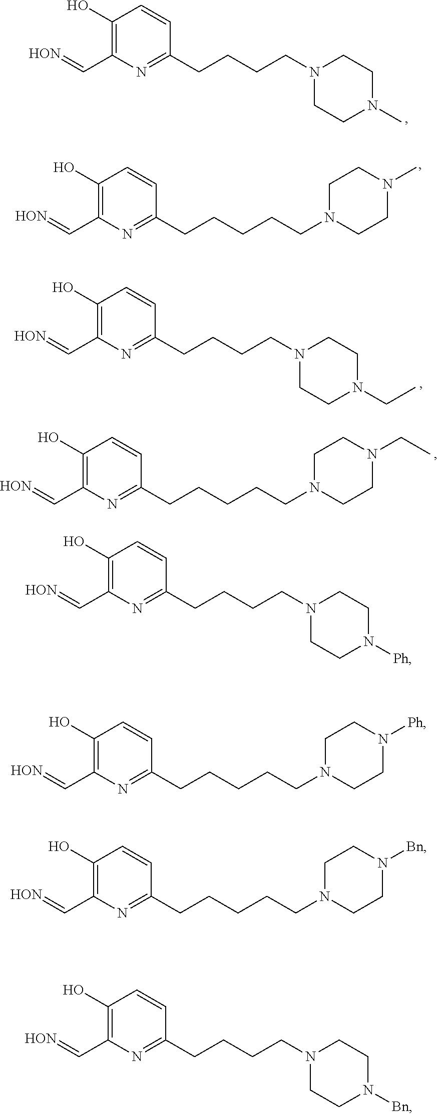

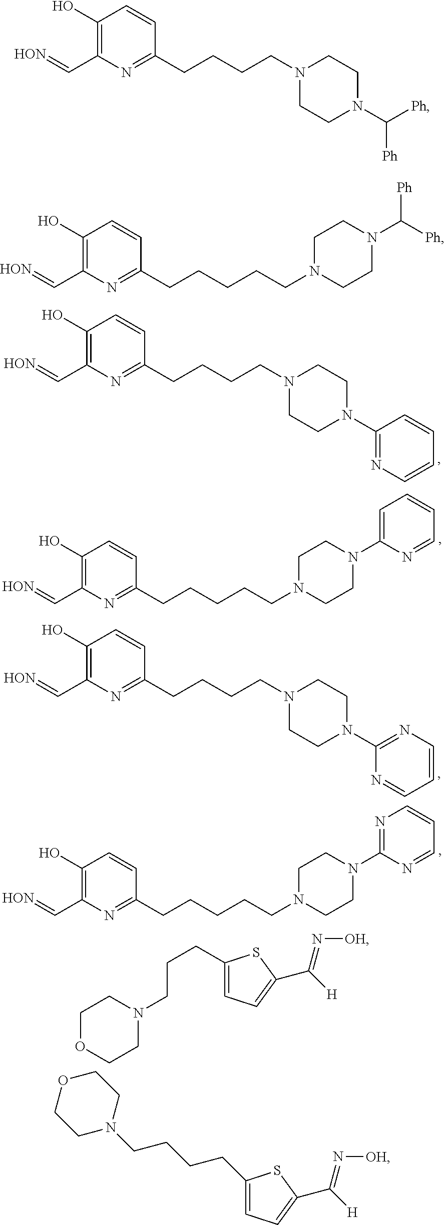

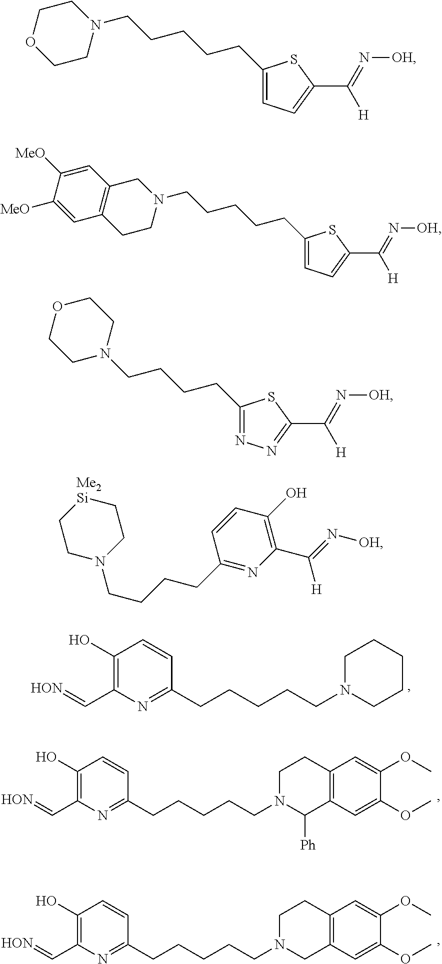

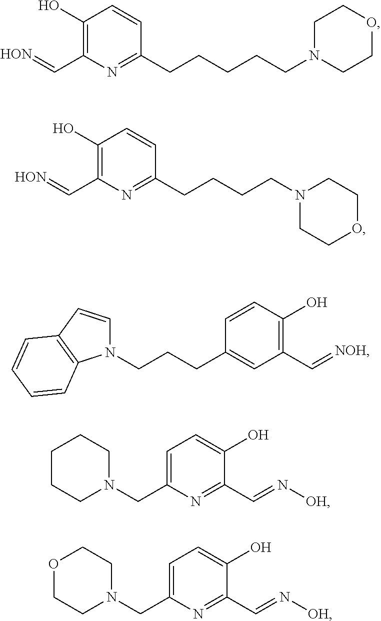

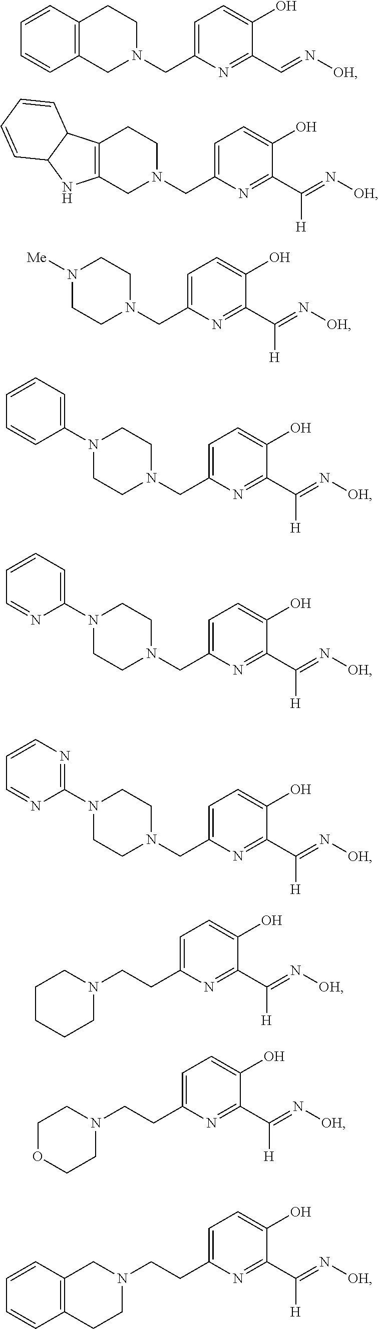

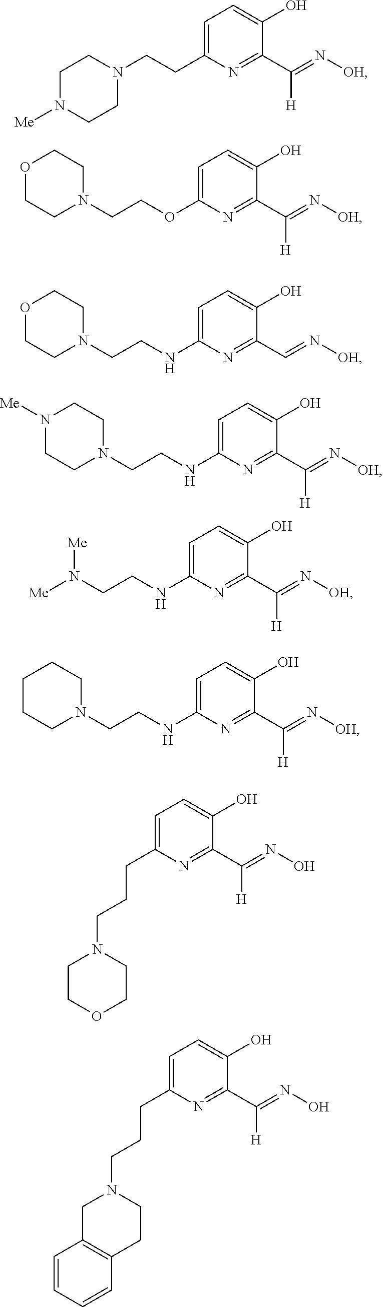

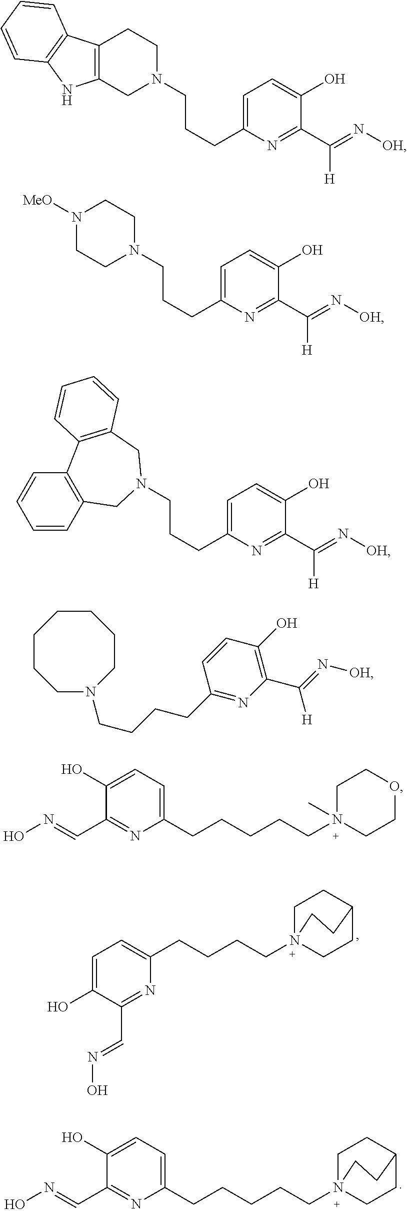

9. The method according to claim 1, wherein said organophosphorus nerve agent is selected from warfare agents such as Tammelin esters including O-ethyl-S-[2-(diisopropylamino)ethyl]methylphosphonothioate (VX), O-Ethyl-S-2-(diisopropylamino)ethylethylphosphonothiolate (VS), amiton (VG), 2-[ethoxy(ethyl)phosphoryl]sulfanyl-N,N-diethylethanamine (VE), edemo (VM), N,N-diethyl-2-(methyl-(2-methylpropoxy)phosphoryl)sulfanylethanamine (VR) and O-cyclopentyl S-(2-diethylaminoethyl) methylphosphonothiolate (EA-3148); tabun; sarin; cyclosarin; soman; Novichok agents; and pesticides such as paraoxon, parathion, tetraethyl pyrophosphate (TEPP), dichlorvos, phosmet, malathion, fenitrothion, methyl parathion, tetrachlorvinphos, chlorpyrifos, azamethiphos, diazinon, azinphos-methyl, terbufos.

10. A compound of following formula (II) or a pharmaceutical composition comprising said compound of formula (II) in admixture with at least one pharmaceutically acceptable excipient: 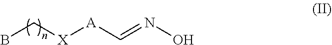
wherein:
n is an integer from 1 to 6;
at least one of the carbon atoms of 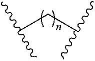
being optionally replaced by an atom chosen from nitrogen, oxygen and sulfur, said nitrogen being optionally substituted by a methyl or ethyl group;
X is a single bond or chosen from —O—, —S—, —NH—, and —NR_{c}—, with R_{c} being methyl or ethyl;
A is chosen from the group comprising arene diyles and 5 to 6 membered heteroarene diyles, said heteroarene being chosen from the group comprising pyridine, thiophene, thiadiazole, oxathiazole, in particular pyridine;
A is optionally substituted by a group R chosen from —OH, C₁-C₆ alkyl, —O—C₁-C₆ alkyl, -halogen, notably —Cl, —Br, —F, in particular —OH;
B is chosen from:
—NR₁R₂ groups wherein R₁ and R₂ form together with the nitrogen to which they are attached a cycle B chosen from the heterocyclic groups with 4 to 10 carbon atoms, cycle B not being piperidine or morpholine; and
heteroaryl groups chosen from pyrrazole, imidazole, oxazole, thiazole, oxadiazole and thiadiazole;
the cycle B is optionally fused with at least an arene, in particular a benzene, to form a polycycle B′;
the cycle B or B′ is optionally substituted by at least one group Z chosen from C₁-C₆ alkyl, in particular methyl or ethyl; O—C₁-C₆ alkyl, in particular —OMe; aryl, in particular phenyl; heteroaryl, in particular pyridyl, pyrimidinyl; benzyl; benzhydryl; and —NRₐR_{b} groups, wherein Rₐ and R_{b} are independently chosen from H and C₁-C₆ alkyls, Rₐ and R_{b} being in particular H; with the proviso that, in particular when A is a pyridine:
cycle B is fused and/or substituted when said cycle B is a piperidine;
cycle B′ is not substituted by one or more O—C₁-C₆ alkyl groups when B′ represents a tetrahydroisoquinoline;
or when n is an integer is 1, 2 or 3, in particular 1, or X is chosen from —O—, —S—, —NH—, and —NR_{c}—, or at least one of the carbon atoms of 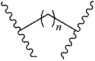
is replaced by an atom chosen from nitrogen, oxygen and sulfur, said nitrogen being optionally substituted by a methyl or ethyl group, or said nitrogen of group B forms a quaternary ammonium, B is chosen:
(i) from heterocyclic groups with 4 to 10 carbon atoms comprising at least one nitrogen atom, and heteroaryl groups chosen from indole, pyrrazole, imidazole, oxazole, thiazole, oxadiazole and thiadiazole,
the cycle B is optionally fused with at least an arene, in particular a benzene, to form a polycycle B′;
the cycle B or B′ is optionally substituted by at least one group Z chosen from C₁-C₆ alkyl, in particular methyl or ethyl; O—C₁-C₆ alkyl, in particular —OMe; aryl, in particular phenyl; heteroaryl, in particular pyridyl, pyrimidinyl; benzyl; benzhydryl; and —NRₐR_{b} groups, wherein Rₐ and R_{b} are independently chosen from H and C₁-C₆ alkyls, Rₐ and R_{b} being in particular H;
wherein said nitrogen optionally forms a quaternary ammonium by being further substituted by a C₁-C₆ alkyl or by a C₁-C₆ alkane diyl also bound to said cycle B or B′, in particular to said heterocyclic groups with 4 to 10 carbon atoms comprising at least one nitrogen atom;
(ii) from —NR_{Y}R_{Z}, wherein R_{Y} and R_{Z} are independently chosen from H and C₁-C₆ alkyl groups, in particular from C₁-C₆ alkyl groups;
and wherein said nitrogen optionally forms a quaternary ammonium by being further substituted by a C₁-C₆ alkyl; and
(iii) when n is an integer is 1 or 2, in particular 1, or X is chosen from —O—, —S—, —NH—, and —NR_{c}—, or at least one of the carbon atoms of 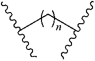
is replaced by an atom chosen from nitrogen, oxygen and sulfur, said nitrogen being optionally substituted by a methyl or ethyl group, or said nitrogen of group B forms a quaternary ammonium, from heterocyclic groups with 4 to 10 carbon atoms comprising at least one nitrogen atom, said cycle B being fused with at least a heteroarene, in particular an indole;
wherein said nitrogen optionally forms a quaternary ammonium by being further substituted by a C₁-C₆ alkyl or by a C₁-C₆ alkane diyl also bound to said cycle B or B′, in particular to said heterocyclic groups with 4 to 10 carbon atoms comprising at least one nitrogen atom;
or a stereoisomeric form, a mixture of stereoisomeric forms or a pharmaceutically acceptable salt or solvate form thereof,
for use in the treatment or prevention of a nervous and/or respiratory failure due to intoxication with at least one organophosphorus nerve agent.

11-12. (canceled)

13. Kit comprising a butyrylcholinesterase, and a compound of formula (I) as defined in claim 1, optionally aimed for simultaneous, sequential or separate use in the treatment or prevention of a nervous and/or respiratory failure due to intoxication with at least one organophosphorus nerve agent.

14-16. (canceled)

17. The method according to claim 1, comprising administering an effective dose of said compound of formula (I) to a subject in need thereof for treatment or prevention of an intoxication with said organophosphorus nerve agent.
